(19) United States
(12) Patent Application Publication
(10) Pub. No.: US 2009/0023741 A1
(43) Pub. Date: Jan. 22, 2009

(54) ARYL/HETARYLAMIDES AS MODULATORS OF THE EP2 RECEPTOR

(76) Inventors: Bernd Buchmann, Hohen Neuendorf (DE); Nico Braeuer, Jena (DE); Olaf Peters, Tabarz (DE); Knut Eis, Berlin (DE); Marcus Koppitz, Berlin (DE); Bernhard Lindenthal, Berlin (DE); Gernot Langer, Falkensee (DE); Tim Wintermantel, Berlin (DE); Antonius Ter Laak, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/137,931

(22) Filed: Jun. 12, 2008

Related U.S. Application Data
(60) Provisional application No. 60/943,656, filed on Jun. 13, 2007.

(30) Foreign Application Priority Data
Jun. 13, 2007 (EP) 07090121.0

(51) Int. Cl. A61K 31/404; C07D 209/16; C07D 241/40; A61P 15/00; A61K 31/498; C07D 495/04

(52) U.S. Cl. 514/249; 548/504; 544/353; 544/350; 514/415

(57) ABSTRACT

The present invention relates to aryl/hetarylamide derivatives of the general formula I, process for their preparation, and the use thereof for the manufacture of pharmaceutical compositions for the treatment of disorders and indications connected with the EP₂ receptor.

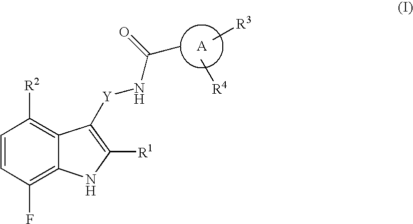

This application claims the benefit of the filing date of U.S. Provisional Application Ser. No. 60/943,656 filed Jun. 13, 2007.

The present invention relates to aryl/hetarylamide derivatives as EP₂ receptor modulators, process for their preparation, and their use as medicaments.

It has long been known that prostaglandins are key molecules in the processes of female reproductive biology such as, for example, control of ovulation, of fertilization, of nidation, of decidualization (e.g. placenta formation) and of menstruation. Prostaglandins likewise play an important part in the pathological changes in the reproductive tract, including menorrhagia, dysmenorrhea, endometriosis and cancer. The mechanism by which prostaglandins bring about these changes has not yet been completely elucidated. Recent results indicate that prostaglandins, their receptors and signal transduction pathways thereof are involved in processes such as angiogenesis, apoptosis, proliferation, and in inflammatory/antiinflammatory and immunological processes.

The effects of prostaglandins are mediated by their G protein-coupled receptors which are located on the cell surface. Prostaglandin E₂ (PGE₂) is of particular interest, having a wide variety of cellular effects through binding to functionally different receptor subtypes, namely the EP₁, EP₂, EP₃ and EP₄ receptors. The receptor subtypes to which prostaglandin E₂ binds appear to be of particular interest for the receptor-mediated effects which are involved in the control of fertility. It has thus been possible to show that the reproductive functions in EP₂ knockout mice (EP₂^{-/-}), i.e. in mice no longer having a functional PGE₂ receptor of the EP₂ subtype, are impaired, and that these animals have a smaller “litter size” (Matsumoto et al., 2001, Biology of Reproduction 64, 1557-1565). It was likewise possible to show that these EP₂ knockout mice (Hizaki et al. Proc Natl Acad Sci U.S.A. 1999 Aug. 31; 96(18):10501-10506) show distinctly reduced cumulus expansion and severe subfertility, which is to be regarded as causally connected with diminished reproductive processes such as ovulation and fertilization.

The EP₂ receptor accordingly represents an important target for developing medicaments for controlling female fertility. The existence of the 4 subclasses of the PGE₂ receptor opens up the possibility of targeted development of selectively active compounds. However, to date, scarcely any selective EP₂ receptor ligands which bind to the EP₂ subtypes of the PGE₂ receptor are known, since most known compounds also bind to the other PGE₂ receptor subtypes such as, for example, to the EP₄ receptor.

EP₂ receptor antagonists are described, for example in the application US2005059742 (Jabbour, Medical Research Concil). A method in which an EP₂ and/or an EP₄ antagonist can be employed for the treatment of menorrhagia and dysmenorrhea is claimed. AH6809 is disclosed as antagonist of the EP₂ or EP₄ receptor, but no other specific antagonists and no new compounds are disclosed.

In an earlier application of the same group (EP1467738), EP₂ or EP₄ antagonists are claimed for the treatment of pathological conditions such as, for example, allergic disorders, Alzheimer's disease, pain, abortion, painful menstruation, menorrhagia and dysmenorrhea, endometriosis, bone disorders, ischemia etc. The described compounds are, however, distinguished by a particularly high affinity for the EP₃ receptor. A further application (WO04/032964) describes novel compounds which are likewise distinguished by a particularly high affinity for the EP₃ receptor, but also have EP₂-antagonistic effects and which are used for the treatment and prophylaxis of allergic disorders.

Ono Pharmaceutical claims in the application WO03/016254 the preparation of benzene acid or saturated carboxylic acid derivatives which are substituted by aryl or heterocycles, inter alia as PGE₂ receptor antagonists. The disclosed compounds are claimed for the treatment of a large number of disorders, including allergic disorders, Alzheimer's disease, pain, abortion, painful menstruation, menorrhagia and dysmenorrhea, endometriosis, bone disorders, ischemia etc. The described compounds are, however, distinguished by a particularly high affinity for the EP₃ receptor. A further application (WO04/032964) describes novel compounds which are likewise distinguished by a particularly high affinity for the EP₃ receptor, but also have EP₂-antagonistic effects and which are used for the treatment and prophylaxis of allergic disorders.

The application WO04/39807 of Merck Frosst, Canada, discloses the preparation of pyridopyrrolizines and pyridoindolizines. However, these compounds are distinguished by good binding to the PGD₂ receptor, and this receptor represents a different subtype of the prostaglandin receptor.

Naphthalene derivatives as EP₄ receptor ligands are disclosed in application US2004102508 of SmithKline Beecham Corporation. The claimed compounds are used for the treatment or prophylaxis of pain, allergic reactions and neurodegenerative disorders.

EP₄ antagonists (γ-lactams) are claimed in the application WO03/103604 (Applied Research Systems). The compounds bind approximately 60-fold better to the EP₄ than to the EP₂ receptor and are claimed inter alia for the treatment of premature labor, dysmenorrhea, asthma, infertility or fertility impairments. The same company claims in the applications WO03/053923 (substituted pyrrolidines) or WO03/035064 (substituted pyrazolidinones) compounds for the treatment of disorders associated with prostaglandins, such as, for example, infertility, hypertension and osteoporosis. The compounds bind to the EP₄- and to the EP₂ receptor subtypes. The application WO03/037433 claims ω-cycloalkyl, 17 heteroaryl prostaglandin derivatives as EP₂ receptor antagonists, in particular for the treatment of elevated intraocular pressure.

The application WO03/064391 (Pfizer Products) describes metabolites of [3-[[N-(4-tert-butylbenzyl)(pyridin-3-ylsulfonyl)amino]methyl]acetic acid which inhibit the binding of [³H] prostaglandin E₂ to the EP₂ receptor. The use of these metabolites for the treatment of osteoporosis is disclosed.

Tani et al. claim in the application US2005124577 8-azaprostaglandin derivatives for the treatment of immunological disorders, allergic disorders, premature labor, abortion, etc. The compounds bind to the EP₂ and to the EP₄ receptor.

European patent application EP 1306087 describes EP₂ receptor agonists which are used for the treatment of erectile dysfunction (Ono Pharmaceuticals). The same class of structures is described in European patent EP 860430 (Ono Pharmaceuticals), and their use for the manufacture of a medicament for the treatment of immunological disorders, asthma and abortion is claimed. WO04/009117 describes EP₂ and EP₄ receptor agonists for the treatment of disorders caused by uterine contraction, for example painful menstruation (Ono Pharmaceuticals).

The applications WO03/74483 and WO03/09872 describe agonists which bind equally to the EP₂ and to the EP₄ receptor (Ono Pharmaceuticals).

Agonists of the EP₂ and of the EP₄ receptors are frequently described in connection with the treatment of osteoporosis (WO99/19300 (Pfizer), US2003/0166631 (Dumont Francis), WO03/77910 (Pfizer), WO03/45371 (Pfizer), WO03/74483 and WO03/09872 (Ono Pharmaceuticals)) and for glaucoma treatment (WO04/37813, WO04/37786, WO04/19938, WO03/103772, WO03/103664, WO03/40123, WO03/47513, WO03/47417 (Merck Frosst Canada)) and U.S. Pat. No. 6,410,591 and U.S. Pat. No. 6,747,037 (Allergan).

The patent application WO04/12656 (Applied Research Systems) claims EP₂ receptor agonists in connection with inflammation.

The patent application WO03/77919 (Merck & Co. Inc.) claims EP₄ receptor agonists for the treatment of fertility.

However, to date, no selective EP₂ receptor agonists and antagonists which control the processes which are ultimately responsible for ovulation, fertilization, nidation and decidualization and thus contribute to promoting or inhibiting fertility are known.

It is therefore an object of the present invention to provide stable EP₂ receptor antagonists.

This object is achieved by providing the compounds of the general formula I

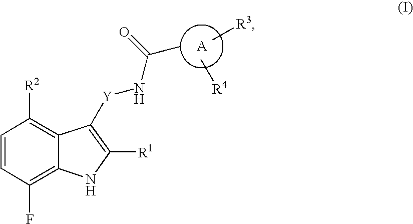

in which
A is an aryl or heteroaryl radical which may optionally be substituted one or more times by R³ and/or R⁴,
R¹ is a hydrogen, a C₁-C₆-alkyl radical which may optionally be substituted,
R² is a hydrogen, halogen, cyano, an —S(O)_{q}—CH₃, where q is 0-2, a C₁-C₄-alkoxy radical or C₁-C₆-alkyl, where this radical can be substituted in any way,
R³ is a hydrogen, halogen, amino, an —S(O)ₚ—C₁-C₆-alkyl group, where p is 0-2,
   a C₁-C₆-acyl, NH—CO—NH₂, —O—CO—NH(C₁-C₆-alkyl), —O—CO—N(C₁-C₆-alkyl)₂ or NH—CO—C₁-C₆-alkyl radical,
   a C₁-C₆-alkyl which may optionally be substituted one or more times, identically or differently, by C₁-C₆-acyl, C₁-C₆-alkoxy, hydroxy, cyano, CO₂—(C₁-C₆-alkyl), N—(C₁-C₆-alkyl)₂, C₅-C₁₂-heteroaryl, COOH, CO—NH₂, CO—NH(C₁-C₆-alkyl) or by CO—N(C₁-C₆-alkyl)₂,
   a C₁-C₆-alkoxy which may optionally be substituted one or more times, identically or differently, by hydroxy, cyano, CO₂—(C₁-C₆-alkyl), N—(C₁-C₆-alkyl)₂, NH—C₃-C₆-cycloalkyl, COOH, CO—NH₂, CO—NH(C₁-C₆-alkyl) or by CO—N(C₁-C₆-alkyl)₂,
   an O—C₆-C₁₂-aryl which may optionally be substituted by hydroxy, cyano, COOH or CO—NH₂,
   a CH₂O—C₆-C₁₂-aryl which may optionally be substituted by hydroxy, cyano, COOH or CO—NH₂,
   an O—C₅-C₁₆-heteroaryl which may optionally be substituted by hydroxy, cyano, COOH or CO—NH₂,
   a hydroxy, cyano, O—CO—(C₁-C₆-alkyl), CO—NH(C₅-C₁₂-heteroaryl), NH—(C₁-C₆-alkyl), N—(C₁-C₆-alkyl)₂,
   a C₆-C₁₂-aryl which may optionally be substituted one or more times, identically or differently, by halogen, by C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl), CH₂—NH—CO(C₅-C₁₂-heteroaryl), styryl, or an —S(O)ᵣ—CH₃, where r is 0-2, or two adjacent positions may be substituted by —O—CH₂—O— or —O—C(CH₃)₂—O—,
   a monocyclic C₅-C₇-heteroaryl which may optionally be substituted one or more times, identically or differently, by C₁-C₆-alkyl, if R² is cyano or if R¹ and/or R² is identically or differently a C₁-C₆-alkyl radical, where at least one of the radicals is substituted at least once, or
   if R⁴ is —S(O)ₚ—C₁-C₆-alkyl, where p is 0-2, a C₁-C₆-acyl-, —O—CO—NH(C₁-C₆-alkyl), —O—CO—N(C₁-C₆-alkyl)₂, C₆-C₁₂-aryloxy, C₅-C₁₆-heteroaryloxy, hydroxy, cyano or N—(C₁-C₆-alkyl)₂,
   a monocyclic C₅-C₇-heteroaryl which may be substituted at least one or more times, identically or differently, by halogen, CF₃, C₁-C₆-acyl, C₁-C₆-alkoxy, hydroxy, CH₂—OH, cyano, CO₂—(C₁-C₆-alkyl), N—(C₁-C₆-alkyl)₂, COOH, CO—NH₂, CO—NH(C₁-C₆-alkyl) or CO—N(C₁-C₆-alkyl)₂,
   a bi- or tricyclic C₈-C₁₂-heteroaryl which may optionally be substituted one or more times, identically or differently, by halogen, by C₁-C₆-alkyl, C₁-C₆-acyl, C₁-C₆-alkoxy, hydroxy, cyano, CO₂—(C₁-C₆-alkyl), N—(C₁-C₆-alkyl)₂, COOH, CO—NH₂, CO—NH(C₁-C₆-alkyl) or CO—N(C₁-C₆-alkyl)₂, or
   a C₃-C₆-cycloalkyl which may optionally be substituted one or more times, identically or differently, by halogen, by C₁-C₆-alkyl, hydroxy, cyano, CO₂—(C₁-C₆-alkyl), C₁-C₆-acyl, N—(C₁-C₆-alkyl)₂, COOH, CO—NH₂, CO—NH(C₁-C₆-alkyl), CO—N(C₁-C₆-alkyl)₂ or C₁-C₆-alkoxy,
R⁴ is a hydrogen, halogen, amino, —S(O)ₚ—C₁-C₆-alkyl, where p is 0-2,
   a C₁-C₆-acyl, NH—CO—NH₂, NH—CO—C₁-C₆-alkyl, —O—CO—NH(C₁-C₆-alkyl), —O—CO—N(C₁-C₆-alkyl)₂ or C₁-C₆-alkyl group which may optionally be substituted one or more times, identically or differently, by C₁-C₆-acyl, C₁-C₆-alkoxy, hydroxy, cyano, CO₂—(C₁-C₆-alkyl), N—(C₁-C₆-alkyl)₂, C₅-C₁₂-heteroaryl, COOH, CO—NH₂, CO—NH(C₁-C₆-alkyl) or by CO—N(C₁-C₆-alkyl)₂,
   a C₁-C₆-alkoxy which may optionally be substituted one or more times, identically or differently, by hydroxy, cyano, CO₂—(C₁-C₆-alkyl), N—(C₁-C₆-alkyl)₂, NH—C₃-C₆-cycloalkyl, COOH, CO—NH₂, CO—NH(C₁-C₆-alkyl) or by CO—N(C₁-C₆-alkyl)₂,
   an O—C₆-C₁₂-aryl which may optionally be substituted by hydroxy, cyano, COOH or CO—NH₂,
   a CH₂O—C₆-C₁₂-aryl which may optionally be substituted by hydroxy, cyano, COOH or CO—NH₂,
   an O—C₅-C₁₆-heteroaryl which may optionally be substituted by hydroxy, cyano, COOH or CO—NH₂,
   a hydroxy, cyano, O—CO—(C₁-C₆-alkyl), CO—NH(C₅-C₁₂-heteroaryl), NH—(C₁-C₆-alkyl), N—(C₁-C₆-alkyl)₂,
   a C₆-C₁₂-aryl which may optionally be substituted one or more times, identically or differently, by halogen, by C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl), CH₂—NH—CO(C₅-C₁₂-heteroaryl), styryl, or an —S(O)ᵣ—CH₃, where r is 0-2, or two adjacent positions may be substituted by —O—CH₂—O— or —O—C(CH₃)₂—O—,
   a monocyclic C₅-C₇-heteroaryl which may optionally be substituted one or more times, identically or differently, by C₁-C₆-alkyl, if R² is cyano or if R¹ and/or R² is identically or differently a C₁-C₆-alkyl radical, where at least one of the radicals is substituted at least once,
   a monocyclic C₅-C₇-heteroaryl which may be substituted at least one or more times, identically or differently, by halogen, CF₃, C₁-C₆-acyl, C₁-C₆-alkoxy, hydroxy, CH₂—OH, cyano, CO₂—(C₁-C₆-alkyl), N—(C₁-C₆-alkyl)₂, COOH, CO—NH₂, CO—NH(C₁-C₆-alkyl) or CO—N(C₁-C₆-alkyl)₂,
   a bi- or tricyclic C₈-C₁₂-heteroaryl which may optionally be substituted one or more times, identically or differently, by halogen, by C₁-C₆-alkyl, C₁-C₆-acyl, C₁-C₆-alkoxy, hydroxy, cyano, CO₂—(C₁-C₆-alkyl), N—(C₁-C₆-alkyl)₂, COOH, CO—NH₂, CO—NH(C₁-C₆-alkyl) or CO—N(C₁-C₆-alkyl)₂, or
   a C₃-C₆-cycloalkyl which may optionally be substituted one or more times, identically or differently, by halogen, by C₁-C₆-alkyl, hydroxy, cyano, CO₂—(C₁-C₆-alkyl), C₁-C₆-acyl, N—(C₁-C₆-alkyl)₂, COOH, CO—NH₂, CO—NH(C₁-C₆-alkyl), CO—N(C₁-C₆-alkyl)₂ or C₁-C₆-alkoxy,
R³ and R⁴ are either in ortho, meta position or meta, para position relative to one another and together have the meaning —O—CO—S—, —S—CO—O—, CH₂—CO—O—, O—CO—CH₂—, —CH₂—CO—NH—, —NH—CO—CH₂—, —O—CO—NH—, NH—CO—O—, —CO—CH₂—(CH₂)ₘ—, —CH₂—(CH₂)ₘ—CO—, —O—(CH₂)ₘ—O—, —O—C—(CH₃)₂—O—, —CH₂—(CH₂)ₘ—CH₂—, where m is 1-3,
Y is a —(CH₂)ₙ— group, where n is 1-3,
   and the isomers, diastereomers, enantiomers and salts thereof, and cyclodextrin clathrates, which overcome the known disadvantages and have improved properties, i.e. good activity, good solubility and stability, where the following compounds are excluded:
N-[2-(7-fluoro-2,4-dimethyl-1H-indol-3-yl)ethyl]-3,4-dimethoxybenzamide
N-[2-(7-fluoro-2,4-dimethyl-1H-indol-3-yl)ethyl]-2-methylbenzamide
4-bromo-N-[2-(7-fluoro-2,4-dimethyl-1H-indol-3-yl)ethyl]benzamide.

The compounds of the invention have an antagonistic effect on the EP₂ receptor and thus serve to control female fertility.

C₁-C₄-Alkyl or C₁-C₆-alkyl means in each case a straight-chain or branched alkyl radical such as, for example, methyl, ethyl, propyl, isopropyl, butyl, isobutyl, sec-butyl, tert-butyl, pentyl, isopentyl and hexyl.

The alkyl radicals may optionally be substituted one or more times, identically or differently, by halogen.

C₁-C₄-Alkoxy or C₁-C₆-alkoxy means in each case a straight-chain or branched alkoxy radical such as, for example, methoxy-, ethoxy-, n-propoxy-, isopropoxy-, n-butoxy-, sec-butoxy-, isobutoxy-, tert-butyloxy-, pentoxy-, isopentoxy- and hexoxy-.

The alkoxy radicals may optionally be substituted one or more times, identically or differently, by halogen.

C₁-C₄-Acyl or C₁-C₆-acyl means in each case a straight-chain or branched radical such as, for example, formyl, acetyl, propionyl, butyryl, isobutyryl, valeryl and benzoyl.

The acyl radicals may optionally be substituted one or more times, identically or differently, by halogen.

C₃-C₆-Cycloalkyl means monocyclic alkyl rings such as cyclopropyl, cyclobutyl, cyclopentyl and cyclohexyl.

The cycloalkyl radicals may, instead of the carbon atoms, comprise one or more heteroatoms such as oxygen, sulfur and/or nitrogen. Preferred heterocycloalkyls are those having 3 to 6 ring atoms, such as, for example, aziridinyl, azetidinyl, pyrrolidinyl, piperidinyl, piperazinyl, morpholinyl. Ring systems in which optionally one or more possible double bonds may be contained in the ring are for example cycloalkenyls such as cyclopropenyl, cyclobutenyl, cyclopentenyl, cyclopentadienyl, cyclohexenyl, cycloheptenyl, with the connection possibly taking place either at the double bond or at the single bonds.

Halogen means in each case fluorine, chlorine, bromine or iodine.

The C₆-C₁₂-aryl radical includes in each case 6-12 carbon atoms and may for example be benzo-fused. Examples which may be mentioned are: phenyl, tropyl, cyclooctadienyl, indenyl, naphthyl, biphenyl, fluorenyl, anthracenyl etc.

The monocyclic C₅-C₇-heteroaryl radical, the tricyclic C₈-C₁₂-heteroaryl radical and the C₅-C₁₆-heteroaryl radical mean ring systems which comprise in each case 5-16 ring atoms and which may, instead of the carbon, comprise one or more, identical or different, heteroatoms such as oxygen, sulfur or nitrogen, and where the C₅-C₁₆-heteroaryl radical may be mono-, bi- or tricyclic and may additionally in each case be benzo-fused.

Examples which may be mentioned are:

thienyl, furanyl, pyrrolyl, oxazolyl, imidazolyl, pyrazolyl, isoxazolyl, isothiazolyl, oxadiazolyl, triazolyl, thiadiazolyl, etc. and benzo derivatives thereof, such as, for example, benzofuranyl, benzothienyl, benzooxazolyl, benzimdazolyl, indazolyl, indolyl, isoindolyl, etc; or pyridinyl, pyridazinyl, pyrimidinyl, pyrazinyl, triazinyl, etc. and benzo derivatives thereof such as, for example, quinolyl, isoquinolyl, etc; or azocinyl, indolizinyl, purinyl, etc. and benzo derivatives thereof; or quinolinyl, isoquinolinyl, cinnolinyl, phthalazinyl, quinazolinyl, quinoxalinyl, naphthyridinyl, pteridinyl, carbazolyl, acridinyl, phenazinyl, phenothiazinyl, phenoxazinyl, xanthenyl, oxepinyl, benzotriazolyl, etc.

The heteroaryl radical may in each case be benzo-fused. Examples of 5-membered heteroaromatic rings which may be mentioned are: thiophene, furan, oxazole, thiazole, imidazole, pyrazole and benzo derivatives thereof, and of 6-membered heteroaromatic rings pyridine, pyrimidine, triazine, quinoline, isoquinoline and benzo derivatives.

Heteroatoms mean oxygen, nitrogen or sulfur atoms.

If an acidic function is present, suitable salts are the physiologically tolerated salts of organic and inorganic bases, such as, for example, the readily soluble alkali metal and alkaline earth metal salts, and N-methylglucamine, dimethylglucamine, ethylglucamine, lysine, 1,6-hexanediamine, ethanolamine, glucosamine, sarcosine, serinol, tris-hydroxymethylaminomethane, aminopropanediol, Sovak base, 1-amino-2,3,4-butanetriol.

If a basic function is present, the physiologically tolerated salts of organic and inorganic acids are suitable, such as hydrochloric acid, sulfuric acid, phosphoric acid, citric acid, tartaric acid inter alia.

Preference is given to those compounds of the general formula (I)

where
A is an aryl or heteroaryl radical which may optionally be substituted one or more times by R⁴ and/or R³,
R¹ is a hydrogen or C₁-C₆-alkyl radical which may be substituted one or more times by halogen,
R² is a hydrogen, halogen, cyano, an —S(O)_{q}—CH₃, where q is 0-2, a C₁-C₄-alkoxy radical or C₁-C₆-alkyl radical which may be substituted one or more times by halogen,
R³ is a hydrogen, halogen, amino, —S(O)ₚ—C₁-C₆-alkyl, where p is 0-2,
   a C₁-C₆-acyl, NH—CO—NH₂, NH—CO—C₁-C₆-alkyl, —O—CO—NH(C₁-C₆-alkyl), —O—CO—N(C₁-C₆-alkyl)₂, or C₁-C₆-alkyl group which may optionally be substituted one or more times, identically or differently, by C₁-C₆-acyl, C₁-C₆-alkoxy, hydroxy, cyano, CO₂—(C₁-C₆-alkyl), N—(C₁-C₆-alkyl)₂, C₅-C₁₂-heteroaryl, COOH, CO—NH₂, CO—NH(C₁-C₆-alkyl) or by CO—N(C₁-C₆-alkyl)₂,
   a C₁-C₆-alkoxy which may optionally be substituted one or more times, identically or differently, by hydroxy, cyano, CO₂—(C₁-C₆-alkyl), N—(C₁-C₆-alkyl)₂, NH—C₃-C₆-cycloalkyl, COOH, CO—NH₂, CO—NH(C₁-C₆-alkyl) or by CO—N(C₁-C₆-alkyl)₂,
   an O—C₆-C₁₂-aryl which may optionally be substituted by hydroxy, cyano, COOH or CO—NH₂,
   a CH₂O—C₆-C₁₂-aryl which may optionally be substituted by hydroxy, cyano, COOH or CO—NH₂,
   an O—C₅-C₁₆-heteroaryl which may optionally be substituted by hydroxy, cyano, COOH or CO—NH₂,
   a hydroxy, cyano, O—CO—(C₁-C₆-alkyl), CO—NH(C₅-C₁₂-heteroaryl), NH—(C₁-C₆-alkyl), N—(C₁-C₆-alkyl)₂,
   a C₆-C₁₂-aryl which may optionally be substituted one or more times, identically or differently, by halogen, by C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl), CH₂—NH—CO(C₅-C₁₂-heteroaryl), styryl, or an —S(O)ᵣ—CH₃, where r is 0-2, or two adjacent positions may be substituted by —O—CH₂—O— or —O—C(CH₃)₂—O—,
   a monocyclic C₅-C₇-heteroaryl which may optionally be substituted one or more times, identically or differently, by C₁-C₆-alkyl, if R² is cyano or if R¹ and/or R² is identically or differently a C₁-C₆-alkyl radical, where at least one of the radicals is substituted at least once, or
   if R⁴ is —S(O)ₚ—C₁-C₆-alkyl, where p is 0-2, a C₁-C₆-acyl-, —O—CO—NH(C₁-C₆-alkyl), —O—CO—N(C₁-C₆-alkyl)₂, C₆-C₁₂-aryloxy, C₅-C₁₆-heteroaryloxy, hydroxy, cyano or N—(C₁-C₆-alkyl)₂,
   a monocyclic C₅-C₇-heteroaryl which may be substituted at least one or more times, identically or differently, by halogen, by CF₃, C₁-C₆-acyl, C₁-C₆-alkoxy, hydroxy, CH₂—OH, cyano, CO₂—(C₁-C₆-alkyl), N—(C₁-C₆-alkyl)₂, COOH, CO—NH₂, CO—NH(C₁-C₆-alkyl) or CO—N(C₁-C₆-alkyl)₂,
   a bi- or tricyclic C₈-C₁₂-heteroaryl which may optionally be substituted one or more times, identically or differently, by halogen, by C₁-C₆-alkyl, C₁-C₆-acyl, C₁-C₆-alkoxy, hydroxy, cyano, CO₂—(C₁-C₆-alkyl), N—(C₁-C₆-alkyl)₂, COOH, CO—NH₂, CO—NH(C₁-C₆-alkyl) or CO—N(C₁-C₆-alkyl)₂, or
   a C₃-C₆-cycloalkyl which may optionally be substituted one or more times, identically or differently, by halogen, by C₁-C₆-alkyl, hydroxy, cyano, CO₂—(C₁-C₆-alkyl), C₁-C₆-acyl, N—(C₁-C₆-alkyl)₂, COOH, CO—NH₂, CO—NH(C₁-C₆-alkyl), CO—N(C₁-C₆-alkyl)₂ or C₁-C₆-alkoxy,
R⁴ is a hydrogen, halogen, amino, —S(O)ₚ—C₁-C₆-alkyl, where p is 0-2,
   a C₁-C₆-acyl, NH—CO—NH₂, NH—CO—C₁-C₆-alkyl, —O—CO—NH(C₁-C₆-alkyl), —O—CO—N(C₁-C₆-alkyl)₂, or C₁-C₆-alkyl group which may optionally be substituted one or more times, identically or differently, by C₁-C₆-acyl, C₁-C₆-alkoxy, hydroxy, cyano, CO₂—(C₁-C₆-alkyl), N—(C₁-C₆-alkyl)₂, C₅-C₁₂-heteroaryl, COOH, CO—NH₂, CO—NH(C₁-C₆-alkyl) or by CO—N(C₁-C₆-alkyl)₂,
   a C₁-C₆-alkoxy which may optionally be substituted one or more times, identically or differently, by hydroxy, cyano, CO₂—(C₁-C₆-alkyl), N—(C₁-C₆-alkyl)₂, NH—C₃-C₆-cycloalkyl, COOH, CO—NH₂, CO—NH(C₁-C₆-alkyl) or by CO—N(C₁-C₆-alkyl)₂,
   an O—C₆-C₁₂-aryl which may optionally be substituted by hydroxy, cyano, COOH or CO—NH₂,
   a CH₂O—C₆-C₁₂-aryl which may optionally be substituted by hydroxy, cyano, COOH or CO—NH₂,
   an O—C₅-C₁₆-heteroaryl which may optionally be substituted by hydroxy, cyano, COOH or CO—NH₂,
   a hydroxy, cyano, O—CO—(C₁-C₆-alkyl), CO—NH(C₅-C₁₂-heteroaryl), NH—(C₁-C₆-alkyl), N—(C₁-C₆-alkyl)₂,
   a C₆-C₁₂-aryl which may optionally be substituted one or more times, identically or differently, by halogen, by C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl), CH₂—NH—CO(C₅-C₁₂-heteroaryl), styryl, or an —S(O)ᵣ—CH₃, where r is 0-2, or two adjacent positions may be substituted by —O—CH₂—O— or —O—C(CH₃)₂—O—,
   a monocyclic C₅-C₇-heteroaryl which may optionally be substituted one or more times, identically or differently, by C₁-C₆-alkyl, if R² is cyano or if R¹ and/or R² is identically or differently a C₁-C₆-alkyl radical, where at least one of the radicals is substituted at least once,
   a monocyclic C₅-C₇-heteroaryl which may be substituted at least one or more times, identically or differently, by halogen, by CF₃, C₁-C₆-acyl, C₁-C₆-alkoxy, hydroxy, CH₂—OH, cyano, CO₂—(C₁-C₆-alkyl), N—(C₁-C₆-alkyl)₂, COOH, CO—NH₂, CO—NH(C₁-C₆-alkyl) or CO—N(C₁-C₆-alkyl)₂,
   a bi- or tricyclic C₈-C₁₂-heteroaryl which may optionally be substituted one or more times, identically or differently, by halogen, by C₁-C₆-alkyl, C₁-C₆-acyl, C₁-C₆-alkoxy, hydroxy, cyano, CO₂—(C₁-C₆-alkyl), N—(C₁-C₆-alkyl)₂, COOH, CO—NH₂, CO—NH(C₁-C₆-alkyl) or CO—N(C₁-C₆-alkyl)₂, or
   a C₃-C₆-cycloalkyl which may optionally be substituted one or more times, identically or differently, by halogen, by C₁-C₆-alkyl, hydroxy, cyano, CO₂—(C₁-C₆-alkyl), C₁-C₆-acyl, N—(C₁-C₆-alkyl)₂, COOH, CO—NH₂, CO—NH(C₁-C₆-alkyl), CO—N(C₁-C₆-alkyl)₂ or C₁-C₆-alkoxy,
R³ and R⁴ are either in ortho, meta position or meta, para position relative to one another and together have the meaning of —O—CO—S—, —S—CO—O—, CH₂—CO—O—, O—CO—CH₂—, —CH₂—CO—NH—, —NH—CO—CH₂—, —O—CO—NH—, —NH—CO—O—, —CO—CH₂—(CH₂)ₘ—, —CH₂—(CH₂)ₘ—CO—, O—O—(CH₂)ₘ—O—, —O—C—(CH₃)₂—O—, —CH₂—(CH₂)ₘ—CH₂—, where m is 1-3,
Y is a —(CH₂)ₙ— group, where n is 1-3,
   and the isomers, diastereomers, enantiomers and salts thereof, and cyclodextrin clathrates.

Preference is likewise given to those compounds of the general formula (I)

where
A is an aryl or heteroaryl radical which may optionally be substituted one or more times by R⁴ and/or R³,
R¹ is a hydrogen or a C₁-C₆-alkyl group which is substituted one or more times by halogen,
R² is a hydrogen, halogen, cyano, an —S(O)_{q}—CH₃, where q is 0-2, a C₁-C₄-alkoxy radical or a C₁-C₆-alkyl group which is substituted one or more times by halogen,
R³ is a hydrogen, halogen, amino, —S(O)ₚ—CH₃, where p is 0-2,
   an —S—CF₃, SO₂NH₂, C₁-C₆-acyl, NH—CO—NH₂, NH—CO—C₁-C₆-alkyl, —O—CO—NH(C₁-C₆-alkyl), —O—CO—N(C₁-C₆-alkyl)₂, or C₁-C₆-alkyl group which may optionally be substituted one or more times, identically or differently, by C₁-C₄-acyl, C₁-C₄-alkoxy, hydroxy, cyano, CO₂—(C₁-C₄-alkyl), N—(C₁-C₄-alkyl)₂, C₅-C₁₂-heteroaryl, COOH, CO—NH₂, CO—NH(C₁-C₄-alkyl) or by CO—N(C₁-C₄-alkyl)₂,
   a C₁-C₆-alkoxy which may optionally be substituted one or more times, identically or differently, by hydroxy, cyano, CO₂—(C₁-C₆-alkyl), N—(C₁-C₆-alkyl)₂, NH—C₃-C₆-cycloalkyl, COOH, CO—NH₂, CO—NH(C₁-C₆-alkyl) or by CO—N(C₁-C₆-alkyl)₂,
   an O—C₆-C₁₂-aryl which may optionally be substituted by hydroxy, cyano, COOH or CO—NH₂,
   a CH₂O—C₆-C₁₂-aryl which may optionally be substituted by hydroxy, cyano, COOH or CO—NH₂,
   an O—C₅-C₁₆-heteroaryl which may optionally be substituted by hydroxy, cyano, COOH or CO—NH₂,
   a hydroxy, cyano, O—CO—(C₁-C₆-alkyl), CO—NH(C₅-C₁₂-heteroaryl), NH—(C₁-C₆-alkyl), N—(C₁-C₆-alkyl)₂,
   a C₆-C₁₂-aryl which may optionally be substituted one or more times, identically or differently, by halogen, by C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl), CH₂—NH—CO(C₅-C₁₂-heteroaryl), styryl, or an —S(O)ᵣ—CH₃, where r is 0-2, or two adjacent positions may be substituted by —O—CH₂—O— or —O—C(CH₃)₂—O—,
   a monocyclic C₅-C₇-heteroaryl which may optionally be substituted one or more times, identically or differently, by C₁-C₆-alkyl, if R² is cyano or if R¹ and/or R² is identically or differently a C₁-C₆-alkyl radical, where at least one of the radicals is substituted at least once by halogen, or if R⁴ is —S(O)ₚ—C₁-C₆-alkyl, where p is 0-2, C₁-C₆-acyl, —O—CO—NH(C₁-C₆-alkyl), —O—CO—N(C₁-C₆-alkyl)₂, C₆-C₁₂-aryloxy, C₅-C₁₆-heteroaryloxy, hydroxy, cyano or N—(C₁-C₆-alkyl)₂,
   a monocyclic C₅-C₇-heteroaryl which may be substituted at least one or more times, identically or differently, by halogen, by CF₃, C₁-C₄-acyl, C₁-C₄-alkoxy, hydroxy, CH₂—OH, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂, or
   a bi- or tricyclic C₈-C₁₂-heteroaryl which may optionally be substituted one or more times, identically or differently, by halogen, by C₁-C₆-alkyl, C₁-C₆-acyl, C₁-C₆-alkoxy, hydroxy, cyano, CO₂—(C₁-C₆-alkyl), N—(C₁-C₆-alkyl)₂, COOH, CO—NH₂, CO—NH(C₁-C₆-alkyl) or CO—N(C₁-C₆-alkyl)₂, or
   a C₃-C₆-cycloalkyl which may optionally be substituted one or more times, identically or differently, by halogen, by C₁-C₄-alkyl, hydroxy, cyano, CO₂—(C₁-C₄-alkyl), C₁-C₄-acyl, N—(C₁-C₄-alkyl)₂, COOH, CO—NH₂, CO—NH(C₁-C₄-alkyl), CO—N(C₁-C₄-alkyl)₂ or C₁-C₄-alkoxy,
R⁴ is a hydrogen, halogen, amino, —S(O)ₚ—C₁-C₆-alkyl, where p is 0-2,
   a C₁-C₆-acyl, NH—CO—NH₂, NH—CO—C₁-C₆-alkyl, —O—CO—NH(C₁-C₆-alkyl), —O—CO—N(C₁-C₆-alkyl)₂, or C₁-C₆-alkyl group which may optionally be substituted one or more times, identically or differently, by C₁-C₆-acyl, C₁-C₆-alkoxy, hydroxy, cyano, CO₂—(C₁-C₆-alkyl), N—(C₁-C₆-alkyl)₂, C₅-C₁₂-heteroaryl, COOH, CO—NH₂, CO—NH(C₁-C₆-alkyl) or by CO—N(C₁-C₆-alkyl)₂,
   a C₁-C₆-alkoxy which may optionally be substituted one or more times, identically or differently, by hydroxy, cyano, CO₂—(C₁-C₆-alkyl), N—(C₁-C₆-alkyl)₂, NH—C₃-C₆-cycloalkyl, COOH, CO—NH₂, CO—NH(C₁-C₆-alkyl) or by CO—N(C₁-C₆-alkyl)₂,
   an O—C₆-C₁₂-aryl which may optionally be substituted by hydroxy, cyano, COOH or CO—NH₂,
   a CH₂O—C₆-C₁₂-aryl which may optionally be substituted by hydroxy, cyano, COOH or CO—NH₂,
   an O—C₅-C₁₆-heteroaryl, which may optionally be substituted by hydroxy, cyano, COOH or CO—NH₂,
   a hydroxy, cyano, O—CO—(C₁-C₆-alkyl), CO—NH(C₅-C₁₂-heteroaryl), NH—(C₁-C₆-alkyl), N—(C₁-C₆-alkyl)₂ or
   a C₆-C₁₂-aryl which may optionally be substituted one or more times, identically or differently, by halogen, by C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl), CH₂—NH—CO(C₅-C₁₂-heteroaryl), styryl, or an —S(O)ᵣ—CH₃, where r is 0-2, or two adjacent positions may be substituted by —O—CH₂—O— or —O—C(CH₃)₂—O—,
   a monocyclic C₅-C₇-heteroaryl which may optionally be substituted one or more times, identically or differently, by C₁-C₆-alkyl, if R² is cyano or if R¹ and/or R² is identically or differently a C₁-C₆-alkyl radical, where at least one of the radicals is substituted at least once by halogen,
   a monocyclic C₅-C₇-heteroaryl which may be substituted at least one or more times, identically or differently, by halogen, by CF₃, C₁-C₆-acyl, C₁-C₆-alkoxy, hydroxy, CH₂—OH, cyano, CO₂—(C₁-C₆-alkyl), N—(C₁-C₆-alkyl)₂, COOH, CO—NH₂, CO—NH(C₁-C₆-alkyl) or CO—N(C₁-C₆-alkyl)₂, or
   a bi- or tricyclic C₈-C₁₂-heteroaryl which may optionally be substituted one or more times, identically or differently, by halogen, by C₁-C₆-alkyl, C₁-C₆-acyl, C₁-C₆-alkoxy, hydroxy, cyano, CO₂—(C₁-C₆-alkyl), N—(C₁-C₆-alkyl)₂, COOH, CO—NH₂, CO—NH(C₁-C₆-alkyl) or CO—N(C₁-C₆-alkyl)₂, or
   a C₃-C₆-cycloalkyl which may optionally be substituted one or more times, identically or differently, by halogen, by C₁-C₆-alkyl, hydroxy, cyano, CO₂—(C₁-C₆-alkyl), C₁-C₆-acyl, N—(C₁-C₆-alkyl)₂, COOH, CO—NH₂, CO—NH(C₁-C₆-alkyl), CO—N(C₁-C₆-alkyl)₂ or C₁-C₆-alkoxy,
R³ and R⁴ are either in ortho, meta position or meta, para position relative to one another and together have the meaning —O—CO—S—, —S—CO—O—, CH₂—CO—O—, O—CO—CH₂—, —CH₂—CO—NH—, —NH—CO—CH₂—, —O—CO—NH-—NH—CO—O—, —CO—CH₂—(CH₂)ₘ—, —CH₂—(CH₂)ₘ—CO—, —O—(CH₂)ₘ—O—, —O—C—(CH₃)₂—O—, —CH₂—(CH₂)ₘ—CH₂—, where m is 1-3,
Y is a —(CH₂)ₙ— group, where n is 1-3,
   and the isomers, diastereomers, enantiomers and salts thereof, and cyclodextrin clathrates.

Preference is likewise given to those compounds of the general formula (I),

where
A is an aryl or heteroaryl radical which may optionally be substituted one or more times by R⁴ and/or R³,
R¹ is a hydrogen or a C₁-C₆-alkyl group which is substituted one or more times by halogen,
R² is a hydrogen, halogen, cyano, an —S(O)_{q}—CH₃, where q is 0-2, a C₁-C₄-alkoxy radical or C₁-C₆-alkyl group which is substituted one or more times by halogen,
R³ is a hydrogen, halogen, amino, —S(O)ₚ—CH₃, where p is 0-2,
   an —S—CF₃, C₁-C₆-acyl, NH—CO—NH₂, NH—CO—C₁-C₆-alkyl, —O—CO—NHCH₃, —O—CO—N(CH₃)₂ or C₁-C₆-alkyl group which may optionally be substituted one or more times, identically or differently, by C₁-C₄-acyl, C₁-C₄-alkoxy, hydroxy, cyano, CO₂—(C₁-C₄-alkyl), N—(C₁-C₄-alkyl)₂, C₅-C₁₂-heteroaryl, COOH, CO—NH₂, CO—NH(C₁-C₄-alkyl) or by CO—N(C₁-C₄-alkyl)₂,
   a C₁-C₆-alkoxy which may optionally be substituted one or more times, identically or differently, by hydroxy, cyano, CO₂—(C₁-C₆-alkyl), N—(C₁-C₆-alkyl)₂, NH—C₃-C₆-cycloalkyl, COOH, CO—NH₂, CO—NH(C₁-C₆-alkyl) or by CO—N(C₁-C₆-alkyl)₂,
   an O—C₆-C₁₂-aryl which may optionally be substituted by hydroxy, cyano, COOH or CO—NH₂,
   a CH₂O—C₆-C₁₂-aryl which may optionally be substituted by hydroxy, cyano, COOH or CO—NH₂,
   an O—C₅-C₁₆-heteroaryl which may optionally be substituted by hydroxy, cyano, COOH or CO—NH₂,
   a hydroxy, cyano, O—CO—(C₁-C₆-alkyl), CO—NH(C₅-C₁₂-heteroaryl), NH—(C₁-C₆-alkyl), N—(C₁-C₆-alkyl)₂ or
   a C₆-C₁₂-aryl which may optionally be substituted one or more times, identically or differently, by halogen, by C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl), CH₂—NH—CO(C₅-C₁₂-heteroaryl), styryl, or an —S(O)ᵣ—CH₃, where r is 0-2, or two adjacent positions may be substituted by —O—CH₂—O— or —O—C(CH₃)₂—O—,
   a monocyclic C₅-C₇-heteroaryl which may optionally be substituted one or more times, identically or differently, by C₁-C₆-alkyl, if R² is cyano or if R¹ and/or R² is identically or differently a C₁-C₆-alkyl radical, where at least one of the radicals is substituted at least once by halogen, or if R⁴ is —S(O)ₚ—C₁-C₆-alkyl, where p is 0-2, C₁-C₆-acyl-, —O—CO—NH(C₁-C₆-alkyl), —O—CO—N(C₁-C₆-alkyl)₂, C₆-C₁₂-aryloxy, C₅-C₁₆-heteroaryloxy, hydroxy, cyano or N—(C₁-C₆-alkyl)₂,
   a monocyclic C₅-C₇-heteroaryl which may be substituted at least one or more times, identically or differently, by halogen, by CF₃, C₁-C₄-acyl, C₁-C₄-alkoxy, hydroxy, CH₂—OH, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂, or
   a bi- or tricyclic C₈-C₁₂-heteroaryl which may optionally be substituted one or more times, identically or differently, by halogen, by C₁-C₆-alkyl, C₁-C₆-acyl, C₁-C₆-alkoxy, hydroxy, cyano, CO₂—(C₁-C₆-alkyl), N—(C₁-C₆-alkyl)₂, COOH, CO—NH₂, CO—NH(C₁-C₆-alkyl) or CO—N(C₁-C₆-alkyl)₂, or
   a C₃-C₆-cycloalkyl which may optionally be substituted one or more times, identically or differently, by halogen, by C₁-C₄-alkyl, hydroxy, cyano, CO₂—(C₁-C₄-alkyl), C₁-C₄-acyl, N—(C₁-C₄-alkyl)₂, COOH, CO—NH₂, CO—NH(C₁-C₄-alkyl), CO—N(C₁-C₄-alkyl)₂ or C₁-C₄-alkoxy,
R⁴ is a hydrogen, halogen, amino, —S(O)ₚ—CH₃, where p is 0-2,
   an —S—CF₃, C₁-C₆-acyl, NH—CO—NH₂, NH—CO—C₁-C₆-alkyl, —O—CO—NHCH₃, —O—CO—N(CH₃)₂, or C₁-C₆-alkyl group which may optionally be substituted one or more times, identically or differently, by C₁-C₄-acyl, C₁-C₄-alkoxy, hydroxy, cyano, CO₂—(C₁-C₄-alkyl), N—(C₁-C₄-alkyl)₂, C₅-C₁₂-heteroaryl, COOH, CO—NH₂, CO—NH(C₁-C₄-alkyl) or by CO—N(C₁-C₄-alkyl)₂,
   a C₁-C₆-alkoxy which may optionally be substituted one or more times, identically or differently, by hydroxy, cyano, CO₂—(C₁-C₆-alkyl), N—(C₁-C₆-alkyl)₂, NH—C₃-C₆-cycloalkyl, COOH, CO—NH₂, CO—NH(C₁-C₆-alkyl) or by CO—N(C₁-C₆-alkyl)₂,
   an O—C₆-C₁₂-aryl which may optionally be substituted by hydroxy, cyano, COOH or CO—NH₂,
   a CH₂O—C₆-C₁₂-aryl which may optionally be substituted by hydroxy, cyano, COOH or CO—NH₂,
   an O—C₅-C₁₆-heteroaryl which may optionally be substituted by hydroxy, cyano, COOH or CO—NH₂,
   a hydroxy, cyano, O—CO—(C₁-C₆-alkyl), CO—NH(C₅-C₁₂-heteroaryl), N—(C₁-C₆-alkyl), N—(C₁-C₆-alkyl)₂ or
   a C₆-C₁₂-aryl which may optionally be substituted one or more times, identically or differently, by halogen, by C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl), CH₂—NH—CO(C₅-C₁₂-heteroaryl), styryl, or an —S(O)ᵣ—CH₃, where r is 0-2, or two adjacent positions may be substituted by —O—CH₂—O— or —O—C(CH₃)₂—O—,
   a monocyclic C₅-C₇-heteroaryl which may optionally be substituted one or more times, identically or differently, by C₁-C₆-alkyl, if R² is cyano or if R¹ and/or R² is identically or differently a C₁-C₆-alkyl radical, where at least one of the radicals is substituted at least once by halogen,
   a monocyclic C₅-C₁₂-heteroaryl which may be substituted at least one or more times, identically or differently, by halogen, by CF₃, C₁-C₄-acyl, C₁-C₄-alkoxy, hydroxy, CH₂—OH, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂,
   a bi- or tricyclic C₈-C₁₂-heteroaryl which may optionally be substituted one or more times, identically or differently, by halogen, by C₁-C₆-alkyl, C₁-C₆-acyl, C₁-C₆-alkoxy, hydroxy, cyano, CO₂—(C₁-C₆-alkyl), N—(C₁-C₆-alkyl)₂, COOH, CO—NH₂, CO—NH(C₁-C₆-alkyl) or CO—N(C₁-C₆-alkyl)₂, or
   a C₃-C₆-cycloalkyl which may optionally be substituted one or more times, identically or differently, by halogen, by C₁-C₄-alkyl, hydroxy, cyano, CO₂—(C₁-C₄-alkyl), C₁-C₄-acyl, N—(C₁-C₄-alkyl)₂, COOH, CO—NH₂, CO—NH(C₁-C₄-alkyl), CO—N(C₁-C₄-alkyl)₂ or C₁-C₄-alkoxy,
R³ and R⁴ are either in ortho, meta position or meta, para position relative to one another and together have the meaning —O—CO—S—, —S—CO—O—, CH₂—CO—O—, O—CO—CH₂—, —CH₂—CO—NH—, —NH—CO—CH₂—, —O—CO—NH—, —NH—CO—O—, —CO—CH₂—(CH₂)ₘ—, —CH₂—(CH₂)ₘ—CO—, —O—(CH₂)ₘ—O—, —O—C—(CH₃)₂—O—, —CH₂—(CH₂)ₘ—CH₂—, where m is 1-3,
Y is a —(CH₂)ₙ— group, where n is 1-3,
   and the isomers, diastereomers, enantiomers and salts thereof, and cyclodextrin clathrates.

Preference is likewise given to those compounds of the general formula (I)

where
A is a phenyl, naphthyl or heteroaryl radical which may optionally be substituted once or twice by R³ and/or R⁴,
R¹ is a hydrogen or a C₁-C₆-alkyl group which may be substituted one or more times by halogen,
R² is a hydrogen, halogen, cyano, an —S(O)_{q}—CH₃, where q is 0-2, a C₁-C₄-alkoxy radical or C₁-C₆-alkyl group,
R³ is a hydrogen, halogen, amino, —S(O)ₚ—CH₃, where p is 0-2,
   an —S—CF₃, C₁-C₆-acyl, NH—CO—NH₂, NH—CO—C₁-C₆-alkyl, —O—CO—NHCH₃, —O—CO—N(CH₃)₂, or C₁-C₆-alkyl group which may optionally be substituted once, twice, three, four or five times, identically or differently, by C₁-C₄-acyl, C₁-C₄-alkoxy, hydroxy, cyano, CO₂—(C₁-C₄-alkyl), N—(C₁-C₄-alkyl)₂, C₅-C₁₂-heteroaryl, COOH, CO—NH₂, CO—NH(C₁-C₄-alkyl) or by CO—N(C₁-C₄-alkyl)₂,
   a C₁-C₆-alkoxy which may optionally be substituted one or more times, identically or differently, by hydroxy, cyano, CO₂—(C₁-C₆-alkyl), N—(C₁-C₆-alkyl)₂, NH—C₃-C₆-cycloalkyl, COOH, CO—NH₂, CO—NH(C₁-C₆-alkyl) or by CO—N(C₁-C₆-alkyl)₂,
   an O—C₆-C₁₂-aryl which may optionally be substituted by hydroxy, cyano, COOH or CO—NH₂,
   a CH₂O—C₆-C₁₂-aryl which may optionally be substituted by hydroxy, cyano, COOH or CO—NH₂,
   an O—C₅-C₁₆-heteroaryl which may optionally be substituted by hydroxy, cyano, COOH or CO—NH₂,
   a hydroxy, cyano, O—CO—(C₁-C₆-alkyl), CO—NH(C₅-C₁₂-heteroaryl), NH—(C₁-C₆-alkyl), N—(C₁-C₆-alkyl)₂,
   a C₆-C₁₂-aryl which may optionally be substituted one or more times, identically or differently, by halogen, by C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl), CH₂—NH—CO(C₅-C₁₂-heteroaryl), styryl, or an —S(O)ᵣ—CH₃, where r is 0-2, or two adjacent positions may be substituted by —O—CH₂—O— or —O—C(CH₃)₂—O—,
   a monocyclic C₅-C₇-heteroaryl which may optionally be substituted one or more times, identically or differently, by C₁-C₆-alkyl, if R² is cyano or if R¹ and/or R² is identically or differently a C₁-C₄-alkyl radical, where at least one of the radicals is substituted at least once by halogen, or if R⁴ is —S(O)ₚ—C₁-C₄-alkyl, where p is 0-2, C₁-C₄-acyl-, —O—CO—NH(C₁-C₄-alkyl), —O—CO—N(C₁-C₄-alkyl)₂, C₆-C₁₂-aryloxy, C₅-C₁₆-heteroaryloxy, hydroxy, cyano or N—(C₁-C₄-alkyl)₂,
   a monocyclic C₅-C₇-heteroaryl which may be substituted at least once or else twice, three, four or five times, identically or differently, by halogen, by CF₃, C₁-C₄-acyl, C₁-C₄-alkoxy, hydroxy, CH₂—OH, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂, or
   a bi- or tricyclic C₈-C₁₂-heteroaryl which may optionally be substituted once, twice, three, four or five times, identically or differently, by halogen, by C₁-C₆-alkyl, C₁-C₆-acyl, C₁-C₆-alkoxy, hydroxy, cyano, CO₂—(C₁-C₆-alkyl), N—(C₁-C₆-alkyl)₂, COOH, CO—NH₂, CO—NH(C₁-C₆-alkyl) or CO—N(C₁-C₆-alkyl)₂,
   a C₃-C₆-cycloalkyl which may optionally be substituted once, twice, three, four or five times, identically or differently, by halogen, by C₁-C₄-alkyl, hydroxy, cyano, CO₂—(C₁-C₄-alkyl), C₁-C₄-acyl, N—(C₁-C₄-alkyl)₂, COOH, CO—NH₂, CO—NH(C₁-C₄-alkyl), CO—N(C₁-C₄-alkyl)₂ or C₁-C₄-alkoxy,
R⁴ is a hydrogen, halogen, amino, —S(O)ₚ—CH₃, where p is 0-2,
   an —S—CF₃, C₁-C₆-acyl, NH—CO—NH₂, NH—CO—C₁-C₆-alkyl, —O—CO—NHCH₃, —O—CO—N(CH₃)₂, or C₁-C₆-alkyl group which may optionally be substituted once, twice, three, four or five times, identically or differently, by C₁-C₄-acyl, C₁-C₄-alkoxy, hydroxy, cyano, CO₂—(C₁-C₄-alkyl), N—(C₁-C₄-alkyl)₂, C₅-C₁₂-heteroaryl, COOH, CO—NH₂, CO—NH(C₁-C₄-alkyl) or by CO—N(C₁-C₄-alkyl)₂,
   a C₁-C₆-alkoxy which may optionally be substituted one or more times, identically or differently, by hydroxy, cyano, CO₂—(C₁-C₆-alkyl), N—(C₁-C₆-alkyl)₂, NH—C₃-C₆-cycloalkyl, COOH, CO—NH₂, CO—NH(C₁-C₆-alkyl) or by CO—N(C₁-C₆-alkyl)₂,
   an O—C₆-C₁₂-aryl which may optionally be substituted by hydroxy, cyano, COOH or CO—NH₂,
   a CH₂O—C₆-C₁₂-aryl which may optionally be substituted by hydroxy, cyano, COOH or CO—NH₂,
   an O—C₅-C₁₆-heteroaryl which may optionally be substituted by hydroxy, cyano, COOH or CO—NH₂,
   a hydroxy, cyano, O—CO—(C₁-C₆-alkyl), CO—NH(C₅-C₁₂-heteroaryl), NH—(C₁-C₆-alkyl), N—(C₁-C₆-alkyl)₂,
   a C₆-C₁₂-aryl which may optionally be substituted one or more times, identically or differently, by halogen, by C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl), CH₂—NH—CO(C₅-C₁₂-heteroaryl), styryl, or an —S(O)ᵣ—CH₃, where r is 0-2, or two adjacent positions may be substituted by —O—CH₂—O— or —O—C(CH₃)₂—O—,
   a monocyclic C₅-C₇-heteroaryl which may optionally be substituted one or more times, identically or differently, by C₁-C₆-alkyl, if R² is cyano or if R¹ and/or R² is identically or differently a C₁-C₄-alkyl radical, where at least one of the radicals is substituted at least once by halogen,
   a monocyclic C₅-C₇-heteroaryl which may be substituted at least once or else twice, three, four or five times, identically or differently, by halogen, by CF₃, C₁-C₄-acyl, C₁-C₄-alkoxy, hydroxy, CH₂—OH, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂,
   a bi- or tricyclic C₈-C₁₂-heteroaryl which may optionally be substituted once, twice, three, four or five times, identically or differently, by halogen, by C₁-C₆-alkyl, C₁-C₆-acyl, C₁-C₆-alkoxy, hydroxy, cyano, CO₂—(C₁-C₆-alkyl), N—(C₁-C₆-alkyl)₂, COOH, CO—NH₂, CO—NH(C₁-C₆-alkyl) or CO—N(C₁-C₆-alkyl)₂,
   a C₃-C₆-cycloalkyl which may optionally be substituted once, twice, three, four or five times, identically or differently, by halogen, by C₁-C₄-alkyl, hydroxy, cyano, CO₂—(C₁-C₄-alkyl), C₁-C₄-acyl, N—(C₁-C₄-alkyl)₂, COOH, CO—NH₂, CO—NH(C₁-C₄-alkyl), CO—N(C₁-C₄-alkyl)₂ or C₁-C₄-alkoxy,
R³ and R⁴ are either in ortho, meta position or meta, para position relative to one another and together have the meaning of —O—CO—S—, —S—CO—O—, CH₂—CO—O—, O—CO—CH₂—, —CH₂—CO—NH—, —NH—CO—CH₂—, —O—CO—NH—, —NH—CO—O—, —CO—CH₂—(CH₂)ₘ, —CH₂—(CH₂)ₘ—CO—, —O—(CH₂)ₘ—O—, —O—C—(CH₃)₂—O—, —CH₂—(CH₂)ₘ—CH₂—, where m is 1-3,
Y is a —(CH₂)ₙ— group, where n is 1-3,
   and the isomers, diastereomers, enantiomers and salts thereof, and cyclodextrin clathrates.

Preference is likewise given to those compounds of the general formula (I),

where
A is a phenyl, naphthyl or heteroaryl radical which may optionally be substituted once or twice by R³ and/or R⁴,
R¹ is a hydrogen or a C₁-C₆-alkyl radical which is substituted one or more times by halogen,
R² is a hydrogen, halogen, cyano, an —S(O)_{q}—CH₃, where q is 0-2, a C₁-C₄-alkoxy radical or C₁-C₆-alkyl radical which is substituted one or more times by halogen,
R³ is a hydrogen, halogen, amino, —S(O)ₚ—CH₃, where p is 0-2,
   an —S—CF₃, C₁-C₆-acyl, NH—CO—NH₂, NH—CO—C₁-C₆-alkyl, —O—CO—NHCH₃, —O—CO—N(CH₃)₂, or C₁-C₆-alkyl group which may optionally be substituted once, twice, three, four or five times, identically or differently, by C₁-C₄-acyl, C₁-C₄-alkoxy, hydroxy, cyano, CO₂—(C₁-C₄-alkyl), N—(C₁-C₄-alkyl)₂, C₅-C₁₂-heteroaryl, COOH, CO—NH₂, CO—NH(C₁-C₄-alkyl) or by CO—N(C₁-C₄-alkyl)₂,
   a C₁-C₄-alkoxy which may optionally be substituted one or more times, identically or differently, by hydroxy, cyano, CO₂—(C₁-C₄-alkyl), N—(C₁-C₄-alkyl)₂, NH—C₃-C₆-cycloalkyl, COOH, CO—NH₂, CO—NH(C₁-C₄-alkyl) or by CO—N(C₁-C₄-alkyl)₂,
   an O—C₆-C₁₂-aryl which may optionally be substituted by hydroxy, cyano, COOH or CO—NH₂,
   a CH₂O—C₆-C₁₂-aryl which may optionally be substituted by hydroxy, cyano, COOH or CO—NH₂,
   an O—C₅-C₁₆-heteroaryl which may optionally be substituted by hydroxy, cyano, COOH or CO—NH₂,
   a hydroxy, cyano, O—CO—(C₁-C₄-alkyl), CO—NH(C₅-C₁₂-heteroaryl), NH—(C₁-C₄-alkyl), N—(C₁-C₄-alkyl)₂ or
   a C₆-C₁₂-aryl which may optionally be substituted one or more times, identically or differently, by halogen, by C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl), CH₂—NH—CO(C₅-C₁₂-heteroaryl), styryl, or an —S(O)ᵣ—CH₃, where r is 0-2, or two adjacent positions may be substituted by —O—CH₂—O— or —O—C(CH₃)₂—O—,
   a monocyclic C₅-C₇-heteroaryl which may optionally be substituted one or more times, identically or differently, by C₁-C₆-alkyl, if R² is cyano or if R¹ and/or R² is identically or differently a C₁-C₄-alkyl radical, where at least one of the radicals is substituted at least once by halogen, or if R⁴ is —S(O)ₚ—C₁-C₄-alkyl, where p is 0-2, C₁-C₄-acyl-, —O—CO—NH(C₁-C₄-alkyl), —O—CO—N(C₁-C₄-alkyl)₂, C₆-C₁₂-aryloxy, C₅-C₁₆-heteroaryloxy, hydroxy, cyano or N—(C₁-C₄-alkyl)₂,
   a monocyclic C₅-C₇-heteroaryl which may be substituted at least once or else twice, three, four or five times, identically or differently, by halogen, by CF₃, C₁-C₄-acyl, C₁-C₄-alkoxy, hydroxy, CH₂—OH, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂, or
   a bi- or tricyclic C₈-C₁₂-heteroaryl which may optionally be substituted once, twice, three, four or five times, identically or differently, by halogen, by C₁-C₆-alkyl, C₁-C₆-acyl, C₁-C₆-alkoxy, hydroxy, cyano, CO₂—(C₁-C₆-alkyl), N—(C₁-C₆-alkyl)₂, COOH, CO—NH₂, CO—NH(C₁-C₆-alkyl) or CO—N(C₁-C₆-alkyl)₂, or
   a C₃-C₆-cycloalkyl which may optionally be substituted once, twice, three, four or five times, identically or differently, by halogen, by C₁-C₄-alkyl, hydroxy, cyano, CO₂—(C₁-C₄-alkyl), C₁-C₄-acyl, N—(C₁-C₄-alkyl)₂, COOH, CO—NH₂, CO—NH(C₁-C₄-alkyl), CO—N(C₁-C₄-alkyl)₂ or C₁-C₄-alkoxy,
R⁴ is a hydrogen, halogen, amino, —S(O)ₚ—CH₃, where p is 0-2,
   an —S—CF₃, C₁-C₆-acyl, NH—CO—NH₂, NH—CO—C₁-C₆-alkyl, —O—CO—NHCH₃, —O—CO—N(CH₃)₂, or C₁-C₆-alkyl group which may optionally be substituted once, twice, three, four or five times, identically or differently, by C₁-C₄-acyl, C₁-C₄-alkoxy, hydroxy, cyano, CO₂—(C₁-C₄-alkyl), N—(C₁-C₄-alkyl)₂, C₅-C₁₂-heteroaryl, COOH, CO—NH₂, CO—NH(C₁-C₄-alkyl) or by CO—N(C₁-C₄-alkyl)₂,
   a C₁-C₄-alkoxy which may optionally be substituted one or more times, identically or differently, by hydroxy, cyano, CO₂—(C₁-C₄-alkyl), N—(C₁-C₄-alkyl)₂, NH—C₃-C₆-cycloalkyl, COOH, CO—NH₂, CO—NH(C₁-C₄-alkyl) or by CO—N(C₁-C₄-alkyl)₂,
   an O—C₆-C₁₂-aryl which may optionally be substituted by hydroxy, cyano, COOH or CO—NH₂,
   a CH₂O—C₆-C₁₂-aryl which may optionally be substituted by hydroxy, cyano, COOH or CO—NH₂,
   an O—C₅-C₁₆-heteroaryl which may optionally be substituted by hydroxy, cyano, COOH or CO—NH₂,
   a hydroxy, cyano, O—CO—(C₁-C₄-alkyl), CO—NH(C₅-C₁₂-heteroaryl), NH—(C₁-C₄-alkyl), N—(C₁-C₄-alkyl)₂ or
   a C₆-C₁₂-aryl which may optionally be substituted one or more times, identically or differently, by halogen, by C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl), CH₂—NH—CO(C₅-C₁₂-heteroaryl), styryl, or an —S(O)ᵣ—CH₃, where r is 0-2, or two adjacent positions may be substituted by —O—CH₂—O— or —O—C(CH₃)₂—O—,
   a monocyclic C₅-C₇-heteroaryl which may optionally be substituted one or more times, identically or differently, by C₁-C₆-alkyl, if R² is cyano or if R¹ and/or R² is identically or differently a C₁-C₄-alkyl radical, where at least one of the radicals is substituted at least once by halogen,
   a monocyclic C₅-C₇-heteroaryl which may be substituted at least once or else twice, three, four or five times, identically or differently, by halogen, by CF₃, C₁-C₄-acyl, C₁-C₄-alkoxy, hydroxy, CH₂—OH, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂,
   a bi- or tricyclic C₈-C₁₂-heteroaryl which may optionally be substituted once, twice, three, four or five times, identically or differently, by halogen, by C₁-C₆-alkyl, C₁-C₆-acyl, C₁-C₆-alkoxy, hydroxy, cyano, CO₂—(C₁-C₆-alkyl), N—(C₁-C₆-alkyl)₂, COOH, CO—NH₂, CO—NH(C₁-C₆-alkyl) or CO—N(C₁-C₆-alkyl)₂,
   a C₃-C₆-cycloalkyl which may optionally be substituted once, twice, three, four or five times, identically or differently, by halogen, by C₁-C₄-alkyl, hydroxy, cyano, CO₂—(C₁-C₄-alkyl), C₁-C₄-acyl, N—(C₁-C₄-alkyl)₂, COOH, CO—NH₂, CO—NH(C₁-C₄-alkyl), CO—N(C₁-C₄-alkyl)₂ or C₁-C₄-alkoxy,
R³ and R⁴ are either in ortho, meta position or meta, para position relative to one another and together have the meaning of —O—CO—S—, —S—CO—O—, CH₂—CO—O—, O—CO—CH₂—, —CH₂—CO—NH—, —NH—CO—CH₂—, —O—CO—NH—, —NH—CO—O—, —CO—CH₂—(CH₂)ₘ, —CH₂—(CH₂)ₘ—CO—, —O—(CH₂)ₘ—O—, —O—C—(CH₃)₂—O—, —CH₂—(CH₂)ₘ—CH₂—, where m is 1-3,
Y is a —(CH₂)ₙ— group, where n is 1-3,
   and the isomers, diastereomers, enantiomers and salts thereof, and cyclodextrin clathrates.

Preference is likewise given to those compounds of the general formula (I)

where
A is a phenyl, naphthyl or heteroaryl radical which may optionally be substituted once or twice by R³ and/or R⁴,
R¹ is a hydrogen or a C₁-C₄-alkyl radical which is substituted once, twice or three times by chlorine, fluorine, or bromine,
R² is a hydrogen, chlorine, fluorine, bromine, cyano, an OCH₃ group or a C₁-C₄-alkyl radical which is substituted once, twice or three times by chlorine, fluorine or bromine,
R³ is a hydrogen, halogen, amino, —S(O)ₚ—CH₃, where p is 0-2,
   an —S—CF₃, C₁-C₆-acyl, NH—CO—NH₂, NH—CO—C₁-C₆-alkyl, —O—CO—NHCH₃, —O—CO—N(CH₃)₂, or C₁-C₆-alkyl group which may optionally be substituted once or twice, identically or differently, by C₁-C₄-acyl, C₁-C₄-alkoxy, hydroxy, cyano, CO₂—(C₁-C₄-alkyl), N—(C₁-C₄-alkyl)₂, C₅-C₁₂-heteroaryl, COOH, CO—NH₂, CO—NH(C₁-C₄-alkyl) or by CO—N(C₁-C₄-alkyl)₂,
   a C₁-C₄-alkoxy which may optionally be substituted one or more times, identically or differently, by hydroxy, cyano, CO₂—(C₁-C₄-alkyl), N—(C₁-C₄-alkyl)₂, NH—C₃-C₆-cycloalkyl, COOH, CO—NH₂, CO—NH(C₁-C₄-alkyl) or by CO—N(C₁-C₄-alkyl)₂,
   an O—C₆-C₁₂-aryl which may optionally be substituted by hydroxy, cyano, COOH or CO—NH₂,
   a CH₂O—C₆-C₁₂-aryl which may optionally be substituted by hydroxy, cyano, COOH or CO—NH₂,
   an O—C₅-C₁₆-heteroaryl which may optionally be substituted by hydroxy, cyano, COOH or CO—NH₂,
   a hydroxy, cyano, O—CO—(C₁-C₄-alkyl), CO—NH(C₅-C₁₂-heteroaryl), NH—(C₁-C₄-alkyl), N—(C₁-C₄-alkyl)₂,
   a C₆-C₁₂-aryl which may optionally be substituted one or more times, identically or differently, by halogen, by C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl), CH₂—NH—CO(C₅-C₁₂-heteroaryl), styryl, or an —S(O)ᵣ—CH₃, where r is 0-2, or two adjacent positions may be substituted by —O—CH₂—O— or —O—C(CH₃)₂—O—,
   a monocyclic C₅-C₇-heteroaryl which may optionally be substituted one or more times, identically or differently, by C₁-C₆-alkyl, if R² is cyano or if R¹ and/or R² is a CF₃ radical, or if R⁴ is —S(O)ₚ—C₁-C₆-alkyl, where p is 0-2, C₁-C₄-acyl-, —O—CO—NH(C₁-C₄-alkyl), —O—CO—N(C₁-C₄-alkyl)₂, C₆-C₁₂-aryloxy, C₅-C₁₆-heteroaryloxy, hydroxy, cyano or N—(C₁-C₄-alkyl)₂,
   a monocyclic C₅-C₇-heteroaryl which may be substituted at least once or twice, identically or differently, by halogen, by CF₃, C₁-C₄-acyl, C₁-C₄-alkoxy, hydroxy, CH₂—OH, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂,
   a bi- or tricyclic C₈-C₁₂-heteroaryl which may optionally be substituted once or twice, identically or differently, by halogen, by C₁-C₆-alkyl, C₁-C₆-acyl, C₁-C₆-alkoxy, hydroxy, cyano, CO₂—(C₁-C₆-alkyl), N—(C₁-C₆-alkyl)₂, COOH, CO—NH₂, CO—NH(C₁-C₆-alkyl) or CO—N(C₁-C₆-alkyl)₂, or
   a C₃-C₆-cycloalkyl which may optionally be substituted once or twice, identically or differently, by halogen, by C₁-C₄-alkyl, hydroxy, cyano, CO₂—(C₁-C₄-alkyl), C₁-C₄-alkyl, N—(C₁-C₄-alkyl)₂, COOH, CO—NH₂, CO—NH(C₁-C₄-alkyl), CO—N(C₁-C₄-alkyl)₂ or C₁-C₄-alkoxy,
R⁴ is a hydrogen, halogen, amino, —S(O)ₚ—CH₃, where p is 0-2,
   an —S—CF₃, C₁-C₆-acyl, NH—CO—NH₂, NH—CO—C₁-C₆-alkyl, —O—CO—NHCH₃, —O—CO—N(CH₃)₂, or C₁-C₆-alkyl group which may optionally be substituted once or twice, identically or differently, by C₁-C₄-acyl, C₁-C₄-alkoxy, hydroxy, cyano, CO₂—(C₁-C₄-alkyl), N—(C₁-C₄-alkyl)₂, C₅-C₁₂-heteroaryl, COOH, CO—NH₂, CO—NH(C₁-C₄-alkyl) or by CO—N(C₁-C₄-alkyl)₂,
   a C₁-C₄-alkoxy which may optionally be substituted one or more times, identically or differently, by hydroxy, cyano, CO₂—(C₁-C₄-alkyl), N—(C₁-C₄-alkyl)₂, NH—C₃-C₆-cycloalkyl, COOH, CO—NH₂, CO—NH(C₁-C₄-alkyl) or by CO—N(C₁-C₄-alkyl)₂,
   an O—C₆-C₁₂-aryl which may optionally be substituted by hydroxy, cyano, COOH or CO—NH₂,
   a CH₂O—C₆-C₁₂-aryl which may optionally be substituted by hydroxy, cyano, COOH or CO—NH₂,
   an O—C₅-C₁₆-heteroaryl which may optionally be substituted by hydroxy, cyano, COOH or CO—NH₂,
   a hydroxy, cyano, O—CO—(C₁-C₄-alkyl), CO—NH(C₅-C₁₂-heteroaryl), NH—(C₁-C₄-alkyl), N—(C₁-C₄-alkyl)₂ or
   a C₆-C₁₂-aryl which may optionally be substituted one or more times, identically or differently, by halogen, by C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂ CH₃, SO₂NH₂, SO₂NH(C₁-C₆-alkyl), SO₂N(C₁-C₆-alkyl)₂, COOH, CO—NH₂, CO—NH(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl), CH₂—NH—CO(C₅-C₁₂-heteroaryl), styryl, or an —S(O)ᵣ—CH₃, where r is 0-2, or two adjacent positions may be substituted by —O—CH₂—O— or —O—C(CH₃)₂—O—,
   a monocyclic C₅-C₇-heteroaryl which may optionally be substituted one or more times, identically or differently, by C₁-C₆-alkyl, if R² is cyano or if R¹ and/or R² is a CF₃ radical,
   a monocyclic C₅-C₇-heteroaryl which may be substituted at least once or twice, identically or differently, by halogen, by CF₃, C₁-C₄-acyl, C₁-C₄-alkoxy, hydroxy, CH₂—OH, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂, or
   a bi- or tricyclic C₈-C₁₂-heteroaryl which may optionally be substituted once or twice, identically or differently, by halogen, by C₁-C₆-alkyl, C₁-C₆-acyl, C₁-C₆-alkoxy, hydroxy, cyano, CO₂—(C₁-C₆-alkyl), N—(C₁-C₆-alkyl)₂, COOH, CO—NH₂, CO—NH(C₁-C₆-alkyl) or CO—N(C₁-C₆-alkyl)₂, or
   a C₃-C₆-cycloalkyl which may optionally be substituted once or twice, identically or differently, by halogen, by C₁-C₄-alkyl, hydroxy, cyano, CO₂—(C₁-C₄-alkyl), C₁-C₄-acyl, N—(C₁-C₄-alkyl)₂, COOH, CO—NH₂, CO—NH(C₁-C₄-alkyl), CO—N(C₁-C₄-alkyl)₂ or C₁-C₄-alkoxy,
R³ and R⁴ are either in ortho, meta position or meta, para position relative to one another and together have the meaning of —O—CO—S—, —S—CO—O—, CH₂—CO—O—, O—CO—CH₂—, —CH₂—CO—NH—, —NH—CO—CH₂—, —O—CO—NH—, —NH—CO—O—, —CO—CH₂—(CH₂)ₘ—, —CH₂—(CH₂)ₘ—CO—, —O(CH₂)ₘ—O—, —O—C—(CH₃)₂—O—, —CH₂—(CH₂)ₘ—CH₂—, where m is 1-3,
Y is a —(CH₂)ₙ— group, where n is 1-3,
   and the isomers, diastereomers, enantiomers and salts thereof, and cyclodextrin clathrates.

The following compounds corresponding to the present invention are very particularly preferred:
1. biphenyl-4-carboxylic acid [2-(7-fluoro-2,4-dimethyl-1H-indol-3-yl)ethyl]amide
2. N-[2-(7-fluoro-2,4-dimethyl-1H-indol-3-yl)ethyl]-3,4,5-trimethoxybenzamide
3. 4-fluoro-N-[2-(7-fluoro-2,4-dimethyl-1H-indol-3-yl)ethyl]benzamide
4. N-[2-(7-fluoro-2,4-dimethyl-1H-indol-3-yl)ethyl]-4-methylbenzamide
5. 2-chloro-N-[2-(7-fluoro-2,4-dimethyl-1H-indol-3-yl)ethyl]benzamide
6. N-[2-(7-fluoro-2,4-dimethyl-1H-indol-3-yl)ethyl]-4-trifluoromethylbenzamide
7. 3-chloro-N-[2-(7-fluoro-2,4-dimethyl-1H-indol-3-yl)ethyl]benzamide
8. N-[2-(7-fluoro-2,4-dimethyl-1H-indol-3-yl)ethyl]-4-methoxybenzamide
9. 4-chloro-N-[2-(7-fluoro-2,4-dimethyl-1H-indol-3-yl)ethyl]benzamide
10. N-[2-(7-fluoro-2,4-dimethyl-1H-indol-3-yl)ethyl]-3-methylbenzamide
11. 4-tert-butyl-N-[2-(7-fluoro-2,4-dimethyl-1H-indol-3-yl)ethyl]benzamide
12. benzo[1,3]dioxole-5-carboxylic acid [2-(7-fluoro-2,4-dimethyl-1H-indol-3-yl)ethyl]amide
13. thiophene-2-carboxylic acid [2-(7-fluoro-2,4-dimethyl-1H-indol-3-yl)ethyl]amide
14. quinoxaline-6-carboxylic acid [2-(7-fluoro-2,4-dimethyl-1H-indol-3-yl)ethyl]amide
15. 5-phenylpyridine-2-carboxylic acid [2-(7-fluoro-2,4-dimethyl-1H-indol-3-yl)ethyl]amide
16. 5-phenyl-1H-pyrrole-2-carboxylic acid [2-(7-fluoro-2,4-dimethyl-1H-indol-3-yl)ethyl]amide
17. N-[2-(4,7-difluoro-2-methyl-1H-indol-3-yl)ethyl]-3,4-dimethoxybenzamide
18. (±)-N-[2-(7-fluoro-2,4-dimethyl-1H-indol-3-yl)ethyl]-4-methanesulfinylbenzamide
19. N-[2-(7-fluoro-1H-indol-3-yl)ethyl]-3,4-dimethoxybenzamide
20. N-[2-(7-fluoro-2,4-dimethyl-1H-indol-3-yl)ethyl]-3-[1,2,4]triazol-1-ylmethylbenzamide
21. thieno[2,3-b]pyrazine-6-carboxylic acid [2-(7-fluoro-2,4-dimethyl-1H-indol-3-yl)ethyl]amide
22. N-[2-(7-fluoro-2-methyl-1H-indol-3-yl)ethyl]-3,4-dimethoxybenzamide
23. N-[2-(4-chloro-7-fluoro-2-methyl-1H-indol-3-yl)ethyl]-3,4-dimethoxybenzamide
24. N-[2-(7-fluoro-2,4-dimethyl-1H-indol-3-yl)ethyl]-4-methanesulfonylbenzamide
25. N-[2-(4-bromo-7-fluoro-2-methyl-1H-indol-3-yl)ethyl]-3,4-dimethoxy-benzamide
26. 1H-benzotriazole-5-carboxylic acid [2-(4,7-difluoro-2-methyl-1H-indol-3-yl)ethyl]amide
27. 1H-indole-2-carboxylic acid [2-(4,7-difluoro-2-methyl-1H-indol-3-yl)ethyl]amide
28. 4′-fluorobiphenyl-4-carboxylic acid [2-(7-fluoro-2,4-dimethyl-1H-indol-3-yl)ethyl]amide
29. N-[2-(7-fluoro-2,4-dimethyl-1H-indol-3-yl)ethyl]-N′-pyridin-3-yl-terephthalamide
30. 4-amino-N-[2-(7-fluoro-2,4-dimethyl-1H-indol-3-yl)ethyl]benzamide
31. 5-bromofuran-2-carboxylic acid [2-(7-fluoro-2,4-dimethyl-1H-indol-3-yl)ethyl]amide
32. N-[2-(7-fluoro-2,4-dimethyl-1H-indol-3-yl)ethyl]-isonicotinamide
33. N-[2-(7-fluoro-2,4-dimethyl-1H-indol-3-yl)ethyl]benzamide
34. 2-fluoro-N-[2-(7-fluoro-2,4-dimethyl-1H-indol-3-yl)ethyl]benzamide
35. 3-fluoro-N-[2-(7-fluoro-2,4-dimethyl-1H-indol-3-yl)ethyl]benzamide
36. 1H-indole-2-carboxylic acid [2-(7-fluoro-2,4-dimethyl-1H-indol-3-yl)ethyl]-amide
37. N-[2-(7-fluoro-2,4-dimethyl-1H-indol-3-yl)ethyl]-6-methylnicotinamide
38. N-[2-(7-fluoro-2,4-dimethyl-1H-indol-3-yl)ethyl]-3-methoxybenzamide
39. 4-ethoxy-N-[2-(7-fluoro-2,4-dimethyl-1H-indol-3-yl)ethyl]benzamide
40. N-[2-(7-fluoro-2,4-dimethyl-1H-indol-3-yl)ethyl]-4-hydroxy-3,5-dimethoxy-benzamide
41. 1H-benzotriazole-5-carboxylic acid [2-(7-fluoro-2,4-dimethyl-1H-indol-3-yl)ethyl]amide
42. 5-methylthiophene-2-carboxylic acid [2-(7-fluoro-2,4-dimethyl-1H-indol-3-yl)ethyl]amide
43. 1H-pyrrole-2-carboxylic acid [2-(7-fluoro-2,4-dimethyl-1H-indol-3-yl)ethyl]amide
44. N-[2-(7-fluoro-2,4-dimethyl-1H-indol-3-yl)ethyl]-2-methylaminobenzamide
45. thiophene-3-carboxylic acid [2-(7-fluoro-2,4-dimethyl-1H-indol-3-yl)ethyl]-amide
46. 6-cyano-N-[2-(7-fluoro-2,4-dimethyl-1H-indol-3-yl)ethyl]nicotinamide
47. 1H-benzoimidazole-5-carboxylic acid [2-(7-fluoro-2,4-dimethyl-1H-indol-3-yl)ethyl]amide
48. N-[2-(7-fluoro-2,4-dimethyl-1H-indol-3-yl)ethyl]-3-trifluoromethylbenzamide
49. N-[2-(7-fluoro-2,4-dimethyl-1H-indol-3-yl)ethyl]-4-hydroxy-3-methoxy-benzamide
50. 4-dimethylamino-N-[2-(7-fluoro-2,4-dimethyl-1H-indol-3-yl)ethyl]benzamide
51. 4-cyano-N-[2-(7-fluoro-2,4-dimethyl-1H-indol-3-yl)ethyl]benzamide
52. isoxazole-5-carboxylic acid [2-(7-fluoro-2,4-dimethyl-1H-indol-3-yl)ethyl]-amide
53. 4-acetyl-N-[2-(7-fluoro-2,4-dimethyl-1H-indol-3-yl)ethyl]benzamide
54. 4-chloro-3-fluoro-N-[2-(7-fluoro-2,4-dimethyl-1H-indol-3-yl)ethyl]benzamide
55. 4-chloromethyl-N-[2-(7-fluoro-2,4-dimethyl-1H-indol-3-yl)ethyl]benzamide
56. N-[2-(7-fluoro-2,4-dimethyl-1H-indol-3-yl)ethyl]-3,5-dimethylbenzamide
57. 3,4-difluoro-N-[2-(7-fluoro-2,4-dimethyl-1H-indol-3-yl)ethyl]benzamide
58. N-[2-(7-fluoro-2,4-dimethyl-1H-indol-3-yl)ethyl]-4-propylbenzamide
59. N-[2-(7-fluoro-2,4-dimethyl-1H-indol-3-yl)ethyl]-3-hydroxy-4-methylbenzamide
60. 2,3-difluoro-N-[2-(7-fluoro-2,4-dimethyl-1H-indol-3-yl)ethyl]benzamide
61. 3,5-difluoro-N-[2-(7-fluoro-2,4-dimethyl-1H-indol-3-yl)ethyl]benzamide
62. naphthalene-2-carboxylic acid [2-(7-fluoro-2,4-dimethyl-1H-indol-3-yl)ethyl]-amide
63. 5-chlorothiophene-2-carboxylic acid [2-(7-fluoro-2,4-dimethyl-1H-indol-3-yl)ethyl]amide
64. 6-fluoro-N-[2-(7-fluoro-2,4-dimethyl-1H-indol-3-yl)ethyl]nicotinamide
65. 3-chloromethyl-N-[2-(7-fluoro-2,4-dimethyl-1H-indol-3-yl)ethyl]benzamide
66. 4-butoxy-N-[2-(7-fluoro-2,4-dimethyl-1H-indol-3-yl)ethyl]benzamide
67. 4-acetoxy-N-[2-(7-fluoro-2,4-dimethyl-1H-indol-3-yl)ethyl]benzamide
68. N-[2-(7-fluoro-2,4-dimethyl-1H-indol-3-yl)ethyl]-4-methylsulfanylbenzamide
69. 4-cyano-2-fluoro-N-[2-(7-fluoro-2,4-dimethyl-1H-indol-3-yl)ethyl]benzamide
70. isoquinoline-1-carboxylic acid [2-(7-fluoro-2,4-dimethyl-1H-indol-3-yl)ethyl]-amide
71. isoquinoline-1-carboxylic acid [2-(7-fluoro-2,4-dimethyl-1H-indol-3-yl)ethyl]-amide
72. isoquinoline-1-carboxylic acid [2-(7-fluoro-2,4-dimethyl-1H-indol-3-yl)ethyl]-amide
73. 3,5-dichloro-N-[2-(7-fluoro-2,4-dimethyl-1H-indol-3-yl)ethyl]benzamide
74. quinoline-4-carboxylic acid [2-(7-fluoro-2,4-dimethyl-1H-indol-3-yl)ethyl]-amide
75. quinoline-4-carboxylic acid [2-(7-fluoro-2,4-dimethyl-1H-indol-3-yl)ethyl]-amide
76. 4-hydroxy-2-phenyl-2H-pyrazole-3-carboxylic acid [2-(7-fluoro-2,4-dimethyl-1H-indol-3-yl)ethyl]amide
77. benzo[b]thiophene-3-carboxylic acid [2-(7-fluoro-2,4-dimethyl-1H-indol-3-yl)ethyl]amide
78. N-[2-(7-fluoro-2,4-dimethyl-1H-indol-3-yl)ethyl]-3-hydroxybenzamide
79. pyrazine-2-carboxylic acid [2-(7-fluoro-2,4-dimethyl-1H-indol-3-yl)ethyl]amide
80. furan-2-carboxylic acid [2-(7-fluoro-2,4-dimethyl-1H-indol-3-yl)ethyl]amide
81. quinoline-3-carboxylic acid [2-(7-fluoro-2,4-dimethyl-1H-indol-3-yl)ethyl]-amide
82. 2-chloro-N-[2-(7-fluoro-2,4-dimethyl-1H-indol-3-yl)ethyl]nicotinamide
83. 4-benzyloxy-N-[2-(7-fluoro-2,4-dimethyl-1H-indol-3-yl)ethyl]benzamide
84. 5-bromo-N-[2-(7-fluoro-2,4-dimethyl-1H-indol-3-yl)ethyl]nicotinamide
85. 1H-indole-3-carboxylic acid [2-(7-fluoro-2,4-dimethyl-1H-indol-3-yl)ethyl]-amide
86. 3-bromothiophene-2-carboxylic acid [2-(7-fluoro-2,4-dimethyl-1H-indol-3-yl)ethyl]amide
87. 2-chloro-N-[2-(7-fluoro-2,4-dimethyl-1H-indol-3-yl)ethyl]-3,4-dimethoxy-benzamide
88. 2-methylfuran-3-carboxylic acid [2-(7-fluoro-2,4-dimethyl-1H-indol-3-yl)ethyl]-amide
89. 1H-imidazole-4-carboxylic acid [2-(7-fluoro-2,4-dimethyl-1H-indol-3-yl)ethyl]-amide
90. 4-oxo-4,5,6,7-tetrahydrobenzo[b]thiophene-2-carboxylic acid [2-(7-fluoro-2,4-dimethyl-1H-indol-3-yl)ethyl]amide
91. 4′-bromobiphenyl-2-carboxylic acid [2-(7-fluoro-2,4-dimethyl-1H-indol-3-yl)ethyl]amide
92. 2-fluoro-N-[2-(7-fluoro-2,4-dimethyl-1H-indol-3-yl)ethyl]-6-iodobenzamide
93. 2,3-dihydrobenzofuran-7-carboxylic acid [2-(7-fluoro-2,4-dimethyl-1H-indol-3-yl)ethyl]amide
94. 3-fluoro-N-[2-(7-fluoro-2,4-dimethyl-1H-indol-3-yl)ethyl]-2-methylbenzamide
95. N-[2-(7-fluoro-2,4-dimethyl-1H-indol-3-yl)ethyl]-2,5-dimethylbenzamide
96. 5-acetylthiophene-2-carboxylic acid [2-(7-fluoro-2,4-dimethyl-1H-indol-3-yl)ethyl]amide
97. quinoline-8-carboxylic acid [2-(7-fluoro-2,4-dimethyl-1H-indol-3-yl)ethyl]-amide
98. 2-phenyl-2H-pyrazole-3-carboxylic acid [2-(7-fluoro-2,4-dimethyl-1H-indol-3-yl)ethyl]amide
99. 6-phenylpyrimidine-4-carboxylic acid [2-(7-fluoro-2,4-dimethyl-1H-indol-3-yl)ethyl]amide
100. 1-methyl-1H-indole-3-carboxylic acid [2-(7-fluoro-2,4-dimethyl-1H-indol-3-yl)ethyl]amide
101. 2-pyridin-3-ylthiazole-4-carboxylic acid [2-(7-fluoro-2,4-dimethyl-1H-indol-3-yl)ethyl]amide
102. 2,5-dimethyl-2H-pyrazole-3-carboxylic acid [2-(7-fluoro-2,4-dimethyl-1H-indol-3-yl)ethyl]amide
103. N-[2-(7-fluoro-2,4-dimethyl-1H-indol-3-yl)ethyl]-2-phenoxymethylbenzamide
104. 2,3-dihydrobenzofuran-5-carboxylic acid [2-(7-fluoro-2,4-dimethyl-1H-indol-3-yl)ethyl]amide
105. 1H-indole-6-carboxylic acid [2-(7-fluoro-2,4-dimethyl-1H-indol-3-yl)ethyl]-amide
106. 2-bromo-N-[2-(7-fluoro-2,4-dimethyl-1H-indol-3-yl)ethyl]-4,5-dimethoxy-benzamide
107. N-[2-(7-fluoro-2,4-dimethyl-1H-indol-3-yl)ethyl]-4-pyrrolidin-1-ylbenzamide
108. quinoline-5-carboxylic acid [2-(7-fluoro-2,4-dimethyl-1H-indol-3-yl)ethyl]-amide
109. 5-phenyl-1H-pyrazole-4-carboxylic acid [2-(7-fluoro-2,4-dimethyl-1H-indol-3-yl)ethyl]amide
110. pyridazine-4-carboxylic acid [2-(7-fluoro-2,4-dimethyl-1H-indol-3-yl)ethyl]-amide
111. 5-phenyl-2H-pyrazole-3-carboxylic acid [2-(7-fluoro-2,4-dimethyl-1H-indol-3-yl)ethyl]amide
112. N-[2-(7-fluoro-2,4-dimethyl-1H-indol-3-yl)ethyl]-5-pyrrol-1-ylnicotinamide
113. pyrimidine-5-carboxylic acid [2-(7-fluoro-2,4-dimethyl-1H-indol-3-yl)ethyl]-amide
114. benzo[b]thiophene-5-carboxylic acid [2-(7-fluoro-2,4-dimethyl-1H-indol-3-yl)ethyl]amide
115. N-[2-(7-fluoro-2,4-dimethyl-1H-indol-3-yl)ethyl]-3-piperidin-1-ylbenzamide
116. pyrazolo[1,5-a]pyridine-2-carboxylic acid [2-(7-fluoro-2,4-dimethyl-1H-indol-3-yl)ethyl]amide
117. quinoxaline-2-carboxylic acid [2-(7-fluoro-2,4-dimethyl-1H-indol-3-yl)ethyl]amide
118. 3-fluoro-N-[2-(7-fluoro-2,4-dimethyl-1H-indol-3-yl)ethyl]-2-methoxy-benzamide
119. 3-chloro-N-[2-(7-fluoro-2,4-dimethyl-1H-indol-3-yl)ethyl]-2-methylbenzamide
120. N-[2-(7-fluoro-2,4-dimethyl-1H-indol-3-yl)ethyl]-4-phenoxbenzamide
121. thiazole-4-carboxylic acid [2-(7-fluoro-2,4-dimethyl-1H-indol-3-yl)ethyl]-amide
122. 2-chloro-N-[2-(7-fluoro-2,4-dimethyl-1H-indol-3-yl)ethyl]-3-methylbenzamide
123. 3-chloro-2-fluoro-N-[2-(7-fluoro-2,4-dimethyl-1H-indol-3-yl)ethyl]-benzamide
124. 5-methoxy-1H-indole-2-carboxylic acid [2-(7-fluoro-2,4-dimethyl-1H-indol-3-yl)ethyl]amide
125. 5-fluoro-1H-indole-2-carboxylic acid [2-(7-fluoro-2,4-dimethyl-1H-indol-3-yl)ethyl]amide
126. 5-methyl-1H-indole-2-carboxylic acid [2-(7-fluoro-2,4-dimethyl-1H-indol-3-yl)ethyl]amide
127. 4-methyl-1H-indole-2-carboxylic acid [2-(7-fluoro-2,4-dimethyl-1H-indol-3-yl)ethyl]amide
128. 6-methoxy-1H-indole-2-carboxylic acid [2-(7-fluoro-2,4-dimethyl-1H-indol-3-yl)ethyl]amide
129. 4-methoxy-1H-indole-2-carboxylic acid [2-(7-fluoro-2,4-dimethyl-1H-indol-3-yl)ethyl]amide
130. 4-fluoro-1H-indole-2-carboxylic acid [2-(7-fluoro-2,4-dimethyl-1H-indol-3-yl)ethyl]amide
131. 7-methyl-1H-indole-2-carboxylic acid [2-(7-fluoro-2,4-dimethyl-1H-indol-3-yl)ethyl]amide
132. 6-fluoro-1H-indole-2-carboxylic acid [2-(7-fluoro-2,4-dimethyl-1H-indol-3-yl)ethyl]amide
133. 6-methyl-1H-indole-2-carboxylic acid [2-(7-fluoro-2,4-dimethyl-1H-indol-3-yl)ethyl]amide
134. 5-methoxy-1H-indole-2-carboxylic acid [2-(7-fluoro-1H-indol-3-yl)ethyl]-amide
135. 1H-indole-2-carboxylic acid [2-(7-fluoro-1H-indol-3-yl)ethyl]amide
136. 5-fluoro-1H-indole-2-carboxylic acid [2-(7-fluoro-1H-indol-3-yl)ethyl]amide
137. 5-methyl-1H-indole-2-carboxylic acid [2-(7-fluoro-1H-indol-3-yl)ethyl]-amide
138. 6-methoxy-1H-indole-2-carboxylic acid [2-(7-fluoro-1H-indol-3-yl)ethyl]-amide
139. 4-methyl-1H-indole-2-carboxylic acid [2-(7-fluoro-1H-indol-3-yl)ethyl]-amide
140. 4-dimethylamino-N-[2-(7-fluoro-1H-indol-3-yl)ethyl]benzamide
141. N-[2-(7-fluoro-1H-indol-3-yl)ethyl]-4-pyrrolidin-1-ylbenzamide
142. 1H-indole-6-carboxylic acid [2-(7-fluoro-1H-indol-3-yl)ethyl]amide
143. 4-methoxy-1H-indole-2-carboxylic acid [2-(7-fluoro-1H-indol-3-yl)ethyl]-amide
144. 4-fluoro-1H-indole-2-carboxylic acid [2-(7-fluoro-1H-indol-3-yl)ethyl]amide
145. 6-fluoro-1H-indole-2-carboxylic acid [2-(7-fluoro-1H-indol-3-yl)ethyl]amide
146. 6-methyl-1H-indole-2-carboxylic acid [2-(7-fluoro-1H-indol-3-yl)ethyl]-amide
147. 7-methyl-1H-indole-2-carboxylic acid [2-(7-fluoro-1H-indol-3-yl)ethyl]-amide
148. 5-bromo-2,3-dihydrobenzofuran-7-carboxylic acid [2-(7-fluoro-2,4-dimethyl-1H-indol-3-yl)ethyl]amide
149. 4-(1H-benzoimidazol-2-yl)-N-[2-(7-fluoro-1H-indol-3-yl)ethyl]benzamide
150. 4-(1H-benzoimidazol-2-yl)-N-[2-(7-fluoro-2,4-dimethyl-1H-indol-3-yl)ethyl]benzamide
151. N-[2-(4-cyano-7-fluoro-2-methyl-1H-indol-3-yl)ethyl]-3,4-dimethoxy-benzamide
152. [1,1′; 4′,1″]terphenyl-4-carboxylic acid [2-(7-fluoro-2,4-dimethyl-1H-indol-3-yl)ethyl]amide
153. 3′-methylbiphenyl-4-carboxylic acid [2-(7-fluoro-2,4-dimethyl-1H-indol-3-yl)ethyl]amide
154. 3′-fluoro-4′-methylbiphenyl-4-carboxylic acid [2-(7-fluoro-2,4-dimethyl-1H-indol-3-yl)ethyl]amide
155. 2′-fluoro-4′-methylbiphenyl-4-carboxylic acid [2-(7-fluoro-2,4-dimethyl-1H-indol-3-yl)ethyl]amide
156. 4′-hydroxymethylbiphenyl-4-carboxylic acid [2-(7-fluoro-2,4-dimethyl-1H-indol-3-yl)ethyl]amide
157. 4′-[2-(7-fluoro-2,4-dimethyl-1H-indol-3-yl)ethylcarbamoyl]-biphenyl-4-carboxylic acid
158. 4′-tert-butylbiphenyl-4-carboxylic acid [2-(7-fluoro-2,4-dimethyl-1H-indol-3-yl)ethyl]amide
159. 4′-chlorobiphenyl-4-carboxylic acid [2-(7-fluoro-2,4-dimethyl-1H-indol-3-yl)ethyl]amide
160. 3′,4′,5′-trimethoxybiphenyl-4-carboxylic acid [2-(7-fluoro-2,4-dimethyl-1H-indol-3-yl)ethyl]amide
161. 3′-trifluoromethoxybiphenyl-4-carboxylic acid [2-(7-fluoro-2,4-dimethyl-1H-indol-3-yl)ethyl]amide
162. 4′-trifluoromethoxybiphenyl-4-carboxylic acid [2-(7-fluoro-2,4-dimethyl-1H-indol-3-yl)ethyl]amide
163. 3′-hydroxybiphenyl-4-carboxylic acid [2-(7-fluoro-2,4-dimethyl-1H-indol-3-yl)ethyl]amide
164. 4′-methanesulfinylbiphenyl-4-carboxylic acid [2-(7-fluoro-2,4-dimethyl-1H-indol-3-yl)ethyl]amide
165. 3′-cyanomethylbiphenyl-4-carboxylic acid [2-(7-fluoro-2,4-dimethyl-1H-indol-3-yl)ethyl]amide
166. 2′-acetylaminobiphenyl-4-carboxylic acid [2-(7-fluoro-2,4-dimethyl-1H-indol-3-yl)ethyl]amide
167. 3′-fluoro-4′-methoxybiphenyl-4-carboxylic acid [2-(7-fluoro-2,4-dimethyl-1H-indol-3-yl)ethyl]amide
168. 3′-chloro-4′-fluorobiphenyl-4-carboxylic acid [2-(7-fluoro-2,4-dimethyl-1H-indol-3-yl)ethyl]amide
169. 3′,4′-difluorobiphenyl-4-carboxylic acid [2-(7-fluoro-2,4-dimethyl-1H-indol-3-yl)ethyl]amide
170. 3′,5′-difluorobiphenyl-4-carboxylic acid [2-(7-fluoro-2,4-dimethyl-1H-indol-3-yl)ethyl]amide
171. N-[2-(7-fluoro-2,4-dimethyl-1H-indol-3-yl)ethyl]-4-(5-hydroxymethyl-thiophen-2-yl)-benzamide
172. 3′-methanesulfonylbiphenyl-4-carboxylic acid [2-(7-fluoro-2,4-dimethyl-1H-indol-3-yl)ethyl]amide
173. 4-fluoro-4′-[2-(7-fluoro-2,4-dimethyl-1H-indol-3-yl)ethylcarbamoyl]-biphenyl-3-carboxylic acid
174. 4′-[2-(7-fluoro-2,4-dimethyl-1H-indol-3-yl)ethylcarbamoyl]-3-methoxy-biphenyl-4-carboxylic acid methyl ester
175. 5-fluoro-4′-[2-(7-fluoro-2,4-dimethyl-1H-indol-3-yl)ethylcarbamoyl]-biphenyl-3-carboxylic acid
176. 3-chlorobiphenyl-4,4′-dicarboxylic acid 4-amide 4′-{[2-(7-fluoro-2,4-dimethyl-1H-indol-3-yl)ethyl]amide}
177. 3-chlorobiphenyl-4,4′-dicarboxylic acid 4′-{[2-(7-fluoro-2,4-dimethyl-1H-indol-3-yl)ethyl]amide}-4-methylamide
178. 3′-dimethylsulfamoylbiphenyl-4-carboxylic acid [2-(7-fluoro-2,4-dimethyl-1H-indol-3-yl)ethyl]amide
179. biphenyl-3,4′-dicarboxylic acid 4′-{[2-(7-fluoro-2,4-dimethyl-1H-indol-3-yl)ethyl]amide}3-thiazol-2-ylamide
180. 4′-methylsulfamoylbiphenyl-4-carboxylic acid [2-(7-fluoro-2,4-dimethyl-1H-indol-3-yl)ethyl]amide
181. 4′-dimethylsulfamoylbiphenyl-4-carboxylic acid [2-(7-fluoro-2,4-dimethyl-1H-indol-3-yl)ethyl]amide
182. biphenyl-3,4′-dicarboxylic acid 3-diethylamide 4′-{[2-(7-fluoro-2,4-dimethyl-1H-indol-3-yl)ethyl]amide}
183. biphenyl-4,4′-dicarboxylic acid 4-diethylamide 4′-{[2-(7-fluoro-2,4-dimethyl-1H-indol-3-yl)ethyl]amide}
184. 4′-[2-(7-fluoro-2,4-dimethyl-1H-indol-3-yl)ethylcarbamoyl]biphenyl-3-carboxylic acid
185. biphenyl-4,4′-dicarboxylic acid 4-amide 4′-{[2-(7-fluoro-2,4-dimethyl-1H-indol-3-yl)ethyl]amide}
186. 3′-methylsulfamoylbiphenyl-4-carboxylic acid [2-(7-fluoro-2,4-dimethyl-1H-indol-3-yl)ethyl]amide
187. 3′-trifluoromethylbiphenyl-4-carboxylic acid [2-(7-fluoro-2,4-dimethyl-1H-indol-3-yl)ethyl]amide
188. 4′-methylsulfanylbiphenyl-4-carboxylic acid [2-(7-fluoro-2,4-dimethyl-1H-indol-3-yl)ethyl]amide
189. 4′-acetylbiphenyl-4-carboxylic acid [2-(7-fluoro-2,4-dimethyl-1H-indol-3-yl)ethyl]amide
190. 3′-aminobiphenyl-4-carboxylic acid [2-(7-fluoro-2,4-dimethyl-1H-indol-3-yl)ethyl]amide
191. 3′-acetylbiphenyl-4-carboxylic acid [2-(7-fluoro-2,4-dimethyl-1H-indol-3-yl)ethyl]amide
192. 3′-fluorobiphenyl-4-carboxylic acid [2-(7-fluoro-2,4-dimethyl-1H-indol-3-yl)ethyl]amide
193. [1,1′; 3′,1″]terphenyl-4-carboxylic acid [2-(7-fluoro-2,4-dimethyl-1H-indol-3-yl)ethyl]amide
194. 3′-hydroxymethylbiphenyl-4-carboxylic acid [2-(7-fluoro-2,4-dimethyl-1H-indol-3-yl)ethyl]amide
195. 4-benzo[b]thiophen-3-yl-N-[2-(7-fluoro-2,4-dimethyl-1H-indol-3-yl)ethyl]benzamide
196. 4′-trifluoromethylbiphenyl-4-carboxylic acid [2-(7-fluoro-2,4-dimethyl-1H-indol-3-yl)ethyl]amide
197. N-[2-(7-fluoro-2,4-dimethyl-1H-indol-3-yl)ethyl]-4-((E)-styryl)benzamide
198. N-[2-(7-fluoro-2,4-dimethyl-1H-indol-3-yl)ethyl]-4-quinolin-6-ylbenzamide
199. N-[2-(7-fluoro-2,4-dimethyl-1H-indol-3-yl)ethyl]-4-(6-methoxypyridin-3-yl)-benzamide
200. biphenyl-3,4′-dicarboxylic acid 4′-{[2-(7-fluoro-2,4-dimethyl-1H-indol-3-yl)ethyl]amide}3-methylamide
201. biphenyl-4,4′-dicarboxylic acid 4′-{[2-(7-fluoro-2,4-dimethyl-1H-indol-3-yl)ethyl]amide}-4-methylamide
202. 2′-fluorobiphenyl-4-carboxylic acid [2-(7-fluoro-2,4-dimethyl-1H-indol-3-yl)ethyl]amide
203. 2′-methylbiphenyl-4-carboxylic acid [2-(7-fluoro-2,4-dimethyl-1H-indol-3-yl)ethyl]amide
204. 3′-acetylaminobiphenyl-4-carboxylic acid [2-(7-fluoro-2,4-dimethyl-1H-indol-3-yl)ethyl]amide
205. 4-benzo[1,3]dioxol-5-yl-N-[2-(7-fluoro-2,4-dimethyl-1H-indol-3-yl)ethyl]-benzamide
206. 3′-cyanobiphenyl-4-carboxylic acid [2-(7-fluoro-2,4-dimethyl-1H-indol-3-yl)ethyl]amide
207. 4′-cyanomethylbiphenyl-4-carboxylic acid [2-(7-fluoro-2,4-dimethyl-1H-indol-3-yl)ethyl]amide
208. biphenyl-3,4′-dicarboxylic acid 3-amide 4′-{[2-(7-fluoro-2,4-dimethyl-1H-indol-3-yl)ethyl]amide}
209. 3′,5′-dimethylbiphenyl-4-carboxylic acid [2-(7-fluoro-2,4-dimethyl-1H-indol-3-yl)ethyl]amide
210. N-[2-(7-fluoro-2,4-dimethyl-1H-indol-3-yl)ethyl]-4-quinolin-3-ylbenzamide
211. 4′-acetylaminobiphenyl-4-carboxylic acid [2-(7-fluoro-2,4-dimethyl-1H-indol-3-yl)ethyl]amide
212. 3′-fluoro-5′-methoxybiphenyl-4-carboxylic acid [2-(7-fluoro-2,4-dimethyl-1H-indol-3-yl)ethyl]amide
213. 5-fluorobiphenyl-3,4′-dicarboxylic acid 4′-{[2-(7-fluoro-2,4-dimethyl-1H-indol-3-yl)ethyl]amide}3-methylamide
214. 3′-(acetylaminomethyl)biphenyl-4-carboxylic acid [2-(7-fluoro-2,4-dimethyl-1H-indol-3-yl)ethyl]amide
215. N-[2-(7-fluoro-2,4-dimethyl-1H-indol-3-yl)ethyl]-4-(1-methyl-1H-indol-5-yl)benzamide
216. N-[2-(7-fluoro-2,4-dimethyl-1H-indol-3-yl)ethyl]-4-(1-methyl-1H-indol-2-yl)benzamide
217. N-[2-(7-fluoro-2,4-dimethyl-1H-indol-3-yl)ethyl]-5-pyrrol-1-ylnicotinamide
218. N-[2-(4-cyano-7-fluoro-2-methyl-1H-indol-3-yl)ethyl]-3,4-dimethoxy-benzamide
219. 5-benzyloxy-1H-indole-2-carboxylic acid [2-(7-fluoro-2,4-dimethyl-1H-indol-3-yl)ethyl]amide
220. 5-hydroxy-1H-indole-2-carboxylic acid [2-(7-fluoro-2,4-dimethyl-1H-indol-3-yl)ethyl]amide
221. 5-methoxybenzofuran-2-carboxylic acid [2-(7-fluoro-2,4-dimethyl-1H-indol-3-yl)ethyl]amide
222. 6-hydroxy-1H-indole-2-carboxylic acid [2-(7-fluoro-2,4-dimethyl-1H-indol-3-yl)ethyl]amide
223. N-[2-(7-fluoro-2-methyl-4-trifluoromethyl-1H-indol-3-yl)ethyl]-3,4-dimethoxybenzamide
224. 3H-benzotriazole-5-carboxylic acid [2-(7-fluoro-2-methyl-4-trifluoromethyl-1H-indol-3-yl)ethyl]amide
225. 5-fluoro-1H-indole-2-carboxylic acid [2-(7-fluoro-2-methyl-4-trifluoromethyl-1H-indol-3-yl)ethyl]amide
226. quinoxaline-6-carboxylic acid [2-(7-fluoro-2-methyl-4-trifluoromethyl-1H-indol-3-yl)ethyl]amide
227. {2-[2-(7-fluoro-2,4-dimethyl-1H-indol-3-yl)ethylcarbamoyl]-1H-indol-5-yloxy}acetic acid methyl ester
228. {2-[2-(7-fluoro-2,4-dimethyl-1H-indol-3-yl)ethylcarbamoyl]-1H-indol-5-yloxy}butanoic acid ethyl ester
229. {2-[2-(7-fluoro-2,4-dimethyl-1H-indol-3-yl)ethylcarbamoyl]-1H-indol-5-yloxy}pentanoic acid ethyl ester
230. {2-[2-(7-fluoro-2,4-dimethyl-1H-indol-3-yl)ethylcarbamoyl]-1H-indol-5-yloxy}hexanoic acid ethyl ester
231. {2-[2-(7-fluoro-2,4-dimethyl-1H-indol-3-yl)ethylcarbamoyl]-1H-indol-6-yloxy}acetic acid methyl ester
232. {2-[2-(7-fluoro-2,4-dimethyl-1H-indol-3-yl)ethylcarbamoyl]-1H-indol-6-yloxy}butanoic acid ethyl ester
233. {2-[2-(7-fluoro-2,4-dimethyl-1H-indol-3-yl)ethylcarbamoyl]-1H-indol-6-yloxy}pentanoic acid ethyl ester
234. {2-[2-(7-fluoro-2,4-dimethyl-1H-indol-3-yl)ethylcarbamoyl]-1H-indol-6-yloxy}hexanoic acid ethyl ester
235. 6-bromopyridine-2-carboxylic acid [2-(7-fluoro-2,4-dimethyl-1H-indol-3-yl)ethyl]amide
236. 5-fluoro-1H-indole-2-carboxylic acid [2-(7-fluoro-2-methyl-1H-indol-3-yl)ethyl]amide
237. 4-bromopyridine-2-carboxylic acid [2-(7-fluoro-2,4-dimethyl-1H-indol-3-yl)ethyl]amide
238. 6-bromo-N-[2-(7-fluoro-2,4-dimethyl-1H-indol-3-yl)ethyl]nicotinamide
239. {3-[2-(7-fluoro-2,4-dimethyl-1H-indol-3-yl)ethylcarbamoyl]phenoxy}acetic acid methyl ester
240. 4-{3-[2-(7-fluoro-2,4-dimethyl-1H-indol-3-yl)ethylcarbamoyl]-phenoxy}butanoic acid ethyl ester
241. 5-{3-[2-(7-fluoro-2,4-dimethyl-1H-indol-3-yl)ethylcarbamoyl]-phenoxy}pentanoic acid ethyl ester
242. 2-[2-(7-fluoro-2,4-dimethyl-1H-indol-3-yl)ethylcarbamoyl]-1H-indol-5-yloxy}acetic acid
243. 4-{2-[2-(7-fluoro-2,4-dimethyl-1H-indol-3-yl)ethylcarbamoyl]-1H-indol-5-yloxy}butanoic acid
244. 5-{2-[2-(7-fluoro-2,4-dimethyl-1H-indol-3-yl)ethylcarbamoyl]-1H-indol-6-yloxy}pentanoic acid
245. 6-{3-[2-(7-fluoro-2,4-dimethyl-1H-indol-3-yl)ethylcarbamoyl]-phenoxy}hexanoic acid ethyl ester
246. 5-{2-[2-(7-fluoro-2,4-dimethyl-1H-indol-3-yl)ethylcarbamoyl]-1H-indol-5-yloxy}pentanoic acid
247. 6-{2-[2-(7-fluoro-2,4-dimethyl-1H-indol-3-yl)ethylcarbamoyl]-1H-indol-5-yloxy}hexanoic acid
248. 4-{2-[2-(7-fluoro-2,4-dimethyl-1H-indol-3-yl)ethylcarbamoyl]-1H-indol-6-yloxy}butanoic acid
249. 6-{2-[2-(7-fluoro-2,4-dimethyl-1H-indol-3-yl)ethylcarbamoyl]-1H-indol-6-yloxy}hexanoic acid
250. 4-{3-[2-(7-fluoro-2,4-dimethyl-1H-indol-3-yl)ethylcarbamoyl]-phenoxy}butanoic acid
251. {2-[2-(7-fluoro-2,4-dimethyl-1H-indol-3-yl)ethylcarbamoyl]-1H-indol-6-yloxy}acetic acid
252. 4-{4-[2-(7-fluoro-2,4-dimethyl-1H-indol-3-yl)ethylcarbamoyl]-phenoxy}butanoic acid ethyl ester
253. 5-{4-[2-(7-fluoro-2,4-dimethyl-1H-indol-3-yl)ethylcarbamoyl]-phenoxy}pentanoic acid ethyl ester
254. 6-{4-[2-(7-fluoro-2,4-dimethyl-1H-indol-3-yl)ethylcarbamoyl]-phenoxy}hexanoic acid ethyl ester
255. 6-{3-[2-(7-fluoro-2,4-dimethyl-1H-indol-3-yl)ethylcarbamoyl]-phenoxy}hexanoic acid
256. 4-{4-[2-(7-fluoro-2,4-dimethyl-1H-indol-3-yl)ethylcarbamoyl]-phenoxy}butanoic acid
257. 5-{4-[2-(7-fluoro-2,4-dimethyl-1H-indol-3-yl)ethylcarbamoyl]-phenoxy}pentanoic acid
258. 6-{4-[2-(7-fluoro-2,4-dimethyl-1H-indol-3-yl)ethylcarbamoyl]-phenoxy}hexanoic acid
259. 5-{3-[2-(7-fluoro-2,4-dimethyl-1H-indol-3-yl)ethylcarbamoyl]-phenoxy}pentanoic acid
260. 2-bromo-N-[2-(7-fluoro-2,4-dimethyl-1H-indol-3-yl)ethyl]isonicotinamide
261. {4-[2-(7-fluoro-2,4-dimethyl-1H-indol-3-yl)ethylcarbamoyl]phenoxy}acetic acid
262. 5-bromo-1H-indole-2-carboxylic acid [2-(7-fluoro-2,4-dimethyl-1H-indol-3-yl)ethyl]amide
263. 6-bromo-1H-indole-2-carboxylic acid [2-(7-fluoro-2,4-dimethyl-1H-indol-3-yl)ethyl]amide
264. 5-bromopyridine-2-carboxylic acid [2-(7-fluoro-2,4-dimethyl-1H-indol-3-yl)ethyl]amide
265. 6-(3-carbamoylphenyl)pyridine-2-carboxylic acid [2-(7-fluoro-2,4-dimethyl-1H-indol-3-yl)ethyl]amide
266. 6-(3-methylcarbamoylphenyl)pyridine-2-carboxylic acid [2-(7-fluoro-2,4-dimethyl-1H-indol-3-yl)ethyl]amide
267. 6-(3-hydroxyphenyl)pyridine-2-carboxylic acid [2-(7-fluoro-2,4-dimethyl-1H-indol-3-yl)ethyl]amide
268. 4-(3-carbamoylphenyl)pyridine-2-carboxylic acid [2-(7-fluoro-2,4-dimethyl-1H-indol-3-yl)ethyl]amide
269. 4-(3-methylcarbamoylphenyl)pyridine-2-carboxylic acid [2-(7-fluoro-2,4-dimethyl-1H-indol-3-yl)ethyl]amide
270. 4-(3-hydroxyphenyl)pyridine-2-carboxylic acid [2-(7-fluoro-2,4-dimethyl-1H-indol-3-yl)ethyl]amide
271. 4-(4-methylcarbamoylphenyl)pyridine-2-carboxylic acid [2-(7-fluoro-2,4-dimethyl-1H-indol-3-yl)ethyl]amide
272. 5-chloro-1H-indole-2-carboxylic acid [2-(7-fluoro-2,4-dimethyl-1H-indol-3-yl)ethyl]amide
273. benzofuran-2-carboxylic acid [2-(7-fluoro-2,4-dimethyl-1H-indol-3-yl)ethyl]amide
274. 5-chloro-benzofuran-2-carboxylic acid [2-(7-fluoro-2,4-dimethyl-1H-indol-3-yl)ethyl]amide
275. 4-bromo-3-fluoro-N-[2-(7-fluoro-2,4-dimethyl-1H-indol-3-yl)ethyl]-benzamide
276. 4-chloro-4′-[2-(7-fluoro-2,4-dimethyl-1H-indol-3-yl)ethylcarbamoyl]-biphenyl-3-carboxylic acid
277. {3-[2-(7-fluoro-2,4-dimethyl-1H-indol-3-yl)ethylcarbamoyl]phenoxy}acetic acid
278. 5-(3-carbamoylphenyl)pyridine-2-carboxylic acid [2-(7-fluoro-2,4-dimethyl-1H-indol-3-yl)ethyl]amide
279. 5-(3-methylcarbamoylphenyl)pyridine-2-carboxylic acid [2-(7-fluoro-2,4-dimethyl-1H-indol-3-yl)ethyl]amide
280. 5-(3-hydroxyphenyl)pyridine-2-carboxylic acid [2-(7-fluoro-2,4-dimethyl-1H-indol-3-yl)ethyl]amide
281. 6-(3-carbamoylphenyl)-N-[2-(7-fluoro-2,4-dimethyl-1H-indol-3-yl)ethyl]-nicotinamide
282. N-[2-(7-fluoro-2,4-dimethyl-1H-indol-3-yl)ethyl]-6-(3-methylcarbamoyl-phenyl)nicotinamide
283. N-[2-(7-fluoro-2,4-dimethyl-1H-indol-3-yl)ethyl]-6-(3-hydroxyphenyl)-nicotinamide
284. N-[2-(7-fluoro-2,4-dimethyl-1H-indol-3-yl)ethyl]-6-(4-methylcarbamoyl-phenyl)nicotinamide
285. 5-(3-carbamoylphenyl)-N-[2-(7-fluoro-2,4-dimethyl-1H-indol-3-yl)ethyl]-nicotinamide
286. N-[2-(7-fluoro-2,4-dimethyl-1H-indol-3-yl)ethyl]-5-(3-methylcarbamoyl-phenyl)nicotinamide
287. N-[2-(7-fluoro-2,4-dimethyl-1H-indol-3-yl)ethyl]-5-(3-hydroxyphenyl)-nicotinamide
288. N-[2-(7-fluoro-2,4-dimethyl-1H-indol-3-yl)ethyl]-5-(4-methylcarbamoyl-phenyl)nicotinamide
289. 2-(3-carbamoylphenyl)-N-[2-(7-fluoro-2,4-dimethyl-1H-indol-3-yl)ethyl]-isonicotinamide
290. N-[2-(7-fluoro-2,4-dimethyl-1H-indol-3-yl)ethyl]-2-(3-methylcarbamoyl-phenyl)isonicotinamide
291. N-[2-(7-fluoro-2,4-dimethyl-1H-indol-3-yl)ethyl]-2-(3-hydroxyphenyl)-isonicotinamide
292. N-[2-(7-fluoro-2,4-dimethyl-1H-indol-3-yl)ethyl]-2-(4-methylcarbamoyl-phenyl)isonicotinamide
293. benzo[b]thiophene-2-carboxylic acid [2-(7-fluoro-2,4-dimethyl-1H-indol-3-yl)ethyl]amide
294. quinoline-2-carboxylic acid [2-(7-fluoro-2,4-dimethyl-1H-indol-3-yl)ethyl]amide
295. [1,8]naphthyridine-2-carboxylic acid [2-(7-fluoro-2,4-dimethyl-1H-indol-3-yl)ethyl]amide
296. isoquinoline-3-carboxylic acid [2-(7-fluoro-2,4-dimethyl-1H-indol-3-yl)ethyl]amide
297. 5-pyridin-2-yl-thiophene-2-carboxylic acid [2-(7-fluoro-2,4-dimethyl-1H-indol-3-yl)ethyl]amide
298. 5-trifluoromethoxy-1H-indole-2-carboxylic acid [2-(7-fluoro-2,4-dimethyl-1H-indol-3-yl)ethyl]amide
299. 5-fluoro-1H-indole-2-carboxylic acid [2-(4,7-difluoro-2-methyl-1H-indol-3-yl)ethyl]amide
300. biphenyl-3,4′-dicarboxylic acid 4′-{[2-(4,7-difluoro-2-methyl-1H-indol-3-yl)ethyl]amide}3-methylamide
301. 6-(3-trifluoromethoxyphenyl)-1H-indole-2-carboxylic acid [2-(7-fluoro-2,4-dimethyl-1H-indol-3-yl)ethyl]amide
302. 5-(4-methylcarbamoylphenyl)pyridine-2-carboxylic acid [2-(7-fluoro-2,4-dimethyl-1H-indol-3-yl)ethyl]amide
303. 4′-methoxybiphenyl-4-carboxylic acid [2-(7-fluoro-2,4-dimethyl-1H-indol-3-yl)ethyl]amide
304. 4′-[2-(7-fluoro-2,4-dimethyl-1H-indol-3-yl)ethylcarbamoyl]-3-methoxybiphenyl-4-carboxylic acid
305. 4′-methoxybiphenyl-3-carboxylic acid [2-(7-fluoro-2,4-dimethyl-1H-indol-3-yl)ethyl]amide
306. 4-(4-methylcarbamoylphenyl)thiophene-2-carboxylic acid [2-(7-fluoro-2,4-dimethyl-1H-indol-3-yl)ethyl]amide
307. 4-(3-methylcarbamoylphenyl)thiophene-2-carboxylic acid [2-(7-fluoro-2,4-dimethyl-1H-indol-3-yl)ethyl]amide
308. 4-(3-methylcarbamoylphenyl)thiophene-2-carboxylic acid [2-(7-fluoro-2,4-dimethyl-1H-indol-3-yl)ethyl]amide
309. 4-(3-hydroxyphenyl)thiophene-2-carboxylic acid [2-(7-fluoro-2,4-dimethyl-1H-indol-3-yl)ethyl]amide
310. 4-(3-carbamoylphenyl)thiophene-2-carboxylic acid [2-(7-fluoro-2,4-dimethyl-1H-indol-3-yl)ethyl]amide
311. 4-bromothiophene-2-carboxylic acid [2-(7-fluoro-2,4-dimethyl-1H-indol-3-yl)ethyl]amide
312. 2-fluorobiphenyl-4,4′-dicarboxylic acid 4-{[2-(7-fluoro-2,4-dimethyl-1H-indol-3-yl)ethyl]amide}-4′-methylamide
313. 2′-fluorobiphenyl-3,4′-dicarboxylic acid 3-amide 4′-{[2-(7-fluoro-2,4-dimethyl-1H-indol-3-yl)ethyl]amide}
314. 2-fluoro-3′-hydroxybiphenyl-4-carboxylic acid [2-(7-fluoro-2,4-dimethyl-1H-indol-3-yl)ethyl]amide
315. 2′-fluorobiphenyl-3,4′-dicarboxylic acid 4′-{[2-(7-fluoro-2,4-dimethyl-1H-indol-3-yl)ethyl]amide}3-methylamide
316. 5-(3-methylcarbamoylphenyl)-1H-indole-2-carboxylic acid [2-(7-fluoro-2,4-dimethyl-1H-indol-3-yl)ethyl]amide
317. 5-bromothiophene-2-carboxylic acid [2-(7-fluoro-2,4-dimethyl-1H-indol-3-yl)ethyl]amide
318. 3′-hydroxybiphenyl-4-carboxylic acid [2-(7-fluoro-2-methyl-1H-indol-3-yl)ethyl]amide
319. biphenyl-4,4′-dicarboxylic acid 4′-{[2-(7-fluoro-2-methyl-1H-indol-3-yl)ethyl]amide}4-methylamide
320. 4′-[2-(7-fluoro-2,4-dimethyl-1H-indol-3-yl)ethylcarbamoyl]-3-hydroxybiphenyl-4-carboxylic acid methyl ester
321. N-[2-(7-fluoro-2-methyl-1H-indol-3-yl)ethyl]-6-(3-methylcarbamoylphenyl)-nicotinamide
322. biphenyl-3,4′-dicarboxylic acid 4′-{[2-(7-fluoro-2-methyl-1H-indol-3-yl)ethyl]amide}3-methylamide
323. 5-(3-carbamoylphenyl)-1H-indole-2-carboxylic acid [2-(7-fluoro-2,4-dimethyl-1H-indol-3-yl)ethyl]amide
324. 5-(3-hydroxyphenyl)-1H-indole-2-carboxylic acid [2-(7-fluoro-2,4-dimethyl-1H-indol-3-yl)ethyl]amide
325. 5-(4-methylcarbamoylphenyl)-1H-indole-2-carboxylic acid [2-(7-fluoro-2,4-dimethyl-1H-indol-3-yl)ethyl]amide
326. 5-(3-methylcarbamoylphenyl)thiophene-2-carboxylic acid [2-(7-fluoro-2,4-dimethyl-1H-indol-3-yl)ethyl]amide
327. 5-(3-carbamoylphenyl)thiophene-2-carboxylic acid [2-(7-fluoro-2,4-dimethyl-1H-indol-3-yl)ethyl]amide
328. 5-(3-hydroxyphenyl)thiophene-2-carboxylic acid [2-(7-fluoro-2,4-dimethyl-1H-indol-3-yl)ethyl]amide
329. 5-(4-methylcarbamoylphenyl)thiophene-2-carboxylic acid [2-(7-fluoro-2,4-dimethyl-1H-indol-3-yl)ethyl]amide
330. 6-(3-methylcarbamoylphenyl)-1H-indole-2-carboxylic acid [2-(7-fluoro-2,4-dimethyl-1H-indol-3-yl)ethyl]amide
331. 6-(3-carbamoylphenyl)-1H-indole-2-carboxylic acid [2-(7-fluoro-2,4-dimethyl-1H-indol-3-yl)ethyl]amide
332. 6-(3-hydroxyphenyl)-1H-indole-2-carboxylic acid [2-(7-fluoro-2,4-dimethyl-1H-indol-3-yl)ethyl]amide
333. 6-(4-methylcarbamoylphenyl)-1H-indole-2-carboxylic acid [2-(7-fluoro-2,4-dimethyl-1H-indol-3-yl)ethyl]amide
334. 3′-cyano-2′-fluorobiphenyl-4-carboxylic acid [2-(7-fluoro-2,4-dimethyl-1H-indol-3-yl)ethyl]amide
335. 5-chloro-1H-indole-2-carboxylic acid [2-(7-fluoro-2-methyl-1H-indol-3-yl)ethyl]amide
336. 5-chloro-1H-indole-2-carboxylic acid [2-(4,7-difluoro-2-methyl-1H-indol-3-yl)ethyl]amide
337. 5-chloro-1H-indole-2-carboxylic acid [2-(4-chloro-7-fluoro-2-methyl-1H-indol-3-yl)ethyl]amide
338. 5-trifluoromethyl-1H-indole-2-carboxylic acid [2-(7-fluoro-2,4-dimethyl-1H-indol-3-yl)ethyl]amide
339. 6-methanesulfonyl-1H-indole-2-carboxylic acid [2-(7-fluoro-2,4-dimethyl-1H-indol-3-yl)ethyl]amide
340. 7-chloro-1H-indole-2-carboxylic acid [2-(7-fluoro-2,4-dimethyl-1H-indol-3-yl)ethyl]amide
341. 4-chloro-1H-indole-2-carboxylic acid [2-(7-fluoro-2,4-dimethyl-1H-indol-3-yl)ethyl]amide
342. 6-chloro-1H-indole-2-carboxylic acid [2-(7-fluoro-2,4-dimethyl-1H-indol-3-yl)ethyl]amide
343. 7-fluoro-1H-indole-2-carboxylic acid [2-(7-fluoro-2,4-dimethyl-1H-indol-3-yl)ethyl]amide
344. 5-bromo-1H-indole-2-carboxylic acid [2-(7-fluoro-4-methyl-2-trifluoromethyl-1H-indol-3-yl)ethyl]amide
345. 5-trifluoromethyl-1H-indole-2-carboxylic acid [2-(7-fluoro-4-methyl-2-trifluoromethyl-1H-indol-3-yl)ethyl]amide
346. 5-chloro-1H-indole-2-carboxylic acid [2-(7-fluoro-4-methyl-2-trifluoromethyl-1H-indol-3-yl)ethyl]amide
347. 5-trifluoromethoxy-1H-indole-2-carboxylic acid [2-(7-fluoro-4-methyl-2-trifluoromethyl-1H-indol-3-yl)ethyl]amide
348. N-[2-(7-fluoro-4-methyl-2-trifluoromethyl-1H-indol-3-yl)ethyl]-3,4-dimethoxybenzamide
349. biphenyl-3,4′-dicarboxylic acid 4′-{[2-(4-chloro-7-fluoro-2-methyl-1H-indol-3-yl)ethyl]amide}3-methylamide
350. 5-fluoro-1H-indole-2-carboxylic acid [2-(4-chloro-7-fluoro-2-methyl-1H-indol-3-yl)ethyl]amide
351. 5-trifluoromethoxy-1H-indole-2-carboxylic acid [2-(4-chloro-7-fluoro-2-methyl-1H-indol-3-yl)ethyl]amide
352. 4′-[2-(7-fluoro-2,4-dimethyl-1H-indol-3-yl)ethylcarbamoyl]-3-hydroxybiphenyl-4-carboxylic acid
353. 4-bromo-N-[2-(4-fluoro-2,7-dimethyl-1H-indol-3-yl)ethyl]benzamide
354. 4,5,6,7-tetrahydrobenzo[b]thiophene-2-carboxylic acid [2-(7-fluoro-2,4-dimethyl-1H-indol-3-yl)ethyl]amide
355. N-[2-(7-fluoro-2,4-dimethyl-1H-indol-3-yl)ethyl]-4-hydroxybenzamide
356. 3′-(2,5-dioxoimidazolidin-4-yl)biphenyl-4-carboxylic acid [2-(7-fluoro-2,4-dimethyl-1H-indol-3-yl)ethyl]amide
357. 3-bromo-N-[2-(7-fluoro-2,4-dimethyl-1H-indol-3-yl)ethyl]benzamide
358. 5-bromobenzo[b]thiophene-2-carboxylic acid [2-(7-fluoro-2,4-dimethyl-1H-indol-3-yl)ethyl]amide
359. 6-bromobenzo[b]thiophene-2-carboxylic acid [2-(7-fluoro-2,4-dimethyl-1H-indol-3-yl)ethyl]amide
360. 6-trifluoromethylbenzo[b]thiophene-2-carboxylic acid [2-(7-fluoro-2,4-dimethyl-1H-indol-3-yl)ethyl]amide
361. 6-trifluoromethoxybenzo[b]thiophene-2-carboxylic acid [2-(7-fluoro-2,4-dimethyl-1H-indol-3-yl)ethyl]amide
362. 5-trifluoromethoxybenzo[b]thiophene-2-carboxylic acid [2-(7-fluoro-2,4-dimethyl-1H-indol-3-yl)ethyl]amide
363. benzothiazole-2-carboxylic acid [2-(7-fluoro-2,4-dimethyl-1H-indol-3-yl)ethyl]amide
364. 5-chlorobenzothiazole-2-carboxylic acid [2-(7-fluoro-2,4-dimethyl-1H-indol-3-yl)ethyl]amide
365. 6-chlorobenzothiazole-2-carboxylic acid [2-(7-fluoro-2,4-dimethyl-1H-indol-3-yl)ethyl]amide
366. 5-fluorobenzothiazole-2-carboxylic acid [2-(7-fluoro-2,4-dimethyl-1H-indol-3-yl)ethyl]amide
367. 6-fluorobenzothiazole-2-carboxylic acid [2-(7-fluoro-2,4-dimethyl-1H-indol-3-yl)ethyl]amide
368. 5-trifluoromethylbenzothiazole-2-carboxylic acid [2-(7-fluoro-2,4-dimethyl-1H-indol-3-yl)ethyl]amide
369. 6-trifluoromethylbenzothiazole-2-carboxylic acid [2-(7-fluoro-2,4-dimethyl-1H-indol-3-yl)ethyl]amide
370. 5-trifluoromethoxybenzothiazole-2-carboxylic acid [2-(7-fluoro-2,4-dimethyl-1H-indol-3-yl)ethyl]amide
371. 6-trifluoromethoxybenzothiazole-2-carboxylic acid [2-(7-fluoro-2,4-dimethyl-1H-indol-3-yl)ethyl]amide
372. 5-bromobenzothiazole-2-carboxylic acid [2-(7-fluoro-2,4-dimethyl-1H-indol-3-yl)ethyl]amide
373. 6-bromobenzothiazole-2-carboxylic acid [2-(7-fluoro-2,4-dimethyl-1H-indol-3-yl)ethyl]amide
374. benzoxazole-2-carboxylic acid [2-(7-fluoro-2,4-dimethyl-1H-indol-3-yl)ethyl]amide
375. 5-chlorobenzoxazole-2-carboxylic acid [2-(7-fluoro-2,4-dimethyl-1H-indol-3-yl)ethyl]amide
376. 6-chlorobenzoxazole-2-carboxylic acid [2-(7-fluoro-2,4-dimethyl-1H-indol-3-yl)ethyl]amide
377. 5-fluorobenzoxazole-2-carboxylic acid [2-(7-fluoro-2,4-dimethyl-1H-indol-3-yl)ethyl]amide
378. 6-fluorobenzoxazole-2-carboxylic acid [2-(7-fluoro-2,4-dimethyl-1H-indol-3-yl)ethyl]amide
379. 5-trifluoromethylbenzoxazole-2-carboxylic acid [2-(7-fluoro-2,4-dimethyl-1H-indol-3-yl)ethyl]amide
380. 6-trifluoromethylbenzoxazole-2-carboxylic acid [2-(7-fluoro-2,4-dimethyl-1H-indol-3-yl)ethyl]amide
381. 5-trifluoromethoxybenzoxazole-2-carboxylic acid [2-(7-fluoro-2,4-dimethyl-1H-indol-3-yl)ethyl]amide
382. 6-trifluoromethoxybenzoxazole-2-carboxylic acid [2-(7-fluoro-2,4-dimethyl-1H-indol-3-yl)ethyl]amide
383. 5-bromobenzoxazole-2-carboxylic acid [2-(7-fluoro-2,4-dimethyl-1H-indol-3-yl)ethyl]amide
384. 6-bromobenzoxazole-2-carboxylic acid [2-(7-fluoro-2,4-dimethyl-1H-indol-3-yl)ethyl]amide
385. 1H-benzimidazole-2-carboxylic acid [2-(7-fluoro-2,4-dimethyl-1H-indol-3-yl)ethyl]amide
386. 5-chloro-1H-benzimidazole-2-carboxylic acid [2-(7-fluoro-2,4-dimethyl-1H-indol-3-yl)ethyl]amide
387. 6-chloro-1H-benzimidazole-2-carboxylic acid [2-(7-fluoro-2,4-dimethyl-1H-indol-3-yl)ethyl]amide
388. 5-fluoro-1H-benzimidazole-2-carboxylic acid [2-(7-fluoro-2,4-dimethyl-1H-indol-3-yl)ethyl]amide
389. 6-fluoro-1H-benzimidazole-2-carboxylic acid [2-(7-fluoro-2,4-dimethyl-1H-indol-3-yl)ethyl]amide
390. 5-trifluoromethyl-1H-benzimidazole-2-carboxylic acid [2-(7-fluoro-2,4-dimethyl-1H-indol-3-yl)ethyl]amide
391. 6-trifluoromethyl-1H-benzimidazole-2-carboxylic acid [2-(7-fluoro-2,4-dimethyl-1H-indol-3-yl)ethyl]amide
392. 5-trifluoromethoxy-1H-benzimidazole-2-carboxylic acid [2-(7-fluoro-2,4-dimethyl-1H-indol-3-yl)ethyl]amide
393. 6-trifluoromethoxy-1H-benzimidazole-2-carboxylic acid [2-(7-fluoro-2,4-dimethyl-1H-indol-3-yl)ethyl]amide
394. 5-bromo-1H-benzimidazole-2-carboxylic acid [2-(7-fluoro-2,4-dimethyl-1H-indol-3-yl)ethyl]amide
395. 6-bromo-1H-benzimidazole-2-carboxylic acid [2-(7-fluoro-2,4-dimethyl-1H-indol-3-yl)ethyl]amide
396. 5-trifluoromethylsulfanyl-1H-indole-2-carboxylic acid [2-(7-fluoro-2,4-dimethyl-1H-indol-3-yl)ethyl]amide
397. 5,6-dichloro-1H-indole-2-carboxylic acid [2-(7-fluoro-2,4-dimethyl-1H-indol-3-yl)ethyl]amide
398. 5-chloro-6-fluoro-1H-indole-2-carboxylic acid [2-(7-fluoro-2,4-dimethyl-1H-indol-3-yl)ethyl]amide
399. 5-fluoro-6-chloro-1H-indole-2-carboxylic acid [2-(7-fluoro-2,4-dimethyl-1H-indol-3-yl)ethyl]amide
400. 5,6-difluoro-1H-indole-2-carboxylic acid [2-(7-fluoro-2,4-dimethyl-1H-indol-3-yl)ethyl]amide
401. 4,6-dichloro-1H-indole-2-carboxylic acid [2-(7-fluoro-2,4-dimethyl-1H-indol-3-yl)ethyl]amide
402. 4,6-difluoro-1H-indole-2-carboxylic acid [2-(7-fluoro-2,4-dimethyl-1H-indol-3-yl)ethyl]amide
403. 5-acetylamino-1H-indole-2-carboxylic acid [2-(7-fluoro-2,4-dimethyl-1H-indol-3-yl)ethyl]amide
404. 6-acetylamino-1H-indole-2-carboxylic acid [2-(7-fluoro-2,4-dimethyl-1H-indol-3-yl)ethyl]amide
405. 5-(2,2-dimethylpropionylamino)-1H-indole-2-carboxylic acid [2-(7-fluoro-2,4-dimethyl-1H-indol-3-yl)ethyl]amide
406. 6-(2,2-dimethylpropionylamino)-1H-indole-2-carboxylic acid [2-(7-fluoro-2,4-dimethyl-1H-indol-3-yl)ethyl]amide
407. 5-trifluoroacetylamino-1H-indole-2-carboxylic acid [2-(7-fluoro-2,4-dimethyl-1H-indol-3-yl)ethyl]amide
408. 6-trifluoroacetylamino-1H-indole-2-carboxylic acid [2-(7-fluoro-2,4-dimethyl-1H-indol-3-yl)ethyl]amide
409. 5-isopropyloxy-1H-indole-2-carboxylic acid [2-(7-fluoro-2,4-dimethyl-1H-indol-3-yl)ethyl]amide
410. 6-isopropyloxy-1H-indole-2-carboxylic acid [2-(7-fluoro-2,4-dimethyl-1H-indol-3-yl)ethyl]amide
411. 5-isopropyl-1H-indole-2-carboxylic acid [2-(7-fluoro-2,4-dimethyl-1H-indol-3-yl)ethyl]amide
412. 6-trifluoromethyl-1H-indole-2-carboxylic acid [2-(7-fluoro-2,4-dimethyl-1H-indol-3-yl)ethyl]amide
413. 4,5,6,7-tetrahydro-1H-indole-2-carboxylic acid [2-(7-fluoro-2,4-dimethyl-1H-indol-3-yl)ethyl]amide
414. 3-methyl-1H-indole-2-carboxylic acid [2-(7-fluoro-2,4-dimethyl-1H-indol-3-yl)ethyl]amide
415. 5-trifluoromethylbenzo[b]thiophene-2-carboxylic acid [2-(7-fluoro-2,4-dimethyl-1H-indol-3-yl)ethyl]amide
416. 5-fluorobenzo[b]thiophene-2-carboxylic acid [2-(7-fluoro-2,4-dimethyl-1H-indol-3-yl)ethyl]amide
417. 5-amino-1H-indole-2-carboxylic acid [2-(7-fluoro-2,4-dimethyl-1H-indol-3-yl)ethyl]amide
418. 6-amino-1H-indole-2-carboxylic acid [2-(7-fluoro-2,4-dimethyl-1H-indol-3-yl)ethyl]amide
419. 6-dimethylcarbamoylmethoxy-1H-indole-2-carboxylic acid [2-(7-fluoro-2,4-dimethyl-1H-indol-3-yl)ethyl]amide
420. 5-dimethylcarbamoylmethoxy-1H-indole-2-carboxylic acid [2-(7-fluoro-2,4-dimethyl-1H-indol-3-yl)ethyl]amide
421. 6-methylcarbamoylmethoxy-1H-indole-2-carboxylic acid [2-(7-fluoro-2,4-dimethyl-1H-indol-3-yl)ethyl]amide
422. 5-methylcarbamoylmethoxy-1H-indole-2-carboxylic acid [2-(7-fluoro-2,4-dimethyl-1H-indol-3-yl)ethyl]amide
423. 6-carbamoylmethoxy-1H-indole-2-carboxylic acid [2-(7-fluoro-2,4-dimethyl-1H-indol-3-yl)ethyl]amide
424. 5-carbamoylmethoxy-1H-indole-2-carboxylic acid [2-(7-fluoro-2,4-dimethyl-1H-indol-3-yl)ethyl]amide
425. 6-tert-butyl-1H-indole-2-carboxylic acid [2-(7-fluoro-2,4-dimethyl-1H-indol-3-yl)ethyl]amide
426. 5-tert-butyl-1H-indole-2-carboxylic acid [2-(7-fluoro-2,4-dimethyl-1H-indol-3-yl)ethyl]amide

The present invention relates to the use of the compounds of the invention for manufacturing medicaments which comprise at least one of the compounds of formula I.

The present invention likewise relates to medicaments which comprise the compounds of the invention with suitable formulating substances and carriers.

Compared with known prostaglandin E₂ ligands, the novel EP₂ agonists and antagonists are distinguished by greater selectivity and stability.

The present invention relates to medicaments for the treatment and prophylaxis of disorders which include fertility impairments, infectious disorders, cancer, viral infections, cardiovascular disorders, elevated intraocular pressure, glaucoma, skeletal system disorders, angiogenetic disorders, uterine contraction impairments, pain, neuroinflammatory disorders, immunomodulatory infections and nephrological disorders.

Fertility impairments mean the disorders which lead to no ovulation taking place, that no nidation of a fertilized oocyte occurs and no decidualization takes place, infectious disorders mean disorders caused by unicellular parasites, cancer means solid tumors and leukemia, viral infections mean for example cytomegalievirus infections, hepatitis, hepatitis B and C and HIV disorders, immunomodulatory infections mean for example avian influenza, cardiovascular disorders mean ischemic reperfusion disorder, stenoses, arterioscleroses and restenoses, angiogenetic disorders mean for example endometriosis and fibrosis, elevated intraocular pressure means glaucoma, uterine contraction impairments mean for example painful menstruation, skeletal system disorders mean osteoporosis, neuroinflammatory disorders mean multiple sclerosis, Alzheimer's disease, pain and nephrological disorders mean glomerulonephritis.

The present invention likewise relates to medicaments for the treatment and prophylaxis of the disorders detailed above, which comprise at least one compound of the general formula I, and medicaments with suitable formulating substances and carriers.

For the compounds of the invention to be used as medicaments they are brought into the form of a pharmaceutical product which, besides the active ingredient, comprises inert organic or inorganic pharmaceutical carrier materials which are suitable for enteral or parenteral administration, such as, for example, water, gelatin, gum arabic, lactose, starch, magnesium stearate, talc, vegetable oils, polyalkylene glycols etc. The pharmaceutical products may be in solid form, for example as tablets, coated tablets, suppositories, capsules, in semisolid form, for example as ointments, creams, gels, suppositiories, emulsions or in liquid form, for example as solutions, suspensions or emulsions.

They comprise where appropriate excipients which are intended to act for example as fillers, binders, disintegrants, lubricants, solvents, solubilizers, masking flavors, colorant, emulsifiers. Examples of types of excipients for the purpose of the invention are saccharides (mono-, di-, tri-, oligo-, and/or polysaccharides), fats, waxes, oils, hydrocarbons, anionic, nonionic, cationic natural, synthetic or semisynthetic surfactants. They additionally comprise where appropriate excipients such as preservatives, stabilizers, wetting agents or emulsifiers; salts to modify the osmotic pressure or buffers. The present invention likewise relates to these pharmaceutical products.

It is expedient to produce aerosol solutions for inhalation.

Suitable for oral use are in particular tablets, coated tablets or capsules with talc and/or hydrocarbon carriers or binders, such as, for example, lactose, corn starch or potato starch. Use can also take place in liquid form, such as, for example, as solution to which, where appropriate, a sweetener is added. Clathrates are likewise also suitable for oral use of such compounds, examples of clathrates which may be mentioned being those with alpha-, beta-, gamma-cyclodextrin or else beta-hydroxypropylcyclodextrin.

Sterile, injectable, aqueous or oily solutions are used for parenteral administration. Particularly suitable are injection solutions or suspensions, especially aqueous solutions of active compounds in polyethoxylated castor oil.

Examples suitable and customary for vaginal administration are pessaries, tampons or intrauterine device.

Appropriately prepared crystal suspensions can be used for intraarticular injection.

It is possible to use for intramuscular injection aqueous and oily injection solutions or suspensions and appropriate depot preparations.

For rectal administration, the novel compounds can be used in the form of suppositories, capsules, solutions (e.g. in the form of enemas) and ointments both for systemic and for local therapy.

The novel compounds can be used in the form of aerosols and inhalations for pulmonary administration.

For local use on the eyes, external auditory canal, middle ear, nasal cavity and paranasal sinuses, the novel compounds can be used as drops, ointments and tinctures in appropriate pharmaceutical preparations.

Formulations possible for topical application are gels, ointments, fatty ointments, creams, pastes, dusting powders, milk and tinctures. The dosage of the compounds of the general formula I should in these preparations be 0.01%-20% in order to achieve an adequate pharmacological effect.

The dosage of the active ingredients may vary depending on the route of administration, age and weight of the patient, nature and severity of the disorder to be treated and similar factors. Treatment can take place by single dosages or by a large number of dosages over a prolonged period. The daily dose is 0.5-1000 mg, preferably 50-200 mg, it being possible to give the dose as a single dose to be administered once or divided into 2 or more daily doses.

Carrier systems which can be used are also surface-active excipients such as salts of bile acids or animal or vegetable phospholipids, but also mixtures thereof, and liposomes or constituents thereof.

The present invention likewise relates to the formulations and dosage forms described above.

Administration of the compounds of the invention can take place by any conventional method, including oral and parenteral, e.g. by subcutaneous or intramuscular injections. The present invention likewise relates to enteral, parenteral, vaginal and oral administrations.

The compounds of the invention of the general formula I bind to the EP₂ receptor and have agonistic or antagonistic effect. It is possible to determine whether an agonistic or an antagonistic effect is present by an agonism test (see Example 1.2.1. of the Biological Examples) or by an antagonism test (see Example 1.2.2. of the Biological Examples).

Antagonists mean molecules which bind to their corresponding receptors and which inhibit the initiation of the signal transduction pathway(s) coupled to the receptor by the naturally occurring ligand(s). The antagonists normally compete with the naturally occurring ligand of the receptor for binding to the receptor. However, other modifications of the receptor are also possible by molecules which prevent the signal transduction pathways coupled to the receptor being activated by the naturally occurring ligand(s) (e.g. non-competitive, steric modifications of the receptor).

Receptor antagonists typically bind selectively to their particular receptor and not to other receptors. They normally have a higher binding affinity than the natural ligand. Although antagonists which have a higher affinity for the receptor than the natural ligand are preferred, it is likewise possible to employ antagonists having a lower affinity.

The antagonists preferably bind reversibly to their corresponding receptors.

The EP₂ receptor antagonist has a preferred affinity for the EP₂ receptor compared with any other EP receptor. The antagonism is measured in the presence of the natural agonist (PGE₂).

Agonists mean molecules which bind to their corresponding receptors and normally compete with the naturally occurring ligand of the receptor for binding to the receptor, and which stimulate the initiation of the signal transduction pathway coupled to the receptor. Agonists may also assist the binding of the natural ligand.

Receptor agonists typically bind selectively to their particular receptor and not to other receptors. They normally have a higher binding affinity than the natural ligand. Although agonists which have a higher affinity for the receptor than the natural ligand are preferred, it is likewise possible to employ agonists having a lower affinity.

The agonists preferably bind reversibly to their corresponding receptors.

The EP₂ receptor agonist has a preferred affinity for the EP₂ receptor compared with any other EP receptor.

Agonists are tested via the initiation of the signal transduction and/or physiological effect mediated by the corresponding receptor.

The compounds or low molecular weight substances which bind to a receptor are referred to as ligands. Their binding is normally reversible. Binding of a ligand to the corresponding receptor activates or inactivates the signal transduction pathway coupled to the receptor. The ligand mediates its intracellular effect in this manner. Ligands mean agonists and antagonists of a receptor.

The substance of Example 29 shows no inhibition in the cellular agonism test but a good activity (IC₅₀=1.2×10 E-6 M) in the antagonism test. The present invention likewise relates to the use of the substances of the invention as EP₂ receptor antagonists for the treatment of disorders which are caused by disturbances in the signal transduction chain in which the EP₂ receptor is involved, such as, for example, pain and fertility impairments, and which are likewise suitable for controlling fertility.

The oocyte is surrounded in the preovulatory antral follicle by cumulus cells which form a dense ring of cells around the oocyte. After the lutenizing hormone peak (LH peak), a series of processes is activated and leads to a large morphological change in this ring of cells composed of cumulus cells. In this case, the cumulus cells form an extracellular matrix which leads to so-called cumulus expansion (Vanderhyden et al. Dev Biol. 1990 August; 140(2):307-317). This cumulus expansion is an important constituent of the ovulatory process and of the subsequent possibility of fertilization.

Prostaglandins, and here prostaglandin E₂, whose synthesis is induced by the LH peak, are of crucial importance in cumulus expansion. Prostanoid EP₂ knockout mice (Hizaki et al. Proc Natl Acad Sci USA. 1999 Aug. 31; 96(18): 10501-6.) show a distinctly reduced cumulus expansion and severe subfertility, demonstrating the importance of the prostanoid EP₂ receptor for this process.

The substances of the invention have inhibitory effects in cumulus expansion tests.

The present invention relates to the use of the substances of the invention for controlling fertility.

Whereas the EP₂ receptor antagonist AH 6809 inhibits cumulus expansion by about only 30% and not until the concentration is 100-200 μM, an about 20% inhibition of cumulus expansion can be achieved in the presence of the substance of Example 29 even at a concentration which is 10-20 times lower (10 μM). In these experiments, the test substances compete with the natural EP₂ receptor agonist PGE₂.

The present invention relates to the use of the substances of the invention for inhibiting cumulus expansion and thus ovulation and fertilization for contraception.

Prostaglandins play an important part in angiogenesis (Sales, Jabbour, 2003, Reproduction 126, 559-567; Kuwano et al., 2004, FASEB J. 18, 300-310; Kamiyama et al., 2006, Oncogene 25, 7019-7028; Chang et al. 2005, Prostaglandins & other Lipid Mediators 76, 48-58).

Endometriosis is a chronic disorder caused by impairments of blood vessels. About 10% of women regularly suffer from heavy bleeding during menstruation, caused by changes in the blood vessels of the endometrium. In addition, structural differences in the blood vessels have been observed, such as, for example, incomplete formation of the smooth muscle cell layer (Abberton et al., 1999, Hum. Reprod. 14, 1072-1079). Since the blood loss during menstruation is partly controlled by constriction of the blood vessels, it is obvious that the defects in the smooth muscles make a substantial contribution to the bleeding.

The present invention relates to the use of the substances of the general formula I for treating endometriosis.

Prostaglandins play an important part in uterine contraction, and excessively strong contractions are responsible for painful menstruation (Sales, Jabbour, 2003, Reproduction 126, 559-567).

The present invention relates to the use of the substances of the general formula I for the treatment of painful menstruation.

Increasing research results also demonstrate the importance of EP receptors, and especially of the EP₂ receptor, in a large number of types of cancer (e.g. breast cancer, colon carcinoma, lung cancer, prostate cancer, leukemia, skin cancer), suggesting future possibilities of employing modulators (antagonists or agonists) of the EP2 receptor for the therapy and prevention (prophylactic and/or adjuvant) of cancer (Fulton et al. Cancer Res 2006; 66(20): 9794-7; Castellone et al. Science VOL 310 2005, 1504-1510; Chang et al. Cancer Res 2005; 65(11): 4496-9); Hull et al. Mol Cancer Ther 2004; 3(8): 1031-9; Richards et al. J Clin Endocrinol Metab 88: 2810-2816, 2003; Sinha et al. 2007, Cancer Res; 67(9):4507-13; Wang et al. 2004, Seminars in Oncology, Vol 31, No 1, Suppl 3: pp 64-73).

The present invention relates to the use of the substances of the general formula I for the treatment and prevention of cancers.

Prostaglandins also play an important part in processes counteracting osteoporosis. The present invention therefore relates to the use of the substances of the invention for the treatment of osteoporosis. Reinold et al. (J. Clin. Invest. 115, 673-679 (2005)) describes PGE₂ receptors of the EP₂ subtype as the key signaling elements in inflammatory hyperalgesia. Mice no longer having this receptor (EP₂−/−) do not experience spinal inflammatory pain. There is evidence that an inflammatory, increased pain sensitivity can be treated by targeted modulation of EP₂ receptors.

The present invention relates to the use of the substances of the invention for the treatment of inflammatory hyperalgesia.

The invention additionally relates to a process for preparing the compounds of the invention of the general formula I, which comprises reacting a compound of the formula II

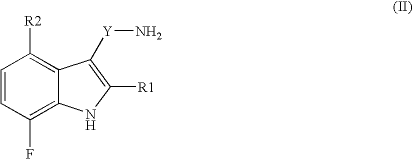

in which R¹, R² and Y have the meanings indicated above, with a carboxylic acid derivative of the general formula III

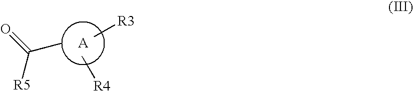

in which A, R³ and R⁴ have the meanings indicated above, and R⁵ may be a hydroxy group, a chlorine or bromine atom or a C₁-C₆-alkyl radical, with preference for hydrogen, chlorine, the methyl or ethyl radical, by methods known to the skilled worker, and subsequently eliminating protective groups required where appropriate.

In the case where R⁵ is a hydroxy group, the reaction can initially take place by activating the acid function, and in this case for example the carboxylic acid of the formula III is initially converted in the presence of a tertiary amine such as, for example, triethylamine with isobutyl chloroformate into the mixed anhydride. Reaction of the mixed anhydride with the alkali metal salt of the appropriate amine takes place in an inert solvent or solvent mixture such as, for example, tetrahydrofuran, dimethoxyethane, dimethylformamide, hexamethylphosphoric triamide, at temperatures between −30° C. and +60° C., preferably at 0° C. to 30° C.

A further possibility is to activate the carboxylic acid by reagents such as, for example, HOBt or HATU. Reaction of the acid takes place for example with HATU in an inert solvent such as, for example, DMF in the presence of the appropriate amine of the general formula III and a tertiary amine such as, for example, ethyldiisopropylamine at temperatures between −50 and +60° C., preferably at 0° C. to 30° C.

In the case where R⁵ is C₁-C₆-alkyl it is also possible for example to carry out a direct amidolysis of the ester with the appropriate amine, possibly with the assistance of trialkylaluminum reagents, preferably trimethylaluminum.

In the case where R⁵ is a chlorine or bromine atom it is possible for example to carry out the reaction for example in pyridine or an inert solvent such as, for example, DMF in the presence of the appropriate amine of the general formula II and a tertiary amine such as, for example, ethyldiisopropylamine at temperatures between −50 and +60° C., preferably at 0° C. to 30° C.

It is possible where appropriate for the compounds of the general formula (I) with R²═CN also to be prepared starting from the corresponding halides, preferably bromine or chlorine, by a Cu- or Pd-catalyzed (e.g. Pd(OAc)₂) cyanide introduction with Zn(CN)₂ or else K₃[Fe(CN)₆] in an inert solvent such as dimethylacetamide, dimethylformamide or N-methylpyrrolidone at temperatures between 60° C. and the boiling point of the respective solvent.

It is possible where appropriate for the compounds of the general formula (I) with R³ or R⁴=aryl or heteroaryl, which may where appropriate be substituted by the radicals indicated previously, to be prepared starting from an appropriate halide, preferably bromine or chlorine, by a Pd-catalyzed (e.g. Pd(OAc)₂, Pd(PPh₃)₄, Pd₂(dba)₃, PdCl₂(dppf)) reaction in the presence of a base such as, for example, sodium carbonate, cesium carbonate, potassium phosphate or ethyldiisopropylamine with an appropriate aryl- or heteroarylboronic acid or boronic acid derivative in a solvent such as, for example, toluene, dioxane, dimethylacetamide, dimethylformamide or N-methylpyrrolidone at temperatures between 60° C. and the boiling point of the respective solvent.

The compounds of the general formula II which serve as starting materials are either known or can be prepared for example by reacting in a manner known per se the known hydrazines IV, where appropriate prepared from the corresponding known anilines by nitrosation followed by a reduction,

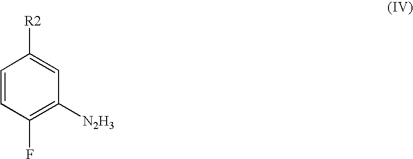

in which R² has the meaning indicated above,
a) with a ketone of the general formula V in which R¹ and Y have the meaning indicated above, and n=2 and 3, in a Fischer indole cyclization

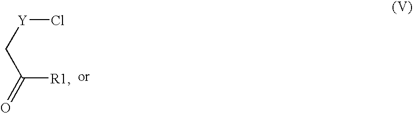

b) with an enol ether of the general formula VI in which R¹ and Y have the meaning indicated above, and n=2 and 3, in a Fischer indole cyclization (Org. Lett. 2004, 79ff),

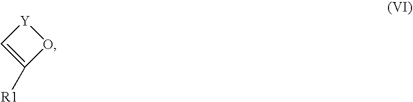

and converting the subsequently obtained alcohol by methods known to the skilled worker by conversion into a leaving group such as tosylate, mesylate, trifluoromesylate, chloride, bromide or iodide and subsequent reaction with, for example, sodium azide followed by a hydrolysis with PPh₃/H₂O in tetrahydrofuran into the amino function,
or
c) with a keto ester of the general formula VII in the case of Y with n=1

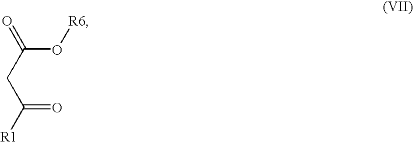

in which R¹ has the meaning indicated above, and R⁶ is a C₁-C₆-alkyl radical, in a Fischer indole cyclization, and subsequently reducing the resulting ester by methods known to the skilled worker such as, for example, diisobutylaluminum hydride in an inert solvent at temperatures between −50 and 25° C., preferably between −30 and 0° C., to the corresponding alcohol which is in turn converted into the amino function by conversion into a leaving group such as tosylate, mesylate, trifluoromesylate, chloride, bromide or iodide and subsequent reaction with, for example, sodium azide, followed by a hydrolysis with PPh₃/H₂O in tetrahydrofuran.

It is possible where appropriate for the compounds of the general formula (I) with R²═CN also to be prepared starting from the corresponding halides, preferably bromine or chlorine, by a Cu- or Pd-catalyzed (e.g. Pd(OAc)₂) cyanide introduction with Zn(CN)₂ or else K₃[Fe(CN)₆] in an inert solvent such as dimethylacetamide, dimethylformamide or N-methylpyrrolidone at temperatures between 60° C. and the boiling point of the respective solvent.

### Preparation of the Compounds of the Invention

The following examples illustrate the preparation of the compounds of the invention of the general formula (I) without restricting the scope of the claimed compounds to these examples.

The compounds of the invention of the general formula (I) can be prepared as described below.

### EXAMPLE 1

### Biphenyl-4-carboxylic acid [2-(7-fluoro-2,4-dimethyl-1H-indol-3-yl)ethyl]amide

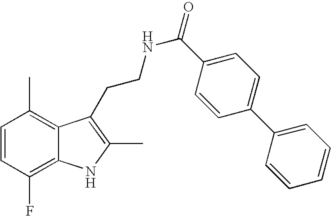

0.10 ml of triethylamine is added to a solution of 70.0 mg of 2-(7-fluoro-2,4-dimethyl-1H-indol-3-yl)ethylamine hydrochloride in 2.1 ml of dimethylformamide, and the mixture is stirred at 25° C. for 10 minutes. Then, at this temperature, 61.9 mg of biphenyl-4-carbonyl chloride are added, and the mixture is stirred at 25° C. for a further 45 minutes. The reaction solution is then added to ice-water and extracted twice with ethyl acetate. The combined organic phases are washed twice with water, dried over sodium sulfate and, after filtration, concentrated in vacuo. The residue obtained in this way is purified by medium pressure chromatography on silica gel with hexane/0-100% ethyl acetate. 72 g of the title compound are obtained in this way.

NMR (300 MHz, DMSO-d6): δ=2.30 (3H), 2.93 (3H), 2.98 (2H), 3.36 (2H), 6.54-6.68 (2H), 7.37 (1H), 7.46 (2H), 7.67-7.78 (4H), 7.92 (2H), 8.67 (1H), 11.10 (1H).

### EXAMPLE 2

### N-[2-(7-Fluoro-2,4-dimethyl-1H-indol-3-yl)ethyl]-3,4,5-trimethoxybenzamide

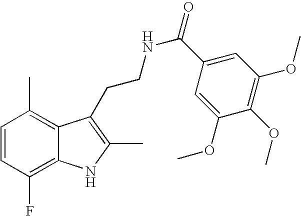

34 mg of the title compound are obtained in analogy to Example 1 from 70.0 mg of 2-(7-fluoro-2,4-dimethyl-1H-indol-3-yl)ethylamine hydrochloride and 65.9 mg of 3,4,5-trimethoxybenzoyl chloride.

NMR (300 MHz, DMSO-d6): δ=2.30 (3H), 2.60 (3H), 2.95 (2H), 3.33 (2H), 3.67 (3H), 3.79 (6H), 6.54-6.67 (2H), 7.15 (2H), 8.55 (1H), 11.11 (1H).

### EXAMPLE 3

### 4-Fluoro-N-[2-(7-fluoro-2,4-dimethyl-1H-indol-3-yl)ethyl]benzamide

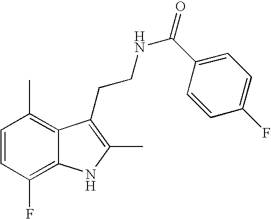

87 mg of the title compound are obtained in analogy to Example 1 from 70.0 mg of 2-(7-fluoro-2,4-dimethyl-1H-indol-3-yl)ethylamine hydrochloride and 33.3 μl of 4-fluorobenzoyl chloride.

NMR (300 MHz, DMSO-d6): δ=2.28 (3H), 2.58 (3H), 2.95 (2H), 3.33 (2H), 6.53-6.65 (2H), 7.26 (2H), 7.88 (2H), 8.63 (1H), 11.10 (1H).

### EXAMPLE 4

### 4-Methyl-N-[2-(7-fluoro-2,4-dimethyl-1H-indol-3-yl)ethyl]benzamide

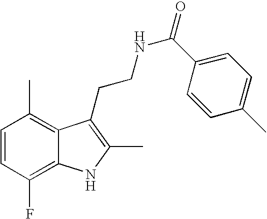

41 mg of the title compound are obtained in analogy to Example 1 from 70.0 mg of 2-(7-fluoro-2,4-dimethyl-1H-indol-3-yl)ethylamine hydrochloride and 37.8 μl of 4-methylbenzoyl chloride.

NMR (300 MHz, DMSO-d6): δ=2.28 (3H), 2.32 (3H), 2.58 (3H), 2.94 (2H), 3.32 (2H), 6.53-6.65 (2H), 7.23 (2H), 7.72 (2H), 8.52 (1H), 11.09 (1H).

### EXAMPLE 5

### 2-Chloro-N-[2-(7-fluoro-2,4-dimethyl-1H-indol-3-yl)ethyl]benzamide

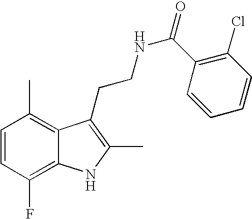

89 mg of the title compound are obtained in analogy to Example 1 from 70.0 mg of 2-(7-fluoro-2,4-dimethyl-1H-indol-3-yl)ethylamine hydrochloride and 36.2 μl of 2-chlorobenzoyl chloride.

NMR (300 MHz, DMSO-d6): δ=2.32 (3H), 2.57 (3H), 2.96 (2H), 3.29 (2H), 6.53-6.66 (2H), 7.32-7.43 (3H), 7.46 (1H), 8.53 (1H), 11.12 (1H).

### EXAMPLE 6

### N-[2-(7-Fluoro-2,4-dimethyl-1H-indol-3-yl)ethyl]-4-trifluoromethylbenzamide

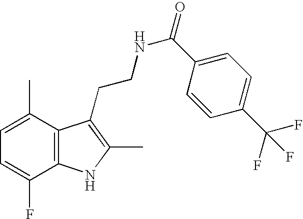

96 mg of the title compound are obtained in analogy to Example 1 from 70.0 mg of 2-(7-fluoro-2,4-dimethyl-1H-indol-3-yl)ethylamine hydrochloride and 42.5 μl of 4-trifluoromethylbenzoyl chloride.

NMR (300 MHz, DMSO-d6): δ=2.28 (3H), 2.59 (3H), 2.97 (2H), 3.36 (2H), 6.53-6.66 (2H), 7.82 (2H), 8.01 (2H), 8.85 (1H), 11.11 (1H).

### EXAMPLE 7

### 3-Chloro-N-[2-(7-fluoro-2,4-dimethyl-1H-indol-3-yl)ethyl]benzamide

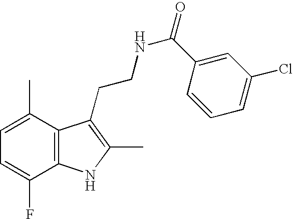

76 mg of the title compound are obtained in analogy to Example 1 from 70.0 mg of 2-(7-fluoro-2,4-dimethyl-1H-indol-3-yl)ethylamine hydrochloride and 36.7 μl of 3-chlorobenzoyl chloride.

NMR (300 MHz, DMSO-d6): δ=2.32 (3H), 2.63 (3H), 2.99 (2H), 3.37 (2H), 6.56-6.71 (2H), 7.52 (1H), 7.62 (1H), 7.83 (1H), 7.90 (1H), 8.80 (1H), 11.17 (1H).

### EXAMPLE 8

### N-[2-(7-Fluoro-2,4-dimethyl-1H-indol-3-yl)ethyl]-4-methoxybenzamide

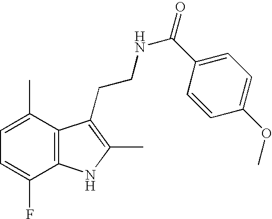

78 mg of the title compound are obtained in analogy to Example 1 from 70.0 mg of 2-(7-fluoro-2,4-dimethyl-1H-indol-3-yl)ethylamine hydrochloride and 48.1 mg of 4-methoxybenzoyl chloride.

NMR (300 MHz, DMSO-d6): δ=2.27 (3H), 2.58 (3H), 2.93 (2H), 3.31 (2H), 3.77 (3H), 6.53-6.66 (2H), 6.95 (2H), 7.79 (2H), 8.47 (1H), 11.11 (1H).

### EXAMPLE 9

### 4-Chloro-N-[2-(7-fluoro-2,4-dimethyl-1H-indol-3-yl)ethyl]benzamide

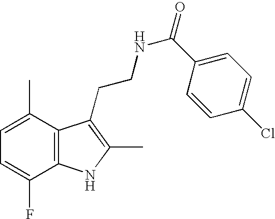

39 mg of the title compound are obtained in analogy to Example 1 from 70.0 mg of 2-(7-fluoro-2,4-dimethyl-1H-indol-3-yl)ethylamine hydrochloride and 36.3 μl of 4-chlorobenzoyl chloride.

NMR (300 MHz, DMSO-d6): δ=2.27 (3H), 2.58 (3H), 2.94 (2H), 3.32 (2H), 6.53-6.66 (2H), 7.51 (2H), 7.83 (2H), 8.72 (1H), 11.12 (1H).

### EXAMPLE 10

### N-[2-(7-Fluoro-2,4-dimethyl-1H-indol-3-yl)ethyl]-3-methylbenzamide

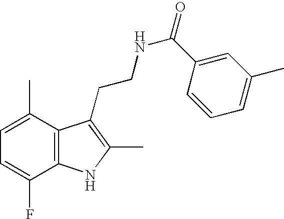

68 mg of the title compound are obtained in analogy to Example 1 from 70.0 mg of 2-(7-fluoro-2,4-dimethyl-1H-indol-3-yl)ethylamine hydrochloride and 37.6 μl of 3-methylbenzoyl chloride.

NMR (300 MHz, DMSO-d6): δ=2.33 (3H), 2.37 (3H), 2.63 (3H), 2.99 (2H), 3.36 (2H), 6.54-6.72 (2H), 7.31-7.40 (2H), 7.61-7.71 (2H), 8.61 (1H), 11.16 (1H).

### EXAMPLE 11

### 4-tert-Butyl-N-[2-(7-fluoro-2,4-dimethyl-1H-indol-3-yl)ethyl]benzamide

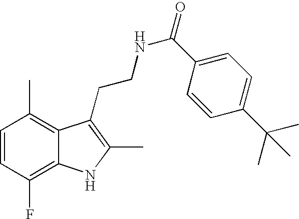

72 mg of the title compound are obtained in analogy to Example 1 from 70.0 mg of 2-(7-fluoro-2,4-dimethyl-1H-indol-3-yl)ethylamine hydrochloride and 52.4 μl of 4-tert-butylbenzoyl chloride.

NMR (300 MHz, DMSO-d6): δ=1.26 (9H), 2.28 (3H), 2.58 (3H), 2.94 (2H), 3.32 (2H), 6.52-6.67 (2H), 7.43 (2H), 7.74 (2H), 8.54 (1H), 11.11 (1H).

### EXAMPLE 12

### Benzo[1,3]dioxole-5-carboxylic acid [2-(7-fluoro-2,4-dimethyl-1H-indol-3-yl)ethyl]amide

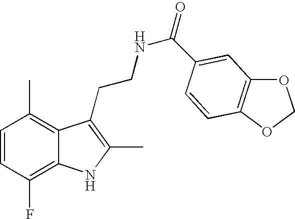

172 mg of N—[(dimethylamino)-1H-1,2,3-triazolo[4,5-b]pyridin-1-ylmethylene]-N-methylmethanaminium hexafluorophosphate N-oxide (HATU) and 100 mg of 2-(7-fluoro-2,4-dimethyl-1H-indol-3-yl)ethylamine hydrochloride are added to a solution of 68.4 mg of benzo[1,3]dioxole-5-carboxylic acid in 3 ml of dimethylformamide. Then, at 0° C., 0.15 ml of ethyldiisopropylamine is added dropwise, and the mixture is stirred at 25° C. for 20 hours. Then 40 ml of a mixture of ice and conc. aqueous bicarbonate solution are added, and the mixture is extracted three times with ethyl acetate. The combined organic phases are washed once with saturated sodium chloride solution, dried over sodium sulfate and, after filtration, concentrated in vacuo. The residue obtained in this way is purified by medium pressure chromatography on silica gel with hexane/0-100% ethyl acetate. 122 mg of the title compound are obtained in this way.

NMR (300 MHz, DMSO-d6): δ=2.27 (3H), 2.58 (3H), 2.92 (2H), 3.30 (2H), 6.06 (2H), 6.52-6.66 (2H), 6.95 (1H), 7.34 (1H), 7.41 (1H), 8.47 (1H), 11.11 (1H).

### EXAMPLE 13

### Thiophene-2-carboxylic acid [2-(7-fluoro-2,4-dimethyl-1H-indol-3-yl)ethyl]amide

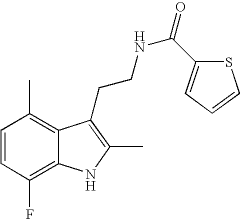

92 mg of the title compound are obtained in analogy to Example 1 from 75.0 mg of 2-(7-fluoro-2,4-dimethyl-1H-indol-3-yl)ethylamine hydrochloride and 33.0 μl of 2-thiophenecarbonyl chloride.

NMR (300 MHz, DMSO-d6): δ=2.27 (3H), 2.58 (3H), 2.93 (2H), 3.29 (2H), 6.53-6.67 (2H), 7.11 (1H), 7.68 (1H), 7.70 (1H), 8.64 (1H), 11.13 (1H).

### EXAMPLE 14

### Quinoxaline-6-carboxylic acid [2-(7-fluoro-2,4-dimethyl-1H-indol-3-yl)ethyl]amide

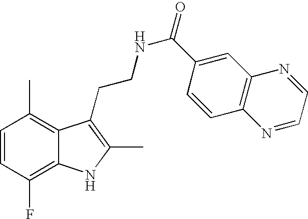

67 mg of the title compound are obtained in analogy to Example 12 from 75.0 mg of 2-(7-fluoro-2,4-dimethyl-1H-indol-3-yl)ethylamine hydrochloride and 59.5 mg of quinoxaline-6-carboxylic acid.

NMR (300 MHz, DMSO-d6): δ=2.30 (3H), 2.61 (3H), 3.01 (2H), 3.41 (2H), 6.54-6.68 (2H), 8.15 (1H), 8.26 (1H), 8.57 (1H), 9.00 (2H), 9.07 (1H), 11.15 (1H).

### EXAMPLE 15

### 5-Phenylpyridine-2-carboxylic acid [2-(7-fluoro-2,4-dimethyl-1H-indol-3-yl)ethyl]amide

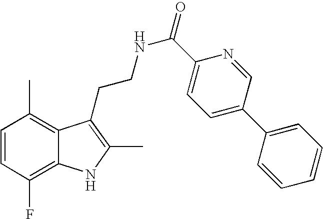

91 mg of the title compound are obtained in analogy to Example 12 from 100 mg of 2-(7-fluoro-2,4-dimethyl-1H-indol-3-yl)ethylamine hydrochloride and 90.3 mg of 5-phenylpyridine-2-carboxylic acid.

NMR (300 MHz, DMSO-d6): δ=2.27 (3H), 2.65 (3H), 2.81 (2H), 3.20 (2H), 6.50-6.65 (2H), 7.28-7.39 (5H), 7.53 (1H), 7.81 (1H), 8.54 (1H), 8.65 (1H), 11.12 (1H).

### EXAMPLE 16

### 5-Phenyl-1H-pyrrole-2-carboxylic acid [2-(7-fluoro-2,4-dimethyl-1H-indol-3-yl)ethyl]amide

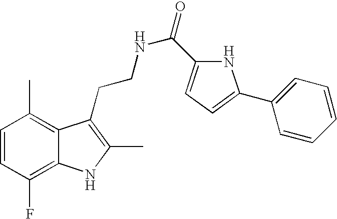

74 mg of the title compound are obtained in analogy to Example 12 from 100 mg of 2-(7-fluoro-2,4-dimethyl-1H-indol-3-yl)ethylamine hydrochloride and 84.8 mg of 5-phenyl-1H-pyrrole-2-carboxylic acid.

NMR (300 MHz, DMSO-d6): δ=2.30 (3H), 2.69 (3H), 2.95 (2H), 3.29 (2H), 6.51-6.68 (2H), 6.77 (1H), 7.17 (1H), 7.32 (2H), 7.77 (2H), 7.91 (1H) 8.23 (1H), 11.13 (1H).

### EXAMPLE 17

### N-[2-(4,7-Difluoro-2-methyl-1H-indol-3-yl)ethyl]-3,4-dimethoxybenzamide

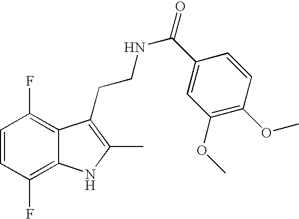

60 mg of the title compound are obtained in analogy to Example 1 from 100 mg of 2-(4,7-difluoro-2-methyl-1H-indol-3-yl)ethylamine and 143 mg of 3,4-dimethoxybenzoyl chloride.

NMR (300 MHz, DMSO-d6): δ=2.24 (3H), 2.88 (2H), 3.37 (2H), 3.74 (3H), 3.76 (3H), 6.60 (1H), 6.72 (1H), 6.96 (1H), 7.37 (1H), 7.40 (1H), 8.38 (1H), 11.43 (1H).

The starting material for the above title compound is prepared as follows:

### 17a) 2-(4,7-Difluoro-2-methyl-1H-indol-3-yl)ethylamine

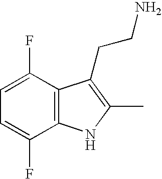

2.0 g of 2,5-difluorophenylhydrazine are dissolved in 45 ml of a mixture of ethanol and water in the ratio 14:1 at 120° C. Then, when boiling, 1.59 ml of 5-chloro-2-pentanone dissolved in 2 ml of ethanol are added, and the mixture is stirred at this temperature for 16 hours. Cooling is followed by concentration in vacuo, and the resulting residue is purified by column chromatography on silica gel with methylene chloride/0-20% methanol/0.5% triethylamine. 516 mg of the title compound are obtained in this way.

NMR (300 MHz, DMSO-d6): δ=2.31 (3H), 2.85 (4H), 6.61 (1H), 6.74 (1H), 11.56 (1H).

### EXAMPLE 18

### (±)-N-[2-(7-Fluoro-2,4-dimethyl-1H-indol-3-yl)ethyl]-4-methanesulfinylbenzamide

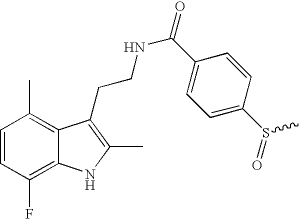

61 mg of the title compound are obtained in analogy to Example 12 from 100 mg of 2-(7-fluoro-2,4-dimethyl-1H-indol-3-yl)ethylamine hydrochloride and 83.5 mg of (±)-4-methanesulfinylbenzoic acid.

NMR (300 MHz, DMSO-d6): δ=2.28 (3H), 2.59 (3H), 2.75 (3H), 2.96 (2H), 3.34 (2H), 6.52-6.68 (2H), 7.74 (2H), 7.98 (2H), 8.79 (1H), 11.12 (1H).

### EXAMPLE 19

### N-[2-(7-Fluoro-1H-indol-3-yl)ethyl]-3,4-dimethoxybenzamide

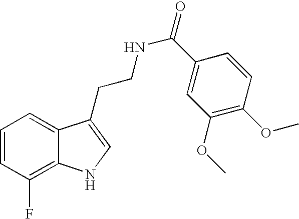

128 mg of the title compound are obtained in analogy to Example 12 from 100 mg of 2-(7-fluoro-1H-indol-3-yl)ethylamine and 113 mg of 3,4-dimethoxy-benzoic acid.

NMR (300 MHz, DMSO-d6): δ=2.91 (2H), 3.49 (2H), 3.76 (6H), 6.85 (1H), 6.91 (1H), 6.97 (1H), 7.20 (1H), 7.38 (1H), 7.39 (1H), 7.43 (1H), 8.44 (1H), 11.27 (1H).

### EXAMPLE 20

### N-[2-(7-Fluoro-2,4-dimethyl-1H-indol-3-yl)ethyl]-3-[1,2,4]triazol-1-ylmethylbenzamide

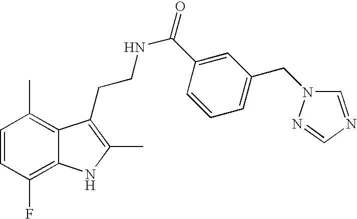

52 mg of the title compound are obtained in analogy to Example 12 from 100 mg of 2-(7-fluoro-2,4-dimethyl-1H-indol-3-yl)ethylamine hydrochloride and 83.7 mg of 3-[1,2,4]triazol-1-ylmethylbenzoic acid.

NMR (300 MHz, DMSO-d6): δ=2.27 (3H), 2.65 (3H), 2.94 (2H), 3.32 (2H), 5.44 (2H), 6.52-6.67 (2H), 7.34-7.47 (2H), 7.71-7.78 (2H), 7.97 (1H), 8.65 (1H), 8.66 (1H), 11.12 (1H).

### EXAMPLE 21

### Thieno[2,3-b]pyrazine-6-carboxylic acid [2-(7-fluoro-2,4-dimethyl-1H-indol-3-yl)ethyl]amide

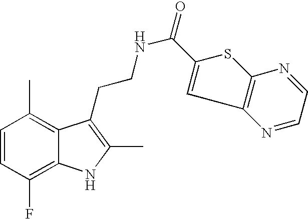

55 mg of the title compound are obtained in analogy to Example 12 from 100 mg of 2-(7-fluoro-2,4-dimethyl-1H-indol-3-yl)ethylamine hydrochloride and 74.2 mg of thieno[2,3-b]pyrazine-6-carboxylic acid.

NMR (300 MHz, DMSO-d6): δ=2.29 (3H), 2.59 (3H), 2.99 (2H), 3.39 (2H), 6.53-6.69 (2H), 8.20 (1H), 8.68 (1H), 8.79 (1H), 9.20 (1H), 11.16 (1H).

### EXAMPLE 22

### N-[2-(7-Fluoro-2-methyl-1H-indol-3-yl)ethyl]-3,4-dimethoxybenzamide

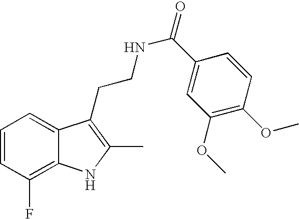

69 mg of the title compound are obtained in analogy to Example 1 from 79 mg of 2-(7-fluoro-2-methyl-1H-indol-3-yl)ethylamine and 123 mg of 3,4-dimethoxy-benzoyl chloride.

NMR (300 MHz, DMSO-d6): δ=2.28 (3H), 2.83 (2H), 3.35 (2H), 3.75 (3H), 3.76 (3H), 6.76 (1H), 6.85 (1H), 6.97 (1H), 7.27 (1H), 7.38 (1H), 7.41 (1H), 8.42 (1H), 11.13 (1H).

### EXAMPLE 23

### N-[2-(4-Bromo-7-fluoro-2-methyl-1H-indol-3-yl)ethyl]-3,4-dimethoxybenzamide

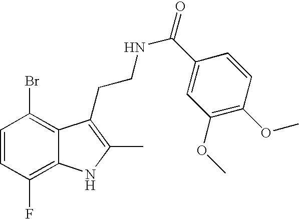

38 mg of the title compound are obtained in analogy to Example 1 from 100 mg of 2-(4-bromo-7-fluoro-2-methyl-1H-indol-3-yl)ethylamine and 111 mg of 3,4-dimethoxybenzoyl chloride.

NMR (300 MHz, DMSO-d6): δ=2.26 (3H), 3.06 (2H), 3.42 (2H), 3.75 (3H), 3.76 (3H), 6.74 (1H), 6.96 (1H), 7.04 (1H), 7.38 (1H), 7.42 (1H), 8.38 (1H), 11.54 (1H).

The starting material for the above title compound is prepared as follows:

### 23a) 2-Fluoro-5-bromophenylhydrazine hydrochloride

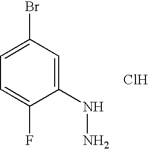

A solution of 2.8 g of sodium nitrite in 14 ml of water is added dropwise over the course of 30 minutes to a solution of 7.59 g of 2-fluoro-5-bromoaniline in 25 ml of hydrochloric acid (37% strength) at 0° C. Then, at 0° C., a solution of 24.6 g of tin chloride in 21 ml of hydrochloric acid (37% strength) is added dropwise, and the mixture is stirred at this temperature for a further 1.5 hours. Addition of 60 ml of sodium hydroxide solution (50% strength) and 60 ml of ice-water (pH >10) is followed by dilution with 150 ml of water and extraction three times with 100 ml of ether each time. The combined organic phases are washed with half-saturated sodium chloride solution, dried over sodium sulfate. The filtrate is acidified with 20 ml of 4.0M HCl in 1,4-dioxane solution, and the resulting precipitate is then filtered off and dried. 8.28 g of the title compound are obtained in this way.

NMR (300 MHz, DMSO-d6): δ=7.10-7.18 (1H), 7.21 (1H), 7.40 (1H), 8.59 (1H), 10.44 (3H).

### 23b) 2-(4-Bromo-7-fluoro-2-methyl-1H-indol-3-yl)ethylamine

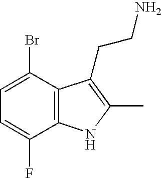

3.7 g of the title compound are obtained in analogy to Example 17a from 8.0 g of hydrazine hydrochloride prepared in Example 23a) and 3.8 ml of 5-chloro-2-pentanone.

NMR (300 MHz, DMSO-d6): δ=2.31 (3H), 2.67 (2H), 2.85 (2H), 6.71 (1H), 7.00 (1H), 11.55 (1H).

### EXAMPLE 24

### N-[2-(4-Chloro-7-fluoro-2-methyl-1H-indol-3-yl)ethyl]-3,4-dimethoxybenzamide

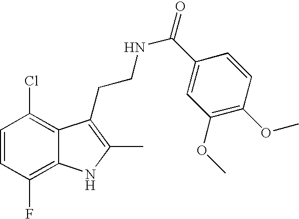

88 mg of the title compound are obtained in analogy to Example 1 from 100 mg of 2-(4-chloro-7-fluoro-2-methyl-1H-indol-3-yl)ethylamine and 133 mg of 3,4-dimethoxybenzoyl chloride.

NMR (300 MHz, DMSO-d6): δ=2.26 (3H), 3.04 (2H), 3.41 (2H), 3.75 (3H), 3.76 (3H), 6.78 (1H), 6.87 (1H), 6.96 (1H), 7.38 (1H), 7.42 (1H), 8.39 (1H), 11.54 (1H).

The starting material for the above title compound is prepared as follows:

### 24a) 2-Fluoro-5-chlorophenylhydrazine hydrochloride

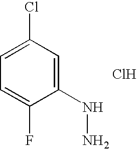

12.2 g of the title compound are obtained in analogy to Example 23a from 10 g of 2-fluoro-5-chloroaniline.

NMR (300 MHz, DMSO-d6): δ=6.96 (1H), 7.21 (1H), 7.17-7.26 (2H), 8.57 (1H), 10.42 (3H).

### 24b) 2-(4-Chloro-7-fluoro-2-methyl-1H-indol-3-yl)ethylamine

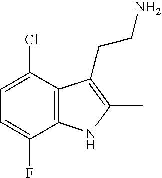

4.7 g of the title compound are obtained in analogy to Example 17a from 12.2 g of the hydrazine hydrochloride prepared in Example 24a) and 7.1 ml of 5-chloro-2-pentanone.

NMR (300 MHz, DMSO-d6): δ=2.31 (3H), 2.70 (2H), 2.86 (2H), 6.76 (1H), 6.85 (1H), 11.53 (1H).

### EXAMPLE 25

### N-[2-(7-Fluoro-2,4-dimethyl-1H-indol-3-yl)ethyl]-4-methanesulfonylbenzamide

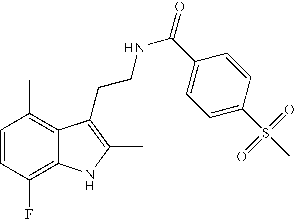

88 mg of the title compound are obtained in analogy to Example 12 from 100 mg of 2-(7-fluoro-2,4-dimethyl-1H-indol-3-yl)ethylamine hydrochloride and 91 mg of 4-methanesulfonylbenzoic acid.

NMR (300 MHz, DMSO-d6): δ=2.28 (3H), 2.59 (3H), 2.97 (2H), 3.24 (3H), 3.36 (2H), 6.53-6.67 (2H), 7.99 (2H), 8.04 (2H), 8.91 (1H), 11.14 (1H).

### EXAMPLE 26

### 1H-Benzotriazole-5-carboxylic acid [2-(4,7-difluoro-2-methyl-1H-indol-3-yl)ethyl]amide

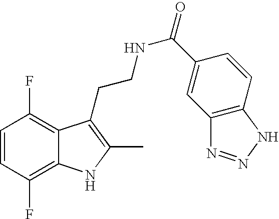

25 mg of the title compound are obtained in analogy to Example 12 from 50 mg of 2-(4,7-difluoro-2-methyl-1H-indol-3-yl)ethylamine and 39 mg of 1H-benzotriazole-5-carboxylic acid.

NMR (300 MHz, DMSO-d6): δ=2.24 (3H), 2.93 (2H), 3.44 (2H), 6.60 (1H), 6.72 (1H), 7.88 (2H), 7.91 (1H), 8.35 (1H), 8.71 (1H), 11.44 (1H).

### EXAMPLE 27

### 1H-Indole-2-carboxylic acid [2-(4,7-difluoro-2-methyl-1H-indol-3-yl)ethyl]amide

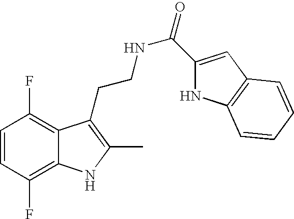

37 mg of the title compound are obtained in analogy to Example 12 from 50 mg of 2-(4,7-difluoro-2-methyl-1H-indol-3-yl)ethylamine and 38 mg of 1H-indole-2-carboxylic acid.

NMR (300 MHz, DMSO-d6): δ=2.24 (3H), 2.91 (2H), 3.43 (2H), 6.60 (1H), 6.72 (1H), 6.98 (1H), 7.01 (1H), 7.12 (1H), 7.37 (1H), 7.55 (1H), 8.51 (1H), 11.42 (1H), 11.49 (1H).

### EXAMPLE 28

### N-[2-(7-Fluoro-2,4-dimethyl-1H-indol-3-yl)ethyl]-3,4-dimethoxybenzamide

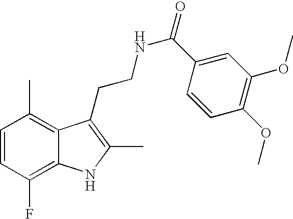

750 mg of the title compound are obtained in analogy to Example 1 from 500 mg of 2-(7-fluoro-2,4-dimethyl-1H-indol-3-yl)ethylamine hydrochloride and 413 mg of 3,4-dimethoxybenzoyl chloride.

NMR (300 MHz, DMSO-d6): δ=2.28 (3H), 2.59 (3H), 2.93 (2H), 3.31 (2H), 3.76 (3H), 3.77 (3H), 6.52-6.67 (2H), 6.98 (1H), 7.40 (1H), 7.44 (1H), 8.49 (1H), 11.12 (1H).

### EXAMPLE 29

### 4′-Fluorobiphenyl-4-carboxylic acid [2-(7-fluoro-2,4-dimethyl-1H-indol-3-yl)ethyl]amide

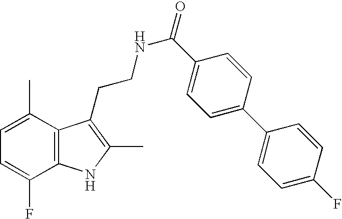

0.51 ml of a 1 molar sodium carbonate solution and 30.5 mg of tetrakistriphenylphosphinepalladium are added to a mixture of 100 mg of the bromide from Example 29a) and 54 mg of 4-fluorophenylboronic acid in 3 ml of a mixture of ethanol and toluene in the ratio 1:1. This suspension is heated in a microwave (CEM) at 120° C./100 W under nitrogen for 15 min. The reaction mixture is added to 50 ml of saturated sodium bicarbonate solution and extracted three times with 50 ml of ethyl acetate each time. The combined organic phases are washed once with 50 ml of saturated sodium chloride solution, dried over sodium sulfate and, after filtration, concentrated in vacuo. The crude product obtained in this way is purified by medium pressure chromatography on silica gel with hexane/0-100% ethyl acetate. 37.9 mg of the title compound are obtained in this way.

NMR (300 MHz, DMSO-d6): δ=2.29 (3H), 2.60 (3H), 2.97 (2H), 3.35 (2H), 6.53-6.68 (2H), 7.28 (2H), 7.69-7.79 (4H), 7.91 (2H), 8.69 (1H), 11.13 (1H).

The starting material for the above title compound is prepared as follows:

### 29a) 4-Bromo-N-[2-(7-fluoro-2,4-dimethyl-1H-indol-3-yl)ethyl]benzamide

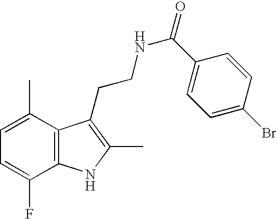

975 mg of the title compound are obtained in analogy to Example 1 from 1.0 g of 2-(7-fluoro-2,4-dimethyl-1H-indol-3-yl)ethylamine hydrochloride and 895 mg of 4-bromobenzoyl chloride.

NMR (300 MHz, DMSO-d6): δ=2.27 (3H), 2.58 (3H), 2.94 (2H), 3.31 (2H), 6.52-6.67 (2H), 7.65 (2H), 7.76 (2H), 8.71 (1H), 11.12 (1H).

### EXAMPLE 30

### N-[2-(7-Fluoro-2,4-dimethyl-1H-indol-3-yl)ethyl]-N′-pyridin-3-yl-terephthalamide

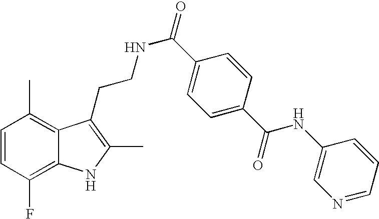

46 mg of the title compound are obtained in analogy to Example 12 from 50 mg of 2-(4,7-difluoro-2-methyl-1H-indol-3-yl)ethylamine and 57 mg of N-pyridin-3-yl-terephthalamic acid.

NMR (300 MHz, DMSO-d6): δ=2.34 (3H), 2.65 (3H), 3.02 (2H), 3.42 (2H), 6.58-6.72 (2H), 6.88 (1H), 7.42 (1H), 8.01 (2H), 8.07 (2H), 8.21 (1H), 8.34 (1H), 8.87 (1H), 8.95 (1H), 10.57 (1H).

Required starting materials for compounds in the table which follows:

### A) 2-(7-fluoro-2-methyl-4-trifluoromethyl-1H-indol-3-yl)ethylamine

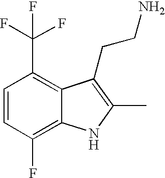

A1) In analogy to example 17a), 7.07 g of 2-fluoro-5-trifluoromethylphenylamine affords 9.1 g of (2-fluoro-5-trifluoromethylphenyl)hydrazine hydrochloride.

A2) In analogy to example 23a), 1 g of the hydrazine prepared above, by heating in a microwave at 120° C. for one hour, affords 460 mg of 2-(7-fluoro-2-methyl-4-trifluoromethyl-1H-indol-3-yl)ethylamine.

NMR (300 MHz, DMSO-d6): δ=2.46 (3H), 2.80 (2H), 3.03 (2H), 7.01 (1H), 7.38 (1H), 8.09 (2H), 12.14 (1H).

### B) 2-(7-fluoro-2-trifluoromethyl-4-methyl-1H-indol-3-yl)ethylamine

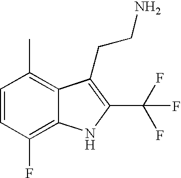

### B1) N-(2-fluoro-5-methylphenyl)-2-[(E)-hydroxyimino]acetamide

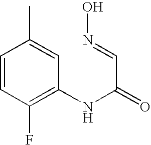

To a solution at 100° C. of 36.4 g of chloral hydrate and 230 g of sodium sulfate in 780 ml of water is added a solution of 25 g of 2-fluoro-4-methyl-aniline, 17 ml of concentrated hydrochloric acid in 120 ml of water and a hot solution of hydroxylamine hydrochloride in 100 ml of water. This mixture is left to stand at 25° C. for 5 hours and then the precipitate formed is filtered off. The solid is washed with cold water and dried under air. In this way, 38 g of N-(2-fluoro-5-methylphenyl)-2-[(E)-hydroxyimino]acetamide are obtained as a slightly brownish solid.

NMR (300 MHz, DMSO-d6): δ=2.25 (3H), 7.00 (1H), 7.05 (1H), 7.67 (2H), 9.67 (1H).

### B2) 7-fluoro-4-methyl-1H-indole-2,3-dione

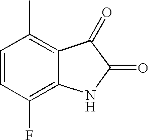

To a mixture of 386 g of 98% sulfuric acid and 43 ml of water are slowly added 36 g of the compound prepared above. During the addition, the temperature of the reaction mixture is kept between 75 and 80° C. and, after the addition, it is stirred at 80° C. for 15 minutes. Subsequently, the reaction mixture is added to 2 liters of ice-water and the precipitate formed is filtered off. The solid is washed with cold water and dried under air. In this way, 28.2 g of the title compound are obtained as a dark red solid.

NMR (300 MHz, DMSO-d6): δ=2.48 (3H), 6.82 (1H), 7.35 (1H), 11.43 (1H).

### B3) (2-amino-3-fluoro-6-methylphenyl)methanol

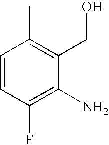

To a solution of 37.4 g of sodium hydroxide in 800 ml of water are added 27 g of the compound prepared above. To this mixture is added dropwise an aqueous H₂O₂ solution (prepared from 41.6 ml of 30% H₂O₂ solution and 360 ml of water), and the temperature is maintained between 25 and 30° C. during the dropwise addition. Subsequently, the mixture is stirred at 25° C. for 16 hours, acidified to pH approx. 5 with 36% hydrochloric acid and then concentrated under reduced pressure. The crude product thus obtained (2-amino-3-fluoro-6-methylbenzoic acid) is used further without additional purification. To a solution of 10 g of lithium aluminum hydride in 1 liter of tetrahydrofuran is slowly added, in portions of approx. 2 g, the acid prepared above at 10 to 15° C. After the complete addition, the reaction mixture is heated at reflux for 2 hours. After cooling, 10 ml of cold water are very cautiously added dropwise, followed by a solution of 3.3 g of sodium hydroxide in 10 ml of water. The mixture is heated at reflux and, after cooling, the precipitate formed is filtered off. The filtrate is concentrated under reduced pressure and the residue thus obtained is purified by column chromatography on silica gel with an eluent mixture of chloroform/methanol=19:1. In this way, 9 g of the title compound are obtained as a white solid.

NMR (300 MHz, DMSO-d6): δ=2.20 (3H), 4.45 (2H), 4.86 (3H), 6.46 (1H), 6.82 (1H).

### B4) N-(6-fluoro-2-hydroxymethyl-3-methylphenyl)trifluoroacetamide

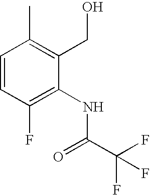

To a solution of 1.0 g of the aniline prepared above in 30 ml of methylene chloride are added dropwise 2.68 g of trifluoroacetic anhydride at 0° C. with stirring. On completion of addition, the mixture is allowed to warm up to 25° C. and is stirred at this temperature for 16 hours. The organic phase is then washed with 15% potassium carbonate solution, dried over sodium sulfate and, after filtration, concentrated under reduced pressure. The residue thus obtained is extracted with hot hexane and then the hexane phases are concentrated cautiously under reduced pressure. The trifluoroacetamide thus obtained is used in the next stage without further purification.

NMR (300 MHz, DMSO-d6): δ=2.33 (3H), 5.41 (2H), 7.37 (2H), 11.25 (1H).

### B5) 7-fluoro-4-methyl-2-trifluoromethyl-1H-indole

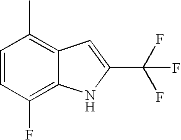

A mixture of 1 g of the compound prepared above and 1.51 g of triphenylphosphine hydrobromide in 50 ml of acetonitrile is heated under reflux for 17 hours. Subsequently, this mixture is concentrated under reduced pressure and washed with 50 ml of diethyl ether, and the residue is dried under air. In this way, 1.88 g of (2-trifluoroacetylamino-3-fluoro-6-methylbenzyl)triphenylphosphonium bromide are obtained as a yellow solid which is used in the next stage without further purification.

A solution of 18.9 g of the phosphonium salt prepared above in 600 ml of DMF is heated under reflux for 20 hours. After cooling, the mixture is concentrated under reduced pressure and the residue thus obtained is purified by means of column chromatography on silica gel with hexane. In this way, 4.3 g of the title compound are obtained as a pale yellow oil.

NMR (300 MHz, DMSO-d6): δ=2.45 (3H), 6.84 (1H), 6.98 (1H), 7.20 (1H), 12.72 (1H).

### B6) (7-fluoro-4-methyl-2-trifluoromethyl-1H-indol-3-ylmethyl)dimethylamine

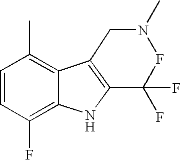

To a solution of 0.47 g of potassium carbonate in 7.5 ml of acetic acid are added, at −10° C., 0.56 g of dimethylamine hydrochloride in 7.5 ml of dioxane, followed by 0.42 ml of 40% formaldehyde solution and 1 g of the indole prepared above in 7.5 ml of dioxane. Subsequently, this mixture is stirred at 25° C. for 2 hours and then heated to 80° C. for a further 5 hours. After cooling, the reaction mixture is then concentrated under reduced pressure and added to 15% potassium carbonate solution. After extraction three times with 30 ml each time of ethyl acetate, the combined organic phases are dried over sodium sulfate. After filtration, the mixture is concentrated under reduced pressure and the residue thus obtained is purified by means of column chromatography on silica gel with 19:1 hexane/ethyl acetate. In this way, 0.5 g of the title compound is obtained as a white solid.

NMR (300 MHz, DMSO-d6): δ=2.18 (6H), 2.71 (3H), 3.58 (2H), 6.78 (1H), 6.98 (1H), 7.20 (1H), 12.41 (1H).

### B7) (7-fluoro-4-methyl-2-trifluoromethyl-1H-indol-3-yl)acetonitrile

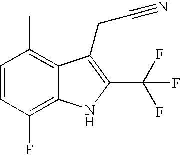

To a solution of 0.51 g of the amine prepared above in 10 ml of DMF is added a solution of 1.24 g of potassium cyanide in 100 ml of water, and this mixture is heated under reflux for 2 hours. After cooling, the mixture is concentrated under reduced pressure and the residue thus obtained is purified by column chromatography on silica gel with 9:1 hexane/ethyl acetate. In this way, 0.22 g of the title compound is obtained as a white solid.

NMR (300 MHz, DMSO-d6): δ=2.72 (3H), 4.30 (2H), 6.89 (1H), 7.07 (1H), 12.90 (1H).

### B8) 2-(7-fluoro-2-trifluoromethyl-4-methyl-1H-indol-3-yl)ethylamine

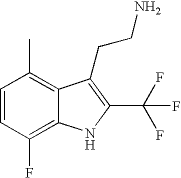

To a solution of 1.8 g of the nitrile prepared above in 40 ml of ethanol are added, with stirring at 25° C., a solution of 3.49 g of cobalt diacetate tetrahydrate in 40 ml of ethanol, followed by 2.65 g of sodium borohydride, and then the mixture is stirred at this temperature for 17 hours. The mixture is then concentrated under reduced pressure and the residue thus obtained is purified by column chromatography on silica gel with chloroform/methanol/aq. NH₃ in a ratio of 100:10:1. In this way, 1.37 g of the title compound are obtained as a gray-yellow solid.

NMR (300 MHz, DMSO-d6): δ=2.63 (3H), 2.77 (2H), 3.00 (2H), 4.98 (2H), 6.76 (1H), 6.95 (1H), 12.90 (1H).

The following examples are prepared in analogy to Example 1 or 29 and purified by HPLC:

HPLC-method:

Instrument: analytical 4-channel MUX system with CTC Pal injector, Waters 1525 pumps, Waters 2488 UV detector and Waters ZQ 2000 single quad MS detector.

Column X-Bridge RP C18 4.6×50 3.5 μm; detection wavelength 214 nm; flow rate 2 ml/min; eluents A: 0.1% TFA in H2O, B 0.1% TFA in ACN; gradient based in each case on B: 1% to 99% (5′) to 99% (1′) to 1% (0.25°) to 1% (1.75°)

or
in the case of the retention times below 2 minutes: detection: UV=200-400 nm (Waters Acquity HPLC)/MS 100-800 Daltons; 20 V (Micromass/Waters ZQ 4000); column: X Bridge (Waters), 2.1×50 mm, BEH 1.7 μm; eluents: A: H2O/0.05% HCOOH, B: CH3CN/0.05% HCOOH. Gradient: 10-90% B in 1.7 min, 90% B for 0.2 min, 98-2% B in 0.6 min; flow rate: 1.3 ml/min.

| | | | Theoret- | | |
|---|---|---|---|---|---|
| | | | ical | Mass | Reten- |
| Ex- | | | mass | found | tion |
| am- | | | m/z | m/z | time |
| ple | Structure | Name | [M + H]⁺ | [M + H]⁺ | [min.] |
| 31 | 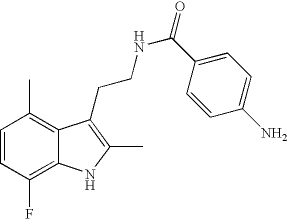 | 4-amino-N-[2-(7-fluoro-2,4-dimethyl-1H-indol-3-yl)ethyl]benzamide | 326 | 327 | 6.84 |
| 32 | 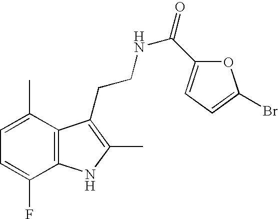 | 5-bromofuran-2-carboxylic acid [2-(7-fluoro-2,4-dimethyl-1H-indol-3-yl)ethyl]-amide | 379 | 380 | 9.29 |
| 33 | 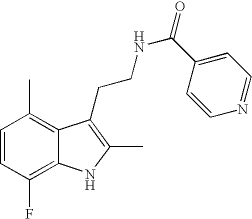 | N-[2-(7-fluoro-2,4dimethyl-1H-indol-3-yl)ethyl]-isonicotinamide | 312 | 313 | 6.25 |
| 34 | 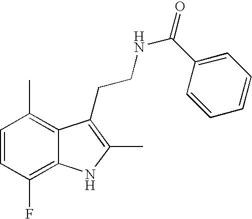 | N-[2-(7-fluoro-2,4dimethyl-1H-indol-3-yl)ethyl]benzamide | 311 | 312 | 9.04 |
| 35 | 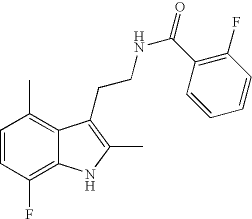 | 2-fluoro-N-[2-(7-fluoro-2,4-dimethyl-1H-indol-3-yl)-ethyl]benzamide | 329 | 330 | 9.3 |
| 36 | 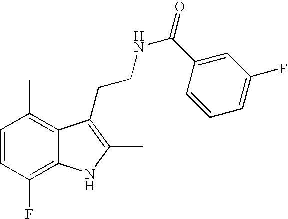 | 3-fluoro-N-]2-(7-fluoro-2,4-dimethyl-1H-indol-3-yl)ethyl]benzamide | 329 | 330 | 9.27 |
| 37 | 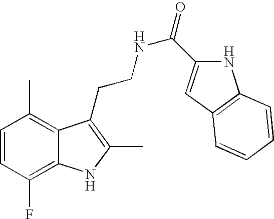 | 1H-indole-2-carboxylic acid [2-(7-fluoro-2,4-dimethyl-1H-indol-3-yl)ethyl]-amide | 350 | 351 | 9.42 |
| 38 | 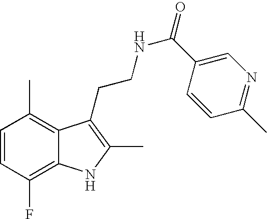 | N-[2-(7-fluoro-2,4-dimethyl-1H-indol-3-yl)ethyl]-6-methyl-nicotinamide | 326 | 327 | 6.25 |
| 39 | 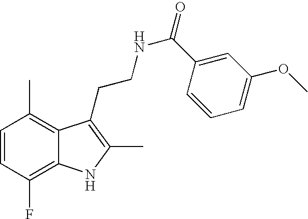 | N-[2-(7-fluoro-2,4-dimethyl-1H-indol-3-yl)ethyl]-3-methoxy-benazmide | 341 | 342 | 9.15 |
| 40 | 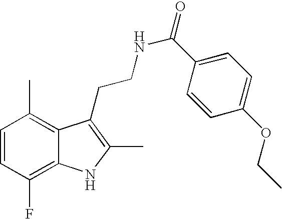 | 4-ethoxy-N-[2-(7-fluoro-2,4-dimethyl- 1H-indol-3-yl)ethyl]benzamide | 355 | 356 | 9.5 |
| 41 | 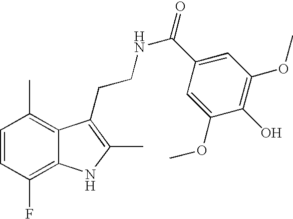 | N-[2-(7-fluoro-2,4-dimethyl-1H-indol-3-yl)ethyl]-4-hydroxy-3,5-dimethoxy-benzamide | 387 | 388 | 7.94 |
| 42 | 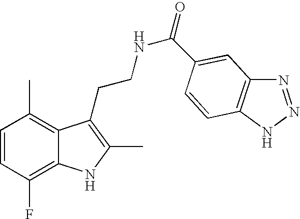 | 1H-benzotriazole-5-carboxylic acid [2-(7-fluoro-2,4-dimethyl-1H-indol-3-yl)ethyl]-amide | 352 | 353 | 7.45 |
| 43 | 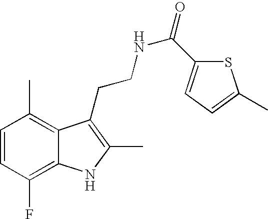 | 5-methylthiophene-2-carboxylic acid [2-(7-fluoro-2,4-dimethyl-1H-indol-3-yl)ethyl]amide | 331 | 332 | 9.37 |
| 44 | 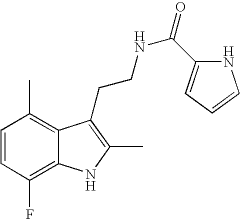 | 1H-pyrrole-2-carboxylic acid [2-(7-fluoro-2,4-dimethyl-1H-indol-3-yl)ethyl]-amide | 300 | 301 | 8.25 |
| 45 | 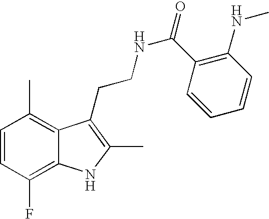 | N-[2-(7-fluoro-2,4-dimethyl-1H-indol-3-yl)ethyl]-2-methylamino-benzamide | 340 | 341 | 8.24 |
| 46 | 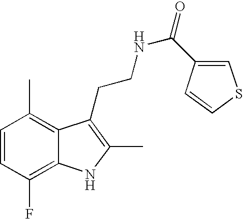 | thiophene-3-carboxylic acid [2-(7-fluoro-2,4-dimethyl-1H-indol-3-yl)ethyl]-amide | 317 | 318 | 8.84 |
| 47 | 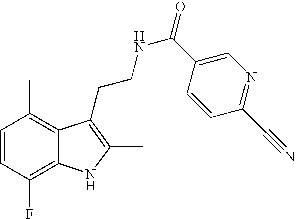 | 6-cyano-N-[2-(7-fluoro-2,4-dimethyl-1H-indol-3-yl)ethyl]-nicotinamide | 337 | 338 | 8.65 |
| 48 | 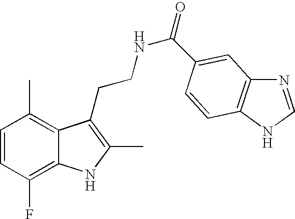 | 1H-benzoimidazole-5-carboxylic acid [2-(7-fluoro-2,4-dimethyl-1H-indol-3-yl)ethyl]amide | 351 | 352 | 6.28 |
| 49 | 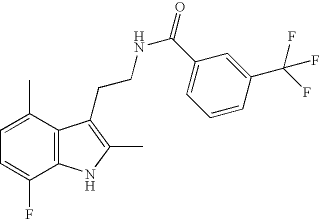 | N-[2-(7-fluoro-2,4-dimethyl-1H-indol-3-yl)ethyl]-3-trifluoro-methylbenzamide | 379 | 380 | 9.94 |
| 50 | 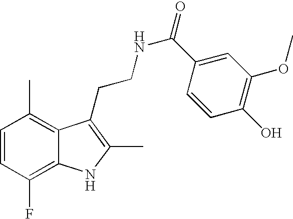 | N-[2-(7-fluoro-2,4-dimethyl-1H-indol-3-yl)ethyl]-4-hydroxy-3-methoxy-benzamide | 357 | 358 | 8.02 |
| 51 | 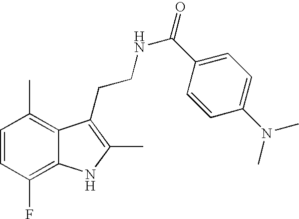 | 4-dimethylamino-N-[2-(7-fluoro-2,4-dimethyl-1H-indol-3-yl)ethyl]benzamide | 354 | 355 | 7.89 |
| 52 | 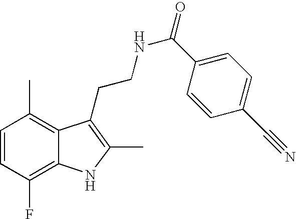 | 4-cyano-N-[2-(7-fluoro-2,4-dimethyl-1H-indol-3-yl)ethyl]benzamide | 336 | 337 | 9.02 |
| 53 | 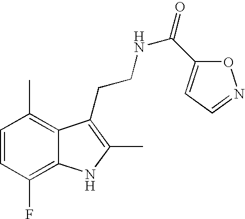 | isoxazole-5-carboxylic acid [2-(7-fluoro-2,4-dimethyl-1H-indol-3-yl)ethyl]-amide | 302 | 303 | 8.17 |
| 54 | 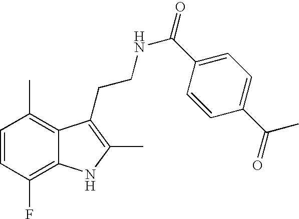 | 4-acetyl-N-[2-(7-fluoro-2,4-dimethyl-1H-indol-3-yl)ethyl]-benzamide | 353 | 354 | 8.75 |
| 55 | 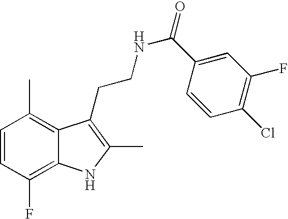 | 4-chloro-3-fluoro-N-[2-(7-fluoro-2,4-dimethyl-1H-indol-3-yl)ethyl]benzamide | 363 | 364 | 10.12 |
| 56 | 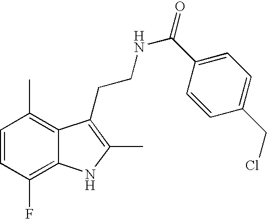 | 4-chloromethyl-N-[2-(7-fluoro-2,4-dimethyl-1H-indol-3-yl)ethyl]benzamide | 359 | 360 | 9.37 |
| 57 | 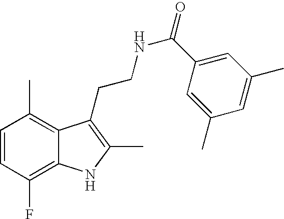 | N-[2-(7-fluoro-2,4-dimethyl-1H-indol-3-yl)ethyl]-3,5-dimethylbenzamide | 339 | 340 | 9.88 |
| 58 | 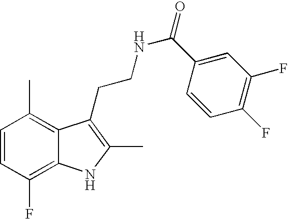 | 3,4-difluoro-N-[2-(7-fluoro-2,4-dimethyl-1H-indol-3-yl)ethyl]-benzamide | 347 | 348 | 9.63 |
| 59 | 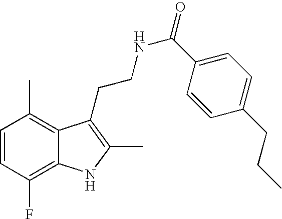 | N-[2-(7-fluoro-2,4-dimethyl-1H-indol-3-yl)ethyl]-4-propylbenzamide | 353 | 354 | 10.44 |
| 60 | 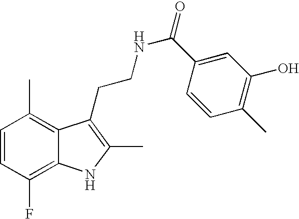 | N-[2-(7-fluoro-2,4-dimethyl-1H-indol-3-yl)ethyl]-3-hydroxy-4-methylbenzamide | 341 | 342 | 8.4 |
| 61 | 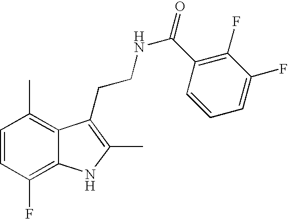 | 2,3-difluoro-N-[2-(7-fluoro-2,4-dimethyl-1H-indol-3-yl)ethyl]benzamide | 347 | 348 | 9.49 |
| 62 | 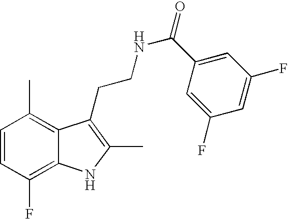 | 3,5-difluoro-N-[2-(7-fluoro-2,4-dimethyl-1H-indol-3-yl)ethyl]benzamide | 347 | 348 | 9.77 |
| 63 | 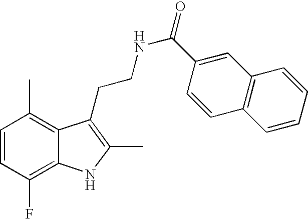 | naphthalene-2-carboxylic acid [2-(7-fluoro-2,4-dimethyl-1H-indol-3-yl)ethyl]-amide | 361 | 362 | 9.92 |
| 64 | 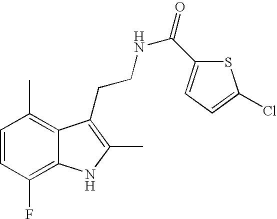 | 5-chlorothiophene-2-carboxylic acid [2-(7-fluoro-2,4-dimethyl-1H-indol-3-yl)ethyl]-amide | 351 | 352 | 10.05 |
| 65 | 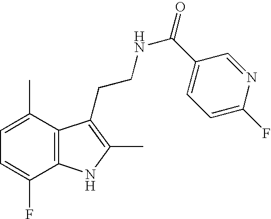 | 6-fluoro-N-[2-(7-fluoro-2,4-dimethyl-1H-indol-3-yl)ethyl]-nicotinamide | 330 | 331 | 8.59 |
| 66 | 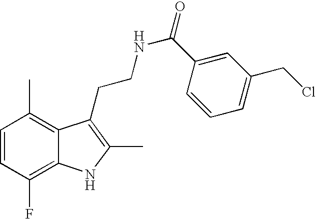 | 3-chloromethyl-N-[2-(7-fluoro-2,4-dimethyl-1H-indol-3-yl)ethyl]benzamide | 359 | 360 | 9.55 |
| 67 | 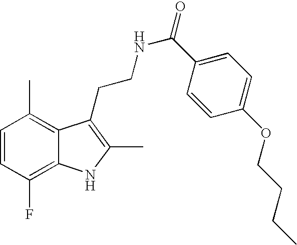 | 4-butoxy-N-[2-(7-fluoro-2,4-dimethyl-1H-indol-3-yl)ethyl]benzamide | 383 | 384 | 10.55 |
| 68 | 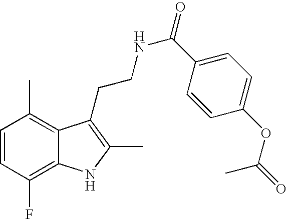 | 4-acetoxy-N-[2-(7-fluoro-2,4-dimethyl-1H-indol-3-yl)ethyl]benzamide | 369 | 370 | 8.85 |
| 69 | 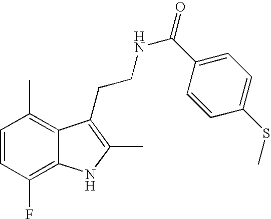 | N-[2-(7-fluoro-2,4-dimethyl-1H-indol-3-yl)ethyl]-4-methylsulfanyl-benzamide | 357 | 358 | 9.55 |
| 70 | 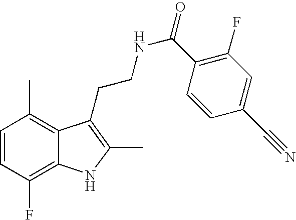 | 4-cyano-2-fluoro-N-[2-(7-fluoro-2,4-dimethyl-1H-indol-3-yl)ethyl]benzamide | 354 | 355 | 9.2 |
| 71 | 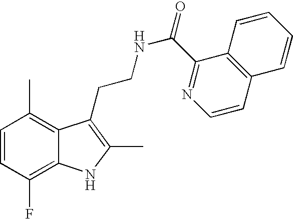 | isoquinoline-1-carboxylic acid [2-(7-fluoro-2,4-dimethyl-1H-indol-3-yl)ethyl]-amide | 362 | 363 | 9.79 |
| 72 | 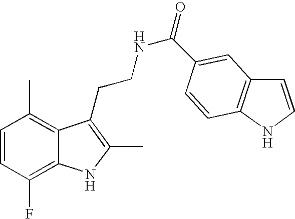 | isoquinoline-1-carboxylic acid [2-(7-fluoro-2,4-dimethyl-1H-indol-3-yl)ethyl]-amide | 350 | 351 | 8.44 |
| 73 | 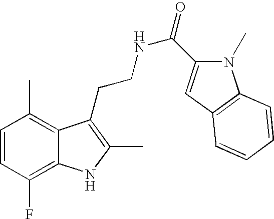 | isoquinoline-1-carboxylic acid [2-(7-fluoro-2,4-dimethyl-1H-indol-3-yl)ethyl]-amide | 364 | 365 | 10.25 |
| 74 | 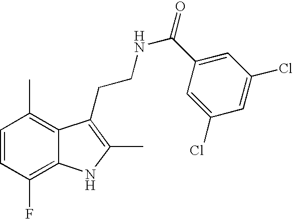 | 3,5-dichloro-N-[2-(7-fluoro-2,4-dimethyl-1H-indol-3-yl)ethyl]benzamide | 379 | 380 | 10.85 |
| 75 | 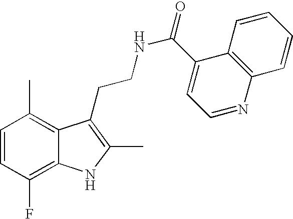 | quinoline-4-carboxylic acid [2-(7-fluoro-2,4-dimethyl-1H-indol-3-yl)ethyl]amide | 361 | 362 | 8.38 |
| 76 | 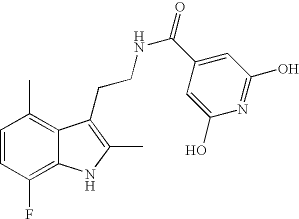 | quinoline-4-carboxylic acid [2-(7-fluoro-2,4-dimethyl-1H-indol-3-yl)ethyl]amide | 343 | 344 | 7.08 |
| 77 | 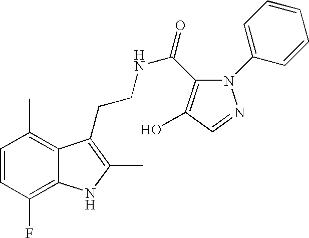 | 4-hydroxy-2-phenyl-2H-pyrazole-3-carboxylic acid [2-(7-fluoro-2,4-dimethyl-1H-indol-3-yl)ethyl]amide | 392 | 393 | 8.61 |
| 78 | 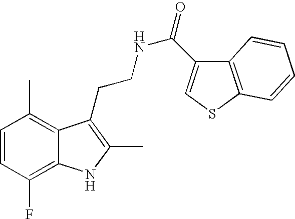 | benzo[b]thiophene-3-carboxylic acid [2-(7-fluoro-2,4-dimethyl-1H-indol-3-yl)ethyl[amide | 366 | 367 | 10.38 |
| 79 | 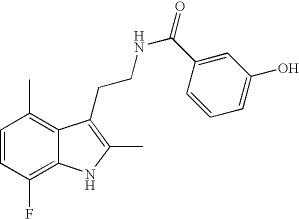 | N-[2-(7-fluoro-2,4-dimethyl-1H-indol-3-yl)ethyl]-3-hydroxybenzamide | 326 | 327 | 8.45 |
| 80 | 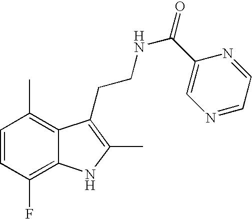 | pyrazine-2-carboxylic acid [2-(7-fluoro-2,4-dimethyl-1H-indol-3-yl)ethyl]amide | 312 | 313 | 8.67 |
| 81 | 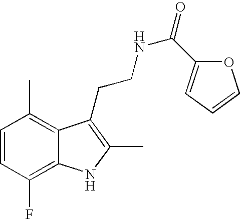 | furan-2-carboxylicacid [2-(7-fluoro-2,4-dimethyl-1H-indol-3-yl)ethyl]amide | 300 | 301 | 8.85 |
| 82 | 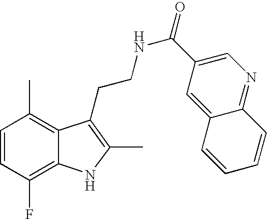 | quinoline-3-carboxylic acid [2-(7-fluoro-2,4-dimethyl-1H-indol-3-yl)ethyl]amide | 361 | 362 | 8.97 |
| 83 | 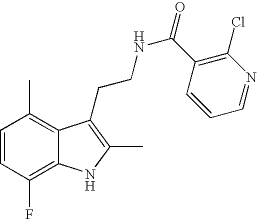 | 2-chloro-N-[2-(7-fluoro-2,4-dimethyl-1H-indol-3-yI)ethyl]-nicotinamide | 346 | 347 | 8.58 |
| 84 | 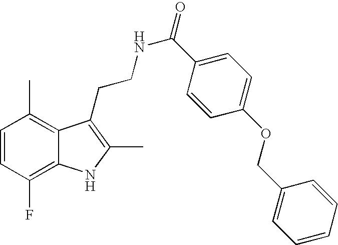 | 4-benzyloxy-N-[2-(7-fluoro-2,4-dimethyl-1H-indol-3-yl)ethyl]benzamide | 416 | 417 | 10.8 |
| 85 | 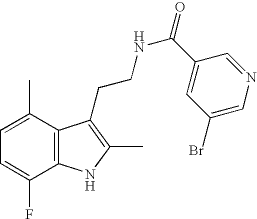 | 5-bromo-N-[2-(7-fluoro-2,4-dimethyl-1H-indol-3-yl)ethyl]-nicotinamide | 390 | 391 | 9.44 |
| 86 | 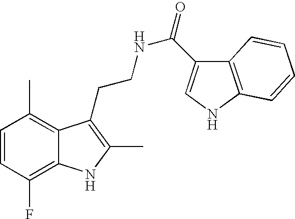 | 1H-indole-3-carboxylic acid [2-(7-fluoro-2,4-dimethyl-1H-indol-3-yl)ethyl]amide | 349 | 350 | 8.98 |
| 87 | 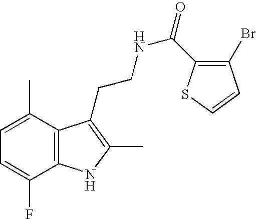 | 3-bromothiophene-2-carboxylic acid [2-(7-fluoro-2,4-dimethyl-1H-indol-3-yl)ethyl]amide | 395 | 396 | 10.25 |
| 88 | 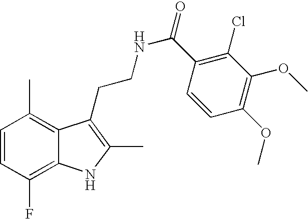 | 2-chloro-N-[2-(7-fluoro-2,4-dimethyl-1H-indol-3-yl)ethyl]-3,4-dimethoxy-benzamide | 405 | 406 | 9.47 |
| 89 | 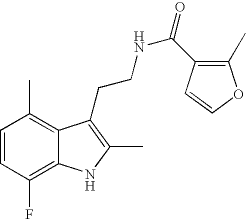 | 2-methylfuran-3-carboxylic acid [2-(7-fluoro-2,4-dimethyl-1H-indol-3-yl)ethyl]amide | 314 | 315 | 9.35 |
| 90 | 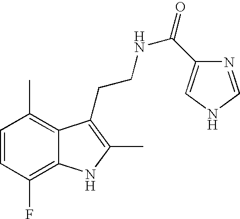 | 1H-imidazole-4-carboxylic acid [2-(7-fluoro-2,4-dimethyl-1H-indol-3-yl)ethyl]amide | 300 | 301 | 6.35 |
| 91 | 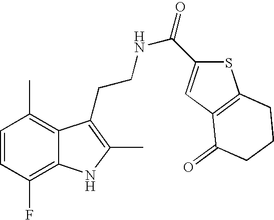 | 4-oxo-4,5,6,7-tetrahydro-benzo[b]thiophene-2-carboxylic acid [2-(7-fluoro-2,4-dimethyl-1H-indol-3-yl)ethyl]amide | 384 | 385 | 9.42 |
| 92 | 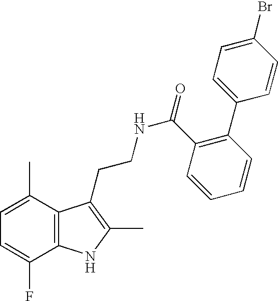 | 4′-bromobiphenyl-2-carboxylic acid [2-(7-fluoro-2,4-dimethyl-1H-indol-3-yl)ethyl]amide | 465 | 466 | 10.83 |
| 93 | 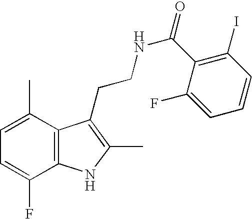 | 2-fluoro-N-[2-(7-fluoro-2,4-dimethyl-1H-indol-3-yl)ethyl]-6-iodobenzamide | 454 | 455 | 9.82 |
| 94 | 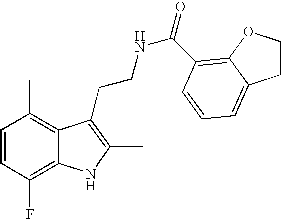 | 2,3-dihydro-benzofuran-7-carboxylic acid [2-(7-fluoro-2,4-dimethyl-1H-indol-3-yl)ethyl]amide | 352 | 353 | 9.86 |
| 95 | 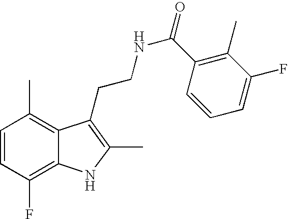 | 3-fluoro-N-[2-(7-fluoro-2,4-dimethyl-1H-indol-3-yl)ethyl]-2-methylbenzamide | 342 | 343 | 9.8 |
| 96 | 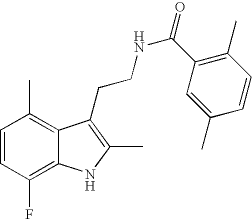 | N-[2-(7-fluoro-2,4-dimethyl-1H-indol-3-yl)ethyl]-2,5-dimethylbenzamide | 338 | 339 | 10.02 |
| 97 | 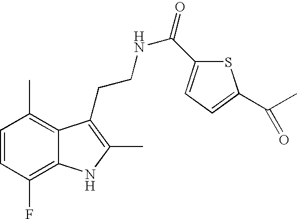 | 5-acetylthiophene-2-carboxylic acid [2-(7-fluoro-2,4-dimethyl-1H-indol-3-yl)ethyl]amide | 358 | 359 | 9.21 |
| 98 | 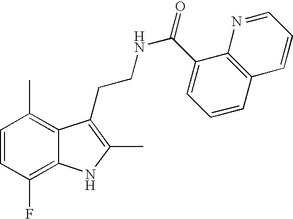 | quinoline-8-carboxylic acid [2-(7-fluoro-2,4-dimethyl-1H-indol-3-yl)ethyl]amide | 361 | 362 | 9.92 |
| 99 | 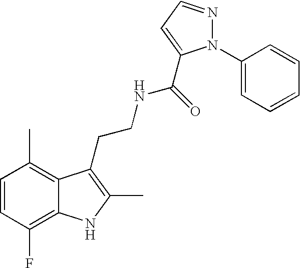 | 2-phenyl-2H-pyrazole-3-carboxylic acid [2-(7-fluoro-2,4-dimethyl-1H-indol-3-yl)ethyl]amide | 376 | 377 | 9.4 |
| 100 | 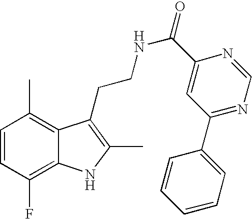 | 6-phenylpyrimidine-4-carboxylic acid [2-(7-fluoro-2,4-dimethyl-1H-indol-3-yl)ethyl]amide | 388 | 389 | 10.98 |
| 101 | 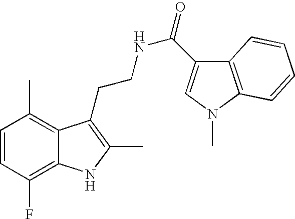 | 1-methyl-1H-indole-3-carboxylic acid [2-(7-fluoro-2,4-dimethyl-1H-indol-3-yl)ethyl]amide | 363 | 364 | 11.32 |
| 102 | 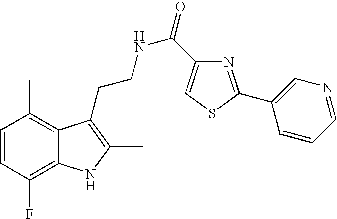 | 2-pyridin-3-ylthiazole-4-carboxylic acid [2-(7-fluoro-2,4-dimethyl-1H-indol-3-yl)ethyl]amide | 394 | 395 | 9.17 |
| 103 | 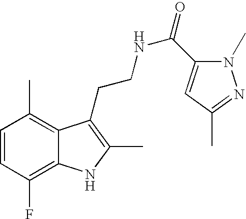 | 2,5-dimethyl-2H-pyrazole-3-carboxylic acid [2-(7-fluoro-2,4-dimethyl-1H-indol-3-yl)ethyl]amide | 328 | 329 | 8.83 |
| 104 | 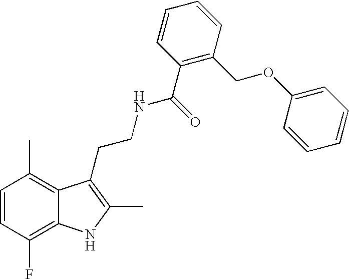 | N-[2-(7-fluoro-2,4-dimethyl-1H-indol-3-yl)ethyl]-2-phenoxymethyl-benzamide | 416 | 417 | 10.67 |
| 105 | 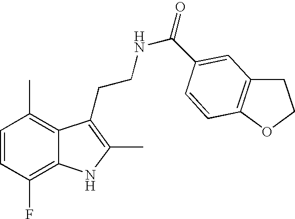 | 2,3-dihydro-benzofuran-5-carboxylic acid [2-(7-1H-indol-3-yl)ethyl]amide | 352 | 353 | 9.33 |
| 106 | 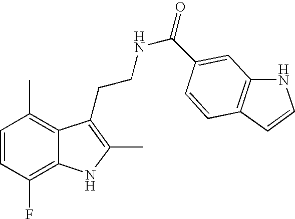 | 1H-indole-6-carboxylic acid [2-(7-fluoro-2,4-dimethyl-1H-indol-3-yl)ethyl]amide | 349 | 350 | 9.17 |
| 107 | 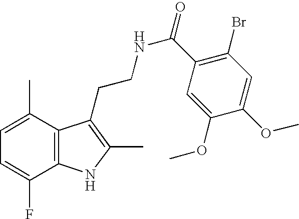 | 2-bromo-N-[2-(7-fluoro-2,4-dimethyl-1H-indol-3-yl)ethyl]-4,5-dimethoxy-benzamide | 449 | 450 | 9.38 |
| 108 | 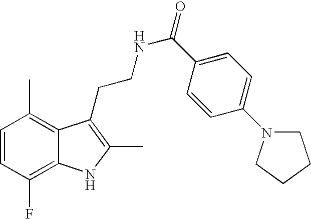 | N-[2-(7-fluoro-2,4-dimethyl-1H-indol-3-yl)ethyl]-4-pyrrolidin-1-ylbenzamide | 379 | 380 | 10.31 |
| 109 | 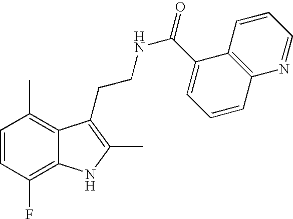 | quinoline-5-carboxylic acid [2-(7-fluoro-2,4-dimethyl-1H-indol-3-yl)ethyl]amide | 361 | 362 | 6.88 |
| 110 | 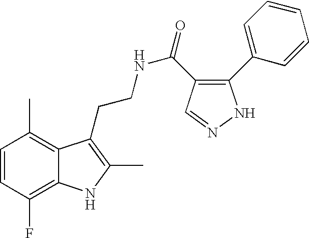 | 5-phenyl-1H-pyrazole-4-carboxylic acid [2-(7-fluoro-2,4-dimethyl-1H-indol-3-yl)ethyl]amide | 376 | 377 | 8.53 |
| 111 | 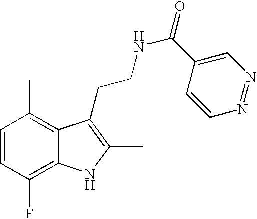 | pyridazine-4-carboxylic acid [2-(7-fluoro-2,4-dimethyl-1H-indol-3-yl)ethyl]amide | 312 | 313 | 7.57 |
| 112 | 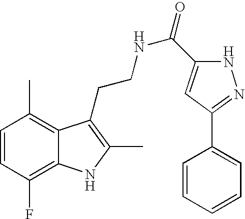 | 5-phenyl-2H-pyrazole-3-carboxylic acid [2-(7-fluoro-2,4-dimethyl-1H-indol-3-yl)ethyl]amide | 376 | 377 | 9.38 |
| 113 | 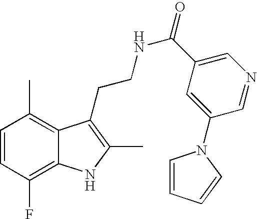 | N-[2-(7-fluoro-2,4-dimethyl-1H-indol-3-yl)ethyl]-5-pyrrol-1-yl-nicotinamide | 376 | 377 | 9.27 |
| 114 | 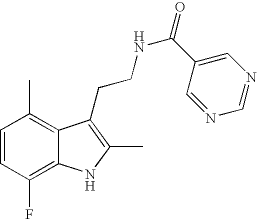 | pyrimidine-5-carboxylic acid [2-(7-fluoro-2,4-dimethyl-1H-indol-3-yl)ethyl]amide | 312 | 313 | 7.85 |
| 115 | 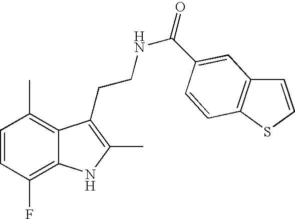 | benzo[b]thiophene-5-carboxylic acid [2-(7-fluoro-2,4-dimethyl-1H-indol-3-yl)ethyl]amide | 366 | 367 | 10.11 |
| 116 | 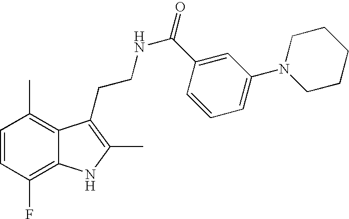 | N-[2-(7-fluoro-2,4-dimethyl-1H-indol-3-yl)ethyl]-3-piperidin-1-ylbenzamide | 394 | 395 | 7.25 |
| 117 | 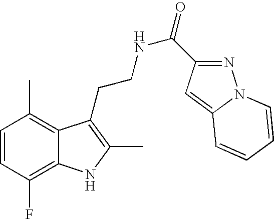 | pyrazolo[1,5-a]pyridine-2-carboxylic acid [2-(7-fluoro-2,4-dimethyl-1H-indol-3-yl)ethyl]amide | 350 | 351 | 9.33 |
| 118 | 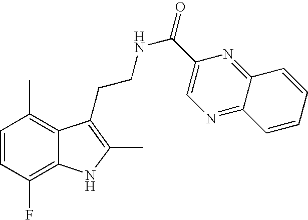 | quinoxaline-2-carboxylic acid [2-(7-fluoro-2,4-dimethyl-1H-indol-3-yl)ethyl]amide | 362 | 363 | 10.13 |
| 119 | 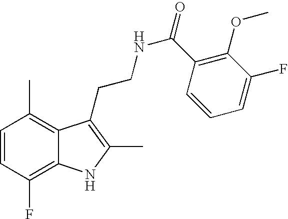 | 3-fluoro-N-[2-(7-fluoro-2,4-dimethyl-1H-indol-3-yl)ethyl]-2-methoxy-benzamide | 358 | 359 | 10.07 |
| 120 | 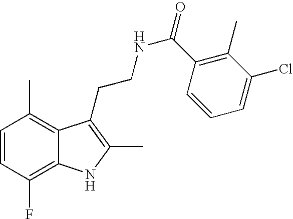 | 3-chloro-N-[2-(7-fluoro-2,4-dimethyl-1H-indol-3-yl)ethyl]-2-methylbenzamide | 359 | 360 | 10.23 |
| 121 | 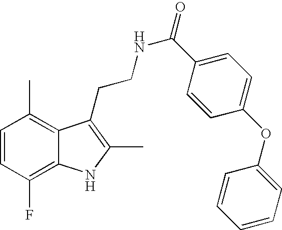 | N-[2-(7-fluoro-2,4-dimethyl-1H-indol-3-yl)ethyl]-4-phenoxbenzamide | 402 | 403 | 10.75 |
| 122 | 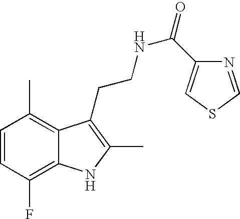 | thiazole-4-carboxylicacid [2-(7-fluoro-2,4-dimethyl-1H-indol-3-yl)ethyl]amide | 317 | 318 | 8.88 |
| 123 | 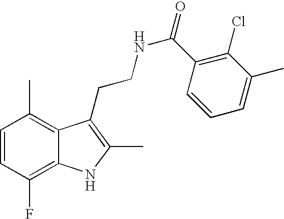 | 2-chloro-N-[2-(7-fluoro-2,4-dimethyl-1H-indol-3-yl)ethyl]-3-methylbenzamide | 359 | 360 | 9.93 |
| 124 | 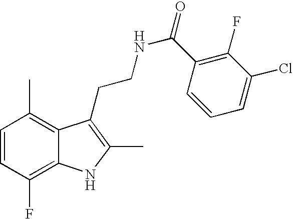 | 3-chloro-2-fluoro-N-[2-(7-fluoro-2,4-dimethyl-1H-indol-3-yl)ethyl]benzamide | 363 | 364 | 10.22 |
| 125 | 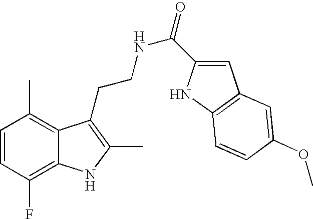 | 5-methoxy-1H-indole-2-carboxylicacid [2-(7-fluoro-2,4-dimethyl-1H-indol-3-yl)ethyl]amide | 379 | 380 | 9.36 |
| 126 | 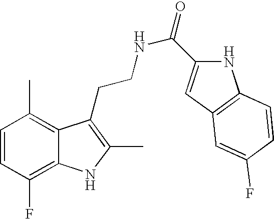 | 5-fluoro-1H-indole-2-carboxylic acid [2-(7-fluoro-2,4-dimethyl-1H-indol-3-yl)ethyl]amide | 367 | 368 | 9.55 |
| 127 | 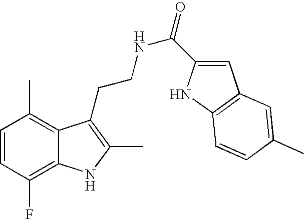 | 5-methyl-1H-indole-2-carboxylic acid [2-(7-fluoro-2,4-dimethyl-1H-indol-3-yl)ethyl]amide | 363 | 364 | 9.84 |
| 128 | 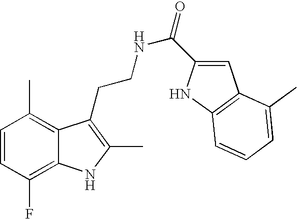 | 4-methyl-1H-indole-2-carboxylic acid [2-(7-fluoro-2,4-dimethyl-1H-indol-3-yl)ethyl]amide | 363 | 364 | 9.83 |
| 129 | 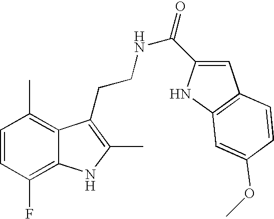 | 6-methoxy-1H-indole-2-carboxylicacid [2-(7-fluoro-2,4-dimethyl-1H-indol-3-yl)ethyl]amide | 379 | 380 | 9.41 |
| 130 | 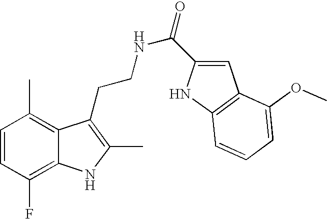 | 4-methoxy-1H-indole-2-carboxylicacid [2-(7-fluoro-2,4-dimethyl-1H-indol-3-yl)ethyl]amide | 379 | 380 | 9.41 |
| 131 | 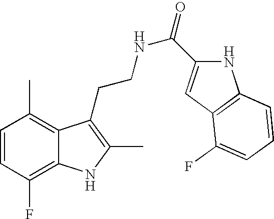 | 4-fluoro-1H-indole-2-carboxylic acid [2-(7-fluoro-2,4-dimethyl-1H-indol-3-yl)ethyl]amide | 367 | 368 | 9.85 |
| 132 | 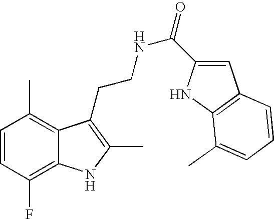 | 7-methyl-1H-indole-2-carboxylic acid [2-(7-fluoro-2,4-dimethyl-1H-indol-3-yl)ethyl]amide | 363 | 364 | 10.67 |
| 133 | 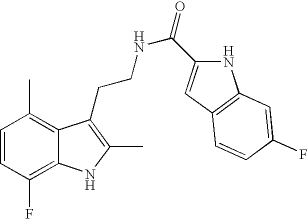 | 6-fluoro-1H-indole-2-carboxylic acid [2-(7-fluoro-2,4-dimethyl-1H-indol-3-yl)ethyl]amide | 367 | 368 | 9.66 |
| 134 | 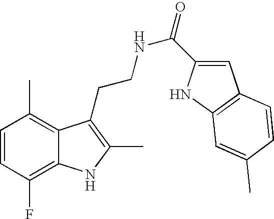 | 6-methyl-1H-indole-2-carboxylic acid [2-7-fluoro-2,4-dimethyl-1H-indol-3-yl)ethyl]amide | 363 | 364 | 9.92 |
| 135 | 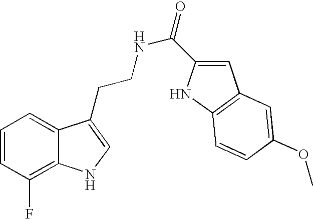 | 5-methoxy-1H-indole-2-carboxylic acid [2-(7-fluoro-1H-indol-3-yl)ethyl]amide | 351 | 352 | 8.73 |
| 136 | 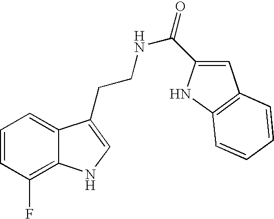 | 1H-indole-2-carboxylic acid [2-(7-fluoro-1H-indol-3-yl)ethyl]amide | 321 | 322 | 8.87 |
| 137 | 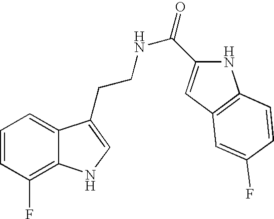 | 5-fluoro-1H-indole-2-carboxylic acid [2-(7-fluoro-1H-indol-3-yl)ethyl]amide | 339 | 340 | 8.95 |
| 138 | 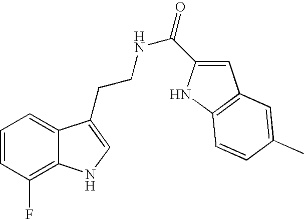 | 5-methyl-1H-indole-2-carboxylic acid [2-(7-fluoro-1H-indol-3-yl)ethyl]amide | 335 | 336 | 9.2 |
| 139 | 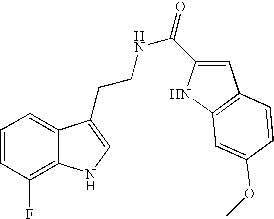 | 6-methoxy-1H-indole-2-carboxylicacid [2-(7-fluoro-1H-indol-3-yl)ethyl]amide | 351 | 352 | 8.74 |
| 140 | 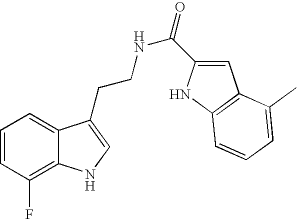 | 4-methyl-1H-indole-2-carboxylic acid [2-(7-fluoro-1H-indol-3-yl)ethyl]amide | 335 | 336 | 9.18 |
| 141 | 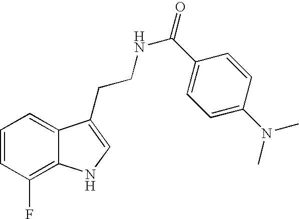 | 4-dimethylamino-N-[2-(7-fluoro-1H-indol-3-yl)ethyl]benzamide | 325 | 326 | 7.63 |
| 142 | 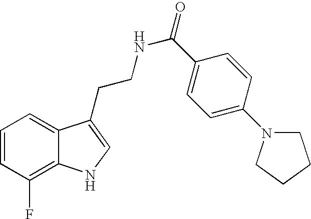 | N-[2-(7-fluoro-1H-indol-3-yl)ethyl]-4-pyrrolidin-1-ylbenzamide | 351 | 352 | 9.22 |
| 143 | 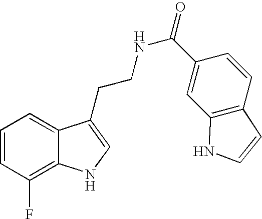 | 1H-indole-6-carboxylic acid [2-(7-fluoro-1H-indol-3-yl)ethyl]amide | 321 | 322 | 8.21 |
| 144 | 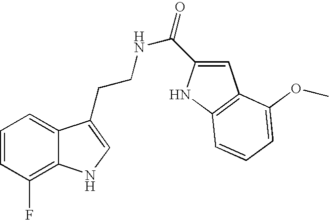 | 4-methoxy-1H-indole-2-carboxylicacid [2-(7-fluoro-1H-indol-3-yl)ethyl]amide | 351 | 352 | 8.88 |
| 145 | 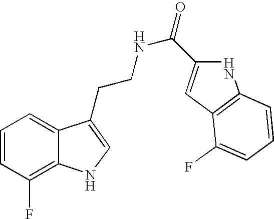 | 4-fluoro-1H-indole-2-carboxylic acid [2-(7-fluoro-1H-indol-3-yl)ethyl]amide | 339 | 340 | 9.02 |
| 146 | 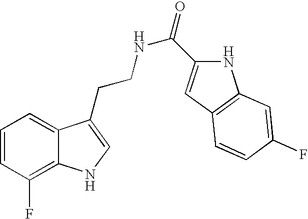 | 6-fluoro-1H-indole-2-carboxylic acid [2-(7-fluoro-1H-indol-3-yl)ethyl]amide | 339 | 340 | 9.03 |
| 147 | 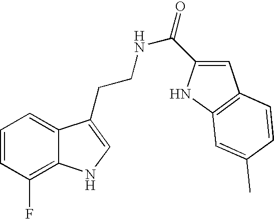 | 6-methyl-1H-indole-2-carboxylic acid [2-(7-fluoro-1H-indol-3-yl)ethyl]amide | 335 | 336 | 9.28 |
| 148 | 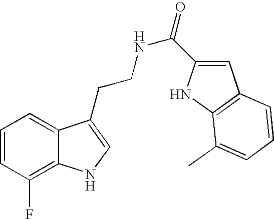 | 7-methyl-1H-indole-2-carboxylic acid [2-(7-fluoro-1H-indol-3-yl)ethyl]amide | 335 | 336 | 9.22 |
| 149 | 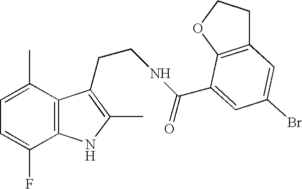 | 5-bromo-2,3-dihydrobenzofuran-7-carboxylic acid [2-(7-fluoro-2,4-dimethyl-1H-indol-3-yl)ethyl]amide | 432 | 433 | 8.04 |
| 150 | 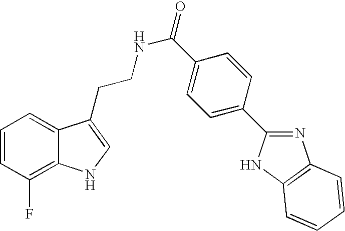 | 4-(1H-benzoimidazol-2-yl)-N-[2-(7-fluoro-1H-indol-3-yl)ethyl]benzamide | 398 | 399 | 6.51 |
| 151 | 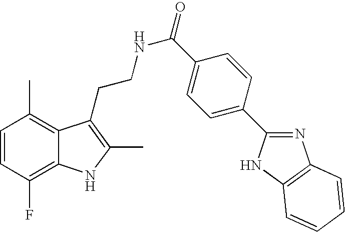 | 4-(1H-benzoimidazol-2-yl)-N-[2-(7-fluoro-2,4-dimethyl-1H-indol-3-yl)ethyl]benzamide | 426 | 427 | 6.95 |
| 152 | 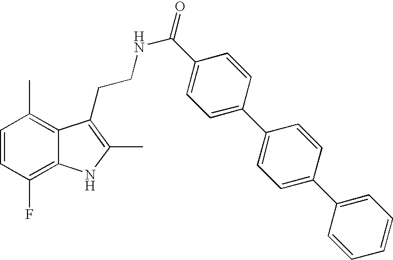 | [1,1′;4′,1″]terphenyl-4-carboxylic acid [2-(7-fluoro-2,4-dimethyl-1H-indol-3-yl)ethyl]amide | 462.5653 | 464 | 4.99 |
| 153 | 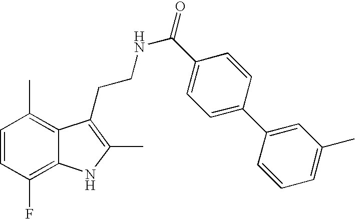 | 3′-methylbiphenyl-4-carboxylic acid [2-(7-fluoro-2,4-dimethyl-1H-indol-3-yl)ethyl]amide | 400.4945 | 401 | 4.69 |
| 154 | 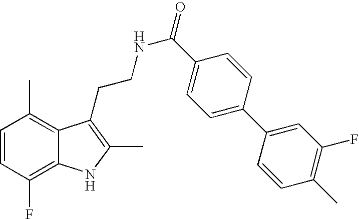 | 3′-fluoro-4′-methyl-biphenyl-4-carboxylic acid [2-(7-fluoro-2,4-dimethyl-1H-indol-3-yl)ethyl]amide | 418.4846 | 419 | 4.72 |
| 155 | 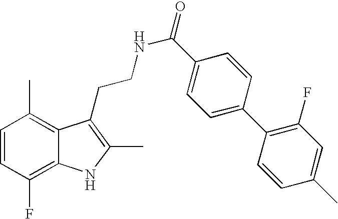 | 2′-fluoro-4′-methyl-biphenyl-4-carboxylic acid [2-(7-fluoro-2,4-dimethyl-1H-indol-3-yl)ethyl]amide | 418.4846 | 419 | 4.72 |
| 156 | 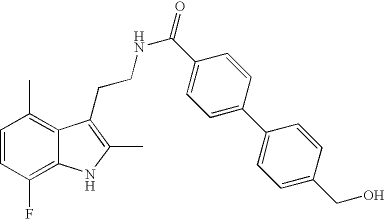 | 4′-hydroxymethyl-biphenyl-4-carboxylic acid [2-(7-fluoro-2,4-dimethyl-1H-indol-3-yl)ethyl]amide | 416.4935 | 417 | 3.81 |
| 157 | 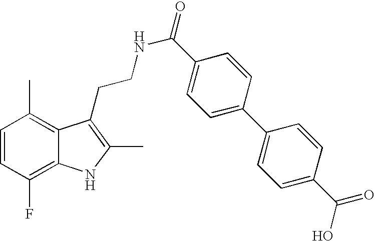 | 4′-[2-(7-fluoro-2,4-dimethyl-1H-indol-3-yl)ethylcarbamoyl]-biphenyl-4-carboxylic acid | 430.4767 | 431 | 3.86 |
| 158 | 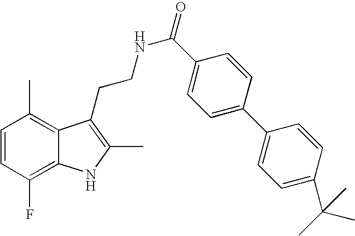 | 4′-tert-butylbiphenyl-4-carboxylic acid [2-(7-fluoro-2,4-dimethyl-1H-indol-3-yl)ethyl]amide | 442.5749 | 444 | 5.12 |
| 159 | 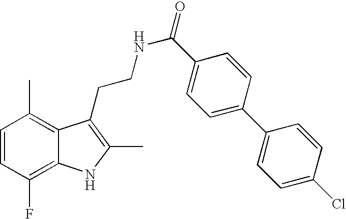 | 4′-chlorobiphenyl-4-carboxylic acid [2-(7-fluoro-2,4-dimethyl-1H-indol-3-yl)ethyl]amide | 420.9128 | 422 | 4.72 |
| 160 | 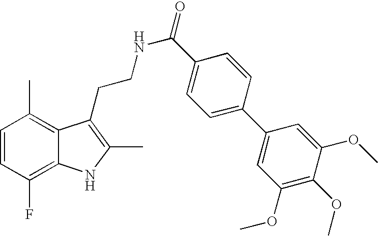 | 3′,4′,5′-trimethoxy-biphenyl-4-carboxylic acid [2-(7-fluoro-2,4-dimethyl-1H-indol-3-yl)ethyl]amide | 476.5451 | 478 | 4.19 |
| 161 | 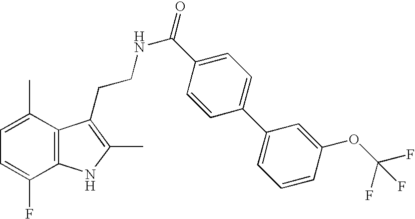 | 3′-trifluoromethoxy-biphenyl-4-carboxylic acid [2-(7-fluoro-2,4-dimethyl-1H-indol-3-yl)ethyl]amide | 470.4638 | 471 | 4.84 |
| 162 | 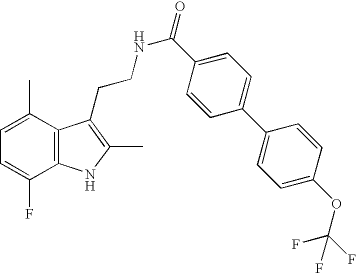 | 4′-trifluoromethoxy-biphenyl-4-carboxylic acid [2-(7-fluoro-2,4-dimethyl-1H-indol-3-yl)ethyl]amide | 470.4638 | 471 | 4.8 |
| 163 | 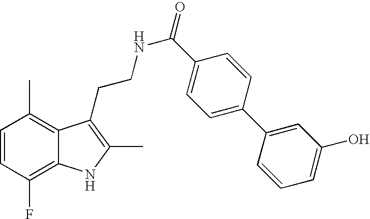 | 3′-hydroxybiphenyl-4-carboxylic acid [2-(7-fluoro-2,4-dimethyl-1H-indol-3-yl)ethyl]amide | 402.4667 | 403 | 3.97 |
| 164 | 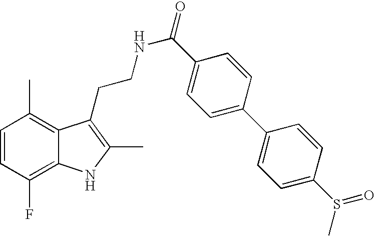 | 4′-methanesulfinyl-biphenyl-4-carboxylic acid [2-(7-fluoro-2,4-dimethyl-1H-indol-3-yl)ethyl amide | 448.5595 | 450 | 3.64 |
| 165 | 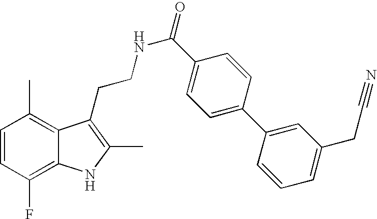 | 3′-cyanomethyl-biphenyl-4-carboxylic acid [2-(7-fluoro-2,4-dimethyl-1H-indol-3-yl)ethyl]amide | 425.5046 | 427 | 4.17 |
| 166 | 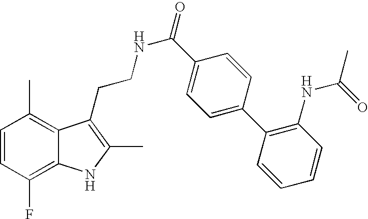 | 2′-acetylamino-biphenyl-4-carboxylic acid [2-(7-fluoro-2,4-dimethyl-1H-indol-3-yl)ethyl]amide | 443.5194 | 445 | 3.72 |
| 167 | 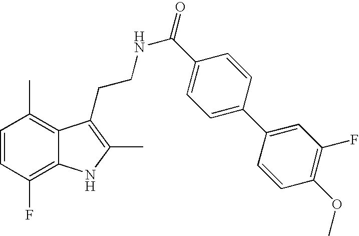 | 3′-fluoro-4′-methoxy-biphenyl-4-carboxylic acid [2-(7-fluoro-2,4-dimethyl-1H-indol-3-yl)ethyl]amide | 434.4836 | 435 | 4.36 |
| 168 | 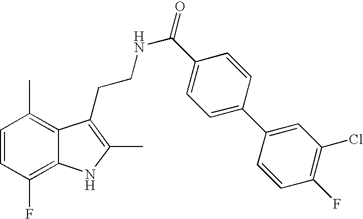 | 3′-chloro-4′-fluoro-biphenyl-4-carboxylic acid [2-(7-fluoro-2,4-dimethyl-1H-indol-3-yl)ethyl]amide | 438.9029 | 440 | 4.74 |
| 169 | 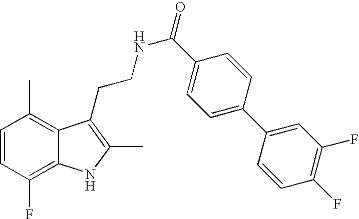 | 3′,4′-difluoro-biphenyl-4-carboxylic acid [2-(7-fluoro-2,4-dimethyl-1H-indol-3-yl)ethyl]amide | 422.4479 | 423 | 4.55 |
| 170 | 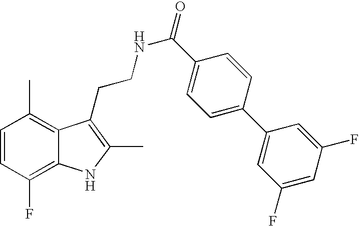 | 3′,5′-difluoro-biphenyl-4-carboxylic acid [2-(7-fluoro-2,4-dimethyl-1H-indol-3-yl)ethyl]amide | 422.4479 | 423 | 4.57 |
| 171 | 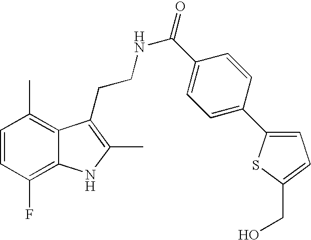 | N-[2-(7-fluoro-2,4-dimethyl-1H-indol-3-yl)ethyl]-4-(5-hydroxymethyl-thiophen-2-yl)-benzamide | 422.5217 | 424 | 3.82 |
| 172 | 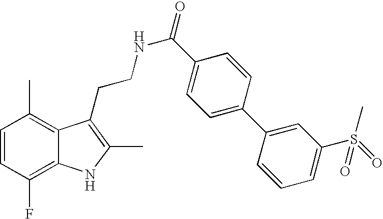 | 3′-methanesulfonyl-biphenyl-4-carboxylic acid [2-(7-fluoro-2,4-dimethyl-1H-indol-3-yl)ethyl]amide | 464.5585 | 466 | 3.94 |
| 173 | 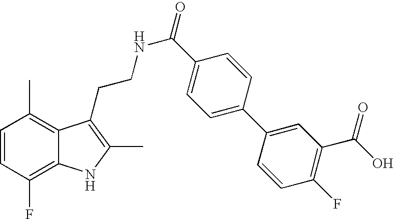 | 4-fluoro-4′-[2-(7-fluoro-2,4-dimethyl-1H-indol-3-yl)ethylcarbamoyl]-biphenyl-3-carboxylic acid | 448.4668 | 449 | 3.89 |
| 174 | 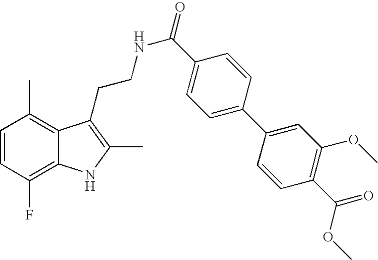 | 4′-[2-(7-fluoro-2,4-dimethyl-1H-indol-3-yl)ethylcarbamoyl]-3-methoxybiphenyl-4-carboxylic acidmethyl ester | 474.5293 | 476 | 4.2 |
| 175 | 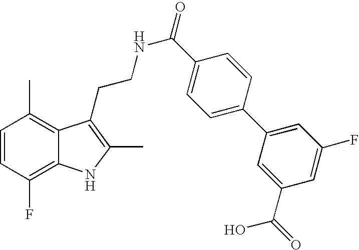 | 5-fluoro-4′-[2-(7-fluoro-2,4-dimethyl-1H-indol-3-yl)ethylcarbamoyl]-biphenyl-3-carboxylic acid | 448.4668 | 449 | 4 |
| 176 | 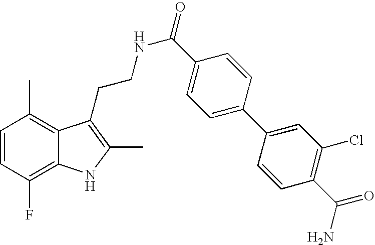 | 3-chlorobiphenyl-4,4′-dicarboxylic acid4-amide 4′-{[2-(7-fluoro-2,4-dimethyl-1H-indol-3-yl)ethyl]amide} | 463.9377 | 465 | 3.69 |
| 177 | 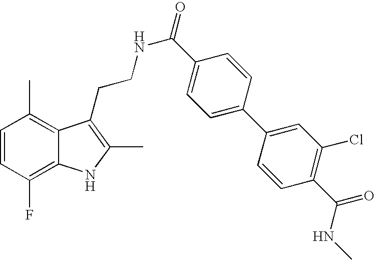 | 3-chlorobiphenyl-4,4′-dicarboxylic acid4′-{[2-(7-fluoro-2,4-dimethyl-1H-indol-3-yl)ethyl]amide} 4-methylamide | 477.9645 | 479 | 3.81 |
| 178 | 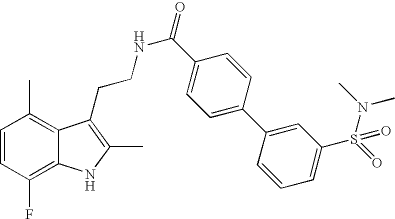 | 3′-dimethyl-sulfamoylbiphenyl-4-carboxylic acid [2-(7-fluoro-2,4-dimethyl-1H-indol-3-yl)ethyl]amide | 493.6002 | 495 | 4.2 |
| 179 | 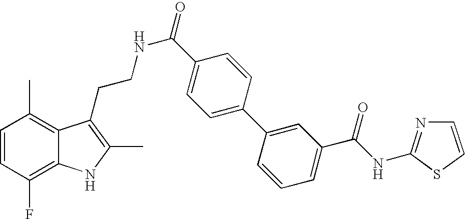 | biphenyl-3,4′-dicarboxylic acid 4′-{[2-(7-fluoro-2,4-dimethyl-1H-indol-3-yl)ethyl]amide} 3-thiazol-2-ylamide | 512.6065 | 514 | 4.15 |
| 180 | 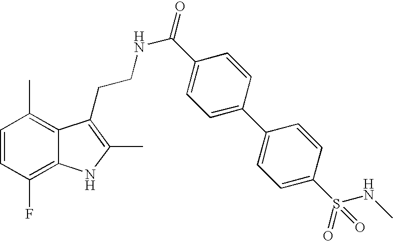 | 4′-methylsulfamoyl-biphenyl-4-carboxylic acid [2-(7-fluoro-2,4-dimethyl-1H-indol-3-yl)ethyl]amide | 479.5734 | 481 | 3.9 |
| 181 | 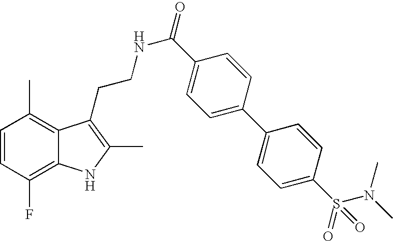 | 4′-dimethyl-sulfamoylbiphenyl-4-carboxylic acid [2-(7-fluoro-2,4-dimethyl-1H-indol-3-yl)ethyl]amide | 493.6002 | 495 | 4.21 |
| 182 | 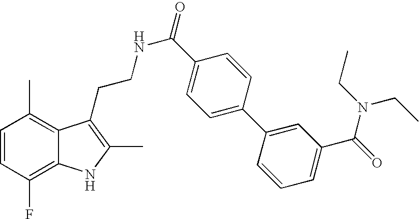 | biphenyl-3,4′-dicarboxylic acid 3-diethylamide 4′-{[2-(7-fluoro-2,4-dimethyl-1H-indol-3-yl)ethyl]amide} | 485.5998 | 487 | 4.14 |
| 183 | 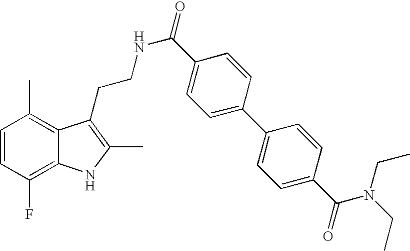 | biphenyl-4,4′-dicarboxylic acid 4- diethylamide 4′-{[2-(7-fluoro-2,4-dimethyl-1H-indol-3-yl)ethyl]amide} | 485.5998 | 487 | 4.1 |
| 184 | 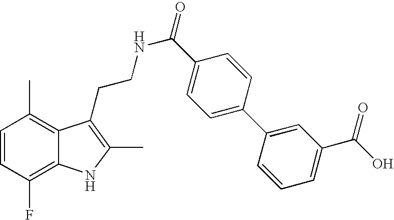 | 4′-[2-(7-fluoro-2,4-dimethyl-1H-indol-3-yl)ethylcarbamoyl]-biphenyl-3-carboxylic acid | 430.4767 | 431 | 3.87 |
| 185 | 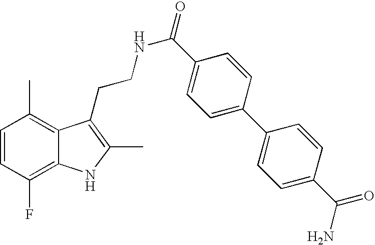 | biphenyl-4,4′-dicarboxylic acid4-amide 4′-{[2-(7-fluoro-2,4-dimethyl-1H-indol-3-yl)ethyl]amide} | 429.4926 | 430 | 3.59 |
| 186 | 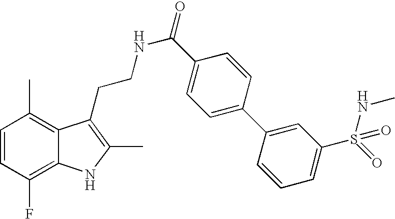 | 3′-methylsulfamoyl-biphenyl-4-carboxylic acid [2-(7-fluoro-2,4-dimethyl-1H-indol-3-yl)ethyl]amide | 479.5734 | 481 | 3.97 |
| 187 | 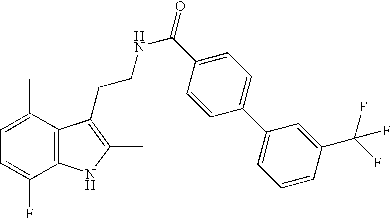 | 3′-trifluoromethyl-biphenyl-4-carboxylic acid [2-(7-fluoro-2,4-dimethyl-1H-indol-3-yl)ethyl]amide | 454.4648 | 455 | 4.73 |
| 188 | 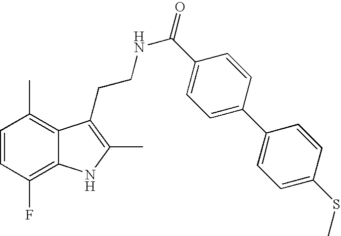 | 4-methylsulfanyl-biphenyl-4-carboxylic acid [2-(7-fluoro-2,4-dimethyl-1H-indol-3-yl)ethyl]amide | 432.5605 | 434 | 4.62 |
| 189 | 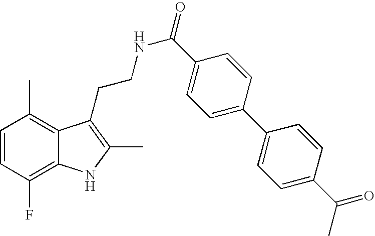 | 4′-acetylbiphenyl-4-carboxylic acid [2-(7-fluoro-2,4-dimethyl-1H-indol-3-yl)ethyl]amide | 428.5045 | 430 | 4.21 |
| 190 | 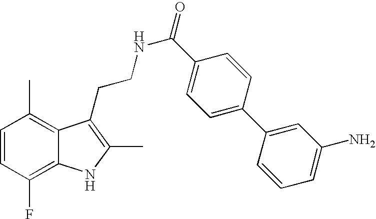 | 3′-aminobiphenyl-4-carboxylic acid [2-(7-fluoro-2,4-dimethyl-1H-indol-3-yl)ethyl]amide | 401.4826 | 402 | 3.27 |
| 191 | 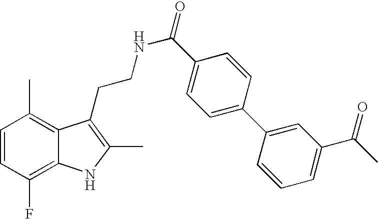 | 3′-acetylbiphenyl-4-carboxylic acid [2-(7-fluoro-2,4-dimethyl-1H-indol-3-yl)ethyl]amide | 428.5045 | 430 | 4.22 |
| 192 | 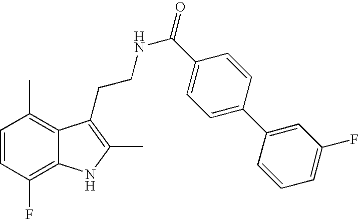 | 3′-fluorobiphenyl-4-carboxylic acid [2-(7-fluoro-2,4-dimethyl-1H-indol-3-yl)ethyl]amide | 404.4578 | 405 | 4.51 |
| 193 | 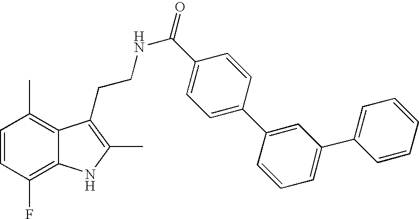 | [1,1′;3′,1″]terphenyl-4-carboxylic acid [2-(7-fluoro-2,4-nl dimethyl-1H-indol-3-yl)ethyl]amide | 462.5653 | 464 | 5.01 |
| 194 | 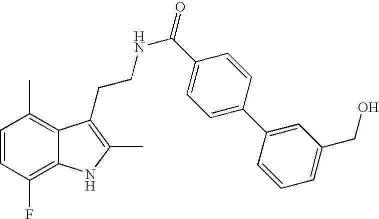 | 3′-hydroxymethyl-biphenyl-4-carboxylic acid [2-(7-fluoro-2,4-dimethyl-1H-indol-3-yl)ethyl]amide | 416.4935 | 417 | 3.87 |
| 195 | 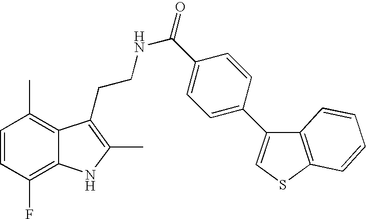 | 4-benzo[b]thiophen-3-yl-N-[2-(7-fluoro-2,4-dimethyl-1H-indol-3-yl)ethyl]benzamide | 442.5557 | 444 | 4.79 |
| 196 | 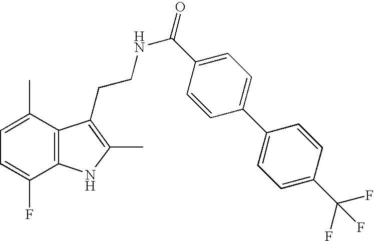 | 4′-trifluoromethyl-biphenyl-4-carboxylic acid [2-(7-fluoro-2,4-dimethyl-1H-indol-3-yl)ethyl]amide | 454.4648 | 455 | 4.76 |
| 197 | 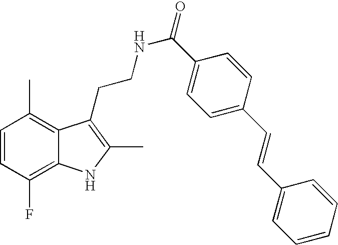 | N-[2-(7-fluoro-2,4-dimethyl-1H-indol-3-yl)ethyl]-4-((E)-styryl)benzamide | 412.5055 | 414 | 4.72 |
| 198 | 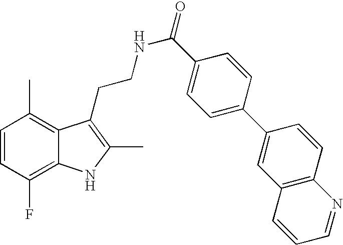 | N-[2-(7-fluoro-2,4-dimethyl-1H-indol-3-yl)ethyl]-4-quinolin-6-ylbenzamide | 437.5156 | 439 | 3.32 |
| 199 | 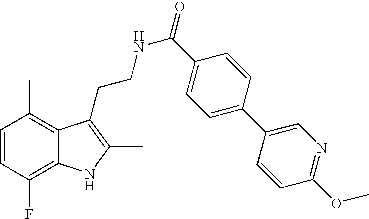 | N-[2-(7-fluoro-2,4-dimethyl-1H-indol-3-yl)ethyl]-4-(6-methoxypyridin-3-yl)-benzamide | 417.4816 | 418 | 4.1 |
| 200 | 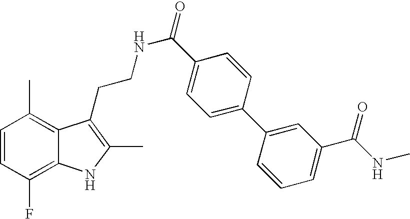 | biphenyl-3,4′-dicarboxylic acid 4′-{[2-(7-fluoro-2,4-dimethyl-1H-indol-3-yl)ethyl]amide} 3-methylamide | 443.5194 | 445 | 3.72 |
| 201 | 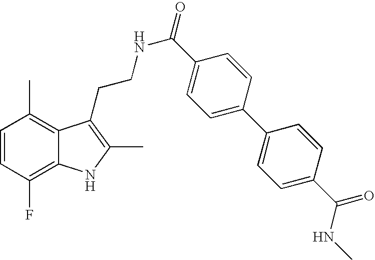 | biphenyl-4,4′-dicarboxylic acid 4′-{[2-(7-fluoro-2,4-dimethyl-1H-indol-3-yl)ethyl]amide} 4-methylamide | 443.5194 | 445 | 3.69 |
| 202 | 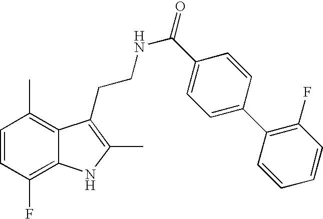 | 2′-fluorobiphenyl-4-carboxylic acid [2-(7-fluoro-2,4-dimethyl-1H-indol-3-yl)ethyl]amide | 404.4578 | 405 | 4.45 |
| 203 | 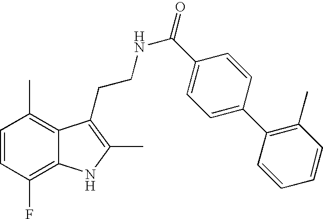 | 2′-methylbiphenyl-4-carboxylic acid [2-(7-fluoro-2,4-dimethyl-1H-indol-3-yl)ethyl]amide | 400.4945 | 401 | 4.62 |
| 204 | 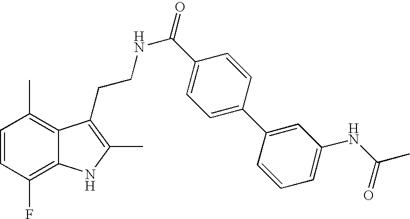 | 3′-acetylamino-biphenyl-4-carboxylic acid [2-(7-fluoro-2,4-dimethyl-1H-indol-3-yl)ethyl]amide | 443.5194 | 445 | 3.84 |
| 205 | 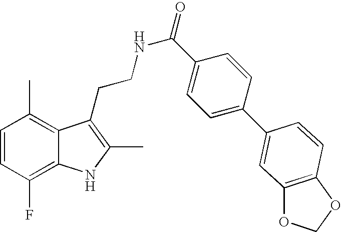 | 4-benzo[1,3]dioxol-5-yl-N-[2-(7-fluoro-2,4-dimethyl-1H-yl)ethyl]benzamide | 430.4767 | 431 | 4.38 |
| 206 | 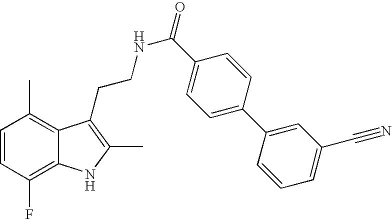 | 3′-cyanobiphenyl-4-carboxylic acid [2-(7-fluoro-2,4-dimethyl-1H-indol-3-yl)ethyl]amide | 411.4778 | 412 | 4.27 |
| 207 | 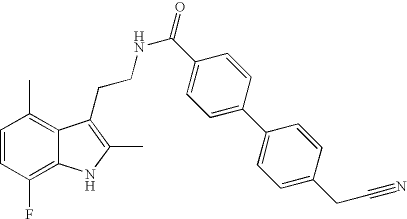 | 4′-cyanomethyl-biphenyl-4-carboxylic acid [2-(7-fluoro-2,4-dimethyl-1H-indol-3-yl)ethyl]amide | 425.5046 | 427 | 4.12 |
| 208 | 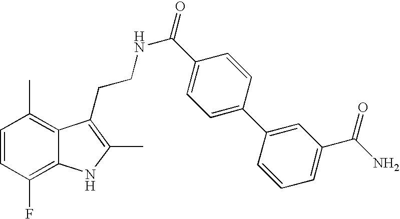 | biphenyl-3,4′-dicarboxylic acid 3-amide 4]-{[2-(7-fluoro-2,4-dimethyl-1H-indol-3-yl)ethyl]amide} | 429.4926 | 430 | 3.79 |
| 209 | 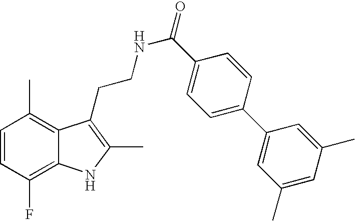 | 3′,5′-dimethyl-biphenyl-4-carboxylic acid [2-(7-fluoro-2,4-dimethyl-1H-indol-3-yl)ethyl]amide | 414.5213 | 416 | 4.89 |
| 210 | 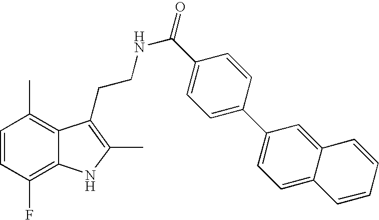 | N-[2-(7-fluoro-2,4-dimethyl-1H-indol-3-yl)ethyl]-4-quinolin-3-ylbenzamide | 437.5156 | 439 | 3.52 |
| 211 | 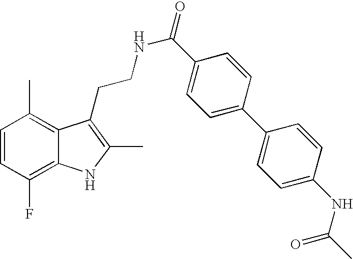 | 4′-acetylamino-biphenyl-4-carboxylic acid [2-(7-fluoro-2,4-dimethyl-1H-indol-3-yl)ethyl]amide | 443.5194 | 445 | 3.75 |
| 212 | 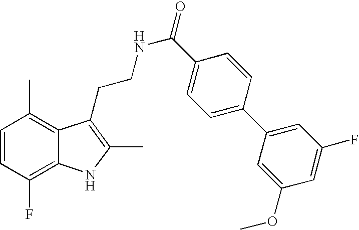 | 3′-fluoro-5′-methoxy-biphenyl-4-carboxylic acid [2-(7-fluoro-2,4-dimethyl-1H-indol-3-yl)ethyl]amide | 434.4836 | 435 | 4.62 |
| 213 | 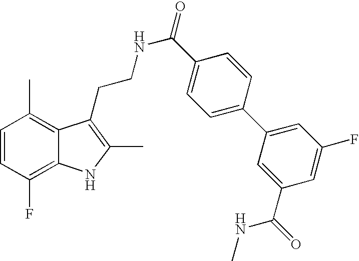 | 5-fluorobiphenyl-3,4′-dicarboxylic acid4′-{[2-(7-fluoro-2,4-dimethyl-1H-indol-3-yl)ethyl]amide} 3-methylamide | 461.5095 | 463 | 3.88 |
| 214 | 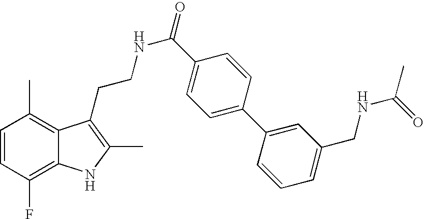 | 3′-(acetylamino-methyl)biphenyl-4-carboxylic acid [2-(7-fluoro-2,4-dimethyl-1H-indol-3-yl)ethyl]amide | 457.5462 | 459 | 3.71 |
| 215 | 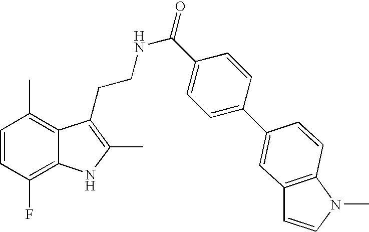 | N-[2-(7-fluoro-2,4-dimethyl-1H-indol-3-yl)ethyl]-4-(1-methyl-1H-indol-5-yl)-benzamide | 439.5314 | 441 | 4.47 |
| 216 | 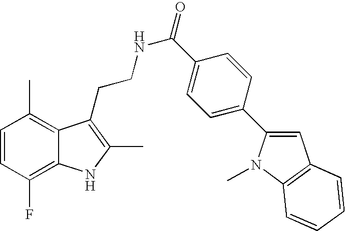 | N-[2-(7-fluoro-2,4-dimethyl-1H-indol-3-yl)ethyl]-4-(1-methyl-1H-indol-2-yl)-benzamide | 439.5314 | 441 | 4.69 |
| 217 | 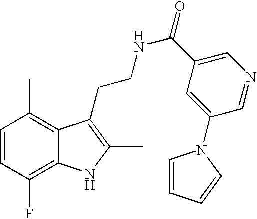 | N-[2-(7-fluoro-2,4-dimethyl-1H-indol-3-yl)ethyl]-5-pyrrol-1-yl-nicotinamide | 376 | 377 | 9.27 |
| 218 | 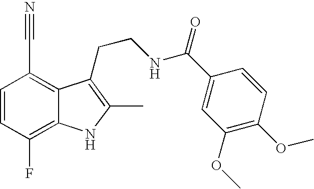 | N-[2-(4-cyano-7-fluoro-2-methyl-1H-indol-3-yl)ethyl]-3,4-dimethoxy-benzamide | 381 | 382 | 1.10 |
| 219 | 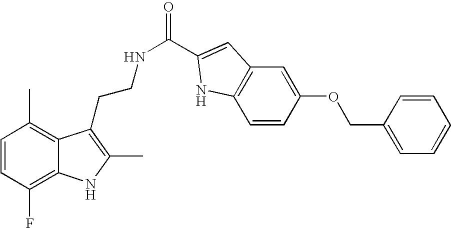 | 5-benzyloxy-1H-indole-2-carboxylicacid [2-(7-fluoro-2,4-dimethyl-1H-indol-3-yl)ethyl]amide | 455 | 456 | 1.44 |
| 220 | 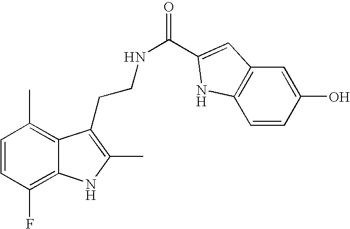 | 5-hydroxy-1H-indole-2-carboxylicacid [2-(7-fluoro-2,4-dimethyl-1H-indol-3-yl)ethyl]amide | 365 | 366 | 1.10 |
| 221 | 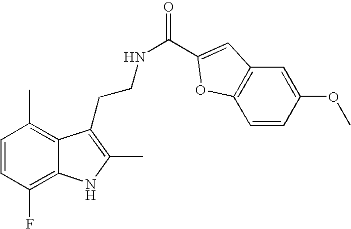 | 5-methoxybenzofuran-2-carboxylic acid [2-(7-fluoro-2,4-dimethyl-1H-indol-3-yl)ethyl]amide | 380 | 381 | 1.35 |
| 222 | 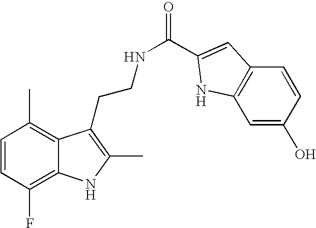 | 6-hydroxy-1H-indole-2-carboxylicacid [2-(7-fluoro-2,4-dimethyl-1H-indol-3-yl)ethyl]amide | 365 | 366 | 1.14 |
| 223 | 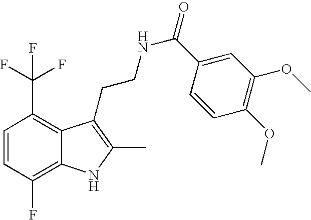 | N-[2-(7-fluoro-2-methyl-4-trifluoromethyl-1H-indol-3-yl)ethyl]-3,4-dimethoxy-benzamide | 424 | 425 | 1.22 |
| 224 | 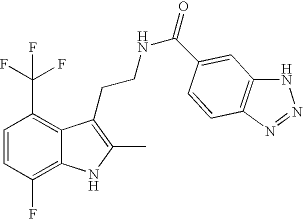 | 3H-benzotriazole-5-carboxylic acid [2-(7-fluoro-2-methyl-4-trifluoromethyl-1H-indol-3-yl)ethyl]amide | 405 | 406 | 1.13 |
| 225 | 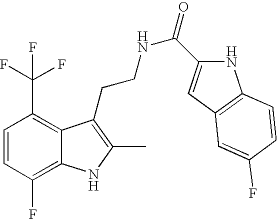 | 5-fluoro-1H-indole-2-carboxylic acid [2-(7-fluoro-2-methyl-4-trifluoromethyl-1H-indol-3-yl)ethyl]amide | 421 | 422 | 1.35 |
| 226 | 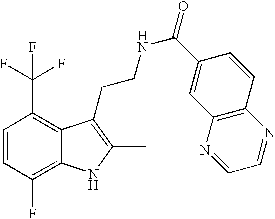 | quinoxaline-6-carboxylic acid [2-(7-fluoro-2-methyl-4-trifluoromethyl-1H-indol-3-yl)ethyl]amide | 416 | 415 | 1.20 |
| 227 | 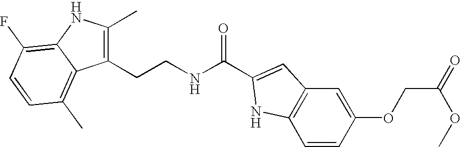 | {2-[2-(7-fluoro-2,4-dimethyl-1H-indol-3-yl)ethylcarbamoyl]-1H-indol-5-yloxy}acetic acidmethyl ester | 437 | 438 | 1.27 |
| 228 | 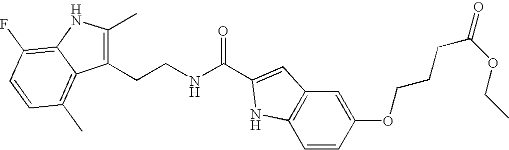 | {2-[2-(7-fluoro-2,4-dimethyl-1H-indol-3-yl)ethylcarbamoyl]-1H-indol-5-yloxy}butanoicacid ethyl ester | 479 | 480 | 1.38 |
| 229 | 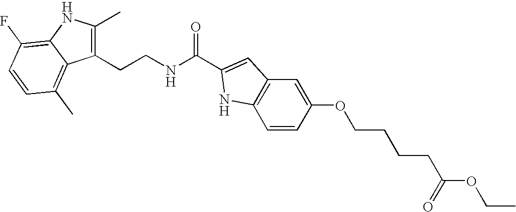 | {2-[2-(7-fluoro-2,4-dimethyl-1H-indol-3-yl)ethylcarbamoyl]-1H-indol-5-yloxy}pentanoicacid ethyl ester | 493 | 494 | 1.43 |
| 230 | 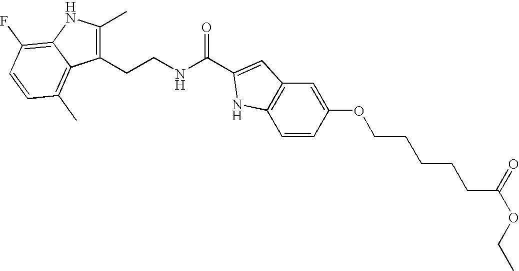 | {2-[2-(7-fluoro-2,4-dimethyl-1H-indol-3-yl)ethylcarbamoyl]-1H-indol-5-yloxy}hexanoicacid ethyl ester | 507 | 508 | 1.49 |
| 231 | 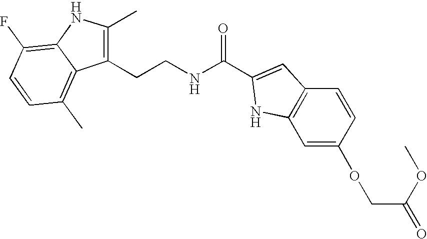 | {2-[2-(7-fluoro-2,4-dimethyl-1H-indol-3-yl)ethylcarbamoyl]-1H-indol-6-yloxy}acetic acidmethyl ester | 437 | 438 | 1.28 |
| 232 | 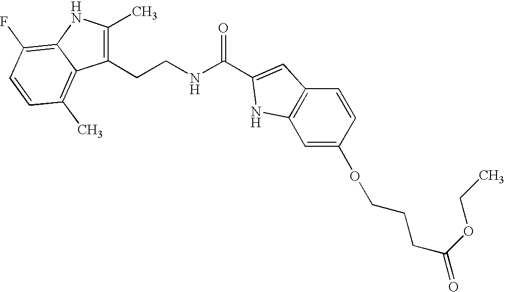 | {2-[2-(7-fluoro-2,4-dimethyl-1H-indol-3-yl)ethylcarbamoyl]-1H-indol-6-yloxy}butanoicacid ethyl ester | 479 | 480 | 1.41 |
| 233 | 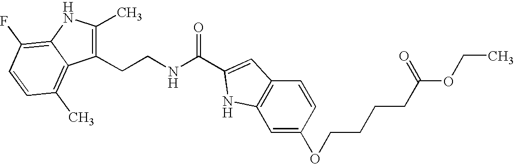 | {2-[2-(7-fluoro-2,4-dimethyl-1H-indol-3-yl)ethylcarbamoyl]-1H-indol-6-yloxy}pentanoicacid ethyl ester | 493 | 494 | 1.45 |
| 234 | 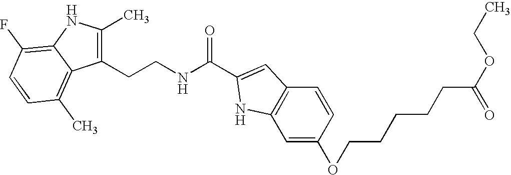 | {2-[2-(7-fluoro-2,4-dimethyl-1H-indol-3-yl)ethylcarbamoyl]-1H-indol-6-yloxy}hexanoicacid ethyl ester | 507 | 508 | 1.50 |
| 235 | 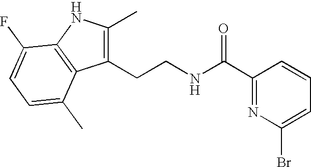 | 6-bromopyridine-2-carboxylic acid [2-(7-fluoro-2,4-dimethyl-1H-indol-3-yl)ethyl]amide | 389 | 390 | 1.45 |
| 236 | 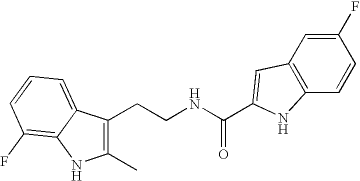 | 5-fluoro-1H-indole-2-carboxylic acid [2-(7-fluoro-2-methyl-1H-indol-3-yl)ethyl]amide | 353 | 354 | 1.20 |
| 237 | 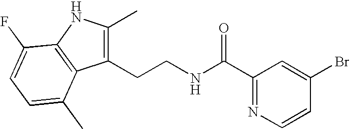 | 4-bromopyridine-2-carboxylic acid [2-(7-fluoro-2,4-dimethyl-1H-indol-3-yl)ethyl]amide | 389 | 390 | 1.31 |
| 238 | 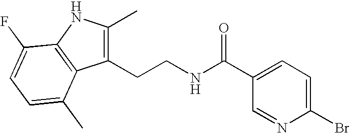 | 6-bromo-N-[2-(7-fluoro-2,4-dimethyl-1H-indol-3-yl)ethyl]nicotinamide | 389 | 390 | 1.20 |
| 239 | 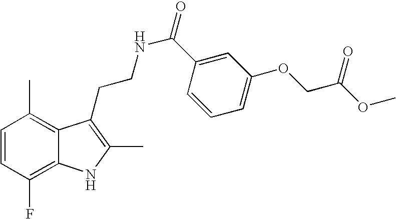 | {3-[2-(7-fluoro-2,4-dimethyl-1H-indol-3-yl)ethylcarbamoyl]phenoxy}acetic acidmethyl ester | 398 | 399 | 1.20 |
| 240 | 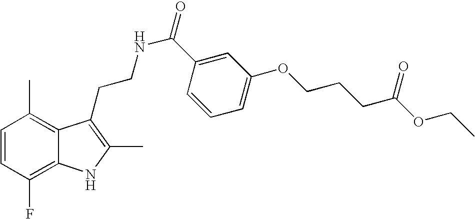 | 4-{3-[2-(7-fluoro-2,4-dimethyl-1H-indol-3-yl)ethylcarbamoyl]-phenoxy}butanoicacid ethyl ester | 440 | 441 | 1.36 |
| 241 | 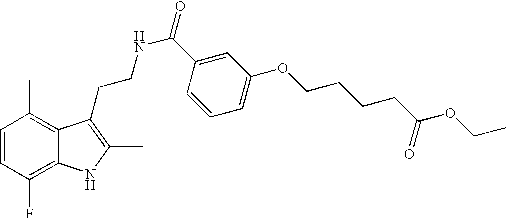 | 5-{3-[2-(7-fluoro-2,4-dimethyl-1H-indol-3-yl)ethylcarbamoyl]-phenoxy}pentanoicacid ethyl ester | 454 | 455 | 1.43 |
| 242 | 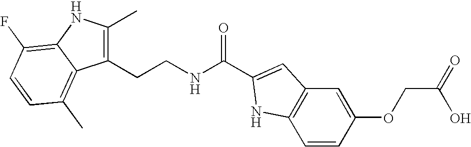 | 2-[2-(7-fluoro-2,4-dimethyl-1H-indol-3-yl)ethylcarbamoyl]-1H-indol-5-yloxy}acetic acid | 423 | 424 | 1.10 |
| 243 | 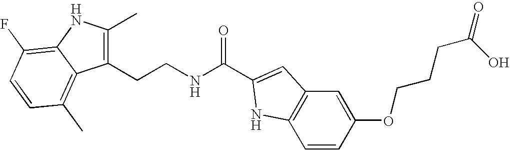 | 4-{2-[2-(7-fluoro-2,4-dimethyl-1H-indol-3-yl)ethylcarbamoyl]-1H-indol-5-yloxy}butanoicacid | 451 | 452 | 1.16 |
| 244 | 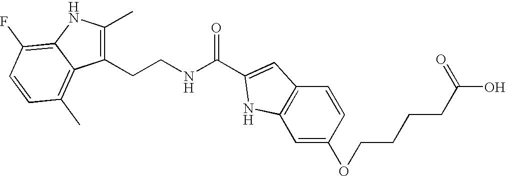 | 5-{2-[2-(7-fluoro-2,4-dimethyl-1H-indol-3-yl)ethylcarbamoyl]-1H-indol-6-yloxy}pentanoicacid | 465 | 466 | 1.22 |
| 245 | 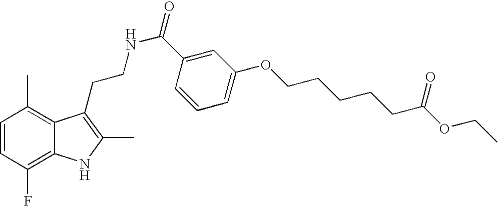 | 6-{3-[2-(7-fluoro-2,4-dimethyl-1H-indol-3-yl)ethylcarbamoyl]-phenoxy}hexanoicacid ethyl ester | 468 | 469 | 1.48 |
| 246 | 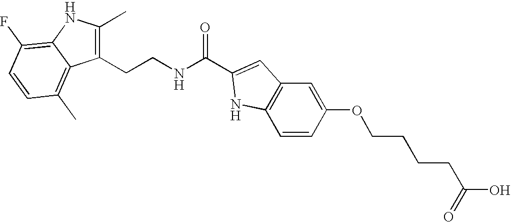 | 5-{2-[2-(7-fluoro-2,4-dimethyl-1H-indol-3-yl)ethylcarbamoyl]-1H-indol-5-yloxy}pentanoicacid | 465 | 466 | 1.20 |
| 247 | 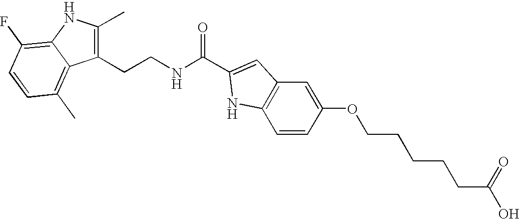 | 6-{2-[2-(7-fluoro-2,4-dimethyl-1H-indol-3-yl)ethylcarbamoyl]-1H-indol-5-yloxy}hexanoicacid | 479 | 480 | 1.26 |
| 248 | 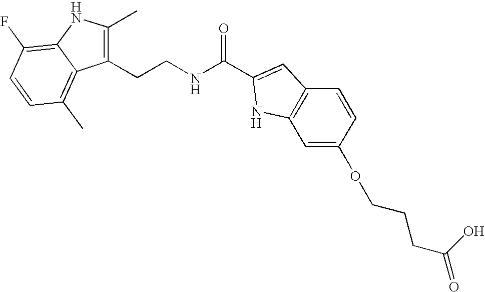 | 4-{2-[2-(7-fluoro-2,4-dimethyl-1H-indol-3-yl)ethylcarbamoyl]-1H-indol-6-yloxy}-butanoic acid | 451 | 452 | 1.22 |
| 249 | 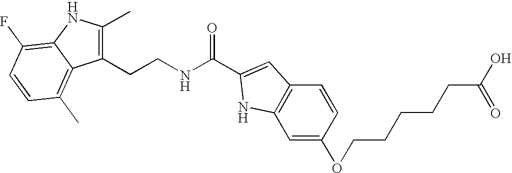 | 6-{2-[2-(7-fluoro-2,4-dimethyl-1H-indol-3-yl)ethylcarbamoyl]-1H-indol-6-yloxy}hexanoicacid | 479 | 480 | 1.29 |
| 250 | 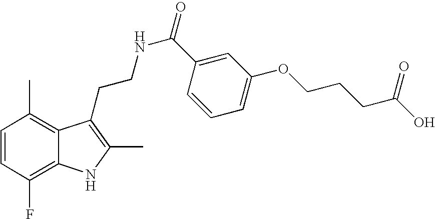 | 4-{3-[2-(7-fluoro-2,4-dimethyl-1H-indol-3-yl)ethylcarbamoyl]-phenoxy}butanoicacid | 412 | 413 | 1.17 |
| 251 | 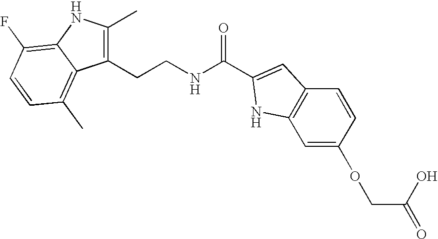 | {2-[2-(7-fluoro-2,4-dimethyl-1 H-indol-3-yl)ethylcarbamoyl]-1H-indol-6-yloxy}acetic acid | 423 | 424 | 1.12 |
| 252 | 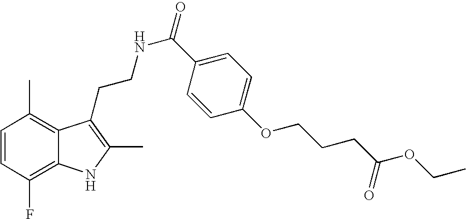 | 4-{4-[2-(7-fluoro-2,4-dimethyl-1H-indol-3-yl)ethylcarbamoyl]-phenoxy}butanoicacid ethyl ester | 440 | 441 | 1.28 |
| 253 | 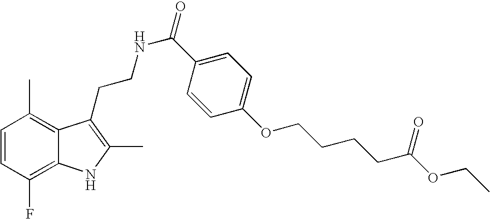 | 5-{4-[2-(7-fluoro-2,4-dimethyl-1H-indol-3-yl)ethylcarbamoyl]-phenoxy}pentanoicacid ethyl ester | 454 | 455 | 1.29 |
| 254 | 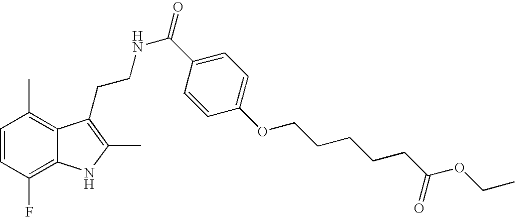 | 6-{4-[2-(7-fluoro-2,4-dimethyl-1H-indol-3-yl)ethylcarbamoyl]-phenoxy}hexanoicacid ethyl ester | 468 | 469 | 1.36 |
| 255 | 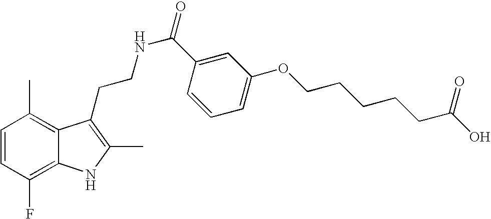 | 6-{3-[2-(7-fluoro-2,4-dimethyl-1H-indol-3-yl)ethylcarbamoyl]-phenoxy}hexanoicacid | 440 | 441 | 1.15 |
| 256 | 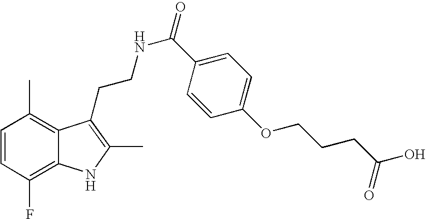 | 4-{4-[2-(7-fluoro-2,4-dimethyl-1H-indol-3-yl)ethylcarbamoyl]-phenoxy}butanoicacid | 412 | 413 | 1.04 |
| 257 | 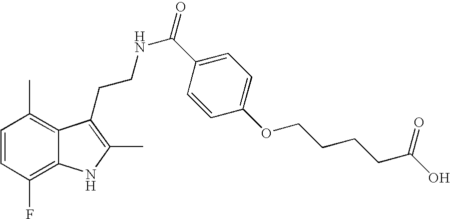 | 5-{4-[2-(7-fluoro-2,4-dimethyl-1H-indol-3-yl)ethylcarbamoyl]-phenoxy}pentanoicacid | 426 | 427 | 1.12 |
| 258 | 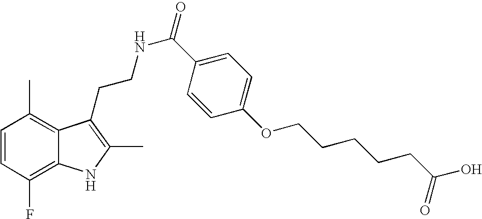 | 6-{4-[2-(7-fluoro-2,4-dimethyl-1H-indol-3-yl)ethylcarbamoyl]-phenoxy}hexanoicacid | 440 | 441 | 1.13 |
| 259 | 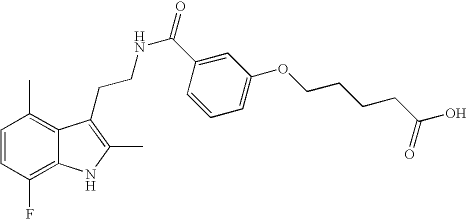 | 5-{3-[2-(7-fluoro-2,4-dimethyl-1H-indol-3-yl)ethylcarbamoyl]-phenoxy}pentanoicacid | 426 | 427 | 1.21 |
| 260 | 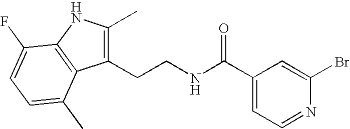 | 2-bromo-N-[2-(7-fluoro-2,4-dimethyl-1H-indol-3-yl)ethyl]isonicotin-amide | 390 | 390 | 1.26 |
| 261 | 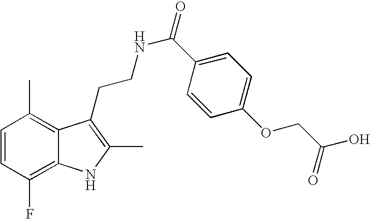 | {4-[2-(7-fluoro-2,4-dimethyl-1H-indol-yl)ethylcarbamoyl]phenoxy}acetic acid | 384 | 385 | 0.90 |
| 262 | 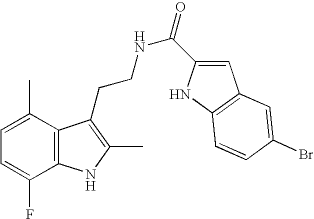 | 5-bromo-1H-indole-2-carboxylic acid [2-(7-fluoro-2,4-dimethyl-1H-indol-3-yl)ethyl]amide | 427 | 426[M − H] | 1.30 |
| 263 | 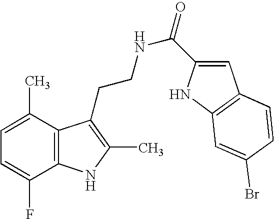 | 6-bromo-1H-indole-2-carboxylic acid [2-(7-fluoro-2,4-diimethyl-1H-indol-3-yl)ethyl]amide | 427 | 426[M − H] | 1.30 |
| 264 | 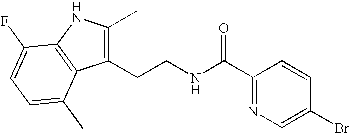 | 5-bromopyridine-2-carboxylic acid [2-(7-fluoro-2,4-dimethyl-1H-indol-3-yl)ethyl]amide | 464 | 465 | 1.26 |
| 265 | 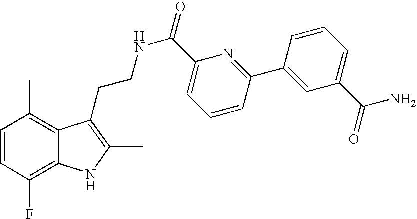 | 6-(3-carbamoyl-phenyl)pyridine-2-carboxylic acid [2-(7-fluoro-2,4-dimethyl-1H-indol-3-yl)-ethyl]amide | 430 | 431 | 1.16 |
| 266 | 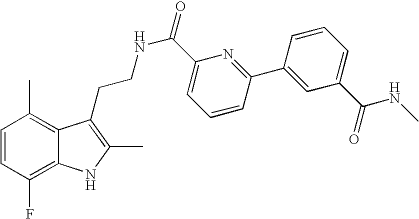 | 6-(3-methyl-carbamoylphenyl)-pyridine-2-carboxylicacid [2-(7-fluoro-2,4-dimethyl-1H-indol-3-yl)ethyl]amide | 444 | 445 | 1.23 |
| 267 | 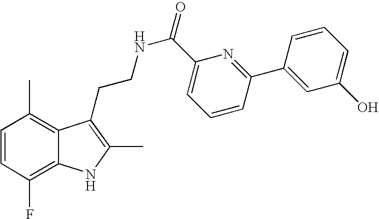 | 6-(3-hydroxy-phenyl)pyridine-2-carboxylic acid [2-(7-fluoro-2,4-dimethyl-1H-indol-3-yl)-ethyl]amide | 403 | 404 | 1.27 |
| 268 | 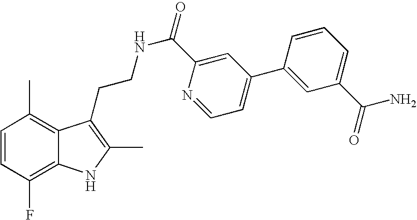 | 4-(3-carbamoyl-phenyl)pyridine-2-carboxylic acid [2-(7-fluoro-2,4-dimethyl-1H-indol-3-yl)-ethyl]amide | 430 | 431 | 1.19 |
| 269 | 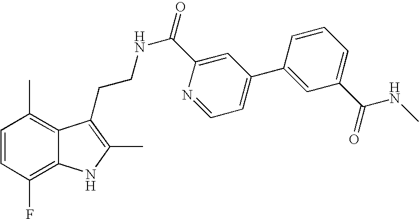 | 4-(3-methyl-carbamoylphenyl)-pyridine-2-carboxylicacid [2-(7-fluoro-2,4-dimethyl-1H-indol-3-yl)ethyl]amide | 444 | 445 | 1.23 |
| 270 | 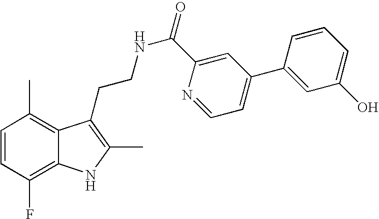 | 4-(3-hydroxyphenyl)-pyridine-2-carboxylicacid [2-(7-fluoro-2,4-dimethyl-1H-indol-3-yl)ethyl]amide | 403 | 404 | 1.30 |
| 271 | 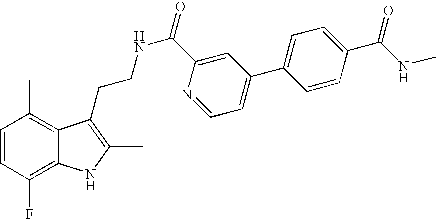 | 4-(4-methyl-carbamoylphenyl)-pyridine-2-carboxylicacid [2-(7-fluoro-2,4-dimethyl-1H-indol-3-yl)ethyl]amide | 444 | 445 | 1.23 |
| 272 | 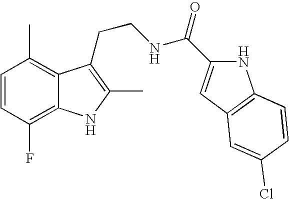 | 5-chloro-1H-indole-2-carboxylic acid[2-(7-fluoro-2,4-dimethyl-1H-indol-3-yl)ethyl]amide | 383 | 384 | 1.39 |
| 273 | 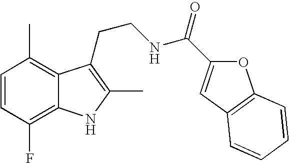 | benzofuran-2-carboxylic acid [2-(7-fluoro-2,4-dimethyl-1H-indol-3-yl)ethyl]amide | 350 | 351 | 1.39 |
| 274 | 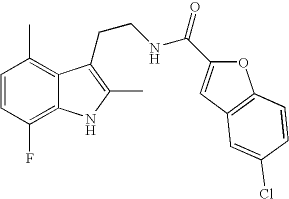 | 5-chlorobenzofuran-2-carboxylic acid [2-(7-fluoro-2,4-dimethyl-1H-indol-3-yl)ethyl]amide | 384 | 385 | 1.48 |
| 275 | 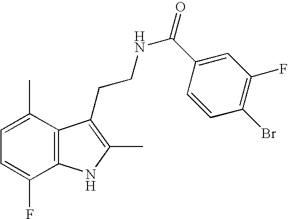 | 4-bromo-3-fluoro-N-[2-(7-fluoro-2,4-dimethyl-1H-indol-3-yl)ethyl]-benzamide | 406 | 407 | 1.39 |
| 276 | 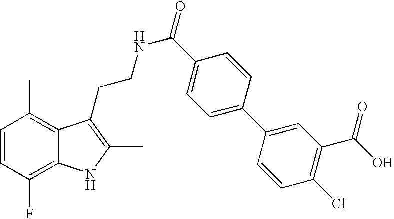 | 4-chloro-4′-[2-(7-fluoro-2,4-dimethyl-1H-indol-3-yl)ethyl-carbamoyl]biphenyl-3-carboxylic acid | 464 | 465 | 1.27 |
| 277 | 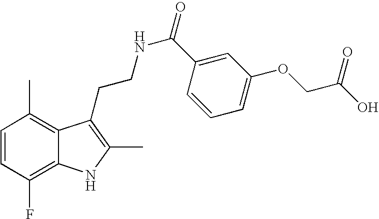 | {3-[2-(7-fluoro-2,4-dimethyl-1H-indol-3-yl)ethyl-carbamoyl]-phenoxy}acetic acid | 384 | 383 | 1.09 |
| 278 | 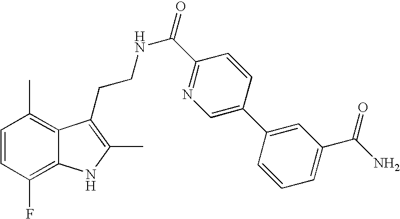 | 5-(3-carbamoyl-phenyl)pyridine-2-carboxylic acid[2-(7-fluoro-2,4-dimethyl-1H-indol-3-yl)ethyl]amide | 430 | 431 | 1.17 |
| 279 | 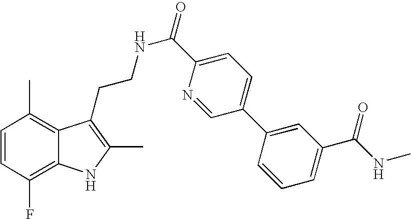 | 5-(3-methyl-carbamoylphenyl)-pyridine-2-carboxylicacid [2-(7-fluoro-2,4-dimethyl-1H-indol-3-yl)ethyl]amide | 444 | 445 | 1.21 |
| 280 | 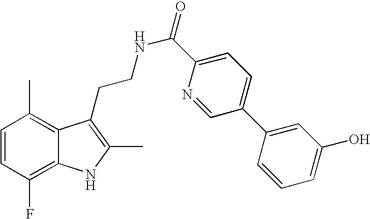 | 5-(3-hydroxyphenyl)-pyridine-2-carboxylicacid [2-(7-fluoro-2,4-dimethyl-1H-indol-3-yl)ethyl]amide | 403 | 404 | 1.27 |
| 281 | 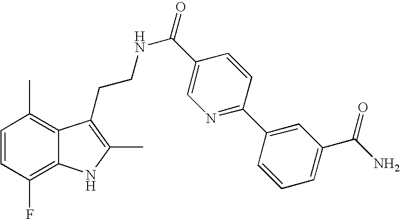 | 6-(3-carbamoyl-phenyl)-N-[2-(7-fluoro-2,4-dimethyl-1H-indol-3-yl)ethyl]-nicotinamide | 430 | 431 | 1.10 |
| 282 | 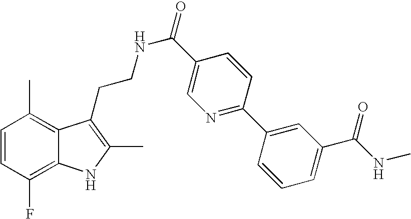 | N-[2-(7-fluoro-2,4-dimethyl-1H-indol-3-yl)ethyl]-6-(3-methylcarbamoyl-phenyl)nicotinamide | 444 | 445 | 1.13 |
| 283 | 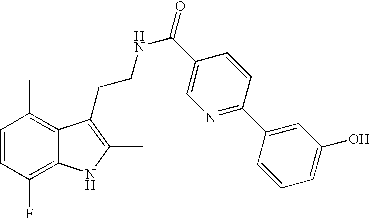 | N-[2-(7-fluoro-2,4-dimethyl-1H-indol-3-yl)ethyl]-6-(3-hydroxyphenyl)-nicotinamide | 403 | 404 | 1.17 |
| 284 | 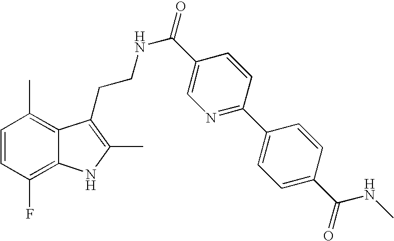 | N-[2-(7-fluoro-2,4-dimethyl-1H-indol-3-yl)ethyl]-6-(4-methylcarbamoyl-phenyl)nicotinamide | 444 | 445 | 1.12 |
| 285 | 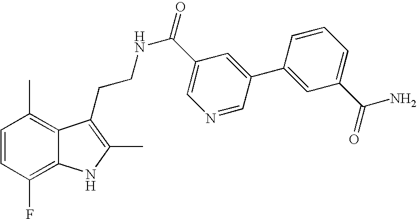 | 5-(3-carbamoyl-phenyl)-N-[2-(7-fluoro-2,4-dimethyl-1H-indol-3-yI)ethyl]-nicotinamide | 430 | 431 | 1.05 |
| 286 | 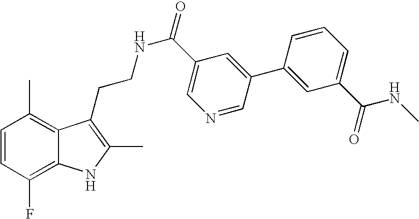 | N-[2-(7-fluoro-2,4-dimethyl-1H-indol-3-yl)ethyl]-5-(3-methylcarbamoyl-phenyl)nicotinamide | 444 | 445 | 1.10 |
| 287 | 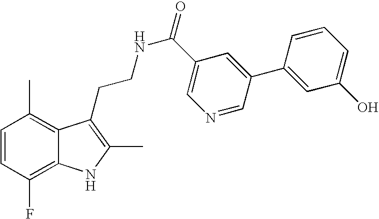 | N-[2-(7-fluoro-2,4-dimethyl-1H-indol-3-yl)ethyl]-5-(3-hydroxyphenyl)-nicotinamide | 403 | 404 | 1.14 |
| 288 | 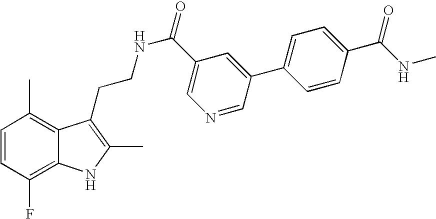 | N-[2-(7-fluoro-2,4-dimethyl-1H-indol-3-yl)ethyl]-5-(4-methylcarbamoyl-phenyl)nicotinamide | 444 | 445 | 1.07 |
| 289 | 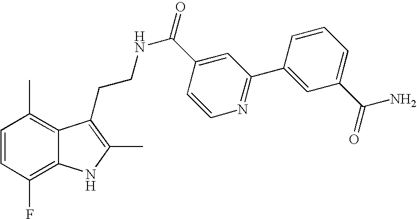 | 2-(3-carbamoyl-phenyl)-N-[2-(7-fluoro-2,4-dimethyl-1H-indol-3-yl)ethyl]-isonicotinamide | 430 | 431 | 1.10 |
| 290 | 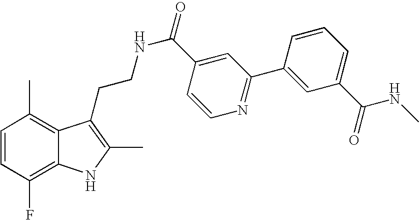 | N-[2-(7-fluoro-2,4-dimethyl-1H-indol-3-yl)ethyl]-(3-methylcarbamoylphenyl)isonicotin-amide | 444 | 445 | 1.14 |
| 291 | 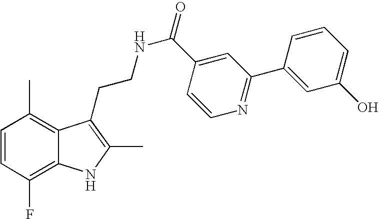 | N-[2-(7-fluoro-2 4-dimethyl-1H-indol-3-yl)ethyl]-2-(3-hydroxyphenyl)-isonicotinamide | 403 | 404 | 1.17 |
| 292 | 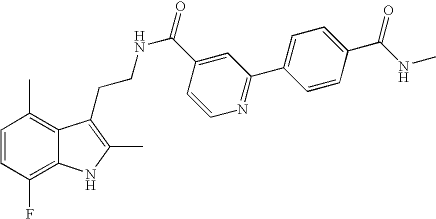 | N-[2-(7-fluoro-2,4-dimethyl-1H-indol-3-yl)ethyl]-2-(4-methylcarbamoylphenyl)isonicotin-amide | 444 | 445 | 1.12 |
| 293 | 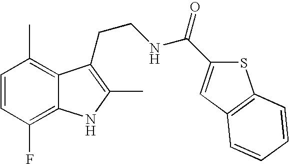 | benzo[b]thiophene-2-carboxylic acid[2-(7-fluoro-2,4-dimethyl-1H-indol-3-yl)ethyl]amide | 366 | 365 | 1.37 |
| 294 | 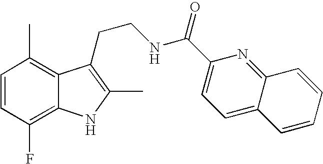 | quinoline-2-carboxylic acid [2-(7-fluoro-2,4-dimethyl-1H-indol-3-yl)ethyl]amide | 361 | 362 | 1.47 |
| 295 | 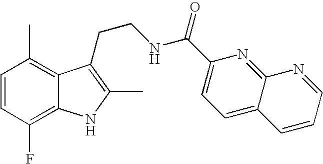 | [1,8]naphthyridine-2-carboxylic acid[2-(7-fluoro-2,4-dimethyl-1H-indol-3-yl)ethyl]amide | 362 | 363 | 1.23 |
| 296 | 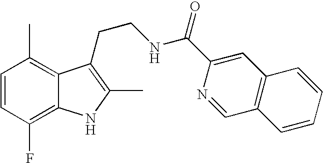 | isoquinoline-3-carboxylic acid [2-(7-fluoro-2,4-dimethyl-1H-indol-3-yl)ethyl]amide | 361 | 362 | 1.37 |
| 297 | 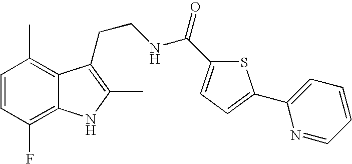 | 5-pyridin-2-yl-thiophene-2-carboxylic acid[2-(7-fluoro-2,4-dimethyl-1H-indol-3-yl)ethyl]amide | 393 | 394 | 1.35 |
| 298 | 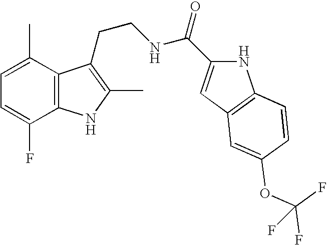 | 5-trifluoromethoxy-1H-indole-2-carboxylic acid[2-(7-fluoro-2,4-dimethyl-1H-indol-3-yl)ethyl]amide | 433 | 432 | 1.33 |
| 299 | 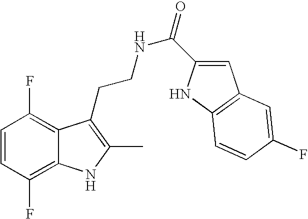 | 5-fluoro-1H-indole-2-carboxylic acid[2-(4,7-difluoro-2-methyl-1H-indol-3-yl)ethyl]amide | 371 | 372 | 1.32 |
| 300 | 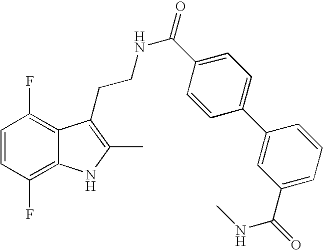 | biphenyl-3,4′-dicarboxylic acid4′-{[2-(4,7-difluoro-2-methyl-1H-indol-3-yl)ethyl]amide}3-methylamide | 447 | 448 | 1.22 |
| 301 | 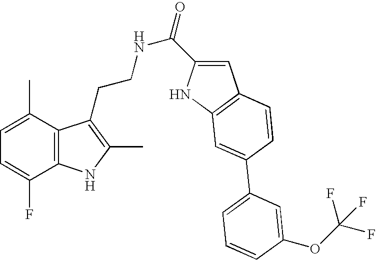 | 6-(3-trifluoro-methoxyphenyl)-1H-indole-2-carboxylicacid [2-(7-fluoro-2,4-dimethyl-1H-indol-3-yl)ethyl]-amide | 509 | 511 | 0.97 |
| 302 | 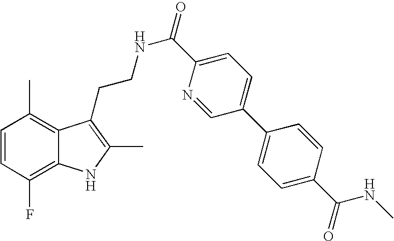 | 5-(4-methyl-carbamoylphenyl)-pyridine-2-carboxylicacid [2-(7-fluoro-2,4-dimethyl-1H-indol-3-yl)ethyl]-amide | 444 | 445 | 1.21 |
| 303 | 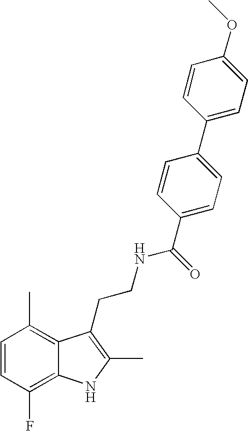 | 4′-methoxybiphenyl-4-carboxylic acid[2-(7-fluoro-2,4-dimethyl-1H-indol-3-yl)ethyl]-amide | 416 | 417 | 4.42 |
| 304 | 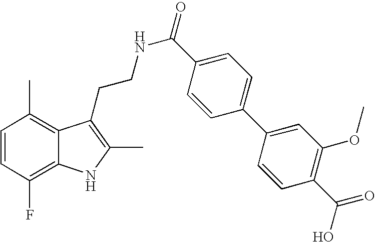 | 4′-[2-(7-fluoro-2,4-dimethyl-1H-indol-3-yl)ethyl-carbamoyl]-3-methoxybiphenyl-4-carboxylic acid | 460 | 462 | 3.84 |
| 305 | 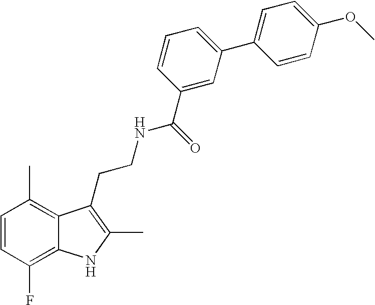 | 4′-methoxybiphenyl-3-carboxylic acid[2-(7-fluoro-2,4-dimethyl-1H- indol-3-yl)ethyl]-amide | 416 | 417 | 4.44 |
| 307 | 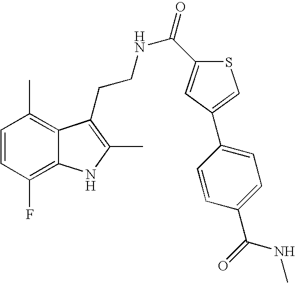 | 4-(4-methyl-carbamoylphenyl)-thiophene-2-carboxylic acid[2-(7-fluoro-2,4-dimethyl-1H-indol-3-yl)ethyl]-amide | 449 | 450 | 1.22 |
| 308 | 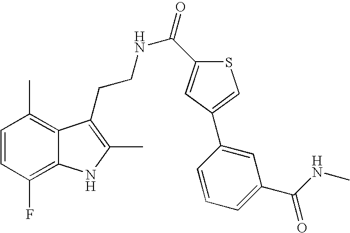 | 4-(3-methyl-carbamoylphenyl)-thiophene-2-carboxylic acid[2-(7-fluoro-2,4-dimethyl-1H-indol-3-yl)ethyl]-amide | 449 | 450 | 1.25 |
| 309 | 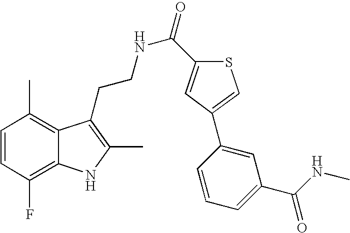 | 4-(3-methyl-carbamoylphenyl)-thiophene-2-carboxylic acid[2-(7-fluoro-2,4-dimethyl-1H-indol-3-yl)ethyl]-amide | 449 | 450 | 1.25 |
| 310 | 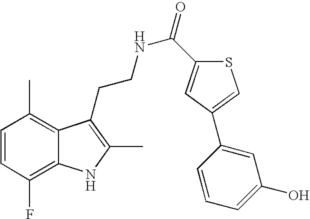 | 4-(3-hydroxyphenyl)-thiophene-2-carboxylic acid[2-(7-fluoro-2,4-dimethyl-1H-indol-3-yl)ethyl]amide | 408 | 409 | 1.30 |
| 311 | 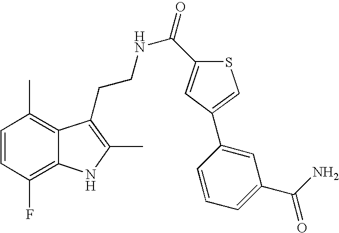 | 4-(3-carbamoyl-phenyl)thiophene-2-carboxylic acid[2-(7-fluoro-2,4-dimethyl-1H-indol-3-yl)ethyl]-amide | 435 | 436 | 1.18 |
| 312 | 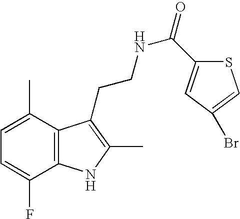 | 4-bromothiophene-2-carboxylic acid[2-(7-fluoro-2,4-dimethyl-1H-indol-3-yl)ethyl]-amide | 395 | 395 | 1.35 |
| 313 | 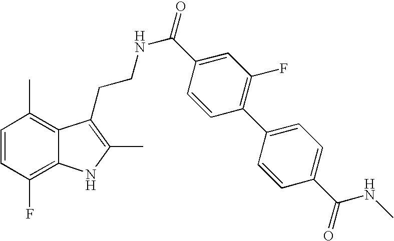 | 2-fluorobiphenyl-4,4′-dicarboxylic acid4-{[2-(7-fluoro-2,4-dimethyl-1H-indol-3-yl)ethyl]-amide} 4′-methyl-amide | 461 | 462 | 1.25 |
| 314 | 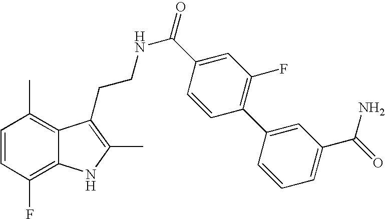 | 2′-fluorobiphenyl-3,4′-dicarboxylic acid3-amide4′-{[2-(7-fluoro-2,4-dimethyl-1H-indol-3-yl)ethyl]-amide} | 447 | 448 | 1.21 |
| 315 | 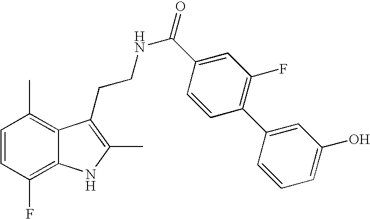 | 2-fluoro-3′-hydroxy-biphenyl-4-carboxylic acid[2-(7-fluoro-2,4-dimethyl-1H-indol-3-yl)ethyl]-amide | 420 | 421 | 1.32 |
| 316 | 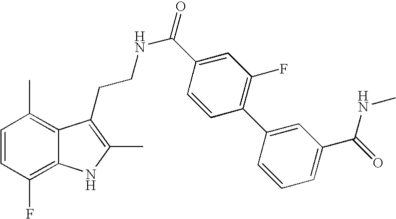 | 2′-fluorobiphenyl-3,4′-dicarboxylic acid4′-{[2-(7-fluoro-2,4-dimethyl-1H-indol-3-yl)ethyl]-amide} 3-methyl-amide | 461 | 462 | 1.25 |
| 317 | 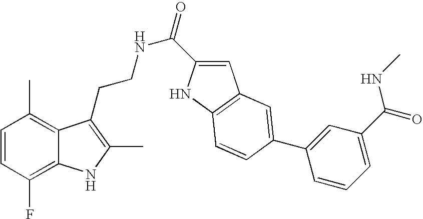 | 5-(3-methyl-carbamoylphenyl)1H-indole-2-carboxylic acid[2-(7-fluoro-2,4-dimethyl-1H-indol-3-yl)ethyl]-amide | 482 | 483 | 1.25 |
| 318 | 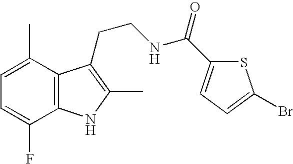 | 5-bromothiophene-2-carboxylic acid[2-(7-fluoro-2,4-dimethyl-1H-indol-3-yl)ethyl]-amide | 394 | 395 | 1.38 |
| 319 | 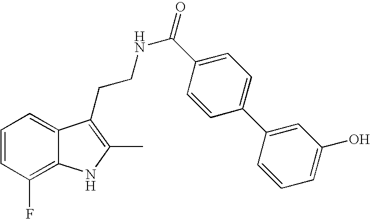 | 3′-hydroxybiphenyl-4-carboxylic acid[2-(7-fluoro-2-methyl-1H-indol-3-yl)ethyl]amide | 388 | 389 | 1.22 |
| 320 | 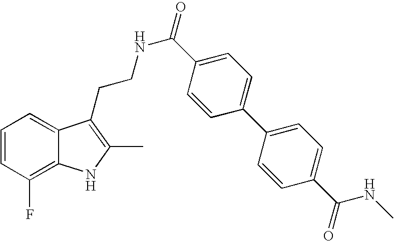 | biphenyl-4,4′-dicarboxylic acid4′-{[2-(7-fluoro-2-methyl-1H-indol-3-yl)ethyl]amide}4-methylamide | 429 | 430 | 1.14 |
| 321 | 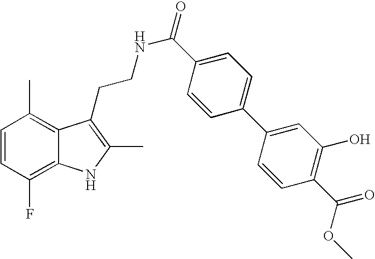 | 4′-[2-(7-fluoro-2,4-dimethyl-1H-indol-3-yl)ethyl-carbamoyl]-3-hydroxybiphenyl-4-carboxylic acidmethyl ester | 460 | 459 | 1.44 |
| 322 | 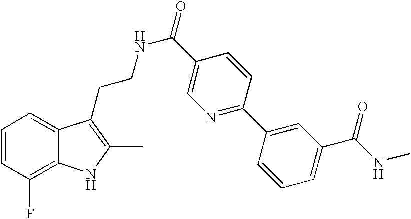 | N-[2-(7-fluoro-2-methyl-1H-indol-3-yl)ethyl]-6-(3-methyl-carbamoylphenyl)-nicotinamide | 430 | 431 | 1.09 |
| 323 | 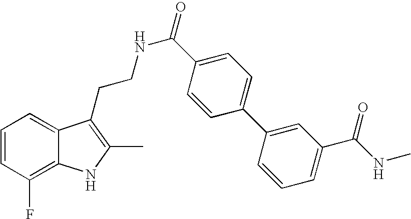 | biphenyl-3,4′-dicarboxylic acid4′-{[2-(8-fluoro-2-methyl-1H-indol-3-yl)ethyl]amide}3-methylamide | 429 | 430 | 1.16 |
| 324 | 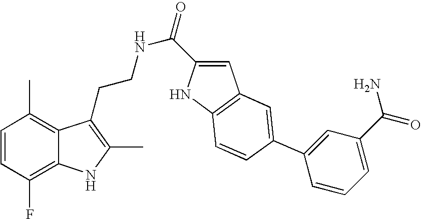 | 5-(3-carbamoyl-phenyl)-1H-indole-2-carboxylic acid[2-(7-fluoro-2,4-dimethyl-1H-indol-3-yl)ethyl]-amide | 468 | 469 | 1.22 |
| 325 | 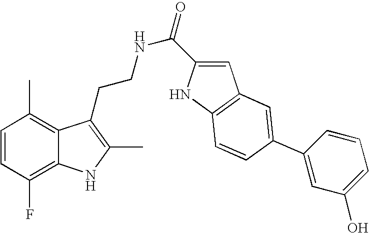 | 5-(3-hydroxyphenyl)-1H-indole-2-carboxylic acid[2-(7-fluoro-2,4-dimethyl-1H-indol-3-yl)ethyl]-amide | 441 | 442 | 1.31 |
| 326 | 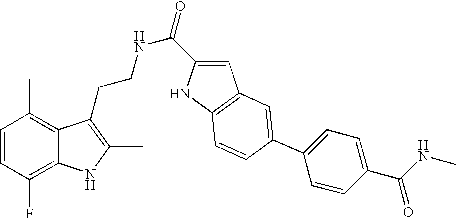 | 5-(4-methyl-carbamoylphenyl)-1H-indole-2-carboxylic acid[2-(7-fluoro-2,4-dimethyl-1H-indol-3-yl)ethyl]-amide | 482 | 483 | 1.24 |
| 327 | 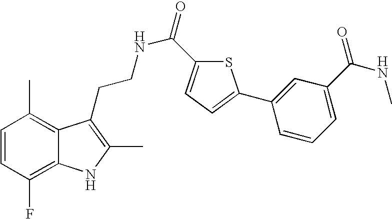 | 5-(3-methyl-carbamoylphenyl)-thiophene-2-carboxylic acid[2-(7-fluoro-2,4-dimethyl-1H-indol-3-yl)ethyl]-amide | 449 | 450 | 1.25 |
| 328 | 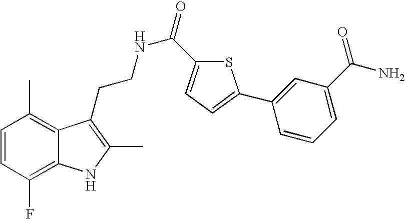 | 5-(3-carbamoyl-phenyl)thiophene-2-carboxylic acid[2-(7-fluoro-2,4-dimethyl-1H-indol-3-yl)ethyl]-amide | 435 | 436 | 1.21 |
| 329 | 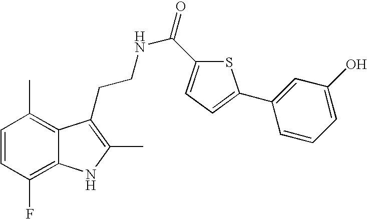 | 5-(3-hydroxy-phenyl)thiophene-2-carboxylic acid[2-(7-fluoro-2,4-dimethyl-1H-indol-3-yl)ethyl]-amide | 408 | 409 | 1.28 |
| 330 | 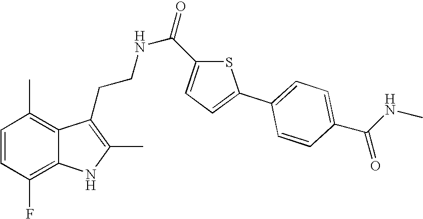 | 5-(4-methyl-carbamoylphenyl)-thiophene-2-carboxylic acid[2-(7-fluoro-2,4-dimethyl-1H-indol-3-yl)ethyl]-amide | 449 | 450 | 1.21 |
| 331 | 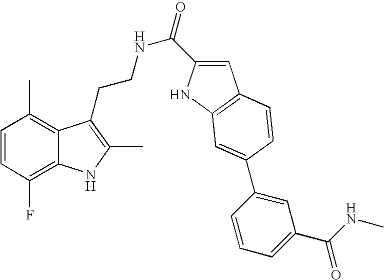 | 6-(3-methyl-carbamoylphenyl)-1H-indole-2-carboxylic acid[2-(7-fluoro-2,4-dimethyl-1H-indol-3-yl)ethyl]-amide | 482 | 483 | 1.28 |
| 332 | 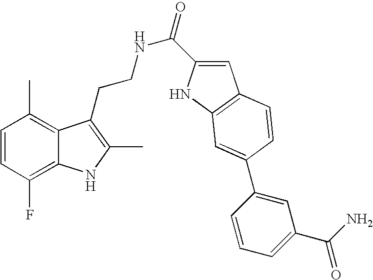 | 6-(3-carbamoyl-phenyl)-1H-indole-2-carboxylic acid[2-(7-fluoro-2,4-dimethyl-1H-indol-3-yl)ethyl]-amide | 468 | 469 | 1.23 |
| 333 | 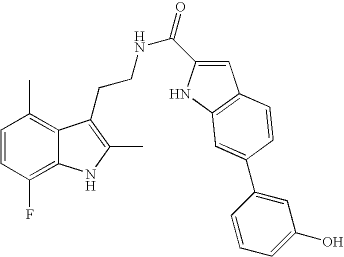 | 6-(3-hydroxyphenyl)-1H-indole-2-carboxylic acid[2-(7-fluoro-2,4-dimethyl-1H-indol-3-yl)ethyl]-amide | 441 | 442 | 1.32 |
| 334 | 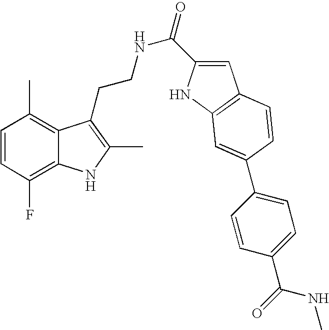 | 6-(4-methyl-carbamoylphenyl)-1H-indole-2-carboxylic acid[2-(7-fluoro-2,4-dimethyl-1H-indol-3-yl)ethyl]-amide | 482 | 483 | 1.26 |
| 335 | 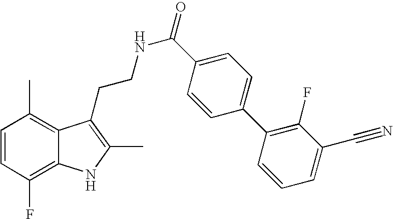 | 3′-cyano-2′-fluorobiphenyl-4-carboxylic acid[2-(7-fluoro-2,4-dimethyl-1H-indol-3-yl)ethyl]-amide | 429 | 430 | 1.38 |
| 336 | 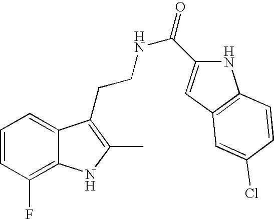 | 5-chloro-1H-indole-2-carboxylic acid[2-(7-fluoro-2-methyl-1H-indol-3-yl)ethyl]amide | 369 | 370 | 1.35 |
| 337 | 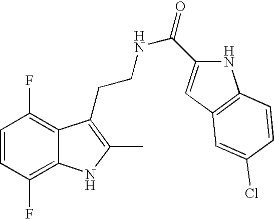 | 5-chloro-1H-indole-2-carboxylic acid[2-(4,7-difluoro-2-methyl-1H-indol-3-yl)ethyl]amide | 387 | 388 | 1.37 |
| 338 | 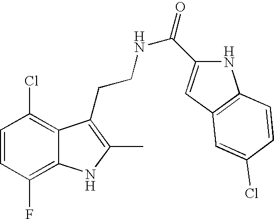 | 5-chloro-1H-indole-2-carboxylic acid[2-(4-chloro-7-fluoro-2-methyl-1H-indol-3-yl)ethyl]amide | 404 | 404 | 1.44 |
| 339 | 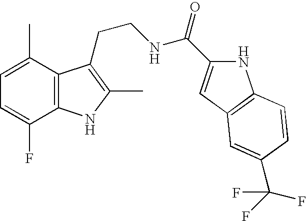 | 5-trifluoromethyl-1H-indole-2-carboxylicacid [2-(7-fluoro-2,4-dimethyl-1H-indol-3-yl)ethyl]-amide | 417 | 416[M − H] | 1.44 |
| 340 | 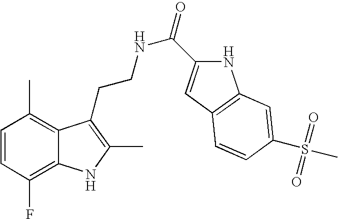 | 6-methanesulfonyl-1H-indole-2-carboxylic acid[2-(7-fluoro-2,4-dimethyl-1H-indol-3-yl)ethyl]-amide | 427 | 428 | 1.19 |
| 341 | 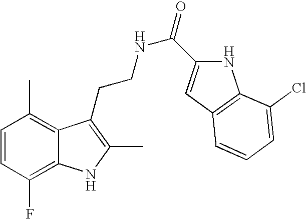 | 7-chloro-1H-indole-2-carboxylic acid[2-(7-fluoro-2,4-dimethyl-1H-indol-3-yl)ethyl]-amide | 383 | 382 | 1.39 |
| 342 | 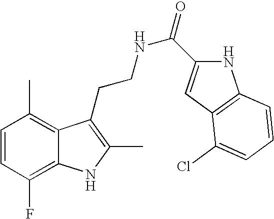 | 4-chloro-1H-indole-2-carboxylic acid[2-(7-fluoro-2,4-dimethyl-1H-indol-3-yl)ethyl]-amide | 383 | 382[M − H] | 1.40 |
| 343 | 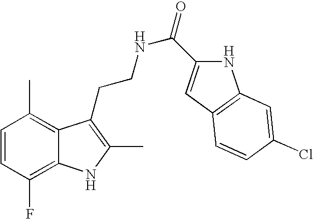 | 6-chloro-1H-indole-2-carboxylic acid[2-(7-fluoro-2,4-dimethyl-1H-indol-3-yl)ethyl]-amide | 383 | 382 | 1.39 |
| 344 | 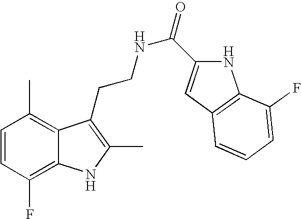 | 7-fluoro-1H-indole-2-carboxylic acid[2-(7-fluoro-2,4-dimethyl-1H-indol-3-yl)ethyl]-amide | 367 | 368 | 1.33 |
| 345 | 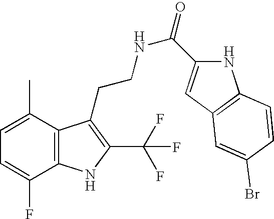 | 5-bromo-1H-indole-2-carboxylic acid[2-(7-fluoro-4-methyl-2-trifluoro-methyl-1H-indol-3-yl)ethyl]amide | 483 | 484 | 1.46 |
| 346 | 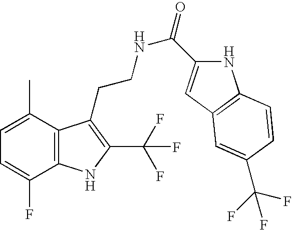 | 5-trifluoromethyl-1H-indole-2-carboxylicacid [2-(7-fluoro-4-methyl-2-trifluoro-methyl-1H-indol-3-yl)ethyl]amide | 471 | 472 | 1.48 |
| 347 | 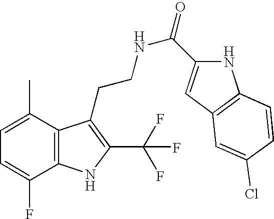 | 5-chloro-1H-indole-2-carboxylic acid[2-(7-fluoro-4-methyl-2-trifluoro-methyl-1H-indol-3-yl)ethyl]amide | 437 | 438 | 1.44 |
| 348 | 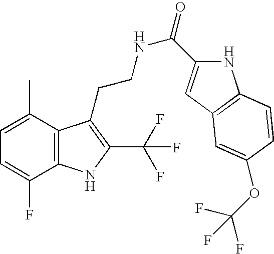 | 5-trifluoromethoxy-1H-indole-2-carboxylic acid[2-(7-fluoro-4-methyl-2-trifluoro-methyl-1H-indol-3-yl)ethyl]amide | 487 | 488 | 1.49 |
| 349 | 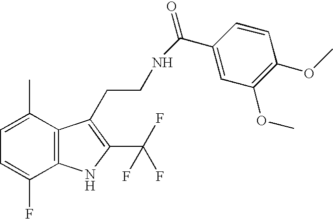 | N-[2-(7-fluoro-4-methyl-2-trifluoro-methyl-1H-indol-3-yl)ethyl]-3,4-dimethoxy-benzamide | 424 | 425 | 1.26 |
| 350 | 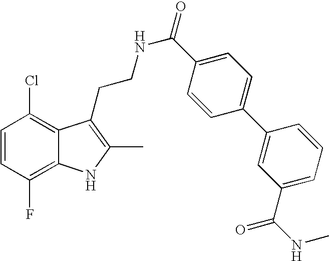 | biphenyl-3,4′-dicarboxylic acid4′-{[2-(4-chloro-7-fluoro-2-methyl-1H-indol-3-yl)ethyl]-amide} 3-methyl-amide | 463 | 464 | 1.25 |
| 351 | 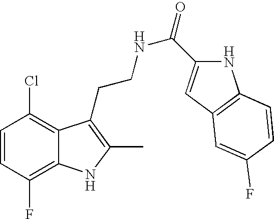 | 5-fluoro-1H-indole-2-carboxylic acid[2-(4-chloro-7-fluoro-2-methyl-1H-indol-3-yl)ethyl]amide | 387 | 388 | 1.36 |
| 352 | 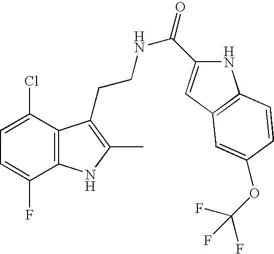 | 5-trifluoromethoxy-1H-indole-2-carboxylic acid[2-(4-chloro-7-fluoro-2-methyl-1H-indol-3-yl)ethyl]amide | 453 | 454 | 1.48 |
| 353 | 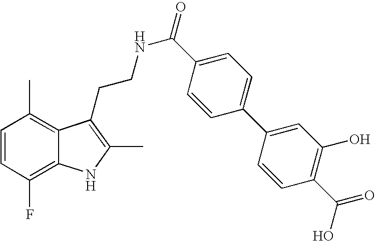 | 4′-[2-(7-fluoro-2,4-dimethyl-1H-indol-3-yl)ethyl-carbamoyl]-3-hydroxybiphenyl-4-carboxylic acid | 446 | 447 | 1.29 |
| 354 | 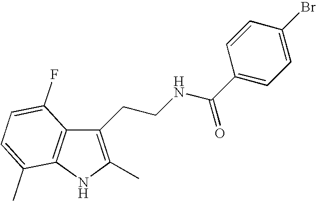 | 4-bromo-N-[2-(4-fluoro-2,7-dimethyl-1H-indol-3-yl)ethyl]-benzamide | 389 | 389 | 1.33 |
| 355 | 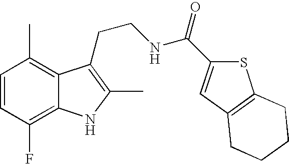 | 4,5,6,7-tetrahydro-benzo[b]thiophene-2-carboxylic acid[2-(7-fluoro-2,4-dimethyl-1H-indol-3-yl)ethyl]-amide | 370 | 371 | 1.43 |
| 356 | 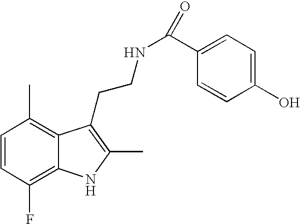 | N-[2-(7-fluoro-2,4-dimethyl-1H-indol-3-yl)ethyl]-4-hydroxy-benzamide | 326 | 327 | 1.10 |
| 357 | 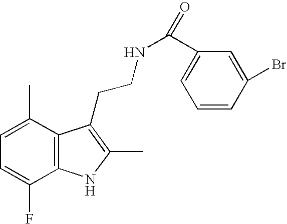 | 3-bromo-N-[2-(7-fluoro-2,4-dimethyl-1H-indol-3-yl)ethyl]-benzamide | 390 | 391 | 1.36 |
| 358 | 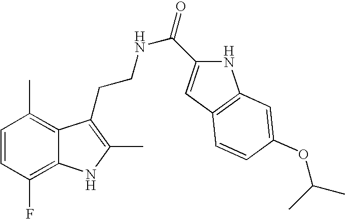 | 6-isopropoxy-1H-indole-2-carboxylicacid [2-(7-fluoro-2,4-dimethyl-1H-indol-3-yl)ethyl]-amide | 407 | 408 | 1.40 |
| 359 | 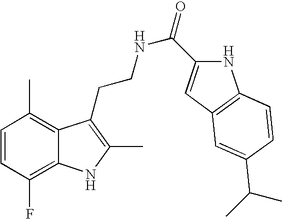 | 5-isopropyl-1H-indol-2-carboxylic acid[2-(7-fluoro-2,4-dimethyl-1H-indol-3-yl)ethyl]-amide | 391 | 392 | 1.49 |
| 360 | 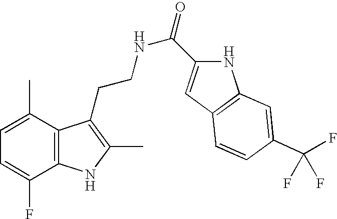 | 6-trifluoromethyl-1H-indole-2-carboxylicacid [2-(7-fluoro-2,4-dimethyl-1H-indol-3-yl)ethyl]-amide | 417 | 418 | 1.44 |
| 361 | 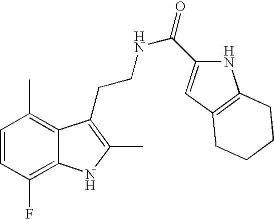 | 4,5,6,7-tetrahydro-1H-indole-2-carboxylic acid[2-(7-fluoro-2,4-dimethyl-1H-indol-3-yl)ethyl]-amide | 353 | 354 | 1.34 |
| 362 | 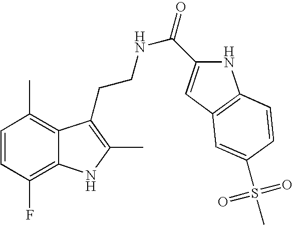 | 5-methanesulfonyl-1H-indole-2-carboxylic acid[2-(7-fluoro-2,4-dimethyl-1H-indol-3-yl)ethyl]-amide | 427 | 428 | 1.18 |
| 363 | 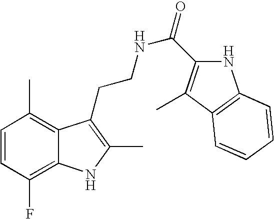 | 3-methyl-1H-indole-2-carboxylic acid[2-(7-fluoro-2,4-dimethyl-1H-indol-3-yl)ethyl]-amide | 363 | 364 | 1.38 |
| 364 | 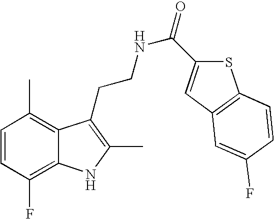 | 5-fluoro-benzo[b]thiophene-2-carboxylic acid[2-(7-fluoro-2,4-dimethyl-1H-indol-3-yl)ethyl]-amide | 384 | 385 | 1.44 |
| 365 | 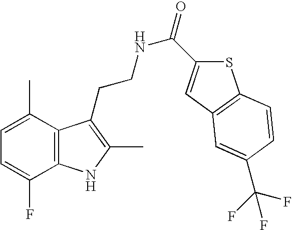 | 5-trifluoromethyl-benzo[b]thiophene-2-carboxylic acid [2-(7-fluoro-2,4-dimethyl-1H-indol-3-yl)ethyl]amide | 434 | 433[M − H] | 1.52 |
| 366 | 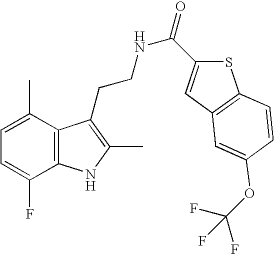 | 5-trifluoromethoxy-benzo[b]thiophene-2-carboxylic acid[2-(7-fluoro-2,4-dimethyl-1H-indol-3-yl)ethyl]-amide | 450 | 449[M − H] | 1.53 |
| 367 | 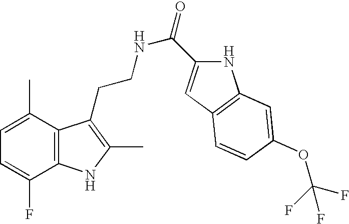 | 6-trifluoromethoxy-1H-indole-2-carboxylic acid[2-(7-fluoro-2,4-dimethyl-1H-indol-3-yl)ethyl]-amide | 433 | 434 | 1.46 |
| 368 | 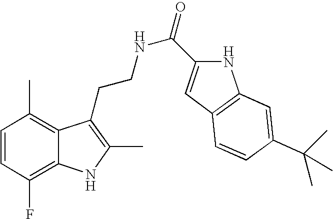 | 6-tert-butyl-1H-indole-2-carboxylicacid [2-(7-fluoro-2,4-dimethyl-1H-indol-3-yl)ethyl]-amide | 405 | 406 | 1.53 |
| 369 | 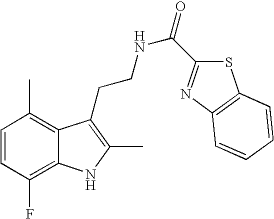 | benzothiazole-2-carboxylic acid[2-(7-fluoro-2,4-dimethyl-1H-indol-3-yl)ethyl]-amide | 367 | 368 | 1.44 |
| 370 | 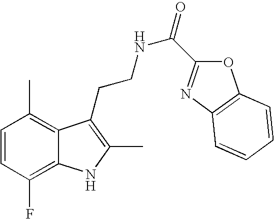 | benzoxazole-2-carboxylic acid[2-(7-fluoro-2,4-dimethyl-1H-indol-3-yl)ethyl]-amide | 351 | 352 | 1.31 |
| 371 | 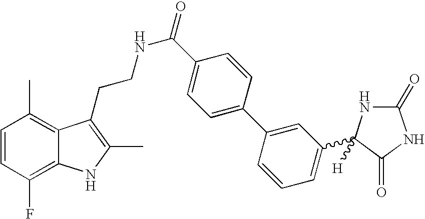 | 3′-(2,5-dioxo-imidazolidin-4-yl)-biphenyl-4-carboxylic acid[2-(7-fluoro-2,4-dimethyl-1H-indol-3-yl)ethyl]-amide | 484 | 485 | 1.16 |
| 372 | 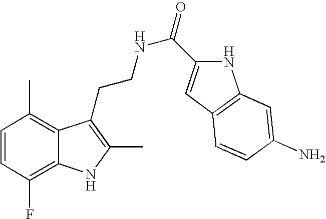 | 6-amino-1H-indole-2-carboxylic acid2-(7-fluoro-2,4-dimethyl-1H-indol-3-yl)ethyl]-amide | 364 | 365 | 0.94 |
| 373 | 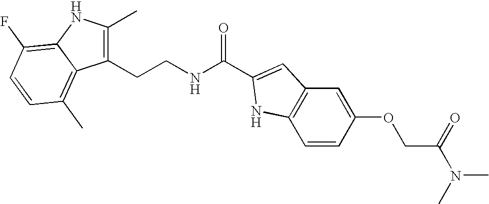 | 5-dimethyl-carbamoylmethoxy-1H-indole-2-carboxylic acid[2-(7-fluoro-2,4-dimethyl-1H-indol-3-yl)ethyl]-amide | 450 | 451 | 1.16 |
| 374 | 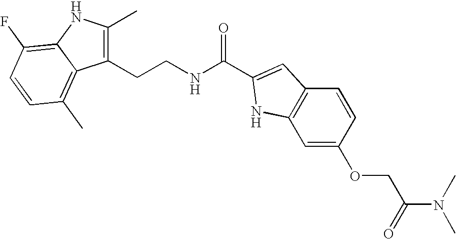 | 6-dimethyl-carbamoylmethoxy-1H-indole-2-carboxylic acid[2-(7-fluoro-2,4-dimethyl-1H-indol-3-yl)ethyl]-amide | 450 | 451 | 1.19 |
| 375 | 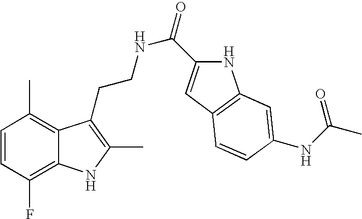 | 6-acetylamino-1H-indole-2-carboxylicacid [2-(7-fluoro-2,4-dimethyl-1H-indol-3-yl)ethyl]-amide | 406 | 407 | 1.14 |
| 376 | 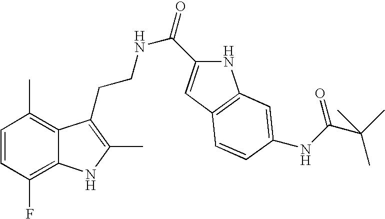 | 6-(2,2-dimethyl-propionylamino)-1H-indole-2-carboxylicacid [2-(7-fluoro-2,4-dimethyl-1H-indol-3-yl)ethyl]-amide | 448 | 449 | 1.30 |
| 377 | 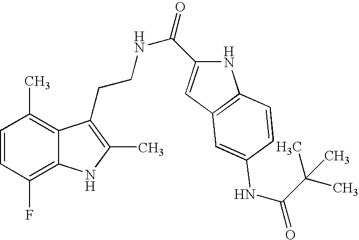 | 5-(2,2-dimethyl-propionylamino)-1H-indole-2-carboxylicacid [2-(7-fluoro-2,4-dimethyl-1H-indol-3-yl)ethyl]-amide | 448 | 449 | 1.28 |
| 378 | 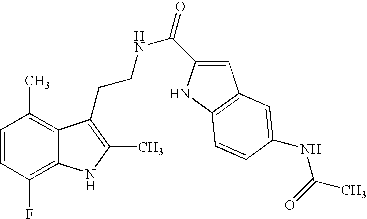 | 5-acetylamino-1H-indole-2-carboxylicacid [2-(7-fluoro-2,4-dimethyl-1H-amide | 406 | 407 | 1.10 |
| 379 | 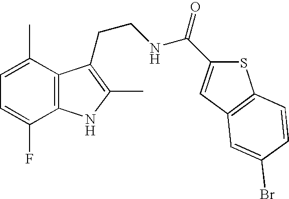 | 5-bromo-benzo[b]thiophene-2-carboxylic acid[2-(7-fluoro-2,4-dimethyl-1H-indol-3-yl)ethyl]-amide | 446 | 447 | 1.51 |
| 380 | 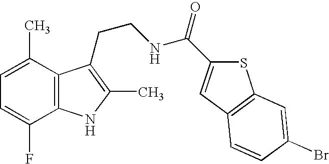 | 6-bromo-benzo[b]thiophene-2-carboxylic acid[2-(7-fluoro-2,4-dimethyl-1H-indol-3-yl)ethyl]-amide | 446 | 447 | 1.51 |
| 381 | 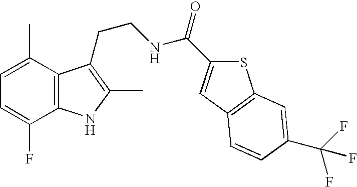 | 6-trifluoromethyl-benzo[b]thiophene-2-carboxylic acid[2-(7-fluoro-2,4-dimethyl-1H-indol-3-yl)ethyl]-amide | 434 | 433 | 1.52 |
| 382 | 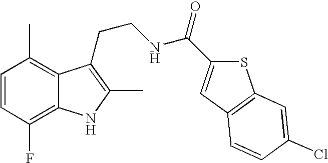 | 6-chloro-benzo[b]thiophene-2-carboxylic acid[2-(7-fluoro-2,4-dimethyl-1H-indol-3-yl)ethyl]-amide | 400 | 399[M − H] | 1.50 |
| 383 | 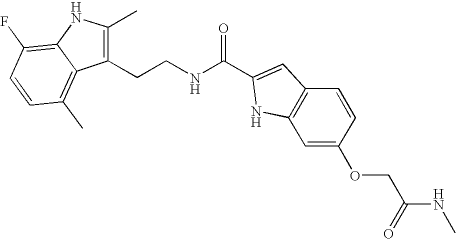 | 6-methylcarbamoyl-methoxy-1H-indole-2-carboxylic acid[2-(7-fluoro-2,4-dimethyl-1H-indol-3-yl)ethyl]-amide | 436 | 437 | 1.17 |
| 384 | 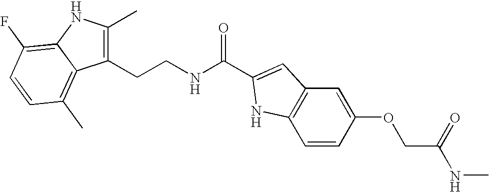 | 5-methylcarbamoyl-methoxy-1H-indole-2-carboxylic acid[2-(7-fluoro-2,4-dimethyl-1H-indol-3-yl)ethyl]-amide | 436 | 437 | 1.15 |

### BIOLOGICAL EXAMPLES

### 1. Detection of the Antagonism of the Human Prostaglandin E₂ (Subtype EP₂ Receptor Signal

### 1.1 Principle of Detection

The binding of PGE₂ to the EP₂ subtype of the human PGE₂ receptor induces activation of membrane-associated adenylate cyclases and leads to the formation of cAMP. In the presence of the phosphodiesterase inhibitor IBMX, cAMP which has accumulated due to this stimulation and been released by cell lysis is employed in a competitive detection method. In this assay, the cAMP in the lysate competes with cAMP-XL665 for binding of an Eu cryptate-labelled anti-cAMP antibody.

This results, in the absence of cellular cAMP, in a maximum signal which derives from coupling of this antibody to the cAMP-XL665 molecule. After excitation at 337 nm, this results in a FRET (fluorescence resonance energy transfer)-based, long-lived emission signal at 665 nm (and at 620 nM). The two signals are measured in a suitable measuring instrument with a time lag, i.e. after the background fluorescence has declined. Any increase in the low FRET signal caused by prostaglandin E₂ addition (measured as well ratio change=emission_{665 nm}/emission_{620 nm}*10 000) shows the effect of antagonists.

### 1.2. Detection Method

### 1.2.1 Antagonism Assay (Data for Each Well of a 384-Well Plate):

The substance solutions (0.75 μl) introduced into an assay plate and 30% DMSO are dissolved in 16 μl of a KRSB+IBMX stimulation solution (1× Krebs-Ringer Bicarbonate Buffer; Sigma-Aldrich # K-4002; including 750 μM 3-isobutyl-1-methylxanthine Sigma-Aldrich # I-7018), and then 15 μl thereof are transferred into a media-free cell culture plate which has been washed with KRSB shortly beforehand.

After preincubation at room temperature (RT) for 30 minutes, 5 μl of a 4×PGE2 solution (11 nM) are added, and incubation is carried out in the presence of the agonist at RT for a further 60 min (volume: ˜20 μl) before the reaction is then stopped by adding 5 μl of lysis buffer and incubated at RT for a further 20 min (volume: 25 μl). The cell lysate is then transferred into a measuring plate and measured in accordance with the manufacturer's information (cyclic AMP kit Cisbio International # 62AMPPEC).

### 1.2.2 Agonism Assay (Data for Each Well of a 384-Well Plate):

The substance solutions (0.75 μl) introduced into an assay plate and 30% DMSO are dissolved in 16 μl of a KRSB+IBMX stimulation solution (1× Krebs-Ringer Bicarbonate Buffer; Sigma-Aldrich # K-4002; including 750 μM 3-isobutyl-1-methylxanthine Sigma-Aldrich # I-7018), and then 15 μl thereof are transferred into a media-free cell culture plate which has been washed with KRSB shortly beforehand.

After incubation at room temperature (RT; volume: ˜15 μl) for 60 minutes, the reaction is then stopped by adding 5 μl of lysis buffer and incubated at RT for a further 20 min (volume: ˜20 μl). The cell lysate is then transferred into a measuring plate and measured in accordance with the manufacturer's information (cyclic AMP kit Cisbio International # 62AMPPEC).

### 2. The EP₂ Subtype of the PGE₂ Receptor and the Preovulatory Cumulus Expansion

### 2.1. Background:

In the preovulatory antral follicle, the oocyte is surrounded by cumulus cells which form a dense ring of cells around the oocyte. After the LH peak (lutenizing hormone), a series of processes is activated and leads to a large morphological change in this ring of cells composed of cumulus cells. In this case, the cumulus cells form an extracellular matrix which leads to so-called cumulus expansion (Vanderhyden et al. Dev Biol. 1990 August; 140(2):307-317). This cumulus expansion is an important component of the ovulatory process and of the subsequent possibility of fertilization.

Prostaglandins, and here prostaglandin E₂, whose synthesis is induced by the LH peak, are of crucial importance in cumulus expansion. Prostanoid EP₂ knockout mice (Hizaki et al. Proc Natl Acad Sci USA. 1999 Aug. 31; 96(18):10501-6.) show a markedly reduced cumulus expansion and severe subfertility, demonstrating the importance of the prostanoid EP₂ receptor for this process.

### 2.2 Cumulus Expansion Assay In Vitro

Folliculogenesis is induced in immature female mice (strain: CD1 (ICR) from Charles River) at an age of 14-18 days by a single dose (intraperitoneal) of 10 I.U. of PMSG (Pregnant Mare Serum Gonadotropine; Sigma G-4877, Lot 68H0909). 47-50 hours after the injection, the ovaries are removed and the cumulus-oocyte complexes are removed. The cumulus complex is not yet expanded at this stage.

The cumulus-oocyte complexes are then incubated with prostaglandin E2 (PGE2) (0.3 μM), vehicle control (ethanol) or test substances for 20-24 hours. Medium: alpha-MEM medium with 0.1 mM IBMX, pyruvates (0.23 mM) glutamines (2 mM), pen/strep 100 IU/ml pen. and 100 μg/ml strep.) and HSA (8 mg/ml)). Cumulus expansion is then established through the division into four stages (according to Vanderhyden et al. Dev Biol. 1990 August; 140(2):307-317).

TABLE 1
Example of the biological activity of the compounds of the invention
(measured by the cAMP antagonism assay):

| Substance of | |
|---|---|
| Example | Antagonism [IC₅₀, μM] |
| 2 | 6.8 |
| 6 | 1.5 |
| 29 | 1.2 |

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding European application No. 07090121.0, filed Jun. 13, 2007 are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A method of using a compound of the formula I 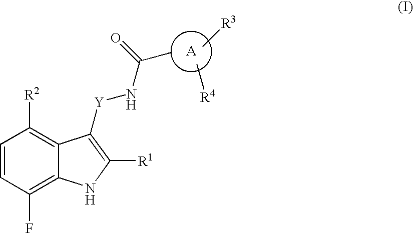
in which
A is an aryl or heteroaryl radical which may optionally be substituted one or more times by R³ and/or R⁴,
R¹ is a hydrogen, a C₁-C₆-alkyl radical which may optionally be substituted,
R² is a hydrogen, halogen, cyano, an —S(O)_{q}—CH₃, where q is 0-2, a C₁-C₄-alkoxy radical or C₁-C₆-alkyl, where this radical can be substituted in any way,
R³ is a hydrogen, halogen, amino, an —S(O)ₚ—C₁-C₆-alkyl group, where p is 0-2,
a C₁-C₆-acyl, NH—CO—NH₂, —O—CO—NH(C₁-C₆-alkyl), —O—CO—N(C₁-C₆-alkyl)₂ or NH—CO—C₁-C₆-alkyl radical,
a C₁-C₆-alkyl which may optionally be substituted one or more times, identically or differently, by C₁-C₆-acyl, C₁-C₆-alkoxy, hydroxy, cyano, CO₂—(C₁-C₆-alkyl), N—(C₁-C₆-alkyl)₂, C₅-C₁₂-heteroaryl, COOH, CO—NH₂, CO—NH(C₁-C₆-alkyl) or by CO—N(C₁-C₆-alkyl)₂,
a C₁-C₆-alkoxy which may optionally be substituted one or more times, identically or differently, by hydroxy, cyano, CO₂—(C₁-C₆-alkyl), N—(C₁-C₆-alkyl)₂, NH—C₃-C₆-cycloalkyl, COOH, CO—NH₂, CO—NH(C₁-C₆-alkyl) or by CO—N(C₁-C₆-alkyl)₂,
an O—C₆-C₁₂-aryl which may optionally be substituted by hydroxy, cyano, COOH or CO—NH₂,
a CH₂O—C₆-C₁₂-aryl which may optionally be substituted by hydroxy, cyano, COOH or CO—NH₂,
an O—C₅-C₁₆-heteroaryl which may optionally be substituted by hydroxy, cyano, COOH or CO—NH₂,
a hydroxy, cyano, O—CO—(C₁-C₆-alkyl), CO—NH(C₅-C₁₂-heteroaryl), NH—(C₁-C₆-alkyl), N—(C₁-C₆-alkyl)₂,
a C₆-C₁₂-aryl which may optionally be substituted one or more times, identically or differently, by halogen, by C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl), CH₂—NH—CO(C₅-C₁₂-heteroaryl), styryl, or an —S(O)ᵣ—CH₃, where r is 0-2, or two adjacent positions may be substituted by —O—CH₂—O— or —O—C(CH₃)₂—O—,
a monocyclic C₅-C₇-heteroaryl which may optionally be substituted one or more times, identically or differently, by C₁-C₆-alkyl, if R² is cyano or if R¹ and/or R² is identically or differently a C₁-C₆-alkyl radical, where at least one of the radicals is substituted at least once, or if R⁴ is —S(O)ₚ—C₁-C₆-alkyl, where p is 0-2, C₁-C₆-acyl-, —O—CO—NH(C₁-C₆-alkyl), —O—CO—N(C₁-C₆-alkyl)₂, C₆-C₁₂-aryloxy, C₅-C₁₆-heteroaryloxy, hydroxy, cyano or N—(C₁-C₆-alkyl)₂,
a monocyclic C₅-C₇-heteroaryl which may be substituted at least one or more times, identically or differently, by halogen, CF₃, C₁-C₆-acyl, C₁-C₆-alkoxy, hydroxy, CH₂—OH, cyano, CO₂—(C₁-C₆-alkyl), N—(C₁-C₆-alkyl)₂, COOH, CO—NH₂, CO—NH(C₁-C₆-alkyl) or CO—N(C₁-C₆-alkyl)₂,
a bi- or tricyclic C₈-C₁₂-heteroaryl which may optionally be substituted one or more times, identically or differently, by halogen, by C₁-C₆-alkyl, C₁-C₆-acyl, C₁-C₆-alkoxy, hydroxy, cyano, CO₂—(C₁-C₆-alkyl), N—(C₁-C₆-alkyl)₂, COOH, CO—NH₂, CO—NH(C₁-C₆-alkyl) or CO—N(C₁-C₆-alkyl)₂, or
a C₃-C₆-cycloalkyl which may optionally be substituted one or more times, identically or differently, by halogen, by C₁-C₆-alkyl, hydroxy, cyano, CO₂—(C₁-C₆-alkyl), C₁-C₆-acyl, N—(C₁-C₆-alkyl)₂, COOH, CO—NH₂, CO—NH(C₁-C₆-alkyl), CO—N(C₁-C₆-alkyl)₂ or C₁-C₆-alkoxy,
R⁴ is a hydrogen, halogen, amino, —S(O)ₚ—C₁-C₆-alkyl, where p is 0-2,
a C₁-C₆-acyl, NH—CO—NH₂, NH—CO—C₁-C₆-alkyl, —O—CO—NH(C₁-C₆-alkyl), —O—CO—N(C₁-C₆-alkyl)₂ or C₁-C₆-alkyl group which may optionally be substituted one or more times, identically or differently, by C₁-C₆-acyl, C₁-C₆-alkoxy, hydroxy, cyano, CO₂—(C₁-C₆-alkyl), N—(C₁-C₆-alkyl)₂, C₅-C₁₂-heteroaryl, COOH, CO—NH₂, CO—NH(C₁-C₆-alkyl) or by CO—N(C₁-C₆-alkyl)₂,
a C₁-C₆-alkoxy which may optionally be substituted one or more times, identically or differently, by hydroxy, cyano, CO₂—(C₁-C₆-alkyl), N—(C₁-C₆-alkyl)₂, NH—C₃-C₆-cycloalkyl, COOH, CO—NH₂, CO—NH(C₁-C₆-alkyl) or by CO—N(C₁-C₆-alkyl)₂, an O—C₆-C₁₂-aryl which may optionally be substituted by hydroxy, cyano, COOH or CO—NH₂,
a CH₂O—C₆-C₁₂-aryl which may optionally be substituted by hydroxy, cyano, COOH or CO—NH₂,
an O—C₅-C₁₆-heteroaryl which may optionally be substituted by hydroxy, cyano, COOH or CO—NH₂,
a hydroxy, cyano, O—CO—(C₁-C₆-alkyl), CO—NH(C₅-C₁₂-heteroaryl), NH—(C₁-C₆-alkyl), N—(C₁-C₆-alkyl)₂,
a C₆-C₁₂-aryl which may optionally be substituted one or more times, identically or differently, by halogen, by C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl), CH₂—NH—CO(C₅-C₁₂-heteroaryl), styryl, or an —S(O)ᵣ—CH₃, where r is 0-2, or two adjacent positions may be substituted by —O—CH₂—O— or —O—C(CH₃)₂—O—,
a monocyclic C₅-C₇-heteroaryl which may optionally be substituted one or more times, identically or differently, by C₁-C₆-alkyl, if R² is cyano or if R¹ and/or R² is identically or differently a C₁-C₆-alkyl radical, where at least one of the radicals is substituted at least once,
a monocyclic C₅-C₇-heteroaryl which may be substituted at least one or more times, identically or differently, by halogen, CF₃, C₁-C₆-acyl, C₁-C₆-alkoxy, hydroxy, CH₂—OH, cyano, CO₂—(C₁-C₆-alkyl), N—(C₁-C₆-alkyl)₂, COOH, CO—NH₂, CO—NH(C₁-C₆-alkyl) or CO—N(C₁-C₆-alkyl)₂,
a bi- or tricyclic C₈-C₁₂-heteroaryl which may optionally be substituted one or more times, identically or differently, by halogen, by C₁-C₆-alkyl, C₁-C₆-acyl, C₁-C₆-alkoxy, hydroxy, cyano, CO₂—(C₁-C₆-alkyl), N—(C₁-C₆-alkyl)₂, COOH, CO—NH₂, CO—NH(C₁-C₆-alkyl) or CO—N(C₁-C₆-alkyl)₂, or
a C₃-C₆-cycloalkyl which may optionally be substituted one or more times, identically or differently, by halogen, by C₁-C₆-alkyl, hydroxy, cyano, CO₂—(C₁-C₆-alkyl), C₁-C₆-acyl, N—(C₁-C₆-alkyl)₂, COOH, CO—NH₂, CO—NH(C₁-C₆-alkyl), CO—N(C₁-C₆-alkyl)₂ or C₁-C₆-alkoxy,
R³ and R⁴ are either in ortho, meta position or meta, para position relative to one another and together have the meaning —O—CO—S—, —S—CO—O—, CH₂—CO—O—, O—CO—CH₂—, —CH₂—CO—NH—, —NH—CO—CH₂—, —O—CO—NH—, —NH—CO—O—, —CO—CH₂—(CH₂)ₘ—, —CH₂—(CH₂)ₘ—CO—, —O—(CH₂)ₘ—O—, —O—C—(CH₃)₂—O—, —CH₂—(CH₂)ₘ—CH₂—, where m is 1-3,
Y is a —(CH₂)ₙ— group, where n is 1-3,
and the isomers, diastereomers, enantiomers and salts thereof, and cyclodextrin clathrates, where the following compounds are excluded:
N-[2-(7-fluoro-2,4-dimethyl-1H-indol-3-yl)ethyl]-3,4-dimethoxybenzamide
N-[2-(7-fluoro-2,4-dimethyl-1H-indol-3-yl)ethyl]-2-methylbenzamide
4-bromo-N-[2-(7-fluoro-2,4-dimethyl-1H-indol-3-yl)ethyl]benzamide.

2. The compound as claimed in claim 1, where
A is an aryl or heteroaryl radical which may optionally be substituted one or more times by R⁴ and/or R³,
is a hydrogen or C₁-C₆-alkyl radical which may be substituted one or more times by halogen,
is a hydrogen, halogen, cyano, an —S(O)_{q}—CH₃, where q is 0-2, a C₁-C₄-alkoxy radical or C₁-C₆-alkyl radical which may be substituted one or more times by halogen,
R² is a hydrogen, halogen, amino, —S(O)ₚ—C₁-C₆-alkyl, where p is 0-2,
a C₁-C₆-acyl, NH—CO—NH₂, NH—CO—C₁-C₆-alkyl, —O—CO—NH(C₁-C₆-alkyl), —O—CO—N(C₁-C₆-alkyl)₂, or C₁-C₆-alkyl group which may optionally be substituted one or more times, identically or differently, by C₁-C₆-acyl, C₁-C₆-alkoxy, hydroxy, cyano, CO₂—(C₁-C₆-alkyl), N—(C₁-C₆-alkyl)₂, C₅-C₁₂-heteroaryl, COOH, CO—NH₂, CO—NH(C₁-C₆-alkyl) or by CO—N(C₁-C₆-alkyl)₂,
a C₁-C₆-alkoxy which may optionally be substituted one or more times, identically or differently, by hydroxy, cyano, CO₂—(C₁-C₆-alkyl), N—(C₁-C₆-alkyl)₂, NH—C₃-C₆-cycloalkyl, COOH, CO—NH₂, CO—NH(C₁-C₆-alkyl) or by CO—N(C₁-C₆-alkyl)₂,
an O—C₆-C₁₂-aryl which may optionally be substituted by hydroxy, cyano, COOH or CO—NH₂,
a CH₂O—C₆-C₁₂-aryl which may optionally be substituted by hydroxy, cyano, COOH or CO—NH₂,
an O—C₅-C₁₆-heteroaryl which may optionally be substituted by hydroxy, cyano, COOH or CO—NH₂,
a hydroxy, cyano, O—CO—(C₁-C₆-alkyl), CO—NH(C₅-C₁₂-heteroaryl), NH—(C₁-C₆-alkyl), N—(C₁-C₆-alkyl)₂,
a C₆-C₁₂-aryl which may optionally be substituted one or more times, identically or differently, by halogen, by C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl), CH₂—NH—CO(C₅-C₁₂-heteroaryl), styryl, or an —S(O)ᵣ—CH₃, where r is 0-2,
or two adjacent positions may be substituted by —O—CH₂—O— or —O—C(CH₃)₂—O—, a monocyclic C₅-C₇-heteroaryl which may optionally be substituted one or more times, identically or differently, by C₁-C₆-alkyl, if R² is cyano or if R¹ and/or R² is identically or differently a C₁-C₆-alkyl radical, where at least one of the radicals is substituted at least once, or if R⁴ is —S(O)ₚ—C₁-C₆-alkyl, where p is 0-2, C₁-C₆-acyl-, —O—CO—NH(C₁-C₆-alkyl), —O—CO—N(C₁-C₆-alkyl)₂, C₆-C₁₂-aryloxy, C₅-C₁₆-heteroaryloxy, hydroxy, cyano or N—(C₁-C₆-alkyl)₂,
a monocyclic C₅-C₇-heteroaryl which may be substituted at least one or more times, identically or differently, by halogen, by CF₃, C₁-C₆-acyl, C₁-C₆-alkoxy, hydroxy, CH₂—OH, cyano, CO₂—(C₁-C₆-alkyl), N—(C₁-C₆-alkyl)₂, COOH, CO—NH₂, CO—NH(C₁-C₆-alkyl) or CO—N(C₁-C₆-alkyl)₂,
a bi- or tricyclic C₈-C₁₂-heteroaryl which may optionally be substituted one or more times, identically or differently, by halogen, by C₁-C₆-alkyl, C₁-C₆-acyl, C₁₋₇ C₆-alkoxy, hydroxy, cyano, CO₂—(C₁-C₆-alkyl), N—(C₁-C₆-alkyl)₂, COOH, CO—NH₂, CO—NH(C₁-C₆-alkyl) or CO—N(C₁-C₆-alkyl)₂, or
a C₃-C₆-cycloalkyl which may optionally be substituted one or more times, identically or differently, by halogen, by C₁-C₆-alkyl, hydroxy, cyano, CO₂—(C₁-C₆-alkyl), C₁-C₆-acyl, N—(C₁-C₆-alkyl)₂, COOH, CO—NH₂, CO—NH(C₁-C₆-alkyl), CO—N(C₁-C₆-alkyl)₂ or C₁-C₆-alkoxy,
R⁴ is a hydrogen, halogen, amino, —S(O)ₚ—C₁-C₆-alkyl, where p is 0-2,
a C₁-C₆-acyl, NH—CO—NH₂, NH—CO—C₁-C₆-alkyl, —O—CO—NH(C₁-C₆-alkyl), —O—CO—N(C₁-C₆-alkyl)₂, or C₁-C₆-alkyl group which may optionally be substituted one or more times, identically or differently, by C₁-C₆-acyl, C₁-C₆-alkoxy, hydroxy, cyano, CO₂—(C₁-C₆-alkyl), N—(C₁-C₆-alkyl)₂, C₅-C₁₂-heteroaryl, COOH, CO—NH₂, CO—NH(C₁-C₆-alkyl) or by CO—N(C₁-C₆-alkyl)₂,
a C₁-C₆-alkoxy which may optionally be substituted one or more times, identically or differently, by hydroxy, cyano, CO₂—(C₁-C₆-alkyl), N—(C₁-C₆-alkyl)₂, NH—C₃-C₆-cycloalkyl, COOH, CO—NH₂, CO—NH(C₁-C₆-alkyl) or by CO—N(C₁-C₆-alkyl)₂,
an O—C₆-C₁₂-aryl which may optionally be substituted by hydroxy, cyano, COOH or CO—NH₂,
a CH₂O—C₆-C₁₂-aryl which may optionally be substituted by hydroxy, cyano, COOH or CO—NH₂,
an O—C₅-C₁₆-heteroaryl which may optionally be substituted by hydroxy, cyano, COOH or CO—NH₂,
a hydroxy, cyano, O—CO—(C₁-C₆-alkyl), CO—NH(C₅-C₁₂-heteroaryl), NH—(C₁-C₆-alkyl), N—(C₁-C₆-alkyl)₂,
a C₆-C₁₂-aryl which may optionally be substituted one or more times, identically or differently, by halogen, by C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl), CH₂—NH—CO(C₅-C₁₂-heteroaryl), styryl, or an —S(O)ᵣ—CH₃, where r is 0-2, or two adjacent positions may be substituted by —O—CH₂—O— or —O—C(CH₃)₂—O—,
a monocyclic C₅-C₇-heteroaryl which may optionally be substituted one or more times, identically or differently, by C₁-C₆-alkyl, if R² is cyano or if R¹ and/or R² is identically or differently a C₁-C₆-alkyl radical, where at least one of the radicals is substituted at least once,
a monocyclic C₅-C₇-heteroaryl which may be substituted at least one or more times, identically or differently, by halogen, by CF₃, C₁-C₆-acyl, C₁-C₆-alkoxy, hydroxy, CH₂—OH, cyano, CO₂—(C₁-C₆-alkyl), N—(C₁-C₆-alkyl)₂, COOH, CO—NH₂, CO—NH(C₁-C₆-alkyl) or CO—N(C₁-C₆-alkyl)₂,
a bi- or tricyclic C₈-C₁₂-heteroaryl which may optionally be substituted one or more times, identically or differently, by halogen, by C₁-C₆-alkyl, C₁-C₆-acyl, C₁-C₆-alkoxy, hydroxy, cyano, CO₂—(C₁-C₆-alkyl), N—(C₁-C₆-alkyl)₂, COOH, CO—NH₂, CO—NH(C₁-C₆-alkyl) or CO—N(C₁-C₆-alkyl)₂, or
a C₃-C₆-cycloalkyl which may optionally be substituted one or more times, identically or differently, by halogen, by C₁-C₆-alkyl, hydroxy, cyano, CO₂—(C₁-C₆-alkyl), C₁-C₆-acyl, N—(C₁-C₆-alkyl)₂, COOH, CO—NH₂, CO—NH(C₁-C₆-alkyl), CO—N(C₁-C₆-alkyl)₂ or C₁-C₆-alkoxy,
R³ and R⁴ are either in ortho, meta position or meta, para position relative to one another and together have the meaning of —O—CO—S—, —S—CO—O—, CH₂—CO—O—, O—CO—CH₂—, —CH₂—CO—NH—, —NH—CO—CH₂—, —O—CO—NH—, —NH—CO—O—, —CO—CH₂—(CH₂)ₘ—, —CH₂—(CH₂)ₘ—CO—, —O—(CH₂)ₘ—O—, —O—C—(CH₃)₂—O—, —CH₂—(CH₂)ₘ—CH₂—, where m is 1-3,
Y is a —(CH₂)ₙ— group, where n is 1-3,
and the isomers, diastereomers, enantiomers and salts thereof, and cyclodextrin clathrates.

3. The compound as claimed in claim 1, where
A is an aryl or heteroaryl radical which may optionally be substituted one or more times by R⁴ and/or R³,
is a hydrogen or a C₁-C₆-alkyl group which is substituted one or more times by halogen, is a hydrogen, halogen, cyano, an —S(O)_{q}—CH₃, where q is 0-2, a C₁-C₄-alkoxy radical or a C₁-C₆-alkyl group which is substituted one or more times by halogen,
R³ is a hydrogen, halogen, amino, —S(O)ₚ—CH₃, where p is 0-2,
an —S—CF₃, SO₂NH₂, C₁-C₆-acyl, NH—CO—NH₂, NH—CO—C₁-C₆-alkyl, —O—CO—NH(C₁-C₆-alkyl), —O—CO—N(C₁-C₆-alkyl)₂, or C₁-C₆-alkyl group which may optionally be substituted one or more times, identically or differently, by C₁-C₄-acyl, C₁-C₄-alkoxy, hydroxy, cyano, CO₂—(C₁-C₄-alkyl), N—(C₁-C₄-alkyl)₂, C₅-C₁₂-heteroaryl, COOH, CO—NH₂, CO—NH(C₁-C₄-alkyl) or by CO—N(C₁-C₄-alkyl)₂,
a C₁-C₆-alkoxy which may optionally be substituted one or more times, identically or differently, by hydroxy, cyano, CO₂—(C₁-C₆-alkyl), N—(C₁-C₆-alkyl)₂, NH—C₃-C₆-cycloalkyl, COOH, CO—NH₂, CO—NH(C₁-C₆-alkyl) or by CO—N(C₁-C₆-alkyl)₂,
an O—C₆-C₁₂-aryl which may optionally be substituted by hydroxy, cyano, COOH or CO—NH₂,
a CH₂O—C₆-C₁₂-aryl which may optionally be substituted by hydroxy, cyano, COOH or CO—NH₂,
an O—C₅-C₁₆-heteroaryl which may optionally be substituted by hydroxy, cyano, COOH or CO—NH₂,
a hydroxy, cyano, O—CO—(C₁-C₆-alkyl), CO—NH(C₅-C₁₂-heteroaryl), NH—(C₁-C₆-alkyl), N—(C₁-C₆-alkyl)₂,
a C₆-C₁₂-aryl which may optionally be substituted one or more times, identically or differently, by halogen, by C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl), CH₂—NH—CO(C₅-C₁₂-heteroaryl), styryl, or an —S(O)ᵣ—CH₃, where r is 0-2, or two adjacent positions may be substituted by —O—CH₂—O— or —O—C(CH₃)₂—O—,
a monocyclic C₅-C₇-heteroaryl which may optionally be substituted one or more times, identically or differently, by C₁-C₆-alkyl, if R² is cyano or if R¹ and/or R² is identically or differently a C₁-C₆-alkyl radical, where at least one of the radicals is substituted at least once by halogen, or if R⁴ is —S(O)ₚ—C₁-C₆-alkyl, where p is 0-2, C₁-C₆-acyl, —O—CO—NH(C₁-C₆-alkyl), —O—CO—N(C₁-C₆-alkyl)₂, C₆-C₁₂-aryloxy, C₅-C₁₆-heteroaryloxy, hydroxy, cyano or N—(C₁-C₆-alkyl)₂,
a monocyclic C₅-C₇-heteroaryl which may be substituted at least one or more times, identically or differently, by halogen, by CF₃, C₁-C₄-acyl, C₁-C₄-alkoxy, hydroxy, CH₂—OH, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂, or
a bi- or tricyclic C₈-C₁₂-heteroaryl which may optionally be substituted one or more times, identically or differently, by halogen, by C₁-C₆-alkyl, C₁-C₆-acyl, C₁-C₆-alkoxy, hydroxy, cyano, CO₂—(C₁-C₆-alkyl), N—(C₁-C₆-alkyl)₂, COOH, CO—NH₂, CO—NH(C₁-C₆-alkyl) or CO—N(C₁-C₆-alkyl)₂, or
a C₃-C₆-cycloalkyl which may optionally be substituted one or more times, identically or differently, by halogen, by C₁-C₄-alkyl, hydroxy, cyano, CO₂—(C₁-C₄-alkyl), C₁-C₄-acyl, N—(C₁-C₄-alkyl)₂, COOH, CO—NH₂, CO—NH(C₁-C₄-alkyl), CO—N(C₁-C₄-alkyl)₂ or C₁-C₄-alkoxy,
R⁴ is a hydrogen, halogen, amino, —S(O)ₚ—C₁-C₆-alkyl, where p is 0-2,
a C₁-C₆-acyl, NH—CO—NH₂, NH—CO—C₁-C₆-alkyl, —O—CO—NH(C₁-C₆-alkyl), —O—CO—N(C₁-C₆-alkyl)₂, or C₁-C₆-alkyl group which may optionally be substituted one or more times, identically or differently, by C₁-C₆-acyl, C₁-C₆-alkoxy, hydroxy, cyano, CO₂—(C₁-C₆-alkyl), N—(C₁-C₆-alkyl)₂, C₅-C₁₂-heteroaryl, COOH, CO—NH₂, CO—NH(C₁-C₆-alkyl) or by CO—N(C₁-C₆-alkyl)₂,
a C₁-C₆-alkoxy which may optionally be substituted one or more times, identically or differently, by hydroxy, cyano, CO₂—(C₁-C₆-alkyl), N—(C₁-C₆-alkyl)₂, NH—C₃-C₆-cycloalkyl, COOH, CO—NH₂, CO—NH(C₁-C₆-alkyl) or by CO—N(C₁-C₆-alkyl)₂,
an O—C₆-C₁₂-aryl which may optionally be substituted by hydroxy, cyano, COOH or CO—NH₂,
a CH₂O—C₆-C₁₂-aryl which may optionally be substituted by hydroxy, cyano, COOH or CO—NH₂,
an O—C₅-C₁₆-heteroaryl, which may optionally be substituted by hydroxy, cyano, COOH or CO—NH₂,
a hydroxy, cyano, O—CO—(C₁-C₆-alkyl), CO—NH(C₅-C₁₂-heteroaryl), NH—(C₁-C₆-alkyl), N—(C₁-C₆-alkyl)₂ or
a C₆-C₁₂-aryl which may optionally be substituted one or more times, identically or differently, by halogen, by C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl), CH₂—NH—CO(C₅-C₁₂-heteroaryl), styryl, or an —S(O)ᵣ—CH₃, where r is 0-2, or two adjacent positions may be substituted by —O—CH₂—O— or —O—C(CH₃)₂—O—,
a monocyclic C₅-C₇-heteroaryl which may optionally be substituted one or more times, identically or differently, by C₁-C₆-alkyl, if R² is cyano or if R¹ and/or R² is identically or differently a C₁-C₆-alkyl radical, where at least one of the radicals is substituted at least once by halogen,
a monocyclic C₅-C₇-heteroaryl which may be substituted at least one or more times, identically or differently, by halogen, by CF₃, C₁-C₆-acyl, C₁-C₆-alkoxy, hydroxy, CH₂—OH, cyano, CO₂—(C₁-C₆-alkyl), N—(C₁-C₆-alkyl)₂, COOH, CO—NH₂, CO—NH(C₁-C₆-alkyl) or CO—N(C₁-C₆-alkyl)₂, or
a bi- or tricyclic C₈-C₁₂-heteroaryl which may optionally be substituted one or more times, identically or differently, by halogen, by C₁-C₆-alkyl, C₁-C₆-acyl, C₁-C₆-alkoxy, hydroxy, cyano, CO₂—(C₁-C₆-alkyl), N—(C₁-C₆-alkyl)₂, COOH, CO—NH₂, CO—NH(C₁-C₆-alkyl) or CO—N(C₁-C₆-alkyl)₂, or
a C₃-C₆-cycloalkyl which may optionally be substituted one or more times, identically or differently, by halogen, by C₁-C₆-alkyl, hydroxy, cyano, CO₂—(C₁-C₆-alkyl), C₁-C₆-acyl, N—(C₁-C₆-alkyl)₂, COOH, CO—NH₂, CO—NH(C₁-C₆-alkyl), CO—N(C₁-C₆-alkyl)₂ or C₁-C₆-alkoxy,
R³ and R⁴ are either in ortho, meta position or meta, para position relative to one another and together have the meaning —O—CO—S—, —S—CO—O—, CH₂—CO—O—, O—CO—CH₂—, —CH₂—CO—NH—, —NH—CO—CH₂—, —O—CO—NH—, —NH—CO—O—, —CO—CH₂—(CH₂)ₘ—, —CH₂—(CH₂)ₘ—CO—, —O—(CH₂)ₘ—O—, —O—C—(CH₃)₂—O—, —CH₂—(CH₂)ₘ—CH₂—, where m is 1-3,
Y is a —(CH₂)ₙ— group, where n is 1-3,
and the isomers, diastereomers, enantiomers and salts thereof, and cyclodextrin clathrates.

4. The compound as claimed in claim 1, where
A is an aryl or heteroaryl radical which may optionally be substituted one or more times by R⁴ and/or R³,
R¹ is a hydrogen or a C₁-C₆-alkyl group which is substituted one or more times by halogen,
R² is a hydrogen, halogen, cyano, an —S(O)_{q}—CH₃, where q is 0-2, a C₁-C₄-alkoxy radical or C₁-C₆-alkyl group which is substituted one or more times by halogen,
R³ is a hydrogen, halogen, amino, —S(O)ₚ—CH₃, where p is 0-2,
an —S—CF₃, C₁-C₆-acyl, NH—CO—NH₂, NH—CO—C₁-C₆-alkyl, —O—CO—NHCH₃, —O—CO—N(CH₃)₂ or C₁-C₆-alkyl group which may optionally be substituted one or more times, identically or differently, by C₁-C₄-acyl, C₁-C₄-alkoxy, hydroxy, cyano, CO₂—(C₁-C₄-alkyl), N—(C₁-C₄-alkyl)₂, C₅-C₁₂-heteroaryl, COOH, CO—NH₂, CO—NH(C₁-C₄-alkyl) or by CO—N(C₁-C₄-alkyl)₂,
a C₁-C₆-alkoxy which may optionally be substituted one or more times, identically or differently, by hydroxy, cyano, CO₂—(C₁-C₆-alkyl), N—(C₁-C₆-alkyl)₂, NH—C₃-C₆-cycloalkyl, COOH, CO—NH₂, CO—NH(C₁-C₆-alkyl) or by CO—N(C₁-C₆-alkyl)₂,
an O—C₆-C₁₂-aryl which may optionally be substituted by hydroxy, cyano, COOH or CO—NH₂,
a CH₂O—C₆-C₁₂-aryl which may optionally be substituted by hydroxy, cyano, COOH or CO—NH₂,
an O—C₅-C₁₆-heteroaryl which may optionally be substituted by hydroxy, cyano, COOH or CO—NH₂,
a hydroxy, cyano, O—CO—(C₁-C₆-alkyl), CO—NH(C₅-C₁₂-heteroaryl), NH—(C₁-C₆-alkyl), N—(C₁-C₆-alkyl)₂,
a C₆-C₁₂-aryl which may optionally be substituted one or more times, identically or differently, by halogen, by C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl), CH₂—NH—CO(C₅-C₁₂-heteroaryl), styryl, or an —S(O)ᵣ—CH₃, where r is 0-2, or two adjacent positions may be substituted by —O—CH₂—O— or —O—C(CH₃)₂—O—,
a monocyclic C₅-C₇-heteroaryl which may optionally be substituted one or more times, identically or differently, by C₁-C₆-alkyl, if R² is cyano or if R¹ and/or R² is identically or differently a C₁-C₆-alkyl radical, where at least one of the radicals is substituted at least once by halogen, or if R⁴ is —S(O)ₚ—C₁-C₆-alkyl, where p is 0-2, C₁-C₆-acyl-, —O—CO—NH(C₁-C₆-alkyl), —O—CO—N(C₁-C₆-alkyl)₂, C₆-C₁₂-aryloxy, C₅-C₁₆-heteroaryloxy, hydroxy, cyano or N—(C₁-C₆-alkyl)₂,
a monocyclic C₅-C₇-heteroaryl which may be substituted at least one or more times, identically or differently, by halogen, by CF₃, C₁-C₄-acyl, C₁-C₄-alkoxy, hydroxy, CH₂—OH, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂,
a bi- or tricyclic C₈-C₁₂-heteroaryl which may optionally be substituted one or more times, identically or differently, by halogen, by C₁-C₆-alkyl, C₁-C₆-acyl, C₁-C₆-alkoxy, hydroxy, cyano, CO₂—(C₁-C₆-alkyl), N—(C₁-C₆-alkyl)₂, COOH, CO—NH₂, CO—NH(C₁-C₆-alkyl) or CO—N(C₁-C₆-alkyl)₂, or
a C₃-C₆-cycloalkyl which may optionally be substituted one or more times, identically or differently, by halogen, by C₁-C₄-alkyl, hydroxy, cyano, CO₂—(C₁-C₄-alkyl), C₁-C₄-acyl, N—(C₁-C₄-alkyl)₂, COOH, CO—NH₂, CO—NH(C₁-C₄-alkyl), CO—N(C₁-C₄-alkyl)₂ or C₁-C₄-alkoxy,
R⁴ is a hydrogen, halogen, amino, —S(O)ₚ—CH₃, where p is 0-2,
an —S—CF₃, C₁-C₆-acyl, NH—CO—NH₂, NH—CO—C₁-C₆-alkyl, —O—CO—NHCH₃, —O—CO—N(CH₃)₂, or C₁-C₆-alkyl group which may optionally be substituted one or more times, identically or differently, by C₁-C₄-acyl, C₁-C₄-alkoxy, hydroxy, cyano, CO₂—(C₁-C₄-alkyl), N—(C₁-C₄-alkyl)₂, C₅-C₁₂-heteroaryl, COOH, CO—NH₂, CO—NH(C₁-C₄-alkyl) or by CO—N(C₁-C₄-alkyl)₂,
a C₁-C₆-alkoxy which may optionally be substituted one or more times, identically or differently, by hydroxy, cyano, CO₂—(C₁-C₆-alkyl), N—(C₁-C₆-alkyl)₂, NH—C₃-C₆-cycloalkyl, COOH, CO—NH₂, CO—NH(C₁-C₆-alkyl) or by CO—N(C₁-C₆-alkyl)₂,
an O—C₆-C₁₂-aryl which may optionally be substituted by hydroxy, cyano, COOH or CO—NH₂,
a CH₂O—C₆-C₁₂-aryl which may optionally be substituted by hydroxy, cyano, COOH or CO—NH₂,
an O—C₅-C₁₆-heteroaryl which may optionally be substituted by hydroxy, cyano, COOH or CO—NH₂,
a hydroxy, cyano, O—CO—(C₁-C₆-alkyl), CO—NH(C₅-C₁₂-heteroaryl), N—(C₁-C₆-alkyl), N—(C₁-C₆-alkyl)₂,
a C₆-C₁₂-aryl which may optionally be substituted one or more times, identically or differently, by halogen, by C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl), CH₂—NH—CO(C₅-C₁₂-heteroaryl), styryl, or an —S(O)ᵣ—CH₃, where r is 0-2, or two adjacent positions may be substituted by —O—CH₂—O— or —O—C(CH₃)₂—O—,
a monocyclic C₅-C₇-heteroaryl which may optionally be substituted one or more times, identically or differently, by C₁-C₆-alkyl, if R² is cyano or if R¹ and/or R² is identically or differently a C₁-C₆-alkyl radical, where at least one of the radicals is substituted at least once by halogen,
a monocyclic C₅-C₁₂-heteroaryl which may be substituted at least one or more times, identically or differently, by halogen, by CF₃, C₁-C₄-acyl, C₁-C₄-alkoxy, hydroxy, CH₂—OH, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂,
a bi- or tricyclic C₈-C₁₂-heteroaryl which may optionally be substituted one or more times, identically or differently, by halogen, by C₁-C₆-alkyl, C₁-C₆-acyl, C₁-C₆-alkoxy, hydroxy, cyano, CO₂—(C₁-C₆-alkyl), N—(C₁-C₆-alkyl)₂, COOH, CO—NH₂, CO—NH(C₁-C₆-alkyl) or CO—N(C₁-C₆-alkyl)₂, or
a C₃-C₆-cycloalkyl which may optionally be substituted one or more times, identically or differently, by halogen, by C₁-C₄-alkyl, hydroxy, cyano, CO₂—(C₁-C₄-alkyl), C₁-C₄-acyl, N—(C₁-C₄-alkyl)₂, COOH, CO—NH₂, CO—NH(C₁-C₄-alkyl), CO—N(C₁-C₄-alkyl)₂ or C₁-C₄-alkoxy,
R³ and R⁴ are either in ortho, meta position or meta, para position relative to one another and together have the meaning —O—CO—S—, —S—CO—O—, CH₂—CO—O—, O—CO—CH₂—, —CH₂—CO—NH—, —NH—CO—CH₂—, —O—CO—NH—, —NH—CO—O—, —CO—CH₂—(CH₂)ₘ—, —CH₂—(CH₂)ₘ—CO—, —O—(CH₂)ₘ—O—, —O—C—(CH₃)₂—O—, —CH₂—(CH₂)ₘ—CH₂—, where m is 1-3,
Y is a —(CH₂)ₙ— group, where n is 1-3,
and the isomers, diastereomers, enantiomers and salts thereof, and cyclodextrin clathrates.

5. The compound as claimed in claim 1, where
A is a phenyl, naphthyl or heteroaryl radical which may optionally be substituted once or twice by R³ and/or R⁴,
R¹ is a hydrogen or a C₁-C₆-alkyl group which may be substituted one or more times by halogen,
R² is a hydrogen, halogen, cyano, an —S(O)_{q}—CH₃, where q is 0-2, a C₁-C₄-alkoxy radical or C₁-C₆-alkyl group,
R³ is a hydrogen, halogen, amino, —S(O)ₚ—CH₃, where p is 0-2,
an —S—CF₃, C₁-C₆-acyl, NH—CO—NH₂, NH—CO—C₁-C₆-alkyl, —O—CO—NHCH₃, —O—CO—N(CH₃)₂, or C₁-C₆-alkyl group which may optionally be substituted once, twice, three, four or five times, identically or differently, by C₁-C₄-acyl, C₁-C₄-alkoxy, hydroxy, cyano, CO₂—(C₁-C₄-alkyl), N—(C₁-C₄-alkyl)₂, C₅-C₁₂-heteroaryl, COOH, CO—NH₂, CO—NH(C₁-C₄-alkyl) or by CO—N(C₁-C₄-alkyl)₂,
a C₁-C₆-alkoxy which may optionally be substituted one or more times, identically or differently, by hydroxy, cyano, CO₂—(C₁-C₆-alkyl), N—(C₁-C₆-alkyl)₂, NH—C₃-C₆-cycloalkyl, COOH, CO—NH₂, CO—NH(C₁-C₆-alkyl) or by CO—N(C₁-C₆-alkyl)₂,
an O—C₆-C₁₂-aryl which may optionally be substituted by hydroxy, cyano, COOH or CO—NH₂,
a CH₂O—C₆-C₁₂-aryl which may optionally be substituted by hydroxy, cyano, COOH or CO—NH₂,
an O—C₅-C₁₆-heteroaryl which may optionally be substituted by hydroxy, cyano, COOH or CO—NH₂,
a hydroxy, cyano, O—CO—(C₁-C₆-alkyl), CO—NH(C₅-C₁₂-heteroaryl), NH—(C₁-C₆-alkyl), N—(C₁-C₆-alkyl)₂,
a C₆-C₁₂-aryl which may optionally be substituted one or more times, identically or differently, by halogen, by C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl), CH₂—NH—CO(C₅-C₁₂-heteroaryl), styryl, or an —S(O)ᵣ—CH₃, where r is 0-2, or two adjacent positions may be substituted by —O—CH₂—O— or —O—C(CH₃)₂—O—,
a monocyclic C₅-C₇-heteroaryl which may optionally be substituted one or more times, identically or differently, by C₁-C₆-alkyl, if R² is cyano or if R¹ and/or R² is identically or differently a C₁-C₄-alkyl radical, where at least one of the radicals is substituted at least once by halogen, or if R⁴ is —S(O)ₚ—C₁-C₄-alkyl, where p is 0-2, C₁-C₄-acyl-, —O—CO—NH(C₁-C₄-alkyl), —O—CO—N(C₁-C₄-alkyl)₂, C₆-C₁₂-aryloxy, C₅-C₁₆-heteroaryloxy, hydroxy, cyano or N—(C₁-C₄-alkyl)₂,
a monocyclic C₅-C₇-heteroaryl which may be substituted at least once or else twice, three, four or five times, identically or differently, by halogen, by CF₃, C₁-C₄-acyl, C₁-C₄-alkoxy, hydroxy, CH₂—OH, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂, or
a bi- or tricyclic C₈-C₁₂-heteroaryl which may optionally be substituted once, twice, three, four or five times, identically or differently, by halogen, by C₁-C₆-alkyl, C₁-C₆-acyl, C₁-C₆-alkoxy, hydroxy, cyano, CO₂—(C₁-C₆-alkyl), N—(C₁-C₆-alkyl)₂, COOH, CO—NH₂, CO—NH(C₁-C₆-alkyl) or CO—N(C₁-C₆-alkyl)₂,
a C₃-C₆-cycloalkyl which may optionally be substituted once, twice, three, four or five times, identically or differently, by halogen, by C₁-C₄-alkyl, hydroxy, cyano, CO₂—(C₁-C₄-alkyl), C₁-C₄-acyl, N—(C₁-C₄-alkyl)₂, COOH, CO—NH₂, CO—NH(C₁-C₄-alkyl), CO—N(C₁-C₄-alkyl)₂ or C₁-C₄-alkoxy,
R⁴ is a hydrogen, halogen, amino, —S(O)ₚ—CH₃, where p is 0-2,
an —S—CF₃, C₁-C₆-acyl, NH—CO—NH₂, NH—CO—C₁-C₆-alkyl, —O—CO—NHCH₃, —O—CO—N(CH₃)₂, or C₁-C₆-alkyl group which may optionally be substituted once, twice, three, four or five times, identically or differently, by C₁-C₄-acyl, C₁-C₄-alkoxy, hydroxy, cyano, CO₂—(C₁-C₄-alkyl), N—(C₁-C₄-alkyl)₂, C₅-C₁₂-heteroaryl, COOH, CO—NH₂, CO—NH(C₁-C₄-alkyl) or by CO—N(C₁-C₄-alkyl)₂,
a C₁-C₆-alkoxy which may optionally be substituted one or more times, identically or differently, by hydroxy, cyano, CO₂—(C₁-C₆-alkyl), N—(C₁-C₆-alkyl)₂, NH—C₃-C₆-cycloalkyl, COOH, CO—NH₂, CO—NH(C₁-C₆-alkyl) or by CO—N(C₁-C₆-alkyl)₂,
an O—C₆-C₁₂-aryl which may optionally be substituted by hydroxy, cyano, COOH or CO—NH₂,
a CH₂O—C₆-C₁₂-aryl which may optionally be substituted by hydroxy, cyano, COOH or CO—NH₂,
an O—C₅-C₁₆-heteroaryl which may optionally be substituted by hydroxy, cyano, COOH or CO—NH₂,
a hydroxy, cyano, O—CO—(C₁-C₆-alkyl), CO—NH(C₅-C₁₂-heteroaryl), NH—(C₁-C₆-alkyl), N—(C₁-C₆-alkyl)₂ or
a C₆-C₁₂-aryl which may optionally be substituted one or more times, identically or differently, by halogen, by C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl), CH₂—NH—CO(C₅-C₁₂-heteroaryl), styryl, or an —S(O)ᵣ—CH₃, where r is 0-2, or two adjacent positions may be substituted by —O—CH₂—O— or —O—C(CH₃)₂—O—,
a monocyclic C₅-C₇-heteroaryl which may optionally be substituted one or more times, identically or differently, by C₁-C₆-alkyl, if R² is cyano or if R¹ and/or R² is identically or differently a C₁-C₄-alkyl radical, where at least one of the radicals is substituted at least once by halogen,
a monocyclic C₅-C₇-heteroaryl which may be substituted at least once or else twice, three, four or five times, identically or differently, by halogen, by CF₃, C₁-C₄-acyl, C₁-C₄-alkoxy, hydroxy, CH₂—OH, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂, or
a bi- or tricyclic C₈-C₁₂-heteroaryl which may optionally be substituted once, twice, three, four or five times, identically or differently, by halogen, by C₁-C₆-alkyl, C₁-C₆-acyl, C₁-C₆-alkoxy, hydroxy, cyano, CO₂—(C₁-C₆-alkyl), N—(C₁-C₆-alkyl)₂, COOH, CO—NH₂, CO—NH(C₁-C₆-alkyl) or CO—N(C₁-C₆-alkyl)₂ or
a C₃-C₆-cycloalkyl which may optionally be substituted once, twice, three, four or five times, identically or differently, by halogen, by C₁-C₄-alkyl, hydroxy, cyano, CO₂—(C₁-C₄-alkyl), C₁-C₄-acyl, N—(C₁-C₄-alkyl)₂, COOH, CO—NH₂, CO—NH(C₁-C₄-alkyl), CO—N(C₁-C₄-alkyl)₂ or C₁-C₄-alkoxy,
R³ and R⁴ are either in ortho, meta position or meta, para position relative to one another and together have the meaning of
—O—CO—S—, —S—CO—O—, CH₂—CO—O—, O—CO—CH₂—, —CH₂—CO—NH—, —NH—CO—CH₂—, —O—CO—NH—, —NH—CO—O—, —CO—CH₂—(CH₂)ₘ—, —CH₂—(CH₂)ₘ—CO—, —O—(CH₂)ₘ—O—, —O—C—(CH₃)₂—O—, —CH₂—(CH₂)ₘ—CH₂—, where m is 1-3,
Y is a —(CH₂)ₙ— group, where n is 1-3,
and the isomers, diastereomers, enantiomers and salts thereof, and cyclodextrin clathrates.

6. The compound as claimed in claim 1, where
A is a phenyl, naphthyl or heteroaryl radical which may optionally be substituted once or twice by R³ and/or R⁴,
R¹ is a hydrogen or a C₁-C₆-alkyl radical which is substituted one or more times by halogen,
R² is a hydrogen, halogen, cyano, an —S(O)_{q}—CH₃, where q is 0-2, a C₁-C₄-alkoxy radical or C₁-C₆-alkyl radical which is substituted one or more times by halogen,
R³ is a hydrogen, halogen, amino, —S(O)ₚ—CH₃, where p is 0-2,
an —S—CF₃, C₁-C₆-acyl, NH—CO—NH₂, NH—CO—C₁-C₆-alkyl, —O—CO—NHCH₃, —O—CO—N(CH₃)₂, or C₁-C₆-alkyl group which may optionally be substituted once, twice, three, four or five times, identically or differently, by C₁-C₄-acyl, C₁-C₄-alkoxy, hydroxy, cyano, CO₂—(C₁-C₄-alkyl), N—(C₁-C₄-alkyl)₂, C₅-C₁₂-heteroaryl, COOH, CO—NH₂, CO—NH(C₁-C₄-alkyl) or by CO—N(C₁-C₄-alkyl)₂,
a C₁-C₄-alkoxy which may optionally be substituted one or more times, identically or differently, by hydroxy, cyano, CO₂—(C₁-C₄-alkyl), N—(C₁-C₄-alkyl)₂, NH—C₃-C₆-cycloalkyl, COOH, CO—NH₂, CO—NH(C₁-C₄-alkyl) or by CO—N(C₁-C₄-alkyl)₂,
an O—C₆-C₁₂-aryl which may optionally be substituted by hydroxy, cyano, COOH or CO—NH₂,
a CH₂O—C₆-C₁₂-aryl which may optionally be substituted by hydroxy, cyano, COOH or CO—NH₂,
an O—C₅-C₁₆-heteroaryl which may optionally be substituted by hydroxy, cyano, COOH or CO—NH₂,
a hydroxy, cyano, O—CO—(C₁-C₄-alkyl), CO—NH(C₅-C₁₂-heteroaryl), NH—(C₁-C₄-alkyl), N—(C₁-C₄-alkyl)₂ or
a C₆-C₁₂-aryl which may optionally be substituted one or more times, identically or differently, by halogen, by C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl), CH₂—NH—CO(C₅-C₁₂-heteroaryl), styryl, or an —S(O)ᵣ—CH₃, where r is 0-2, or two adjacent positions may be substituted by —O—CH₂—O— or —O—C(CH₃)₂—O—,
a monocyclic C₅-C₇-heteroaryl which may optionally be substituted one or more times, identically or differently, by C₁-C₆-alkyl, if R² is cyano or if R¹ and/or R² is identically or differently a C₁-C₄-alkyl radical, where at least one of the radicals is substituted at least once by halogen, or if R⁴ is —S(O)ₚ—C₁-C₄-alkyl, where p is 0-2, C₁-C₄-acyl-, —O—CO—NH(C₁-C₄-alkyl), —O—CO—N(C₁-C₄-alkyl)₂, C₆-C₁₂-aryloxy, C₅-C₁₆-heteroaryloxy, hydroxy, cyano or N—(C₁-C₄-alkyl)₂,
a monocyclic C₅-C₇-heteroaryl which may be substituted at least once or else twice, three, four or five times, identically or differently, by halogen, by CF₃, C₁-C₄-acyl, C₁-C₄-alkoxy, hydroxy, CH₂—OH, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂, or
a bi- or tricyclic C₈-C₁₂-heteroaryl which may optionally be substituted once, twice, three, four or five times, identically or differently, by halogen, by C₁-C₆-alkyl, C₁-C₆-acyl, C₁-C₆-alkoxy, hydroxy, cyano, CO₂—(C₁-C₆-alkyl), N—(C₁-C₆-alkyl)₂, COOH, CO—NH₂, CO—NH(C₁-C₆-alkyl) or CO—N(C₁-C₆-alkyl)₂, or
a C₃-C₆-cycloalkyl which may optionally be substituted once, twice, three, four or five times, identically or differently, by halogen, by C₁-C₄-alkyl, hydroxy, cyano, CO₂—(C₁-C₄-alkyl), C₁-C₄-acyl, N—(C₁-C₄-alkyl)₂, COOH, CO—NH₂, CO—NH(C₁-C₄-alkyl), CO—N(C₁-C₄-alkyl)₂ or C₁-C₄-alkoxy,
R⁴ is a hydrogen, halogen, amino, —S(O)ₚ—CH₃, where p is 0-2,
an —S—CF₃, C₁-C₆-acyl, NH—CO—NH₂, NH—CO—C₁-C₆-alkyl, —O—CO—NHCH₃, —O—CO—N(CH₃)₂, or C₁-C₆-alkyl group which may optionally be substituted once, twice, three, four or five times, identically or differently, by C₁-C₄-acyl, C₁-C₄-alkoxy, hydroxy, cyano, CO₂—(C₁-C₄-alkyl), N—(C₁-C₄-alkyl)₂, C₅-C₁₂-heteroaryl, COOH, CO—NH₂, CO—NH(C₁-C₄-alkyl) or by CO—N(C₁-C₄-alkyl)₂,
a C₁-C₄-alkoxy which may optionally be substituted one or more times, identically or differently, by hydroxy, cyano, CO₂—(C₁-C₄-alkyl), N—(C₁-C₄-alkyl)₂, NH—C₃-C₆-cycloalkyl, COOH, CO—NH₂, CO—NH(C₁-C₄-alkyl) or by CO—N(C₁-C₄-alkyl)₂,
an O—C₆-C₁₂-aryl which may optionally be substituted by hydroxy, cyano, COOH or CO—NH₂,
a CH₂O—C₆-C₁₂-aryl which may optionally be substituted by hydroxy, cyano, COOH or CO—NH₂,
an O—C₅-C₁₆-heteroaryl which may optionally be substituted by hydroxy, cyano, COOH or CO—NH₂,
a hydroxy, cyano, O—CO—(C₁-C₄-alkyl), CO—NH(C₅-C₁₂-heteroaryl), NH—(C₁-C₄-alkyl), N—(C₁-C₄-alkyl)₂ or
a C₆-C₁₂-aryl which may optionally be substituted one or more times, identically or differently, by halogen, by C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl), CH₂—NH—CO(C₅-C₁₂-heteroaryl), styryl, or an —S(O)ᵣ—CH₃, where r is 0-2, or two adjacent positions may be substituted by —O—CH₂—O— or —O—C(CH₃)₂—O—,
a monocyclic C₅-C₇-heteroaryl which may optionally be substituted one or more times, identically or differently, by C₁-C₆-alkyl, if R² is cyano or if R¹ and/or R² is identically or differently a C₁-C₄-alkyl radical, where at least one of the radicals is substituted at least once by halogen,
a monocyclic C₅-C₇-heteroaryl which may be substituted at least once or else twice, three, four or five times, identically or differently, by halogen, by CF₃, C₁-C₄-acyl, C₁-C₄-alkoxy, hydroxy, CH₂—OH, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂, or
a bi- or tricyclic C₈-C₁₂-heteroaryl which may optionally be substituted once, twice, three, four or five times, identically or differently, by halogen, by C₁-C₆-alkyl, C₁-C₆-acyl, C₁-C₆-alkoxy, hydroxy, cyano, CO₂—(C₁-C₆-alkyl), N—(C₁-C₆-alkyl)₂, COOH, CO—NH₂, CO—NH(C₁-C₆-alkyl) or CO—N(C₁-C₆-alkyl)₂ or
a C₃-C₆-cycloalkyl which may optionally be substituted once, twice, three, four or five times, identically or differently, by halogen, by C₁-C₄-alkyl, hydroxy, cyano, CO₂—(C₁-C₄-alkyl), C₁-C₄-acyl, N—(C₁-C₄-alkyl)₂, COOH, CO—NH₂, CO—NH(C₁-C₄-alkyl), CO—N(C₁-C₄-alkyl)₂ or C₁-C₄-alkoxy,
R³ and R⁴ are either in ortho, meta position or meta, para position relative to one another and together have the meaning of
—O—CO—S—, —S—CO—O—, CH₂—CO—O—, O—CO—CH₂—, —CH₂—CO—NH—, —NH—CO—CH₂—, —O—CO—NH—, —NH—CO—O—, —CO—CH₂—(CH₂)ₘ—, —CH₂—(CH₂)ₘ—CO—, —O—(CH₂)ₘ—O—, —O—C—(CH₃)₂—O—, —CH₂—(CH₂)ₘ—CH₂—, where m is 1-3,
Y is a —(CH₂)ₙ— group, where n is 1-3,
and the isomers, diastereomers, enantiomers and salts thereof, and cyclodextrin clathrates.

7. The compound as claimed in claim 1, where
A is a phenyl, naphthyl or heteroaryl radical which may optionally be substituted once or twice by R³ and/or R⁴,
R¹ is a hydrogen or a C₁-C₄-alkyl radical which is substituted once, twice or three times by chlorine, fluorine, or bromine,
R² is a hydrogen, chlorine, fluorine, bromine, cyano, an OCH₃ group or a C₁-C₄-alkyl radical which is substituted once, twice or three times by chlorine, fluorine or bromine,
R³ is a hydrogen, halogen, amino, —S(O)ₚ—CH₃, where p is 0-2,
an —S—CF₃, C₁-C₆-acyl, NH—CO—NH₂, NH—CO—C₁-C₆-alkyl, —O—CO—NHCH₃, —O—CO—N(CH₃)₂, or C₁-C₆-alkyl group which may optionally be substituted once or twice, identically or differently, by C₁-C₄-acyl, C₁-C₄-alkoxy, hydroxy, cyano, CO₂—(C₁-C₄-alkyl), N—(C₁-C₄-alkyl)₂, C₅-C₁₂-heteroaryl, COOH, CO—NH₂, CO—NH(C₁-C₄-alkyl) or by CO—N(C₁-C₄-alkyl)₂,
a C₁-C₄-alkoxy which may optionally be substituted one or more times, identically or differently, by hydroxy, cyano, CO₂—(C₁-C₄-alkyl), N—(C₁-C₄-alkyl)₂, NH—C₃-C₆-cycloalkyl, COOH, CO—NH₂, CO—NH(C₁-C₄-alkyl) or by CO—N(C₁-C₄-alkyl)₂,
an O—C₆-C₁₂-aryl which may optionally be substituted by hydroxy, cyano, COOH or CO—NH₂,
a CH₂O—C₆-C₁₂-aryl which may optionally be substituted by hydroxy, cyano, COOH or CO—NH₂,
an O—C₅-C₁₆-heteroaryl which may optionally be substituted by hydroxy, cyano, COOH or CO—NH₂,
a hydroxy, cyano, O—CO—(C₁-C₄-alkyl), CO—NH(C₅-C₁₂-heteroaryl), NH—(C₁-C₄-alkyl), N—(C₁-C₄-alkyl)₂ or
a C₆-C₁₂-aryl which may optionally be substituted one or more times, identically or differently, by halogen, by C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl), CH₂—NH—CO(C₅-C₁₂-heteroaryl), styryl, or an —S(O)ᵣ—CH₃, where r is 0-2, or two adjacent positions may be substituted by —O—CH₂—O— or —O—C(CH₃)₂—O—,
a monocyclic C₅-C₇-heteroaryl which may optionally be substituted one or more times, identically or differently, by C₁-C₆-alkyl, if R² is cyano or if R¹ and/or R² is a CF₃ radical, or if R⁴ is —S(O)ₚ—C₁-C₆-alkyl, where p is 0-2, C₁-C₄-acyl-, —O—CO—NH(C₁-C₄-alkyl), —O—CO—N(C₁-C₄-alkyl)₂, C₆-C₁₂-aryloxy, C₅-C₁₆-heteroaryloxy, hydroxy, cyano or N—(C₁-C₄-alkyl)₂,
a monocyclic C₅-C₇-heteroaryl which may be substituted at least once or twice, identically or differently, by halogen, by CF₃, C₁-C₄-acyl, C₁-C₄-alkoxy, hydroxy, CH₂—OH, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂ or
a bi- or tricyclic C₈-C₁₂-heteroaryl which may optionally be substituted once or twice, identically or differently, by halogen, by C₁-C₆-alkyl, C₁-C₆-acyl, C₁-C₆-alkoxy, hydroxy, cyano, CO₂—(C₁-C₆-alkyl), N—(C₁-C₆-alkyl)₂, COOH, CO—NH₂, CO—NH(C₁-C₆-alkyl) or CO—N(C₁-C₆-alkyl)₂, or
a C₃-C₆-cycloalkyl which may optionally be substituted once or twice, identically or differently, by halogen, by C₁-C₄-alkyl, hydroxy, cyano, CO₂—(C₁-C₄-alkyl), C₁-C₄-alkyl, N—(C₁-C₄-alkyl)₂, COOH, CO—NH₂, CO—NH(C₁-C₄-alkyl), CO—N(C₁-C₄-alkyl)₂ or C₁-C₄-alkoxy,
R⁴ is a hydrogen, halogen, amino, —S(O)ₚ—CH₃, where p is 0-2,
an —S—CF₃, C₁-C₆-acyl, NH—CO—NH₂, NH—CO—C₁-C₆-alkyl, —O—CO—NHCH₃, —O—CO—N(CH₃)₂, or C₁-C₆-alkyl group which may optionally be substituted once or twice, identically or differently, by C₁-C₄-acyl, C₁-C₄-alkoxy, hydroxy, cyano, CO₂—(C₁-C₄-alkyl), N—(C₁-C₄-alkyl)₂, C₅-C₁₂-heteroaryl, COOH, CO—NH₂, CO—NH(C₁-C₄-alkyl) or by CO—N(C₁-C₄-alkyl)₂,
a C₁-C₄-alkoxy which may optionally be substituted one or more times, identically or differently, by hydroxy, cyano, CO₂—(C₁-C₄-alkyl), N—(C₁-C₄-alkyl)₂, NH—C₃-C₆-cycloalkyl, COOH, CO—NH₂, CO—NH(C₁-C₄-alkyl) or by CO—N(C₁-C₄-alkyl)₂,
an O—C₆-C₁₂-aryl which may optionally be substituted by hydroxy, cyano, COOH or CO—NH₂,
a CH₂O—C₆-C₁₂-aryl which may optionally be substituted by hydroxy, cyano, COOH or CO—NH₂,
an O—C₅-C₁₆-heteroaryl which may optionally be substituted by hydroxy, cyano, COOH or CO—NH₂,
a hydroxy, cyano, O—CO—(C₁-C₄-alkyl), CO—NH(C₅-C₁₂-heteroaryl), NH—(C₁-C₄-alkyl), N—(C₁-C₄-alkyl)₂ or
a C₆-C₁₂-aryl which may optionally be substituted one or more times, identically or differently, by halogen, by C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂ CH₃, SO₂NH₂, SO₂NH(C₁-C₆-alkyl), SO₂N(C₁-C₆-alkyl)₂, COOH, CO—NH₂, CO—NH(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl), CH₂—NH—CO(C₅-C₁₂-heteroaryl), styryl, or an —S(O)ᵣ—CH₃, where r is 0-2, or two adjacent positions may be substituted by —O—CH₂—O— or —O—C(CH₃)₂—O—,
a monocyclic C₅-C₇-heteroaryl which may optionally be substituted one or more times, identically or differently, by C₁-C₆-alkyl, if R² is cyano or if R¹ and/or R² is a CF₃ radical,
a monocyclic C₅-C₇-heteroaryl which may be substituted at least once or twice, identically or differently, by halogen, by CF₃, C₁-C₄-acyl, C₁-C₄-alkoxy, hydroxy, CH₂—OH, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂ or
a bi- or tricyclic C₈-C₁₂-heteroaryl which may optionally be substituted once or twice, identically or differently, by halogen, by C₁-C₆-alkyl, C₁-C₆-acyl, C₁-C₆-alkoxy, hydroxy, cyano, CO₂—(C₁-C₆-alkyl), N—(C₁-C₆-alkyl)₂, COOH, CO—NH₂, CO—NH(C₁-C₆-alkyl) or CO—N(C₁-C₆-alkyl)₂, or
a C₃-C₆-cycloalkyl which may optionally be substituted once or twice, identically or differently, by halogen, by C₁-C₄-alkyl, hydroxy, cyano, CO₂—(C₁-C₄-alkyl), C₁-C₄-acyl, N—(C₁-C₄-alkyl)₂, COOH, CO—NH₂, CO—NH(C₁-C₄-alkyl), CO—N(C₁-C₄-alkyl)₂ or C₁-C₄-alkoxy,
R³ and R⁴ are either in ortho, meta position or meta, para position relative to one another and together have the meaning of
—O—CO—S—, —S—CO—O—, CH₂—CO—O—, O—CO—CH₂—, —CH₂—CO—NH—, —NH—CO—CH₂—, —O—CO—NH—, —NH—CO—O—, —CO—CH₂—(CH₂)ₘ—, —CH₂—(CH₂)ₘ—CO—, —O—(CH₂)ₘ—O—, —O—C—(CH₃)₂—O—, —CH₂—(CH₂)ₘ—CH₂—, where m is 1-3,
Y is a —(CH₂)ₙ— group, where n is 1-3,
and the isomers, diastereomers, enantiomers and salts thereof, and cyclodextrin clathrates.

8. The compound as claimed in claim 1 selected from a group which comprises the following compounds:
1. biphenyl-4-carboxylic acid [2-(7-fluoro-2,4-dimethyl-1H-indol-3-yl)ethyl]amide
2. N-[2-(7-fluoro-2,4-dimethyl-1H-indol-3-yl)ethyl]-3,4,5-trimethoxybenzamide
3. 4-fluoro-N-[2-(7-fluoro-2,4-dimethyl-1H-indol-3-yl)ethyl]benzamide
4. N-[2-(7-fluoro-2,4-dimethyl-1H-indol-3-yl)ethyl]-4-methylbenzamide
5. 2-chloro-N-[2-(7-fluoro-2,4-dimethyl-1H-indol-3-yl)ethyl]benzamide
6. N-[2-(7-fluoro-2,4-dimethyl-1H-indol-3-yl)ethyl]-4-trifluoromethylbenzamide
7. 3-chloro-N-[2-(7-fluoro-2,4-dimethyl-1H-indol-3-yl)ethyl]benzamide
8. N-[2-(7-fluoro-2,4-dimethyl-1H-indol-3-yl)ethyl]-4-methoxybenzamide
9. 4-chloro-N-[2-(7-fluoro-2,4-dimethyl-1H-indol-3-yl)ethyl]benzamide
10. N-[2-(7-fluoro-2,4-dimethyl-1H-indol-3-yl)ethyl]-3-methylbenzamide
11. 4-tert-butyl-N-[2-(7-fluoro-2,4-dimethyl-1H-indol-3-yl)ethyl]benzamide
12. benzo[1,3]dioxole-5-carboxylic acid [2-(7-fluoro-2,4-dimethyl-1H-indol-3-yl)ethyl]amide
13. thiophene-2-carboxylic acid [2-(7-fluoro-2,4-dimethyl-1H-indol-3-yl)ethyl]amide
14. quinoxaline-6-carboxylic acid [2-(7-fluoro-2,4-dimethyl-1H-indol-3-yl)ethyl]amide
15. 5-phenylpyridine-2-carboxylic acid [2-(7-fluoro-2,4-dimethyl-1H-indol-3-yl)ethyl]amide
16. 5-phenyl-1H-pyrrole-2-carboxylic acid [2-(7-fluoro-2,4-dimethyl-1H-indol-3-yl)ethyl]amide
17. N-[2-(4,7-difluoro-2-methyl-1H-indol-3-yl)ethyl]-3,4-dimethoxybenzamide
18. [symbol] -N-[2-(7-fluoro-2,4-dimethyl-1H-indol-3-yl)ethyl]-4-methanesulfinylbenzamide
19. N-[2-(7-fluoro-1H-indol-3-yl)ethyl]-3,4-dimethoxybenzamide
20. N-[2-(7-fluoro-2,4-dimethyl-1H-indol-3-yl)ethyl]-3-[1,2,4]triazol-1-ylmethylbenzamide
21. thieno[2,3-b]pyrazine-6-carboxylic acid [2-(7-fluoro-2,4-dimethyl-1H-indol-3-yl)ethyl]amide
22. N-[2-(7-fluoro-2-methyl-1H-indol-3-yl)ethyl]-3,4-dimethoxybenzamide
23. N-[2-(4-chloro-7-fluoro-2-methyl-1H-indol-3-yl)ethyl]-3,4-dimethoxybenzamide
24. N-[2-(7-fluoro-2,4-dimethyl-1H-indol-3-yl)ethyl]-4-methanesulfonylbenzamide
25. N-[2-(4-bromo-7-fluoro-2-methyl-1H-indol-3-yl)ethyl]-3,4-dimethoxybenzamide
26. 1H-benzotriazole-5-carboxylic acid [2-(4,7-difluoro-2-methyl-1H-indol-3-yl)ethyl]amide
27. 1H-indole-2-carboxylic acid [2-(4,7-difluoro-2-methyl-1H-indol-3-yl)ethyl]amide
28. 4′-fluorobiphenyl-4-carboxylic acid [2-(7-fluoro-2,4-dimethyl-1H-indol-3-yl)ethyl]amide
29. N-[2-(7-fluoro-2,4-dimethyl-1H-indol-3-yl)ethyl]-N′-pyridin-3-yl-terephthalamide
30. 4-amino-N-[2-(7-fluoro-2,4-dimethyl-1H-indol-3-yl)ethyl]benzamide
31. 5-bromofuran-2-carboxylic acid [2-(7-fluoro-2,4-dimethyl-1H-indol-3-yl)ethyl]amide
32. N-[2-(7-fluoro-2,4 dimethyl-1H-indol-3-yl)ethyl]-isonicotinamide
33. N-[2-(7-fluoro-2,4 dimethyl-1H-indol-3-yl)ethyl]benzamide
34. 2-fluoro-N-[2-(7-fluoro-2,4-dimethyl-1H-indol-3-yl)ethyl]benzamide
35. 3-fluoro-N-[2-(7-fluoro-2,4-dimethyl-1H-indol-3-yl)ethyl]benzamide
36. 1H-indole-2-carboxylic acid [2-(7-fluoro-2,4-dimethyl-1H-indol-3-yl)ethyl]amide
37. N-[2-(7-fluoro-2,4-dimethyl-1H-indol-3-yl)ethyl]-6-methylnicotinamide
38. N-[2-(7-fluoro-2,4-dimethyl-1H-indol-3-yl)ethyl]-3-methoxybenzamide
39. 4-ethoxy-N-[2-(7-fluoro-2,4-dimethyl-1H-indol-3-yl)ethyl]benzamide
40. N-[2-(7-fluoro-2,4-dimethyl-1H-indol-3-yl)ethyl]-4-hydroxy-3,5-dimethoxybenzamide
41. 1H-benzotriazole-5-carboxylic acid [2-(7-fluoro-2,4-dimethyl-1H-indol-3-yl)ethyl]amide
42. 5-methylthiophene-2-carboxylic acid [2-(7-fluoro-2,4-dimethyl-1H-indol-3-yl)ethyl]amide
43. 1H-pyrrole-2-carboxylic acid [2-(7-fluoro-2,4-dimethyl-1H-indol-3-yl)ethyl]amide
44. N-[2-(7-fluoro-2,4-dimethyl-1H-indol-3-yl)ethyl]-2-methylaminobenzamide
45. thiophene-3-carboxylic acid [2-(7-fluoro-2,4-dimethyl-1H-indol-3-yl)ethyl]amide
46. 6-cyano-N-[2-(7-fluoro-2,4-dimethyl-1H-indol-3-yl)ethyl]nicotinamide
47. 1H-benzoimidazole-5-carboxylic acid [2-(7-fluoro-2,4-dimethyl-1H-indol-3-yl)ethyl]amide
48. N-[2-(7-fluoro-2,4-dimethyl-1H-indol-3-yl)ethyl]-3-trifluoromethylbenzamide
49. N-[2-(7-fluoro-2,4-dimethyl-1H-indol-3-yl)ethyl]-4-hydroxy-3-methoxybenzamide
50. 4-dimethylamino-N-[2-(7-fluoro-2,4-dimethyl-1H-indol-3-yl)ethyl]benzamide
51. 4-cyano-N-[2-(7-fluoro-2,4-dimethyl-1H-indol-3-yl)ethyl]benzamide
52. isoxazole-5-carboxylic acid [2-(7-fluoro-2,4-dimethyl-1H-indol-3-yl)ethyl]amide
53. 4-acetyl-N-[2-(7-fluoro-2,4-dimethyl-1H-indol-3-yl)ethyl]benzamide
54. 4-chloro-3-fluoro-N-[2-(7-fluoro-2,4-dimethyl-1H-indol-3-yl)ethyl]benzamide
55. 4-chloromethyl-N-[2-(7-fluoro-2,4-dimethyl-1H-indol-3-yl)ethyl]benzamide
56. N-[2-(7-fluoro-2,4-dimethyl-1H-indol-3-yl)ethyl]-3,5-dimethylbenzamide
57. 3,4-difluoro-N-[2-(7-fluoro-2,4-dimethyl-1H-indol-3-yl)ethyl]benzamide
58. N-[2-(7-fluoro-2,4-dimethyl-1H-indol-3-yl)ethyl]-4-propylbenzamide
59. N-[2-(7-fluoro-2,4-dimethyl-1H-indol-3-yl)ethyl]-3-hydroxy-4-methylbenzamide
60. 2,3-difluoro-N-[2-(7-fluoro-2,4-dimethyl-1H-indol-3-yl)ethyl]benzamide
61. 3,5-difluoro-N-[2-(7-fluoro-2,4-dimethyl-1H-indol-3-yl)ethyl]benzamide
62. naphthalene-2-carboxylic acid [2-(7-fluoro-2,4-dimethyl-1H-indol-3-yl)ethyl]amide
63. 5-chlorothiophene-2-carboxylic acid [2-(7-fluoro-2,4-dimethyl-1H-indol-3-yl)ethyl]amide
64. 6-fluoro-N-[2-(7-fluoro-2,4-dimethyl-1H-indol-3-yl)ethyl]nicotinamide
65. 3-chloromethyl-N-[2-(7-fluoro-2,4-dimethyl-1H-indol-3-yl)ethyl]benzamide
66. 4-butoxy-N-[2-(7-fluoro-2,4-dimethyl-1H-indol-3-yl)ethyl]benzamide
67. 4-acetoxy-N-[2-(7-fluoro-2,4-dimethyl-1H-indol-3-yl)ethyl]benzamide
68. N-[2-(7-fluoro-2,4-dimethyl-1H-indol-3-yl)ethyl]-4-methylsulfanylbenzamide
69. 4-cyano-2-fluoro-N-[2-(7-fluoro-2,4-dimethyl-1H-indol-3-yl)ethyl]benzamide
70. isoquinoline-1-carboxylic acid [2-(7-fluoro-2,4-dimethyl-1H-indol-3-yl)ethyl]amide
71. isoquinoline-1-carboxylic acid [2-(7-fluoro-2,4-dimethyl-1H-indol-3-yl)ethyl]amide
72. isoquinoline-1-carboxylic acid [2-(7-fluoro-2,4-dimethyl-1H-indol-3-yl)ethyl]amide
73. 3,5-dichloro-N-[2-(7-fluoro-2,4-dimethyl-1H-indol-3-yl)ethyl]benzamide
74. quinoline-4-carboxylic acid [2-(7-fluoro-2,4-dimethyl-1H-indol-3-yl)ethyl]amide
75. quinoline-4-carboxylic acid [2-(7-fluoro-2,4-dimethyl-1H-indol-3-yl)ethyl]amide
76. 4-hydroxy-2-phenyl-2H-pyrazole-3-carboxylic acid [2-(7-fluoro-2,4-dimethyl-1H-indol-3-yl)ethyl]amide
77. benzo[b]thiophene-3-carboxylic acid [2-(7-fluoro-2,4-dimethyl-1H-indol-3-yl)ethyl]amide
78. N-[2-(7-fluoro-2,4-dimethyl-1H-indol-3-yl)ethyl]-3-hydroxybenzamide
79. pyrazine-2-carboxylic acid [2-(7-fluoro-2,4-dimethyl-1H-indol-3-yl)ethyl]amide
80. furan-2-carboxylic acid [2-(7-fluoro-2,4-dimethyl-1H-indol-3-yl)ethyl]amide
81. quinoline-3-carboxylic acid [2-(7-fluoro-2,4-dimethyl-1H-indol-3-yl)ethyl]amide
82. 2-chloro-N-[2-(7-fluoro-2,4-dimethyl-1H-indol-3-yl)ethyl]nicotinamide
83. 4-benzyloxy-N-[2-(7-fluoro-2,4-dimethyl-1H-indol-3-yl)ethyl]benzamide
84. 5-bromo-N-[2-(7-fluoro-2,4-dimethyl-1H-indol-3-yl)ethyl]nicotinamide
85. 1H-indole-3-carboxylic acid [2-(7-fluoro-2,4-dimethyl-1H-indol-3-yl)ethyl]amide
86. 3-bromothiophene-2-carboxylic acid [2-(7-fluoro-2,4-dimethyl-1H-indol-3-yl)ethyl]amide
87. 2-chloro-N-[2-(7-fluoro-2,4-dimethyl-1H-indol-3-yl)ethyl]-3,4-dimethoxybenzamide
88. 2-methylfuran-3-carboxylic acid [2-(7-fluoro-2,4-dimethyl-1H-indol-3-yl)ethyl]amide
89. 1H-imidazole-4-carboxylic acid [2-(7-fluoro-2,4-dimethyl-1H-indol-3-yl)ethyl]amide
90. 4-oxo-4,5,6,7-tetrahydrobenzo[b]thiophene-2-carboxylic acid [2-(7-fluoro-2,4-dimethyl-1H-indol-3-yl)ethyl]amide
91. 4′-bromobiphenyl-2-carboxylic acid [2-(7-fluoro-2,4-dimethyl-1H-indol-3-yl)ethyl]amide
92. 2-fluoro-N-[2-(7-fluoro-2,4-dimethyl-1H-indol-3-yl)ethyl]-6-iodobenzamide
93. 2,3-dihydrobenzofuran-7-carboxylic acid [2-(7-fluoro-2,4-dimethyl-1H-indol-3-yl)ethyl]amide
94. 3-fluoro-N-[2-(7-fluoro-2,4-dimethyl-1H-indol-3-yl)ethyl]-2-methylbenzamide
95. N-[2-(7-fluoro-2,4-dimethyl-1H-indol-3-yl)ethyl]-2,5-dimethylbenzamide
96. 5-acetylthiophene-2-carboxylic acid [2-(7-fluoro-2,4-dimethyl-1H-indol-3-yl)ethyl]amide
97. quinoline-8-carboxylic acid [2-(7-fluoro-2,4-dimethyl-1H-indol-3-yl)ethyl]amide
98. 2-phenyl-2H-pyrazole-3-carboxylic acid [2-(7-fluoro-2,4-dimethyl-1H-indol-3-yl)ethyl]amide
99. 6-phenylpyrimidine-4-carboxylic acid [2-(7-fluoro-2,4-dimethyl-1H-indol-3-yl)ethyl]amide
100. 1-methyl-1H-indole-3-carboxylic acid [2-(7-fluoro-2,4-dimethyl-1H-indol-3-yl)ethyl]amide
101. 2-pyridin-3-ylthiazole-4-carboxylic acid [2-(7-fluoro-2,4-dimethyl-1H-indol-3-yl)ethyl]amide
102. 2,5-dimethyl-2H-pyrazole-3-carboxylic acid [2-(7-fluoro-2,4-dimethyl-1H-indol-3-yl)ethyl]amide
103. N-[2-(7-fluoro-2,4-dimethyl-1H-indol-3-yl)ethyl]-2-phenoxymethylbenzamide
104. 2,3-dihydrobenzofuran-5-carboxylic acid [2-(7-fluoro-2,4-dimethyl-1H-indol-3-yl)ethyl]amide
105. 1H-indole-6-carboxylic acid [2-(7-fluoro-2,4-dimethyl-1H-indol-3-yl)ethyl]amide
106. 2-bromo-N-[2-(7-fluoro-2,4-dimethyl-1H-indol-3-yl)ethyl]-4,5-dimethoxybenzamide
107. N-[2-(7-fluoro-2,4-dimethyl-1H-indol-3-yl)ethyl]-4-pyrrolidin-1-ylbenzamide
108. quinoline-5-carboxylic acid [2-(7-fluoro-2,4-dimethyl-1H-indol-3-yl)ethyl]amide
109. 5-phenyl-1H-pyrazole-4-carboxylic acid [2-(7-fluoro-2,4-dimethyl-1H-indol-3-yl)ethyl]amide
110. pyridazine-4-carboxylic acid [2-(7-fluoro-2,4-dimethyl-1H-indol-3-yl)ethyl]amide
111. 5-phenyl-2H-pyrazole-3-carboxylic acid [2-(7-fluoro-2,4-dimethyl-1H-indol-3-yl)ethyl]amide
112. N-[2-(7-fluoro-2,4-dimethyl-1H-indol-3-yl)ethyl]-5-pyrrol-1-ylnicotinamide
113. pyrimidine-5-carboxylic acid [2-(7-fluoro-2,4-dimethyl-1H-indol-3-yl)ethyl]amide
114. benzo[b]thiophene-5-carboxylic acid [2-(7-fluoro-2,4-dimethyl-1H-indol-3-yl)ethyl]amide
115. N-[2-(7-fluoro-2,4-dimethyl-1H-indol-3-yl)ethyl]-3-piperidin-1-ylbenzamide
116. pyrazolo[1,5-a]pyridine-2-carboxylic acid [2-(7-fluoro-2,4-dimethyl-1H-indol-3-yl)ethyl]amide
117. quinoxaline-2-carboxylic acid [2-(7-fluoro-2,4-dimethyl-1H-indol-3-yl)ethyl]amide
118. 3-fluoro-N-[2-(7-fluoro-2,4-dimethyl-1H-indol-3-yl)ethyl]-2-methoxybenzamide
119. 3-chloro-N-[2-(7-fluoro-2,4-dimethyl-1H-indol-3-yl)ethyl]-2-methylbenzamide
120. N-[2-(7-fluoro-2,4-dimethyl-1H-indol-3-yl)ethyl]-4-phenoxbenzamide
121. thiazole-4-carboxylic acid [2-(7-fluoro-2,4-dimethyl-1H-indol-3-yl)ethyl]amide
122. 2-chloro-N-[2-(7-fluoro-2,4-dimethyl-1H-indol-3-yl)ethyl]-3-methylbenzamide
123. 3-chloro-2-fluoro-N-[2-(7-fluoro-2,4-dimethyl-1H-indol-3-yl)ethyl]benzamide
124. 5-methoxy-1H-indole-2-carboxylic acid [2-(7-fluoro-2,4-dimethyl-1H-indol-3-yl)ethyl]amide
125. 5-fluoro-1H-indole-2-carboxylic acid [2-(7-fluoro-2,4-dimethyl-1H-indol-3-yl)ethyl]amide
126. 5-methyl-1H-indole-2-carboxylic acid [2-(7-fluoro-2,4-dimethyl-1H-indol-3-yl)ethyl]amide
127. 4-methyl-1H-indole-2-carboxylic acid [2-(7-fluoro-2,4-dimethyl-1H-indol-3-yl)ethyl]amide
128. 6-methoxy-1H-indole-2-carboxylic acid [2-(7-fluoro-2,4-dimethyl-1H-indol-3-yl)ethyl]amide
129. 4-methoxy-1H-indole-2-carboxylic acid [2-(7-fluoro-2,4-dimethyl-1H-indol-3-yl)ethyl]amide
130. 4-fluoro-1H-indole-2-carboxylic acid [2-(7-fluoro-2,4-dimethyl-1H-indol-3-yl)ethyl]amide
131. 7-methyl-1H-indole-2-carboxylic acid [2-(7-fluoro-2,4-dimethyl-1H-indol-3-yl)ethyl]amide
132. 6-fluoro-1H-indole-2-carboxylic acid [2-(7-fluoro-2,4-dimethyl-1H-indol-3-yl)ethyl]amide
133. 6-methyl-1H-indole-2-carboxylic acid [2-(7-fluoro-2,4-dimethyl-1H-indol-3-yl)ethyl]amide
134. 5-methoxy-1H-indole-2-carboxylic acid [2-(7-fluoro-1H-indol-3-yl)ethyl]amide
135. 1H-indole-2-carboxylic acid [2-(7-fluoro-1H-indol-3-yl)ethyl]amide
136. 5-fluoro-1H-indole-2-carboxylic acid [2-(7-fluoro-1H-indol-3-yl)ethyl]amide
137. 5-methyl-1H-indole-2-carboxylic acid [2-(7-fluoro-1H-indol-3-yl)ethyl]amide
138. 6-methoxy-1H-indole-2-carboxylic acid [2-(7-fluoro-1H-indol-3-yl)ethyl]amide
139. 4-methyl-1H-indole-2-carboxylic acid [2-(7-fluoro-1H-indol-3-yl)ethyl]amide
140. 4-dimethylamino-N-[2-(7-fluoro-1H-indol-3-yl)ethyl]benzamide
141. N-[2-(7-fluoro-1H-indol-3-yl)ethyl]-4-pyrrolidin-1-ylbenzamide
142. 1H-indole-6-carboxylic acid [2-(7-fluoro-1H-indol-3-yl)ethyl]amide
143. 4-methoxy-1H-indole-2-carboxylic acid [2-(7-fluoro-1H-indol-3-yl)ethyl]amide
144. 4-fluoro-1H-indole-2-carboxylic acid [2-(7-fluoro-1H-indol-3-yl)ethyl]amide
145. 6-fluoro-1H-indole-2-carboxylic acid [2-(7-fluoro-1H-indol-3-yl)ethyl]amide
146. 6-methyl-1H-indole-2-carboxylic acid [2-(7-fluoro-1H-indol-3-yl)ethyl]amide
147. 7-methyl-1H-indole-2-carboxylic acid [2-(7-fluoro-1H-indol-3-yl)ethyl]amide
148. 5-bromo-2,3-dihydrobenzofuran-7-carboxylic acid [2-(7-fluoro-2,4-dimethyl-1H-indol-3-yl)ethyl]amide
149. 4-(1H-benzoimidazol-2-yl)-N-[2-(7-fluoro-1H-indol-3-yl)ethyl]benzamide
150. 4-(1H-benzoimidazol-2-yl)-N-[2-(7-fluoro-2,4-dimethyl-1H-indol-3-yl)ethyl]benzamide
151. N-[2-(4-cyano-7-fluoro-2-methyl-1H-indol-3-yl)ethyl]-3,4-dimethoxybenzamide
152. [1,1′; 4′,1″]terphenyl-4-carboxylic acid [2-(7-fluoro-2,4-dimethyl-1H-indol-3-yl)ethyl]amide
153. 3′-methylbiphenyl-4-carboxylic acid [2-(7-fluoro-2,4-dimethyl-1H-indol-3-yl)ethyl]amide
154. 3′-fluoro-4′-methylbiphenyl-4-carboxylic acid [2-(7-fluoro-2,4-dimethyl-1H-indol-3-yl)ethyl]amide
155. 2′-fluoro-4′-methylbiphenyl-4-carboxylic acid [2-(7-fluoro-2,4-dimethyl-1H-indol-3-yl)ethyl]amide
156. 4′-hydroxymethylbiphenyl-4-carboxylic acid [2-(7-fluoro-2,4-dimethyl-1H-indol-3-yl)ethyl]amide
157. 4′-[2-(7-fluoro-2,4-dimethyl-1H-indol-3-yl)ethylcarbamoyl]-biphenyl-4-carboxylic acid
158. 4′-tert-butylbiphenyl-4-carboxylic acid [2-(7-fluoro-2,4-dimethyl-1H-indol-3-yl)ethyl]amide
159. 4′-chlorobiphenyl-4-carboxylic acid [2-(7-fluoro-2,4-dimethyl-1H-indol-3-yl)ethyl]amide
160. 3′,4′,5′-trimethoxybiphenyl-4-carboxylic acid [2-(7-fluoro-2,4-dimethyl-1H-indol-3-yl)ethyl]amide
161. 3′-trifluoromethoxybiphenyl-4-carboxylic acid [2-(7-fluoro-2,4-dimethyl-1H-indol-3-yl)ethyl]amide
162. 4′-trifluoromethoxybiphenyl-4-carboxylic acid [2-(7-fluoro-2,4-dimethyl-1H-indol-3-yl)ethyl]amide
163. 3′-hydroxybiphenyl-4-carboxylic acid [2-(7-fluoro-2,4-dimethyl-1H-indol-3-yl)ethyl]amide
164. 4′-methanesulfinylbiphenyl-4-carboxylic acid [2-(7-fluoro-2,4-dimethyl-1H-indol-3-yl)ethyl]amide
165. 3′-cyanomethylbiphenyl-4-carboxylic acid [2-(7-fluoro-2,4-dimethyl-1H-indol-3-yl)ethyl]amide
166. 2′-acetylaminobiphenyl-4-carboxylic acid [2-(7-fluoro-2,4-dimethyl-1H-indol-3-yl)ethyl]amide
167. 3′-fluoro-4′-methoxybiphenyl-4-carboxylic acid [2-(7-fluoro-2,4-dimethyl-1H-indol-3-yl)ethyl]amide
168. 3′-chloro-4′-fluorobiphenyl-4-carboxylic acid [2-(7-fluoro-2,4-dimethyl-1H-indol-3-yl)ethyl]amide
169. 3′,4′-difluorobiphenyl-4-carboxylic acid [2-(7-fluoro-2,4-dimethyl-1H-indol-3-yl)ethyl]amide
170. 3′,5′-difluorobiphenyl-4-carboxylic acid [2-(7-fluoro-2,4-dimethyl-1H-indol-3-yl)ethyl]amide
171. N-[2-(7-fluoro-2,4-dimethyl-1H-indol-3-yl)ethyl]-4-(5-hydroxymethyl-thiophen-2-yl)-benzamide
172. 3′-methanesulfonylbiphenyl-4-carboxylic acid [2-(7-fluoro-2,4-dimethyl-1H-indol-3-yl)ethyl]amide
173. 4-fluoro-4′-[2-(7-fluoro-2,4-dimethyl-1H-indol-3-yl)ethylcarbamoyl]-biphenyl-3-carboxylic acid
174. 4′-[2-(7-fluoro-2,4-dimethyl-1H-indol-3-yl)ethylcarbamoyl]-3-methoxy-biphenyl-4-carboxylic acid methyl ester
175. 5-fluoro-4′-[2-(7-fluoro-2,4-dimethyl-1H-indol-3-yl)ethylcarbamoyl]biphenyl-3-carboxylic acid
176. 3-chlorobiphenyl-4,4′-dicarboxylic acid 4-amide 4′-{[2-(7-fluoro-2,4-dimethyl-1H-indol-3-yl)ethyl]amide}
177. 3-chlorobiphenyl-4,4′-dicarboxylic acid 4′-{[2-(7-fluoro-2,4-dimethyl-1H-indol-3-yl)ethyl]amide}4-methylamide
178. 3′-dimethylsulfamoylbiphenyl-4-carboxylic acid [2-(7-fluoro-2,4-dimethyl-1H-indol-3-yl)ethyl]amide
179. biphenyl-3,4′-dicarboxylic acid 4′-{[2-(7-fluoro-2,4-dimethyl-1H-indol-3-yl)ethyl]amide}3-thiazol-2-ylamide
180. 4′-methylsulfamoylbiphenyl-4-carboxylic acid [2-(7-fluoro-2,4-dimethyl-1H-indol-3-yl)ethyl]amide
181. 4′-dimethylsulfamoylbiphenyl-4-carboxylic acid [2-(7-fluoro-2,4-dimethyl-1H-indol-3-yl)ethyl]amide
182. biphenyl-3,4′-dicarboxylic acid 3-diethylamide 4′-{[2-(7-fluoro-2,4-dimethyl-1H-indol-3-yl)ethyl]amide}
183. biphenyl-4,4′-dicarboxylic acid 4-diethylamide 4′-{[2-(7-fluoro-2,4-dimethyl-1H-indol-3-yl)ethyl]amide}
184. 4′-[2-(7-fluoro-2,4-dimethyl-1H-indol-3-yl)ethylcarbamoyl]biphenyl-3-carboxylic acid
185. biphenyl-4,4′-dicarboxylic acid 4-amide 4′-{[2-(7-fluoro-2,4-dimethyl-1H-indol-3-yl)ethyl]amide}
186. 3′-methylsulfamoylbiphenyl-4-carboxylic acid [2-(7-fluoro-2,4-dimethyl-1H-indol-3-yl)ethyl]amide
187. 3′-trifluoromethylbiphenyl-4-carboxylic acid [2-(7-fluoro-2,4-dimethyl-1H-indol-3-yl)ethyl]amide
188. 4′-methylsulfanylbiphenyl-4-carboxylic acid [2-(7-fluoro-2,4-dimethyl-1H-indol-3-yl)ethyl]amide
189. 4′-acetylbiphenyl-4-carboxylic acid [2-(7-fluoro-2,4-dimethyl-1H-indol-3-yl)ethyl]amide
190. 3′-aminobiphenyl-4-carboxylic acid [2-(7-fluoro-2,4-dimethyl-1H-indol-3-yl)ethyl]amide
191. 3′-acetylbiphenyl-4-carboxylic acid [2-(7-fluoro-2,4-dimethyl-1H-indol-3-yl)ethyl]amide
192. 3′-fluorobiphenyl-4-carboxylic acid [2-(7-fluoro-2,4-dimethyl-1H-indol-3-yl)ethyl]amide
193. [1,1′; 3′,1″]terphenyl-4-carboxylic acid [2-(7-fluoro-2,4-dimethyl-1H-indol-3-yl)ethyl]amide
194. 3′-hydroxymethylbiphenyl-4-carboxylic acid [2-(7-fluoro-2,4-dimethyl-1H-indol-3-yl)ethyl]amide
195. 4-benzo[b]thiophen-3-yl-N-[2-(7-fluoro-2,4-dimethyl-1H-indol-3-yl)ethyl]benzamide
196. 4′-trifluoromethylbiphenyl-4-carboxylic acid [2-(7-fluoro-2,4-dimethyl-1H-indol-3-yl)ethyl]amide
197. N-[2-(7-fluoro-2,4-dimethyl-1H-indol-3-yl)ethyl]-4-((E)-styryl)benzamide
198. N-[2-(7-fluoro-2,4-dimethyl-1H-indol-3-yl)ethyl]-4-quinolin-6-ylbenzamide
199. N-[2-(7-fluoro-2,4-dimethyl-1H-indol-3-yl)ethyl]-4-(6-methoxypyridin-3-yl)-benzamide
200. biphenyl-3,4′-dicarboxylic acid 4′-{[2-(7-fluoro-2,4-dimethyl-1H-indol-3-yl)ethyl]amide}3-methylamide
201. biphenyl-4,4′-dicarboxylic acid 4′-{[2-(7-fluoro-2,4-dimethyl-1H-indol-3-yl)ethyl]amide}4-methylamide
202. 2′-fluorobiphenyl-4-carboxylic acid [2-(7-fluoro-2,4-dimethyl-1H-indol-3-yl)ethyl]amide
203. 2′-methylbiphenyl-4-carboxylic acid [2-(7-fluoro-2,4-dimethyl-1H-indol-3-yl)ethyl]amide
204. 3′-acetylaminobiphenyl-4-carboxylic acid [2-(7-fluoro-2,4-dimethyl-1H-indol-3-yl)ethyl]amide
205. 4-benzo[1,3]dioxol-5-yl-N-[2-(7-fluoro-2,4-dimethyl-1H-indol-3-yl)ethyl]benzamide
206. 3′-cyanobiphenyl-4-carboxylic acid [2-(7-fluoro-2,4-dimethyl-1H-indol-3-yl)ethyl]amide
207. 4′-cyanomethylbiphenyl-4-carboxylic acid [2-(7-fluoro-2,4-dimethyl-1H-indol-3-yl)ethyl]amide
208. biphenyl-3,4′-dicarboxylic acid 3-amide 4′-{[2-(7-fluoro-2,4-dimethyl-1H-indol-3-yl)ethyl]amide}
209. 3′,5′-dimethylbiphenyl-4-carboxylic acid [2-(7-fluoro-2,4-dimethyl-1H-indol-3-yl)ethyl]amide
210. N-[2-(7-fluoro-2,4-dimethyl-1H-indol-3-yl)ethyl]-4-quinolin-3-ylbenzamide
211. 4′-acetylaminobiphenyl-4-carboxylic acid [2-(7-fluoro-2,4-dimethyl-1H-indol-3-yl)ethyl]amide
212. 3′-fluoro-5′-methoxybiphenyl-4-carboxylic acid [2-(7-fluoro-2,4-dimethyl-1H-indol-3-yl)ethyl]amide
213. 5-fluorobiphenyl-3,4′-dicarboxylic acid 4′-{[2-(7-fluoro-2,4-dimethyl-1H-indol-3-yl)ethyl]amide}3-methylamide
214. 3′-(acetylaminomethyl)biphenyl-4-carboxylic acid [2-(7-fluoro-2,4-dimethyl-1H-indol-3-yl)ethyl]amide
215. N-[2-(7-fluoro-2,4-dimethyl-1H-indol-3-yl)ethyl]-4-(1-methyl-1H-indol-5-yl)benzamide
216. N-[2-(7-fluoro-2,4-dimethyl-1H-indol-3-yl)ethyl]-4-(1-methyl-1H-indol-2-yl)benzamide
217. N-[2-(7-fluoro-2,4-dimethyl-1H-indol-3-yl)ethyl]-5-pyrrol-1-ylnicotinamide
218. N-[2-(4-cyano-7-fluoro-2-methyl-1H-indol-3-yl)ethyl]-3,4-dimethoxy-benzamide
219. 5-benzyloxy-1H-indole-2-carboxylic acid [2-(7-fluoro-2,4-dimethyl-1H-indol-3-yl)ethyl]amide
220. 5-hydroxy-1H-indole-2-carboxylic acid [2-(7-fluoro-2,4-dimethyl-1H-indol-3-yl)ethyl]amide
221. 5-methoxybenzofuran-2-carboxylic acid [2-(7-fluoro-2,4-dimethyl-1H-indol-3-yl)ethyl]amide
222. 6-hydroxy-1H-indole-2-carboxylic acid [2-(7-fluoro-2,4-dimethyl-1H-indol-3-yl)ethyl]amide
223. N-[2-(7-fluoro-2-methyl-4-trifluoromethyl-1H-indol-3-yl)ethyl]-3,4-dimethoxybenzamide
224. 3H-benzotriazole-5-carboxylic acid [2-(7-fluoro-2-methyl-4-trifluoromethyl-1H-indol-3-yl)ethyl]amide
225. 5-fluoro-1H-indole-2-carboxylic acid [2-(7-fluoro-2-methyl-4-trifluoromethyl-1H-indol-3-yl)ethyl]amide
226. quinoxaline-6-carboxylic acid [2-(7-fluoro-2-methyl-4-trifluoromethyl-1H-indol-3-yl)ethyl]amide
227. {2-[2-(7-fluoro-2,4-dimethyl-1H-indol-3-yl)ethylcarbamoyl]-1H-indol-5-yloxy}acetic acid methyl ester
228. {2-[2-(7-fluoro-2,4-dimethyl-1H-indol-3-yl)ethylcarbamoyl]-1H-indol-5-yloxy}butanoic acid ethyl ester
229. {2-[2-(7-fluoro-2,4-dimethyl-1H-indol-3-yl)ethylcarbamoyl]-1H-indol-5-yloxy}pentanoic acid ethyl ester
230. {2-[2-(7-fluoro-2,4-dimethyl-1H-indol-3-yl)ethylcarbamoyl]-1H-indol-5-yloxy}hexanoic acid ethyl ester
231. {2-[2-(7-fluoro-2,4-dimethyl-1H-indol-3-yl)ethylcarbamoyl]-1H-indol-6-yloxy}acetic acid methyl ester
232. {2-[2-(7-fluoro-2,4-dimethyl-1H-indol-3-yl)ethylcarbamoyl]-1H-indol-6-yloxy}butanoic acid ethyl ester
233. {2-[2-(7-fluoro-2,4-dimethyl-1H-indol-3-yl)ethylcarbamoyl]-1H-indol-6-yloxy}pentanoic acid ethyl ester
234. {2-[2-(7-fluoro-2,4-dimethyl-1H-indol-3-yl)ethylcarbamoyl]-1H-indol-6-yloxy}hexanoic acid ethyl ester
235. 6-bromopyridine-2-carboxylic acid [2-(7-fluoro-2,4-dimethyl-1H-indol-3-yl)ethyl]amide
236. 5-fluoro-1H-indole-2-carboxylic acid [2-(7-fluoro-2-methyl-1H-indol-3-yl)ethyl]amide
237. 4-bromopyridine-2-carboxylic acid [2-(7-fluoro-2,4-dimethyl-1H-indol-3-yl)ethyl]amide
238. 6-bromo-N-[2-(7-fluoro-2,4-dimethyl-1H-indol-3-yl)ethyl]nicotinamide
239. {3-[2-(7-fluoro-2,4-dimethyl-1H-indol-3-yl)ethylcarbamoyl]phenoxy}acetic acid methyl ester
240. 4-{3-[2-(7-fluoro-2,4-dimethyl-1H-indol-3-yl)ethylcarbamoyl]phenoxy}butanoic acid ethyl ester
241. 5-{3-[2-(7-fluoro-2,4-dimethyl-1H-indol-3-yl)ethylcarbamoyl]phenoxy}pentanoic acid ethyl ester
242. 2-[2-(7-fluoro-2,4-dimethyl-1H-indol-3-yl)ethylcarbamoyl]-1H-indol-5-yloxy}acetic acid
243. 4-{2-[2-(7-fluoro-2,4-dimethyl-1H-indol-3-yl)ethylcarbamoyl]-1H-indol-5-yloxy}butanoic acid
244. 5-{2-[2-(7-fluoro-2,4-dimethyl-1H-indol-3-yl)ethylcarbamoyl]-1H-indol-6-yloxy}pentanoic acid
245. 6-{3-[2-(7-fluoro-2,4-dimethyl-1H-indol-3-yl)ethylcarbamoyl]phenoxy}hexanoic acid ethyl ester
246. 5-{2-[2-(7-fluoro-2,4-dimethyl-1H-indol-3-yl)ethylcarbamoyl]-1H-indol-5-yloxy}pentanoic acid
247. 6-{2-[2-(7-fluoro-2,4-dimethyl-1H-indol-3-yl)ethylcarbamoyl]-1H-indol-5-yloxy}hexanoic acid
248. 4-{2-[2-(7-fluoro-2,4-dimethyl-1H-indol-3-yl)ethylcarbamoyl]-1H-indol-6-yloxy}butanoic acid
249. 6-{2-[2-(7-fluoro-2,4-dimethyl-1H-indol-3-yl)ethylcarbamoyl]-1H-indol-6-yloxy}hexanoic acid
250. 4-{3-[2-(7-fluoro-2,4-dimethyl-1H-indol-3-yl)ethylcarbamoyl]phenoxy}butanoic acid
251. {2-[2-(7-fluoro-2,4-dimethyl-1H-indol-3-yl)ethylcarbamoyl]-1H-indol-6-yloxy}acetic acid
252. 4-{4-[2-(7-fluoro-2,4-dimethyl-1H-indol-3-yl)ethylcarbamoyl]phenoxy}butanoic acid ethyl ester
253. 5-{4-[2-(7-fluoro-2,4-dimethyl-1H-indol-3-yl)ethylcarbamoyl]phenoxy}pentanoic acid ethyl ester
254. 6-{4-[2-(7-fluoro-2,4-dimethyl-1H-indol-3-yl)ethylcarbamoyl]phenoxy}hexanoic acid ethyl ester
255. 6-{3-[2-(7-fluoro-2,4-dimethyl-1H-indol-3-yl)ethylcarbamoyl]phenoxy}hexanoic acid
256. 4-{4-[2-(7-fluoro-2,4-dimethyl-1H-indol-3-yl)ethylcarbamoyl]phenoxy}butanoic acid
257. 5-{4-[2-(7-fluoro-2,4-dimethyl-1H-indol-3-yl)ethylcarbamoyl]phenoxy}pentanoic acid
258. 6-{4-[2-(7-fluoro-2,4-dimethyl-1H-indol-3-yl)ethylcarbamoyl]phenoxy}hexanoic acid
259. 5-{3-[2-(7-fluoro-2,4-dimethyl-1H-indol-3-yl)ethylcarbamoyl]phenoxy}pentanoic acid
260. 2-bromo-N-[2-(7-fluoro-2,4-dimethyl-1H-indol-3-yl)ethyl]isonicotinamide
261. {4-[2-(7-fluoro-2,4-dimethyl-1H-indol-3-yl)ethylcarbamoyl]phenoxy}acetic acid
262. 5-bromo-1H-indole-2-carboxylic acid [2-(7-fluoro-2,4-dimethyl-1H-indol-3-yl)ethyl]amide
263. 6-bromo-1H-indole-2-carboxylic acid [2-(7-fluoro-2,4-dimethyl-1H-indol-3-yl)ethyl]amide
264. 5-bromopyridine-2-carboxylic acid [2-(7-fluoro-2,4-dimethyl-1H-indol-3-yl)ethyl]amide
265. 6-(3-carbamoylphenyl)pyridine-2-carboxylic acid [2-(7-fluoro-2,4-dimethyl-1H-indol-3-yl)ethyl]amide
266. 6-(3-methylcarbamoylphenyl)pyridine-2-carboxylic acid [2-(7-fluoro-2,4-dimethyl-1H-indol-3-yl)ethyl]amide
267. 6-(3-hydroxyphenyl)pyridine-2-carboxylic acid [2-(7-fluoro-2,4-dimethyl-1H-indol-3-yl)ethyl]amide
268. 4-(3-carbamoylphenyl)pyridine-2-carboxylic acid [2-(7-fluoro-2,4-dimethyl-1H-indol-3-yl)ethyl]amide
269. 4-(3-methylcarbamoylphenyl)pyridine-2-carboxylic acid [2-(7-fluoro-2,4-dimethyl-1H-indol-3-yl)ethyl]amide
270. 4-(3-hydroxyphenyl)pyridine-2-carboxylic acid [2-(7-fluoro-2,4-dimethyl-1H-indol-3-yl)ethyl]amide
271. 4-(4-methylcarbamoylphenyl)pyridine-2-carboxylic acid [2-(7-fluoro-2,4-dimethyl-1H-indol-3-yl)ethyl]amide
272. 5-chloro-1H-indole-2-carboxylic acid [2-(7-fluoro-2,4-dimethyl-1H-indol-3-yl)ethyl]amide
273. benzofuran-2-carboxylic acid [2-(7-fluoro-2,4-dimethyl-1H-indol-3-yl)ethyl]amide
274. 5-chloro-benzofuran-2-carboxylic acid [2-(7-fluoro-2,4-dimethyl-1H-indol-3-yl)ethyl]amide
275. 4-bromo-3-fluoro-N-[2-(7-fluoro-2,4-dimethyl-1H-indol-3-yl)ethyl]benzamide
276. 4-chloro-4′-[2-(7-fluoro-2,4-dimethyl-1H-indol-3-yl)ethylcarbamoyl]biphenyl-3-carboxylic acid
277. {3-[2-(7-fluoro-2,4-dimethyl-1H-indol-3-yl)ethylcarbamoyl]phenoxy}acetic acid
278. 5-(3-carbamoylphenyl)pyridine-2-carboxylic acid [2-(7-fluoro-2,4-dimethyl-1H-indol-3-yl)ethyl]amide
279. 5-(3-methylcarbamoylphenyl)pyridine-2-carboxylic acid [2-(7-fluoro-2,4-dimethyl-1H-indol-3-yl)ethyl]amide
280. 5-(3-hydroxyphenyl)pyridine-2-carboxylic acid [2-(7-fluoro-2,4-dimethyl-1H-indol-3-yl)ethyl]amide
281. 6-(3-carbamoylphenyl)-N-[2-(7-fluoro-2,4-dimethyl-1H-indol-3-yl)ethyl]nicotinamide
282. N-[2-(7-fluoro-2,4-dimethyl-1H-indol-3-yl)ethyl]-6-(3-methylcarbamoyl-phenyl)nicotinamide
283. N-[2-(7-fluoro-2,4-dimethyl-1H-indol-3-yl)ethyl]-6-(3-hydroxyphenyl)nicotinamide
284. N-[2-(7-fluoro-2,4-dimethyl-1H-indol-3-yl)ethyl]-6-(4-methylcarbamoyl-phenyl)nicotinamide
285. 5-(3-carbamoylphenyl)-N-[2-(7-fluoro-2,4-dimethyl-1H-indol-3-yl)ethyl]nicotinamide
286. N-[2-(7-fluoro-2,4-dimethyl-1H-indol-3-yl)ethyl]-5-(3-methylcarbamoyl-phenyl)nicotinamide
287. N-[2-(7-fluoro-2,4-dimethyl-1H-indol-3-yl)ethyl]-5-(3-hydroxyphenyl)nicotinamide
288. N-[2-(7-fluoro-2,4-dimethyl-1H-indol-3-yl)ethyl]-5-(4-methylcarbamoyl-phenyl)nicotinamide
289. 2-(3-carbamoylphenyl)-N-[2-(7-fluoro-2,4-dimethyl-1H-indol-3-yl)ethyl]isonicotinamide
290. N-[2-(7-fluoro-2,4-dimethyl-1H-indol-3-yl)ethyl]-2-(3-methylcarbamoyl-phenyl)isonicotinamide
291. N-[2-(7-fluoro-2,4-dimethyl-1H-indol-3-yl)ethyl]-2-(3-hydroxyphenyl)isonicotinamide
292. N-[2-(7-fluoro-2,4-dimethyl-1H-indol-3-yl)ethyl]-2-(4-methylcarbamoyl-phenyl)isonicotinamide
293. benzo[b]thiophene-2-carboxylic acid [2-(7-fluoro-2,4-dimethyl-1H-indol-3-yl)ethyl]amide
294. quinoline-2-carboxylic acid [2-(7-fluoro-2,4-dimethyl-1H-indol-3-yl)ethyl]amide
295. [1,8]naphthyridine-2-carboxylic acid [2-(7-fluoro-2,4-dimethyl-1H-indol-3-yl)ethyl]amide
296. isoquinoline-3-carboxylic acid [2-(7-fluoro-2,4-dimethyl-1H-indol-3-yl)ethyl]amide
297. 5-pyridin-2-yl-thiophene-2-carboxylic acid [2-(7-fluoro-2,4-dimethyl-1H-indol-3-yl)ethyl]amide
298. 5-trifluoromethoxy-1H-indole-2-carboxylic acid [2-(7-fluoro-2,4-dimethyl-1H-indol-3-yl)ethyl]amide
299. 5-fluoro-1H-indole-2-carboxylic acid [2-(4,7-difluoro-2-methyl-1H-indol-3-yl)ethyl]amide
300. biphenyl-3,4′-dicarboxylic acid 4′-{[2-(4,7-difluoro-2-methyl-1H-indol-3-yl)ethyl]amide}3-methylamide
301. 6-(3-trifluoromethoxyphenyl)-1H-indole-2-carboxylic acid [2-(7-fluoro-2,4-dimethyl-1H-indol-3-yl)ethyl]amide
302. 5-(4-methylcarbamoylphenyl)pyridine-2-carboxylic acid [2-(7-fluoro-2,4-dimethyl-1H-indol-3-yl)ethyl]amide
303. 4′-methoxybiphenyl-4-carboxylic acid [2-(7-fluoro-2,4-dimethyl-1H-indol-3-yl)ethyl]amide
304. 4′-[2-(7-fluoro-2,4-dimethyl-1H-indol-3-yl)ethylcarbamoyl]-3-methoxybiphenyl-4-carboxylic acid
305. 4′-methoxybiphenyl-3-carboxylic acid [2-(7-fluoro-2,4-dimethyl-1H-indol-3-yl)ethyl]amide
306. 4-(4-methylcarbamoylphenyl)thiophene-2-carboxylic acid [2-(7-fluoro-2,4-dimethyl-1H-indol-3-yl)ethyl]amide
307. 4-(3-methylcarbamoylphenyl)thiophene-2-carboxylic acid [2-(7-fluoro-2,4-dimethyl-1H-indol-3-yl)ethyl]amide
308. 4-(3-methylcarbamoylphenyl)thiophene-2-carboxylic acid [2-(7-fluoro-2,4-dimethyl-1H-indol-3-yl)ethyl]amide
309. 4-(3-hydroxyphenyl)thiophene-2-carboxylic acid [2-(7-fluoro-2,4-dimethyl-1H-indol-3-yl)ethyl]amide
310. 4-(3-carbamoylphenyl)thiophene-2-carboxylic acid [2-(7-fluoro-2,4-dimethyl-1H-indol-3-yl)ethyl]amide
311. 4-bromothiophene-2-carboxylic acid [2-(7-fluoro-2,4-dimethyl-1H-indol-3-yl)ethyl]amide
312. 2-fluorobiphenyl-4,4′-dicarboxylic acid 4-{[2-(7-fluoro-2,4-dimethyl-1H-indol-3-yl)ethyl]amide}4′-methylamide
313. 2′-fluorobiphenyl-3,4′-dicarboxylic acid 3-amide 4′-{[2-(7-fluoro-2,4-dimethyl-1H-indol-3-yl)ethyl]amide}
314. 2-fluoro-3′-hydroxybiphenyl-4-carboxylic acid [2-(7-fluoro-2,4-dimethyl-1H-indol-3-yl)ethyl]amide
315. 2′-fluorobiphenyl-3,4′-dicarboxylic acid 4′-{[2-(7-fluoro-2,4-dimethyl-1H-indol-3-yl)ethyl]amide}3-methylamide
316. 5-(3-methylcarbamoylphenyl)-1H-indole-2-carboxylic acid [2-(7-fluoro-2,4-dimethyl-1H-indol-3-yl)ethyl]amide
317. 5-bromothiophene-2-carboxylic acid [2-(7-fluoro-2,4-dimethyl-1H-indol-3-yl)ethyl]amide
318. 3′-hydroxybiphenyl-4-carboxylic acid [2-(7-fluoro-2-methyl-1H-indol-3-yl)ethyl]amide
319. biphenyl-4,4′-dicarboxylic acid 4′-{[2-(7-fluoro-2-methyl-1H-indol-3-yl)ethyl]amide}4-methylamide
320. 4′-[2-(7-fluoro-2,4-dimethyl-1H-indol-3-yl)ethylcarbamoyl]-3-hydroxybiphenyl-4-carboxylic acid methyl ester
321. N-[2-(7-fluoro-2-methyl-1H-indol-3-yl)ethyl]-6-(3-methylcarbamoylphenyl)nicotinamide
322. biphenyl-3,4′-dicarboxylic acid 4′-{[2-(7-fluoro-2-methyl-1H-indol-3-yl)ethyl]amide}3-methylamide
323. 5-(3-carbamoylphenyl)-1H-indole-2-carboxylic acid [2-(7-fluoro-2,4-dimethyl-1H-indol-3-yl)ethyl]amide
324. 5-(3-hydroxyphenyl)-1H-indole-2-carboxylic acid [2-(7-fluoro-2,4-dimethyl-1H-indol-3-yl)ethyl]amide
325. 5-(4-methylcarbamoylphenyl)-1H-indole-2-carboxylic acid [2-(7-fluoro-2,4-dimethyl-1H-indol-3-yl)ethyl]amide
326. 5-(3-methylcarbamoylphenyl)thiophene-2-carboxylic acid [2-(7-fluoro-2,4-dimethyl-1H-indol-3-yl)ethyl]amide
327. 5-(3-carbamoylphenyl)thiophene-2-carboxylic acid [2-(7-fluoro-2,4-dimethyl-1H-indol-3-yl)ethyl]amide
328. 5-(3-hydroxyphenyl)thiophene-2-carboxylic acid [2-(7-fluoro-2,4-dimethyl-1H-indol-3-yl)ethyl]amide
329. 5-(4-methylcarbamoylphenyl)thiophene-2-carboxylic acid [2-(7-fluoro-2,4-dimethyl-1H-indol-3-yl)ethyl]amide
330. 6-(3-methylcarbamoylphenyl)-1H-indole-2-carboxylic acid [2-(7-fluoro-2,4-dimethyl-1H-indol-3-yl)ethyl]amide
331. 6-(3-carbamoylphenyl)-1H-indole-2-carboxylic acid [2-(7-fluoro-2,4-dimethyl-1H-indol-3-yl)ethyl]amide
332. 6-(3-hydroxyphenyl)-1H-indole-2-carboxylic acid [2-(7-fluoro-2,4-dimethyl-1H-indol-3-yl)ethyl]amide
333. 6-(4-methylcarbamoylphenyl)-1H-indole-2-carboxylic acid [2-(7-fluoro-2,4-dimethyl-1H-indol-3-yl)ethyl]amide
334. 3′-cyano-2′-fluorobiphenyl-4-carboxylic acid [2-(7-fluoro-2,4-dimethyl-1H-indol-3-yl)ethyl]amide
335. 5-chloro-1H-indole-2-carboxylic acid [2-(7-fluoro-2-methyl-1H-indol-3-yl)ethyl]amide
336. 5-chloro-1H-indole-2-carboxylic acid [2-(4,7-difluoro-2-methyl-1H-indol-3-yl)ethyl]amide
337. 5-chloro-1H-indole-2-carboxylic acid [2-(4-chloro-7-fluoro-2-methyl-1H-indol-3-yl)ethyl]amide
338. 5-trifluoromethyl-1H-indole-2-carboxylic acid [2-(7-fluoro-2,4-dimethyl-1H-indol-3-yl)ethyl]amide
339. 6-methanesulfonyl-1H-indole-2-carboxylic acid [2-(7-fluoro-2,4-dimethyl-1H-indol-3-yl)ethyl]amide
340. 7-chloro-1H-indole-2-carboxylic acid [2-(7-fluoro-2,4-dimethyl-1H-indol-3-yl)ethyl]amide
341. 4-chloro-1H-indole-2-carboxylic acid [2-(7-fluoro-2,4-dimethyl-1H-indol-3-yl)ethyl]amide
342. 6-chloro-1H-indole-2-carboxylic acid [2-(7-fluoro-2,4-dimethyl-1H-indol-3-yl)ethyl]amide
343. 7-fluoro-1H-indole-2-carboxylic acid [2-(7-fluoro-2,4-dimethyl-1H-indol-3-yl)ethyl]amide
344. 5-bromo-1H-indole-2-carboxylic acid [2-(7-fluoro-4-methyl-2-trifluoromethyl-1H-indol-3-yl)ethyl]amide
345. 5-trifluoromethyl-1H-indole-2-carboxylic acid [2-(7-fluoro-4-methyl-2-trifluoromethyl-1H-indol-3-yl)ethyl]amide
346. 5-chloro-1H-indole-2-carboxylic acid [2-(7-fluoro-4-methyl-2-trifluoromethyl-1H-indol-3-yl)ethyl]amide
347. 5-trifluoromethoxy-1H-indole-2-carboxylic acid [2-(7-fluoro-4-methyl-2-trifluoromethyl-1H-indol-3-yl)ethyl]amide
348. N-[2-(7-fluoro-4-methyl-2-trifluoromethyl-1H-indol-3-yl)ethyl]-3,4-dimethoxybenzamide
349. biphenyl-3,4′-dicarboxylic acid 4′-{[2-(4-chloro-7-fluoro-2-methyl-1H-indol-3-yl)ethyl]amide}3-methylamide
350. 5-fluoro-1H-indole-2-carboxylic acid [2-(4-chloro-7-fluoro-2-methyl-1H-indol-3-yl)ethyl]amide
351. 5-trifluoromethoxy-1H-indole-2-carboxylic acid [2-(4-chloro-7-fluoro-2-methyl-1H-indol-3-yl)ethyl]amide
352. 4′-[2-(7-fluoro-2,4-dimethyl-1H-indol-3-yl)ethylcarbamoyl]-3-hydroxybiphenyl-4-carboxylic acid
353. 4-bromo-N-[2-(4-fluoro-2,7-dimethyl-1H-indol-3-yl)ethyl]benzamide
354. 4,5,6,7-tetrahydrobenzo[b]thiophene-2-carboxylic acid [2-(7-fluoro-2,4-dimethyl-1H-indol-3-yl)ethyl]amide
355. N-[2-(7-fluoro-2,4-dimethyl-1H-indol-3-yl)ethyl]-4-hydroxybenzamide
356. 3′-(2,5-dioxoimidazolidin-4-yl)biphenyl-4-carboxylic acid [2-(7-fluoro-2,4-dimethyl-1H-indol-3-yl)ethyl]amide
357. 3-bromo-N-[2-(7-fluoro-2,4-dimethyl-1H-indol-3-yl)ethyl]benzamide
358. 5-bromobenzo[b]thiophene-2-carboxylic acid [2-(7-fluoro-2,4-dimethyl-1H-indol-3-yl)ethyl]amide
359. 6-bromobenzo[b]thiophene-2-carboxylic acid [2-(7-fluoro-2,4-dimethyl-1H-indol-3-yl)ethyl]amide
360. 6-trifluoromethylbenzo[b]thiophene-2-carboxylic acid [2-(7-fluoro-2,4-dimethyl-1H-indol-3-yl)ethyl]amide
361. 6-trifluoromethoxybenzo[b]thiophene-2-carboxylic acid [2-(7-fluoro-2,4-dimethyl-1H-indol-3-yl)ethyl]amide
362. 5-trifluoromethoxybenzo[b]thiophene-2-carboxylic acid [2-(7-fluoro-2,4-dimethyl-1H-indol-3-yl)ethyl]amide
363. benzothiazole-2-carboxylic acid [2-(7-fluoro-2,4-dimethyl-1H-indol-3-yl)ethyl]amide
364. 5-chlorobenzothiazole-2-carboxylic acid [2-(7-fluoro-2,4-dimethyl-1H-indol-3-yl)ethyl]amide
365. 6-chlorobenzothiazole-2-carboxylic acid [2-(7-fluoro-2,4-dimethyl-1H-indol-3-yl)ethyl]amide
366. 5-fluorobenzothiazole-2-carboxylic acid [2-(7-fluoro-2,4-dimethyl-1H-indol-3-yl)ethyl]amide
367. 6-fluorobenzothiazole-2-carboxylic acid [2-(7-fluoro-2,4-dimethyl-1H-indol-3-yl)ethyl]amide
368. 5-trifluoromethylbenzothiazole-2-carboxylic acid [2-(7-fluoro-2,4-dimethyl-1H-indol-3-yl)ethyl]amide
369. 6-trifluoromethylbenzothiazole-2-carboxylic acid [2-(7-fluoro-2,4-dimethyl-1H-indol-3-yl)ethyl]amide
370. 5-trifluoromethoxybenzothiazole-2-carboxylic acid [2-(7-fluoro-2,4-dimethyl-1H-indol-3-yl)ethyl]amide
371. 6-trifluoromethoxybenzothiazole-2-carboxylic acid [2-(7-fluoro-2,4-dimethyl-1H-indol-3-yl)ethyl]amide
372. 5-bromobenzothiazole-2-carboxylic acid [2-(7-fluoro-2,4-dimethyl-1H-indol-3-yl)ethyl]amide
373. 6-bromobenzothiazole-2-carboxylic acid [2-(7-fluoro-2,4-dimethyl-1H-indol-3-yl)ethyl]amide
374. benzoxazole-2-carboxylic acid [2-(7-fluoro-2,4-dimethyl-1H-indol-3-yl)ethyl]amide
375. 5-chlorobenzoxazole-2-carboxylic acid [2-(7-fluoro-2,4-dimethyl-1H-indol-3-yl)ethyl]amide
376. 6-chlorobenzoxazole-2-carboxylic acid [2-(7-fluoro-2,4-dimethyl-1H-indol-3-yl)ethyl]amide
377. 5-fluorobenzoxazole-2-carboxylic acid [2-(7-fluoro-2,4-dimethyl-1H-indol-3-yl)ethyl]amide
378. 6-fluorobenzoxazole-2-carboxylic acid [2-(7-fluoro-2,4-dimethyl-1H-indol-3-yl)ethyl]amide
379. 5-trifluoromethylbenzoxazole-2-carboxylic acid [2-(7-fluoro-2,4-dimethyl-1H-indol-3-yl)ethyl]amide
380. 6-trifluoromethylbenzoxazole-2-carboxylic acid [2-(7-fluoro-2,4-dimethyl-1H-indol-3-yl)ethyl]amide
381. 5-trifluoromethoxybenzoxazole-2-carboxylic acid [2-(7-fluoro-2,4-dimethyl-1H-indol-3-yl)ethyl]amide
382. 6-trifluoromethoxybenzoxazole-2-carboxylic acid [2-(7-fluoro-2,4-dimethyl-1H-indol-3-yl)ethyl]amide
383. 5-bromobenzoxazole-2-carboxylic acid [2-(7-fluoro-2,4-dimethyl-1H-indol-3-yl)ethyl]amide
384. 6-bromobenzoxazole-2-carboxylic acid [2-(7-fluoro-2,4-dimethyl-1H-indol-3-yl)ethyl]amide
385. 1H-benzimidazole-2-carboxylic acid [2-(7-fluoro-2,4-dimethyl-1H-indol-3-yl)ethyl]amide
386. 5-chloro-1H-benzimidazole-2-carboxylic acid [2-(7-fluoro-2,4-dimethyl-1H-indol-3-yl)ethyl]amide
387. 6-chloro-1H-benzimidazole-2-carboxylic acid [2-(7-fluoro-2,4-dimethyl-1H-indol-3-yl)ethyl]amide
388. 5-fluoro-1H-benzimidazole-2-carboxylic acid [2-(7-fluoro-2,4-dimethyl-1H-indol-3-yl)ethyl]amide
389. 6-fluoro-1H-benzimidazole-2-carboxylic acid [2-(7-fluoro-2,4-dimethyl-1H-indol-3-yl)ethyl]amide
390. 5-trifluoromethyl-1H-benzimidazole-2-carboxylic acid [2-(7-fluoro-2,4-dimethyl-1H-indol-3-yl)ethyl]amide
391. 6-trifluoromethyl-1H-benzimidazole-2-carboxylic acid [2-(7-fluoro-2,4-dimethyl-1H-indol-3-yl)ethyl]amide
392. 5-trifluoromethoxy-1H-benzimidazole-2-carboxylic acid [2-(7-fluoro-2,4-dimethyl-1H-indol-3-yl)ethyl]amide
393. 6-trifluoromethoxy-1H-benzimidazole-2-carboxylic acid [2-(7-fluoro-2,4-dimethyl-1H-indol-3-yl)ethyl]amide
394. 5-bromo-1H-benzimidazole-2-carboxylic acid [2-(7-fluoro-2,4-dimethyl-1H-indol-3-yl)ethyl]amide
395. 6-bromo-1H-benzimidazole-2-carboxylic acid [2-(7-fluoro-2,4-dimethyl-1H-indol-3-yl)ethyl]amide
396. 5-trifluoromethylsulfanyl-1H-indole-2-carboxylic acid [2-(7-fluoro-2,4-dimethyl-1H-indol-3-yl)ethyl]amide
397. 5,6-dichloro-1H-indole-2-carboxylic acid [2-(7-fluoro-2,4-dimethyl-1H-indol-3-yl)ethyl]amide
398. 5-chloro-6-fluoro-1H-indole-2-carboxylic acid [2-(7-fluoro-2,4-dimethyl-1H-indol-3-yl)ethyl]amide
399. 5-fluoro-6-chloro-1H-indole-2-carboxylic acid [2-(7-fluoro-2,4-dimethyl-1H-indol-3-yl)ethyl]amide
400. 5,6-difluoro-1H-indole-2-carboxylic acid [2-(7-fluoro-2,4-dimethyl-1H-indol-3-yl)ethyl]amide
401. 4,6-dichloro-1H-indole-2-carboxylic acid [2-(7-fluoro-2,4-dimethyl-1H-indol-3-yl)ethyl]amide
402. 4,6-difluoro-1H-indole-2-carboxylic acid [2-(7-fluoro-2,4-dimethyl-1H-indol-3-yl)ethyl]amide
403. 5-acetylamino-1H-indole-2-carboxylic acid [2-(7-fluoro-2,4-dimethyl-1H-indol-3-yl)ethyl]amide
404. 6-acetylamino-1H-indole-2-carboxylic acid [2-(7-fluoro-2,4-dimethyl-1H-indol-3-yl)ethyl]amide
405. 5-(2,2-dimethylpropionylamino)-1H-indole-2-carboxylic acid [2-(7-fluoro-2,4-dimethyl-1H-indol-3-yl)ethyl]amide
406. 6-(2,2-dimethylpropionylamino)-1H-indole-2-carboxylic acid [2-(7-fluoro-2,4-dimethyl-1H-indol-3-yl)ethyl]amide
407. 5-trifluoroacetylamino-1H-indole-2-carboxylic acid [2-(7-fluoro-2,4-dimethyl-1H-indol-3-yl)ethyl]amide
408. 6-trifluoroacetylamino-1H-indole-2-carboxylic acid [2-(7-fluoro-2,4-dimethyl-1H-indol-3-yl)ethyl]amide
409. 5-isopropyloxy-1H-indole-2-carboxylic acid [2-(7-fluoro-2,4-dimethyl-1H-indol-3-yl)ethyl]amide
410. 6-isopropyloxy-1H-indole-2-carboxylic acid [2-(7-fluoro-2,4-dimethyl-1H-indol-3-yl)ethyl]amide
411. 5-isopropyl-1H-indole-2-carboxylic acid [2-(7-fluoro-2,4-dimethyl-1H-indol-3-yl)ethyl]amide
412. 6-trifluoromethyl-1H-indole-2-carboxylic acid [2-(7-fluoro-2,4-dimethyl-1H-indol-3-yl)ethyl]amide
413. 4,5,6,7-tetrahydro-1H-indole-2-carboxylic acid [2-(7-fluoro-2,4-dimethyl-1H-indol-3-yl)ethyl]amide
414. 3-methyl-1H-indole-2-carboxylic acid [2-(7-fluoro-2,4-dimethyl-1H-indol-3-yl)ethyl]amide
415. 5-trifluoromethylbenzo[b]thiophene-2-carboxylic acid [2-(7-fluoro-2,4-dimethyl-1H-indol-3-yl)ethyl]amide
416. 5-fluorobenzo[b]thiophene-2-carboxylic acid [2-(7-fluoro-2,4-dimethyl-1H-indol-3-yl)ethyl]amide
417. 5-amino-1H-indole-2-carboxylic acid [2-(7-fluoro-2,4-dimethyl-1H-indol-3-yl)ethyl]amide
418. 6-amino-1H-indole-2-carboxylic acid [2-(7-fluoro-2,4-dimethyl-1H-indol-3-yl)ethyl]amide
419. 6-dimethylcarbamoylmethoxy-1H-indole-2-carboxylic acid [2-(7-fluoro-2,4-dimethyl-1H-indol-3-yl)ethyl]amide
420. 5-dimethylcarbamoylmethoxy-1H-indole-2-carboxylic acid [2-(7-fluoro-2,4-dimethyl-1H-indol-3-yl)ethyl]amide
421. 6-methylcarbamoylmethoxy-1H-indole-2-carboxylic acid [2-(7-fluoro-2,4-dimethyl-1H-indol-3-yl)ethyl]amide
422. 5-methylcarbamoylmethoxy-1H-indole-2-carboxylic acid [2-(7-fluoro-2,4-dimethyl-1H-indol-3-yl)ethyl]amide
423. 6-carbamoylmethoxy-1H-indole-2-carboxylic acid [2-(7-fluoro-2,4-dimethyl-1H-indol-3-yl)ethyl]amide
424. 5-carbamoylmethoxy-1H-indole-2-carboxylic acid [2-(7-fluoro-2,4-dimethyl-1H-indol-3-yl)ethyl]amide
425. 6-tert-butyl-1H-indole-2-carboxylic acid [2-(7-fluoro-2,4-dimethyl-1H-indol-3-yl)ethyl]amide
426. 5-tert-butyl-1H-indole-2-carboxylic acid [2-(7-fluoro-2,4-dimethyl-1H-indol-3-yl)ethyl]amide

9. The use of the compounds as claimed in claim 1 for the manufacture of medicaments which comprise at least one of the compounds of the formula I.

10. A medicament as set forth in claim 9 with suitable formulating substances and carriers.

11. A method of using the medicaments as set forth in claim 10, for the treatment and prophylaxis of disorders.

12. A method as in claim 11 for the treatment and prophylaxis of disorders connected with the EP₂ receptor.

13. A method as in claim 11 for the treatment and prophylaxis of fertility impairments.

14. A method as in claim 11 for the treatment and prophylaxis of painful menstruation.

15. A method as in claim 11 for the treatment and prophylaxis of endometriosis.

16. A method of using the compounds as claimed in claim 1 for modulating the EP₂ receptor.

17. A method as in claim 11 for the treatment and prophylaxis of pain.

18. A method of using the compounds and of the medicaments as set forth in claim 10 for controlling fertility/contraception.

19. A method as in claim 11 for the treatment and prophylaxis of osteoporosis.

20. A method as in claim 11 for the treatment and prophylaxis of cancer.

21. A method of using the compounds of the general formula I as claimed in claim 1 in the form of a pharmaceutical product for enteral, parenteral, vaginal and oral administration.
